SEQUENCE LISTING (177 sequences)

SEQ ID NO: 1 - DNA, 31 bp, Artificial Sequence
  Description of Artificial Sequence Synthetic oligonucleotide
agagggtatataatggaagctcgacttccag

SEQ ID NO: 2 - DNA, 52 bp, Artificial Sequence
  Description of Artificial Sequence Synthetic oligonucleotide
gggaatttccggggactttccgggaatttccggggactttccgggaatttcc

SEQ ID NO: 3 - DNA, 85 bp, Artificial Sequence
  Description of Artificial Sequence Synthetic oligonucleotide
caccagacagtgacgtcagctgccagatcccatggccgtcatactgtgacgtctttcagacaccccattgacgtcaatgggagaa

SEQ ID NO: 4 - DNA, 90 bp, Artificial Sequence
  Description of Artificial Sequence Synthetic oligonucleotide
ggaggaaaaactgtttcatacagaaggcgtggaggaaaaactgtttcatacagaaggcgtggaggaaaaactgtttcatacagaaggcgt

SEQ ID NO: 5 - DNA, 115 bp, Artificial Sequence
  Description of Artificial Sequence Synthetic polynucleotide
aggatgtccatattaggacatctaggatgtccatattaggacatctaggatgtccatattaggacatctaggatgtccatattaggacatctaggatgtccatattaggacatct

SEQ ID NO: 6 - DNA, 115 bp, Artificial Sequence
  Description of Artificial Sequence Synthetic polynucleotide
agtatgtccatattaggacatctaccatgtccatattaggacatctactatgtccatattaggacatcttgtatgtccatattaggacatctaaaatgtccatattaggacatct

SEQ ID NO: 7 - DNA, 48 bp, Artificial Sequence
  Description of Artificial Sequence Synthetic oligonucleotide
tgagtcagtgactcagtgagtcagtgactcagtgagtcagtgactcag

SEQ ID NO: 8 - DNA, 120 bp, Artificial Sequence
  Description of Artificial Sequence Synthetic polynucleotide
agatcaaagggtttaagatcaaagggcttaagatcaaagggtataagatcaaagggcctaagatcaaagggactaagatcaaagggtttaagatcaaagggcttaagatcaaagggccta

SEQ ID NO: 9 - DNA, 32 bp, Artificial Sequence
  Description of Artificial Sequence Synthetic oligonucleotide
gtctagacgtctagacgtctagacgtctagac

SEQ ID NO: 10 - DNA, 24 bp, Artificial Sequence
  Description of Artificial Sequence Synthetic oligonucleotide
cagacacagacacagacacagaca

SEQ ID NO: 11 - DNA, 65 bp, Artificial Sequence
  Description of Artificial Sequence Synthetic oligonucleotide
ggatccggtactcgagatctgcgatctaagtaagcttggcattccggtactgttggtaaagccac

SEQ ID NO: 12 - DNA, 588 bp, Artificial Sequence
  Description of Artificial Sequence Synthetic polynucleotide
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 13 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 544 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 15 - DNA, 399 bp, Artificial Sequence
  Description of Artificial Sequence Synthetic polynucleotide
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 16 - DNA, 511 bp, Artificial Sequence
  Description of Artificial Sequence Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 17 - DNA, 408 bp, Artificial Sequence
  Description of Artificial Sequence Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 18 - DNA, 344 bp, Artificial Sequence
  Description of Artificial Sequence Synthetic polynucleotide
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 19 - DNA, 1229 bp, Artificial Sequence
  Description of Artificial Sequence Synthetic polynucleotide
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 20 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 1718 bp, Artificial Sequence
  Description of Artificial Sequence Synthetic polynucleotide
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 212 bp, Artificial Sequence
  Description of Artificial Sequence Synthetic polynucleotide
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 23 - DNA, 2379 bp, Artificial Sequence
  Description of Artificial Sequence Synthetic polynucleotide
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 24 - DNA, 529 bp, Artificial Sequence
  Description of Artificial Sequence Synthetic polynucleotide
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 25 - DNA, 794 bp, Artificial Sequence
  Description of Artificial Sequence Synthetic polynucleotide
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 26 - DNA, 532 bp, Artificial Sequence
  Description of Artificial Sequence Synthetic polynucleotide
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 27 - DNA, 701 bp, Artificial Sequence
  Description of Artificial Sequence Synthetic polynucleotide
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 28 - DNA, 465 bp, Artificial Sequence
  Description of Artificial Sequence Synthetic polynucleotide
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 29 - DNA, 538 bp, Artificial Sequence
  Description of Artificial Sequence Synthetic polynucleotide
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 30 - DNA, 1091 bp, Artificial Sequence
  Description of Artificial Sequence Synthetic polynucleotide
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 31 - DNA, 66 bp, Homo sapiens
atgtgtcaccagcagctcgttatatcctggtttagtttggtgtttctcgcttcacccctggtggca

SEQ ID NO: 32 - DNA, 66 bp, Artificial Sequence
  Description of Artificial Sequence Synthetic oligonucleotide
atgtgccatcagcaactcgtcatctcctggttctcccttgtgttcctcgcttcccctctggtcgcc

SEQ ID NO: 33 - DNA, 42 bp, Artificial Sequence
  Description of Artificial Sequence Synthetic oligonucleotide
atgcaactgctgtcatgtatcgcactcatcctggcgctggta

SEQ ID NO: 34 - DNA, 60 bp, Homo sapiens
atgtatcggatgcaacttttgagctgcatcgcattgtctctggcgctggtgacaaattcc

SEQ ID NO: 35 - DNA, 45 bp, Homo sapiens
atgaatctcttgctcatacttacgtttgtcgctgctgccgttgcg

SEQ ID NO: 36 - DNA, 51 bp, Gaussia princeps
atgggcgtgaaggtcttgtttgcccttatctgcatagctgttgcggaggcg

SEQ ID NO: 37 - DNA, 72 bp, Homo sapiens
atgccgatggggagccttcaacctttggcaacgctttatcttctggggatgttggttgctagttgccttggg

SEQ ID NO: 38 - DNA, 63 bp, Mus sp.
atggaaactgacacgttgttgctgtgggtattgctcttgtgggtcccaggatctacgggcgac

SEQ ID NO: 39 - DNA, 66 bp, Homo sapiens
atggatatgagggttcccgcccagcttttggggctgcttttgttgtggcttcgaggggctcggtgt

SEQ ID NO: 40 - DNA, 48 bp, Unknown
  Description of Unknown VSV-G sequence
atgaagtgtctgttgtacctggcgtttctgttcattggtgtaaactgt

SEQ ID NO: 41 - DNA, 84 bp, Homo sapiens
atgaatatcaaaggaagtccgtggaagggtagtctcctgctgctcctcgtatctaaccttctcctttgtcaatccgtggcaccc

SEQ ID NO: 42 - DNA, 54 bp, Homo sapiens
atgaaatgggtaacattcatatcacttctctttctgttcagctctgcgtattct

SEQ ID NO: 43 - DNA, 57 bp, Homo sapiens
atgacaaggcttactgttttggctctcctcgctggactcttggcttcctcccgagca

SEQ ID NO: 44 - DNA, 51 bp, Homo sapiens
atgagggcttggattttttttctgctctgccttgccggtcgagccctggcg

SEQ ID NO: 45 - DNA, 48 bp, Homo sapiens
atgcctcttctgcttttgcttcctcttttgtgggcaggtgccctcgca

SEQ ID NO: 46 - DNA, 87 bp, Homo sapiens
atgaactctttctcaacctctgcgtttggtccggtcgctttctcccttgggctcctgcttgtcttgccagcagcgtttcctgcgcca

SEQ ID NO: 47 - DNA, 60 bp, Homo sapiens
atgacaagtaaactggcggtagccttgctcgcggcctttttgatttccgcagccctttgt

SEQ ID NO: 48 - DNA, 69 bp, Homo sapiens
atgaaggtaagtgcagcgttgctttgccttctcctcattgcagcgacctttattcctcaagggctggcc

SEQ ID NO: 49 - DNA, 78 bp, Homo sapiens
atgggagcggcagctagaacacttcgacttgcccttgggctcttgctccttgcaaccctccttagacctgccgacgca

SEQ ID NO: 50 - DNA, 63 bp, Homo sapiens
atgtcaccgttgttgcggagattgctgttggccgcacttttgcaactggctcctgctcaagcc

SEQ ID NO: 51 - DNA, 63 bp, Homo sapiens
atgaataacctgctctgttgtgcgctcgtgttcctggacatttctataaaatggacaacgcaa

SEQ ID NO: 52 - DNA, 69 bp, Homo sapiens
atgcaaatgtctcctgcccttacctgtctcgtacttggtcttgcgctcgtatttggagagggatcagcc

SEQ ID NO: 53 - DNA, 102 bp, Homo sapiens
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, Homo sapiens
atgaatgcaaaagtcgtggtcgtgctggttttggttctgacggcgttgtgtcttagtgatggg

SEQ ID NO: 55 - DNA, 66 bp, Artificial Sequence
  Description of Artificial Sequence Synthetic oligonucleotide
atggaacgcattgtgatctgcctgatggtcatcttcctgggcaccttagtgcacaagtcgagcagc

SEQ ID NO: 56 - protein, 22 aa, Homo sapiens
MCHQQLVISWFSLVFLASPLVA

SEQ ID NO: 57 - protein, 22 aa, Artificial Sequence
  Description of Artificial Sequence Synthetic peptide
MCHQQLVISWFSLVFLASPLVA

SEQ ID NO: 58 - protein, 14 aa, Artificial Sequence
  Description of Artificial Sequence Synthetic peptide
MQLLSCIALILALV

SEQ ID NO: 59 - protein, 20 aa, Homo sapiens
MYRMQLLSCIALSLALVTNS

SEQ ID NO: 60 - protein, 15 aa, Homo sapiens
MNLLLILTFVAAAVA

SEQ ID NO: 61 - protein, 17 aa, Gaussia princeps
MGVKVLFALICIAVAEA

SEQ ID NO: 62 - protein, 24 aa, Homo sapiens
MPMGSLQPLATLYLLGMLVASCLG

SEQ ID NO: 63 - protein, 21 aa, Mus sp.
METDTLLLWVLLLWVPGSTGD

SEQ ID NO: 64 - protein, 22 aa, Homo sapiens
MDMRVPAQLLGLLLLWLRGARC

SEQ ID NO: 65 - protein, 16 aa, Unknown
  Description of Unknown VSV-G sequence
MKCLLYLAFLFIGVNC

SEQ ID NO: 66 - protein, 28 aa, Homo sapiens
MNIKGSPWKGSLLLLLVSNLLLCQSVAP

SEQ ID NO: 67 - protein, 18 aa, Homo sapiens
MKWVTFISLLFLFSSAYS

SEQ ID NO: 68 - protein, 19 aa, Homo sapiens
MTRLTVLALLAGLLASSRA

SEQ ID NO: 69 - protein, 17 aa, Homo sapiens
MRAWIFFLLCLAGRALA

SEQ ID NO: 70 - protein, 16 aa, Homo sapiens
MPLLLLLPLLWAGALA

SEQ ID NO: 71 - protein, 29 aa, Homo sapiens
MNSFSTSAFGPVAFSLGLLLVLPAAFPAP

SEQ ID NO: 72 - protein, 20 aa, Homo sapiens
MTSKLAVALLAAFLISAALC

SEQ ID NO: 73 - protein, 23 aa, Homo sapiens
MKVSAALLCLLLIAATFIPQGLA

SEQ ID NO: 74 - protein, 26 aa, Homo sapiens
MGAAARTLRLALGLLLLATLLRPADA

SEQ ID NO: 75 - protein, 21 aa, Homo sapiens
MSPLLRRLLLAALLQLAPAQA

SEQ ID NO: 76 - protein, 21 aa, Homo sapiens
MNNLLCCALVFLDISIKWTTQ

SEQ ID NO: 77 - protein, 23 aa, Homo sapiens
MQMSPALTCLVLGLALVFGEGSA

SEQ ID NO: 78 - protein, 34 aa, Homo sapiens
MARAALSAAPSNPRLLRVALLLLLLVAAGRRAAG

SEQ ID NO: 79 - protein, 21 aa, Homo sapiens
MNAKVVVVLVLVLTALCLSDG

SEQ ID NO: 80 - protein, 22 aa, Artificial Sequence
  Description of Artificial Sequence Synthetic peptide
MERIVICLMVIFLGTLVHKSSS

SEQ ID NO: 81 - protein, 10 aa, Unknown
  Description of Unknown cleavage site sequence
DEMEECSQHL

SEQ ID NO: 82 - protein, 9 aa, Unknown
  Description of Unknown cleavage site sequence
EDVVPCSMG

SEQ ID NO: 83 - protein, 21 aa, Homo sapiens
MALPVTALLLPLALLLHAARP

SEQ ID NO: 84 - DNA, 63 bp, Homo sapiens
atggctctccctgtaactgccctgcttcttccccttgccttgcttctccatgccgctagaccc

SEQ ID NO: 85 - protein, 22 aa, Homo sapiens
MLLLVTSLLLCELPHPAFLLIP

SEQ ID NO: 86 - DNA, 66 bp, Homo sapiens
atgctgctgctggtcacatctctgctgctgtgcgagctgccccatcctgcctttctgctgatccct

SEQ ID NO: 87 - DNA, 66 bp, Homo sapiens
atgctgctgctggttacatctctgctgctgtgcgagctgccccatcctgcctttctgctgatccct

SEQ ID NO: 88 - protein, 176 aa, Artificial Sequence
  Description of Artificial Sequence Synthetic polypeptide
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 89 - protein, 334 aa, Artificial Sequence
  Description of Artificial Sequence Synthetic polypeptide
TCRDYKDHDGDYKDHDIDYKDDDDKMAPKKKRKVGIHGVPGGLEGGGGSGGTEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHYPYDVPDYAGGGGSGGT

SEQ ID NO: 90 - protein, 531 aa, Artificial Sequence
  Description of Artificial Sequence Synthetic polypeptide
EASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 91 - DNA, 3129 bp, Artificial Sequence
  Description of Artificial Sequence Synthetic polynucleotide
atgtctagacccggagagcgcccattccagtgtcggatttgcatgcggaacttttcgagaagacacggcctggacagacatacccgtactcatacaggtgaaaaaccctttcagtgtcggatctgtatgcgaaatttctccgaccacagcagcctgaagagacatctacgtacccacaccggcagccagaagccatttcagtgtcggatctgtatgcggaacttctccgtgagacacaacctgaccagacatctacgtacgcacaccggagagaagccattccaatgccgaatatgcatgcgcaacttcagtgaccacagcaacctgagcagacacctaaaaacccacaccggttcccagaagccatttcagtgtcggatctgtatgcggaacttctcccagcgcagcagcctggtgagacatctacgtacgcacaccggagagaagccattccaatgccgaatatgcatgcgcaacttcagtgagagcggccacctgaagagacacctgcgtacgcacctgaggggatccacctgcagggactacaaagaccatgacggtgattataaagatcatgacatcgattacaaggatgacgatgacaagatggcccccaagaaaaagaggaaggtgggcattcacggggtgccgggtggactcgagggaggcggtggaagcggcggtaccgaggacgtggtgtgctgccactcaatctacggcaagaagaagggtgatatcgacacctaccgatacataggctcttccgggacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagtagaggctctctgctgagccccagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggcggaggatctggcggaacagaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttttga

SEQ ID NO: 92 - DNA, 243 bp, Unknown
  Description of Unknown ACP-binding domain sequence
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 93 - protein, 541 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 94 - DNA, 1626 bp, Artificial Sequence
  Description of Artificial Sequence Synthetic polynucleotide
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagttaa

SEQ ID NO: 95 - DNA, 1626 bp, Artificial Sequence
  Description of Artificial Sequence Synthetic polynucleotide
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagctag

SEQ ID NO: 96 - protein, 65 aa, Artificial Sequence
  Description of Artificial Sequence Synthetic polypeptide
RTLVTFKDVFVDFTREEWKLLDTAQQIVYRNVMLENYKNLVSLGYQLTKPDVILRLEKGEEPWLV

SEQ ID NO: 97 - protein, 45 aa, Artificial Sequence
  Description of Artificial Sequence Synthetic polypeptide
RTLVTFKDVFVDFTREEWKLLDTAQQIVYRNVMLENYKNLVSLGY

SEQ ID NO: 98 - DNA, 195 bp, Artificial Sequence
  Description of Artificial Sequence Synthetic polynucleotide
agaaccctggtcaccttcaaggacgtgttcgtggacttcacccgggaagagtggaagctgctggatacagcccagcagatcgtgtaccggaacgtgatgctggaaaactacaagaatctggtgtccctgggctaccagctgaccaagcctgacgtgatcctgcggctggaaaagggcgaagaaccttggctggtg

SEQ ID NO: 99 - DNA, 135 bp, Artificial Sequence
  Description of Artificial Sequence Synthetic polynucleotide
agaaccctggtcaccttcaaggacgtgttcgtggacttcacccgggaagagtggaagctgctggatacagcccagcagatcgtgtaccggaacgtgatgctggaaaactacaagaatctggtgtccctgggctac

SEQ ID NO: 100 - protein, 42 aa, Artificial Sequence
  Description of Artificial Sequence Synthetic polypeptide
AAAIEVMYPPPYLDNEKSNGTIIHVKGKHLCPSPLFPGPSKP

SEQ ID NO: 101 - protein, 12 aa, Artificial Sequence
  Description of Artificial Sequence Synthetic peptide
ESKYGPPCPSCP

SEQ ID NO: 102 - protein, 12 aa, Artificial Sequence
  Description of Artificial Sequence Synthetic peptide
ESKYGPPAPSAP

SEQ ID NO: 103 - protein, 12 aa, Artificial Sequence
  Description of Artificial Sequence Synthetic peptide
ESKYGPPCPPCP

SEQ ID NO: 104 - protein, 12 aa, Artificial Sequence
  Description of Artificial Sequence Synthetic peptide
EPKSCDKTHTCP

SEQ ID NO: 105 - protein, 86 aa, Artificial Sequence
  Description of Artificial Sequence Synthetic polypeptide
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 106 - protein, 45 aa, Artificial Sequence
  Description of Artificial Sequence Synthetic polypeptide
TTTPAPRPPTPAPTIALQPLSLRPEACRPAAGGAVHTRGLDFACD

SEQ ID NO: 107 - protein, 132 aa, Artificial Sequence
  Description of Artificial Sequence Synthetic polypeptide
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 108 - protein, 34 aa, Artificial Sequence
  Description of Artificial Sequence Synthetic polypeptide
ACPTGLYTHSGECCKACNLGEGVAQPCGANQTVC

SEQ ID NO: 109 - protein, 20 aa, Artificial Sequence
  Description of Artificial Sequence Synthetic peptide
AVGQDTQEVIVVPHSLPFKV

SEQ ID NO: 110 - DNA, 126 bp, Artificial Sequence
  Description of Artificial Sequence Synthetic polynucleotide
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 111 - DNA, 36 bp, Artificial Sequence
  Description of Artificial Sequence Synthetic oligonucleotide
gaaagcaagtacggtccaccttgccctagctgtccg

SEQ ID NO: 112 - DNA, 36 bp, Artificial Sequence
  Description of Artificial Sequence Synthetic oligonucleotide
gaatccaagtacggccccccagcgcctagtgcccca

SEQ ID NO: 113 - DNA, 36 bp, Artificial Sequence
  Description of Artificial Sequence Synthetic oligonucleotide
gaatctaaatatggcccgccatgcccgccttgccca

SEQ ID NO: 114 - DNA, 36 bp, Artificial Sequence
  Description of Artificial Sequence Synthetic oligonucleotide
gaaccgaagtcttgtgataaaactcatacgtgcccg

SEQ ID NO: 115 - DNA, 258 bp, Artificial Sequence
  Description of Artificial Sequence Synthetic polynucleotide
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 116 - DNA, 396 bp, Artificial Sequence
  Description of Artificial Sequence Synthetic polynucleotide
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 117 - DNA, 102 bp, Artificial Sequence
  Description of Artificial Sequence Synthetic polynucleotide
gcctgccctacaggactctacacgcatagcggtgagtgttgtaaagcatgcaacctcggggaaggtgtagcccagccatgcggggctaaccaaaccgtttgc

SEQ ID NO: 118 - DNA, 60 bp, Artificial Sequence
  Description of Artificial Sequence Synthetic oligonucleotide
gctgtgggccaggacacgcaggaggtcatcgtggtgccacactccttgccctttaaggtg

SEQ ID NO: 119 - protein, 5 aa, Artificial Sequence
  Description of Artificial Sequence Synthetic peptide
KNAMN

SEQ ID NO: 120 - protein, 19 aa, Artificial Sequence
  Description of Artificial Sequence Synthetic peptide
RIRNKTNNYATYYADSVKA

SEQ ID NO: 121 - protein, 6 aa, Artificial Sequence
  Description of Artificial Sequence Synthetic peptide
GNSFAY

SEQ ID NO: 122 - protein, 17 aa, Artificial Sequence
  Description of Artificial Sequence Synthetic peptide
KSSQSLLYSSNQKNYLA

SEQ ID NO: 123 - protein, 7 aa, Artificial Sequence
  Description of Artificial Sequence Synthetic peptide
WASSRES

SEQ ID NO: 124 - protein, 9 aa, Artificial Sequence
  Description of Artificial Sequence Synthetic peptide
QQYYNYPLT

SEQ ID NO: 125 - protein, 117 aa, Artificial Sequence
  Description of Artificial Sequence Synthetic polypeptide
EVQLVETGGGMVQPEGSLKLSCAASGFTFNKNAMNWVRQAPGKGLEWVARIRNKTNNYATYYADSVKARFTISRDDSQSMLYLQMNNLKIEDTAMYYCVAGNSFAYWGQGTLVTVSA

SEQ ID NO: 126 - protein, 117 aa, Artificial Sequence
  Description of Artificial Sequence Synthetic polypeptide
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 127 - protein, 113 aa, Artificial Sequence
  Description of Artificial Sequence Synthetic polypeptide
DIVMSQSPSSLVVSIGEKVTMTCKSSQSLLYSSNQKNYLAWYQQKPGQSPKLLIYWASSRESGVPDRFTGSGSGTDFTLTISSVKAEDLAVYYCQQYYNYPLTFGAGTKLELK

SEQ ID NO: 128 - protein, 113 aa, Artificial Sequence
  Description of Artificial Sequence Synthetic polypeptide
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 129 - protein, 119 aa, Artificial Sequence
  Description of Artificial Sequence Synthetic polypeptide
QVQLVESGGGTVQAGGSLKLACAASGLPRTYNVMGWFRQAPGKEREGVAIIYTTTGATYYRDSVKGRATISQDNAKKSVSLQMNSLRPEDTAIYYCVARQPNSGPWEYWGQGTQVTVSS

SEQ ID NO: 130 - protein, 117 aa, Artificial Sequence
  Description of Artificial Sequence Synthetic polypeptide
QVKLEESGGGSVQAGGSLRLSCTTSGYTNSYKWMGWFRQAPGQEREGVAVIYTGNDRTYYSDSVKGRFTISRDNAKNMIYLDMTRLRPEDSAVYECAIGHDGAWRYWGQGTQVTVSS

SEQ ID NO: 131 - protein, 60 aa, Artificial Sequence
  Description of Artificial Sequence Synthetic polypeptide
FNVLMVHKRSHTGERPLQCEICGFTCRQKGNLLRHIKLHTGEKPFKCHLCNYACQRRDAL

SEQ ID NO: 132 - protein, 208 aa, Artificial Sequence
  Description of Artificial Sequence Synthetic polypeptide
MDYKDDDDKDYKDDDDKDYKDDDDKPKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 133 - protein, 45 aa, Artificial Sequence
  Description of Artificial Sequence Synthetic polypeptide
EDVVCCHSIYGKKKGDIDTYRYIGSSGTGCVVIVGRIVLSGSGTS

SEQ ID NO: 134 - protein, 186 aa, Hepacivirus C
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 135 - protein, 34 aa, Artificial Sequence
  Description of Artificial Sequence Synthetic polypeptide
NSSPPAVTLTHPITKIDREVLYQEFDEMEECSQH

SEQ ID NO: 136 - DNA, 3015 bp, Artificial Sequence
  Description of Artificial Sequence Synthetic polynucleotide
atggactacaaggacgacgacgacaaggattacaaggatgatgatgataaggactataaggacgatgatgacaaacccaagaagaagcggaaggtttcccggcctggcgagaggcctttccagtgcagaatctgcatgcggaacttcagcagacggcacggcctggacagacacaccagaacacacacaggcgagaaacccttccagtgccggatctgtatgagaaatttcagcgaccacagcagcctgaagcggcacctgagaacccataccggcagccagaaaccatttcagtgtaggatatgcatgcgcaatttctccgtgcggcacaacctgaccagacacctgaggacacacaccggggagaagccttttcaatgtcgcatatgcatgagaaacttctctgaccactccaacctgagccgccacctcaaaacccacaccggctctcaaaagcccttccaatgtagaatatgtatgaggaactttagccagcggagcagcctcgtgcgccatctgagaactcacactggcgaaaagccgtttcaatgccgtatctgtatgcgcaactttagcgagagcggccacctgaagagacatctgcgcacacacctgagaggcagcgaggatgtcgtgtgctgccacagcatctacggaaagaagaagggcgacatcgacacctatcggtacatcggcagcagcggcacaggctgtgttgtgatcgtgggcagaatcgtgctgagcggctctggaacaagcgcccctatcacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggcacaagaacaatcgcctctccaaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggcagctctggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacgaggcctctggatctggtagagccgacgctctggacgacttcgacctggatatgctgggctctgacgccctggatgattttgacctcgacatgctgggaagcgacgccctcgacgatttcgatttggacatgctcggcagtgatgcactcgatgactttgatcttgatatgctgatcaacagccggtccagcggcagccctaagaaaaaacggaaagtgggcagccagtatctgcccgacaccgacgatcggcaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctccacagccttatcctttcacctctagcctgagcaccatcaactacgacgagttccctaccatggtgttccccagcggccagatctctcaggcatctgctcttgctccagctccacctcaggttctgcctcaagctcctgctccggctcctgcaccagctatggtgtctgcacttgctcaggcaccagctccagtgcctgttcttgctcctggacctcctcaggctgttgctccaccagcacctaaacctacacaggccggcgagggaacactgtctgaagccctgctgcaactccagttcgatgacgaggatctgggcgccctgctgggaaattctacagaccctgccgtgtttaccgatctggccagcgtggacaacagcgagtttcagcagctcctgaaccagggcatcccagtggctcctcacaccacagagcccatgctgatggaataccccgaggccatcaccagactggtcaccggcgcacaaagacctccagatcctgctccagcaccgcttggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtcttacccctgctcctgttcctcagccactggatcccgcaccagctgtgacacctgaagccagccatctgctggaagatcccgacgaagaaacctctcaggccgtgaaggccctgagagaaatggccgacacagtgatccctcagaaagaggaagccgccatttgcggacagatggacctgtctcaccctccaccaagaggccacctggacgagctgaccaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttctga

SEQ ID NO: 137 - DNA, 2372 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatggtggcggcaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 138 - protein, 729 aa, Artificial Sequence
  Description of Artificial Sequence Synthetic polypeptide
MLLLVTSLLLCELPHPAFLLIPHMEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 139 - DNA, 2619 bp, Artificial Sequence
  Description of Artificial Sequence Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatcctacgtagccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatcagaaacaagaccaacaactacgccacctactacgccgacagcgtgaaggccaggttcaccatctccagagatgacagcaagaacagcctgtacctccagatgaactccctgaaaaccgaggacaccgccgtgtactattgcgtggccggcaatagctttgcctactggggacagggcaccctggttacagtttctgctggtggtggaggctcaggagggggaggttccggaggaggcggttcggacatcgtgatgacacagagccctgacagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaagaccaccacaccagctcctcggcctccaactcctgctcctacaattgccagccagcctctgtctctgaggcccgaagcttgtagaccagctgccggcggagctgtgcatacaagaggactggatttcgcctgcgacatctacatatgggcccccctcgccggtacttgcggtgttttgcttttgtcactggtgattacgaagcgcggtcgaaaaaaactcctctacatcttcaaacaacctttcatgcggcctgtccaaacaactcaagaagaggacgggtgttcatgccgctttccagaggaagaggaaggtggctgtgaacttcgggtgaagttctcacgatctgcggacgcccccgcataccaacagggacagaaccaactctacaatgaactgaacctggggcgaagagaagaatatgacgttctggataagcgcagaggccgcgaccccgaaatgggaggtaagccgagaaggaaaaatccacaggaaggactgtataatgagttgcaaaaggataagatggcggaagcatattcagagattgggatgaaaggcgaaagaagaagaggcaaaggacatgatgggctgtatcagggcctttccacggccacaaaagatacgtatgacgcgctgcacatgcaagctcttcctcccaggggaatggtgtccaaaggagaagaactgttcaccggagtggtgccgattctggtggaactggacggcgatgtcaacggacacaagttctcagtcagcggagaaggggaaggggatgctacgtacgggaagctcaccctgaagctcatctgtactactgggaagctgcctgtgccctggccaactctggtgacaaccctcggttacgggttgcagtgcttcgcacgctaccctgaccacatgaagcagcacgatttcttcaaatccgccatgcctgagggttacgtgcaagagcggaccatctttttcaaggacgatggcaactacaagactcgggccgaagtgaaattcgaaggggacactcttgtgaaccgcatcgagttgaagggcattgacttcaaggaagatggcaacattttgggacacaagctggagtacaactacaattcccataacgtgtacatcaccgccgacaagcagaaaaacgggatcaaggccaacttcaagatccgccacaatatcgaggatggcggagtgcagctggctgaccactaccagcagaacacccccatcggtgatggcccagttctgctccccgataaccattacctgtcgtaccaatcggcattgagcaaggaccccaacgagaagcgcgatcacatggtgttgctggaatttgtgaccgctgccggcattaccctcgggatggacgagctctacaagtaa

SEQ ID NO: 140 - DNA, 2849 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccataggtccagggttttcctccacgtctccacacgtcagtagactcccgcgtccctcgccagatcctctcttccgtctactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatggtggcggcaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 141 - protein, 134 aa, Artificial Sequence
  Description of Artificial Sequence Synthetic polypeptide
METDTLLLWVLLLWVPGSTGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 142 - protein, 25 aa, Artificial Sequence
  Description of Artificial Sequence Synthetic peptide
RRKRGSGEGRGSLLTCGDVEENPGP

SEQ ID NO: 143 - DNA, 2404 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatggtggcggcaccggtttaagcgggtcgctgcagggtcgctcggtgttcgaggccacacgcgtcaccttaatatgcgaaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 144 - DNA, 2881 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccataggtccagggttttcctccacgtctccacacgtcagtagactcccgcgtccctcgccagatcctctcttccgtctactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatggtggcggcaccggtttaagcgggtcgctgcagggtcgctcggtgttcgaggccacacgcgtcaccttaatatgcgaaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 145 - protein, 704 aa, Artificial Sequence
  Description of Artificial Sequence Synthetic polypeptide
MSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGSTCRDYKDHDGDYKDHDIDYKDDDDKMAPKKKRKVGIHGVPGGLEGGGGSGGTEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHYPYDVPDYAGGGGSGGTDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSQL

SEQ ID NO: 146 - DNA, 5006 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgatgaatttccaaccatggtgttccccagcggccagatctctcaggcatctgctcttgctccagctccacctcaggttctgcctcaagctcctgctccggctcctgcaccagctatggtgtctgcacttgctcaggcaccagctccagtgcctgttcttgctcctggacctcctcaggctgttgctccaccagcacctaaacctacacaggccggcgagggaacactgtctgaagccctgctgcaactccagttcgatgacgaagatctgggcgccctgctgggaaactctacagatcctgccgtgtttaccgatctggccagcgtggacaacagcgagtttcagcagctcctgaaccagggcatcccagtggctcctcacaccacagagcctatgctgatggaataccccgaggccatcaccagactggtcaccggcgcacaaagaccacctgatcctgctccagcaccgcttggagcacctggactgcctaatggactgctgtctggcgacgaggacttcagttctatcgctgatatggatttctctgcccttctgtctcagattagtagccagctgtaa

SEQ ID NO: 147 - protein, 1042 aa, Artificial Sequence
  Description of Artificial Sequence Synthetic polypeptide
MSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGSTCRDYKDHDGDYKDHDIDYKDDDDKMAPKKKRKVGIHGVPGGLEGGGGSGGTEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHYPYDVPDYAGGGGSGGTEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 148 - DNA, 6020 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttttga

SEQ ID NO: 149 - DNA, 6497 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccataggtccagggttttcctccacgtctccacacgtcagtagactcccgcgtccctcgccagatcctctcttccgtctactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttttga

SEQ ID NO: 150 - DNA, 3785 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgatgaatttccaaccatggtgttccccagcggccagatctctcaggcatctgctcttgctccagctccacctcaggttctgcctcaagctcctgctccggctcctgcaccagctatggtgtctgcacttgctcaggcaccagctccagtgcctgttcttgctcctggacctcctcaggctgttgctccaccagcacctaaacctacacaggccggcgagggaacactgtctgaagccctgctgcaactccagttcgatgacgaagatctgggcgccctgctgggaaactctacagatcctgccgtgtttaccgatctggccagcgtggacaacagcgagtttcagcagctcctgaaccagggcatcccagtggctcctcacaccacagagcctatgctgatggaataccccgaggccatcaccagactggtcaccggcgcacaaagaccacctgatcctgctccagcaccgcttggagcacctggactgcctaatggactgctgtctggcgacgaggacttcagttctatcgctgatatggatttctctgcccttctgtctcagattagtagccagctgtaa

SEQ ID NO: 151 - DNA, 4799 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttttga

SEQ ID NO: 152 - DNA, 66 bp, Artificial Sequence
  Description of Artificial Sequence Synthetic oligonucleotide
gggactttccactggggactttccactggggactttccactggggactttccactggggactttcc

SEQ ID NO: 153 - DNA, 51 bp, Artificial Sequence
  Description of Artificial Sequence Synthetic oligonucleotide
agacgctagcggggggctataaaagggggtgggggcgttcgtcctcactct

SEQ ID NO: 154 - DNA, 140 bp, Artificial Sequence
  Description of Artificial Sequence Synthetic polynucleotide
gggactttccactggggactttccactggggactttccactggggactttccactggggactttccactcctgcaggagctggcgcgccagacgctagcggggggctataaaagggggtgggggcgttcgtcctcactct

SEQ ID NO: 155 - DNA, 25 bp, Artificial Sequence
  Description of Artificial Sequence Synthetic oligonucleotide
tctagagggtatataatgggggcca

SEQ ID NO: 156 - DNA, 2085 bp, Artificial Sequence
  Description of Artificial Sequence Synthetic polynucleotide
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 157 - DNA, 2076 bp, Artificial Sequence
  Description of Artificial Sequence Synthetic polynucleotide
ttaagtcttcaacccattgtgagttgatttttacagatggtgtaaggaaggggtccagtttcaatcttttgcatacagctagccagttatcccagtaccatttattgactgggaagtcctttccctgttgcttgtttttgttaactttgtcaaagttcggatggttgcaggtatgcagcattatttctgagctctctattttgttctattggtctatgagtctgtttttgtaccaaagccatgctgttttggttactgcagccctgtggtatagtttgaagtcatggctccctttttttactcttcttttttctttctcagagaagagcaggtaacaatgtggggttaaaggtgagcaggtgggttagagtagtggagctaaaggcagatagaggtcttgatccaattatcctcattcccttcatttgtccaaccttgcacattcctgtatgcagggcctttgtaagcttcaccttctgtacaggtgcccagcaactgtcaaaccagccagggcttaacatgagcatgggcattccctatggactggctgggacacacccacatccttagattacacattttgcctgtaacatagataaactaattactaggtatataattctgtttcgttttgttttgctttgttttgtttttgggacagggtctcattctgtcaaacaagcgggagtgcagtggcacaatcaccactcactgcagcctggacttttctgggctcaggtgatcttcccacctcagcctactgagcagctgggactacaggtgtgtatcaccactcccagccaaaatatttttttatagagacagtgtttcaccatgttgcccagtctggtatcgaacttctgggctcaagctatctgcctgccttgacctcccaaagtgctgggattacaggtgtgaggccctgtacccagctcaatcctgtttttatactagaagaacattctctatctgggtcttccaaacttgattcttactggttaatttgtcctgttcttttgctggtccaagaaaatatcctgaaatctttatttcttctccgtttcctccttgtcctaggacagactagccctatccccttcctgaattaagtccgaatatagtcagtctttgagtgtggaatagctcctagcagtctatcagtcaacgggttctctttgtggtcacattctatgtttattcaggagactacagcatggaaagaaaatggactttggagtcaggtgaatcttgattcaaatcttggcagtgccattcatcagctctgtggcattgagcgcatcagtggaccactctctgatccccaattgccttgtctgtaaaatgagattagacacccctgctcaagattatggtaggattatatataatgtgtgtaacacagcttttccagtgcctggtacaaggtaagtgtccaataagaagttacaaatgttcctgagccaccctactgggtccctcctcacatccaatgctggactcttcatgcccaaggaaacatataaggcacaaaagtcagtggcactcagctcagcagaagaaggcacaggaaggggatgggggagtcctggctcccactttgttcttggggtcaggcttgtggccagcctggaaagactgacatgaaccctccagcatctgagggtgccaaccaccaagagcagcacagttcttgtctacaagccacttgtagcaggtgtgaacatttcatcttttcctctggggtttgcaactctctccccagtctcggtcactgcctttggctgtaccacttcccttttcttctcgcctgcacctccctcatctttcctctatgatgacatcgccctggggaagagaagctgagaggaactcctcactcagctagcttcaggagcatgacgtcatctctaccatggaaattccactcactctcctgtgcccccacatttgtcctaggcctcagagtccctataaagagagattcccaagtcagtatcagcacaggacacagctgggttctgaagcttctgagttctgcagcctcacctctgagaaaacctcttttccaccaatacc

SEQ ID NO: 158 - DNA, 2071 bp, Artificial Sequence
  Description of Artificial Sequence Synthetic polynucleotide
gggatttggtatataatactgtgcatacataaataacaacaacaaaattggtccactgcccagagagctctcgatccacagagctgccagacatcatgagatttcaaaagatcttggctctaaaatcagaggatgtttggatataaattctagtacaacccagtcatttgacagatgagtaaaccagggatcagaagagtccaggcatgtaattgccccaaggtcactgagtaagtgagtggccgagtgaccactgaaatttagtccatctgagagctcgcccccctgccctgtgcggtgtttttacaagagtgagcacgcaggttccagggtggtcaagaggtgtttactttcattttgtagtcggccttcatacaaggtgggggctgggaagggaagagcagtccaggccattacccccatgaattcatttgacaaatatttattaagcgtctactgcaaacttggcccggtgctggaagctgaagacacaattccagaggcagacaatgactgcccttggcatttccagtggtcaggggttggtgaggaggggcagagacaccaatccaagagtccccgcagtttatacataccacaggcacggaaagcgccaggaggggaaagaacaggatgtttaccagcaccttccacaggcagccacggtcatgggggtcaggacagggtccccagaggaagtgacgattcggctgagctagaaagacatttccacaggaacttacagtaagggctgcaaggacagcctgtcctcccccgcccaccacctcactaaactttcactgtggcaatcggcattccctaagcctgccaggaagcttccagtaaccacttcctgactcctagcatgacacacttcgggccttccaaggttgacgatctaaggcccttacacagcgccagacacgcgcaggcagggaggcaggatgtgcctctccagacaggaatgtggacgccacctgggctcttcccctcagcctccacaggggaagaatattcttgtggggtttttcccttccaaatgtctcagggcgattccagtgttcccgctagttcctccctcccaggctagaacacaaatccttcccactccctgcctggcaaacaccttctgaccctcaggcccaaggcaatggcccacctcctcccaggctggatggggtctcctcctctctgttcccccagcccctgagcttcctgaggaccaagcttgtggcttcttctccttactcttcctccttggtgtctctatgttagagggccgttagcatctgctggggcctggtcgcattcaccctgctctgccactcactggctgtgtgactctggacaaattaacttctctggacctgcagtttctcctctctacaatgagaatactggagagtccttatcttatgggttgctacagaattaagtgacatctcacacacaacacacttcctacagtccctgttacacgctaaaagtactcaacatgcaacggatacgtcatcagtaaccaccccacgggtttactgtgatgctgcacaattattaagccttggctgctacagagttgtaacctgtctgcacttccaaccggtttgggaatgcaagcagcattcccaagtcccgctttcacccgcgcgctaacggctcaggttcgagtacaggacaggagggaggggagctgtgcacacggcggaggcgcacggcgtgggcacccagcacccggtacactgtgtcctcccgctgcacccagccccttccgcgccgaggcgtccccgaggcgcaagtgggccgccttcagggaactgaccgcccgcggcccgtgtgcagagccgggtgcgcccggcccagtgcgcgcggccgggtgtttcgcctggagccgcaagtgactcagcgcggggcgtgtgcaggcagcgcccggccggggcggggcttttgcactcgtcccggctctttctagctataaacactgcttgccgcgctgcactccaccacgcctcctccaagtcccagcgaacccgcgtgcaacctgtcccgactctagccgcctcttcagctcgcc

SEQ ID NO: 159 - protein, 10 aa, Unknown
  Description of Unknown cleavage site sequence
EDVVCCHSIY

SEQ ID NO: 160 - protein, 14 aa, Unknown
  Description of Unknown cleavage site sequence
LYQEFDEMEECSQH

SEQ ID NO: 161 - DNA, 1884 bp, Artificial Sequence
  Description of Artificial Sequence Synthetic polynucleotide
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggccaacgcgtgccgccaccatgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagttaa

SEQ ID NO: 162 - protein, 4 aa, Unknown
  Description of Unknown hairy-related basic helix-loop-helix repressor domain motif
WRPW

SEQ ID NO: 163 - protein, 6 aa, Artificial Sequence
  Description of Artificial Sequence Synthetic peptide
  MOD_RES (4)..(5): Any amino acid
DSGXXS

SEQ ID NO: 164 - protein, 8 aa, Unknown
  Description of Unknown cleavage site sequence
EPLFAERK

SEQ ID NO: 165 - protein, 4 aa, Unknown
  Description of Unknown cleavage site sequence
YVAD

SEQ ID NO: 166 - protein, 5 aa, Unknown
  Description of Unknown cleavage site sequence
VDVAD

SEQ ID NO: 167 - protein, 4 aa, Unknown
  Description of Unknown cleavage site sequence
DEVD

SEQ ID NO: 168 - protein, 4 aa, Unknown
  Description of Unknown cleavage site sequence
VEHD

SEQ ID NO: 169 - protein, 5 aa, Unknown
  Description of Unknown cleavage site sequence
  MOD_RES (5)..(5): Any amino acid
LGHDX

SEQ ID NO: 170 - protein, 5 aa, Unknown
  Description of Unknown cleavage site sequence
LQTDG

SEQ ID NO: 171 - protein, 4 aa, Unknown
  Description of Unknown cleavage site sequence
  MOD_RES (1)..(1): Pyroglutamic acid
  MOD_RES (3)..(3): Any amino acid
EHXG

SEQ ID NO: 172 - protein, 8 aa, Unknown
  Description of Unknown cleavage site sequence
EVNLDAEF

SEQ ID NO: 173 - protein, 7 aa, Unknown
  Description of Unknown cleavage site sequence
PQGIAGQ

SEQ ID NO: 174 - protein, 5 aa, Unknown
  Description of Unknown cleavage site sequence
  MOD_RES (2)..(2): Gly or Ala
GXELR

SEQ ID NO: 175 - protein, 7 aa, Unknown
  Description of Unknown cleavage site sequence
ENLYFQS

SEQ ID NO: 176 - protein, 7 aa, Unknown
  Description of Unknown cleavage site sequence
  MOD_RES (1)..(1): Abz
  MOD_RES (7)..(7): Lys(Dnp)
XHPFHLK

SEQ ID NO: 177 - DNA, 5483 bp, Artificial Sequence
  Description of Artificial Sequence Synthetic polynucleotide
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttagctggtgttgatgaacatctgcacgatgtgcacgaagctctgcaggaactctttgatattcttttcttccagttcctcgcattctttgcagccggactcggtcacattgccgttggaggacaggctgttgttggccaggatgatcaggttttccacggtatcgtggatggaggcgtcgccgctttccaggctgatcacttgcagctcgagcagaaagcacttcatggcggtcactttacagctagggtgcacgtcgctctcggtgtacagtgtggcgtcgatgtgcatgctctggatcagatcctcgatcttcttcagatcgctgatcacgttgacccagttgcctgtagagccgggcacccaaagaagcagcacccacagcagcagtgtgtcggtttccataggtccagggttttcctccacgtctccacacgtcagtagactcccgcgtccctcgccagatcctctcttccgtctactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgagtagacctggcgaaagaccatttcagtgcagaatttgtatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaacccttccagtgccggatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggcagcacctgtagagactacaaggaccacgacggcgattataaggatcacgacatcgactacaaagacgacgatgacaagatggcccctaagaagaagcggaaagtcggcatccatggcgtgccaggtggacttgaaggtggcggaggatctggcggcacagaggatgttgtgtgctgccacagcatctacggcaagaagaagggcgacatcgatacctatcggtacatcggcagcagcggcacaggctgcgttgtgatcgtgggaagaatcgtgctgagcggctctggcacaagcgcccctattacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggacccgtgatccagatgtacaccaacgtggaccaggacctcgttggctggcctgctcctcagggaagtagaagcctgacaccttgcacctgtggctccagcgatctgtacctggtcaccagacacgccgacgtgatccctgtcagaagaagaggagattccagaggcagcctgctgagccctagacctatcagctacctgaagggcagctctggcggacctctgctttgtcctgctggacatgccgtgggcctgtttagagccgccgtgtgtacaagaggcgtggcaaaggccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcactacccctacgacgtgccagattatgctggcggaggtggcagcggaggcaccgatgaatttccaaccatggtgttccccagcggccagatctctcaggcatctgctcttgctccagctccacctcaggttctgcctcaagctcctgctccggctcctgcaccagctatggtgtctgcacttgctcaggcaccagctccagtgcctgttcttgctcctggacctcctcaggctgttgctccaccagcacctaaacctacacaggccggcgagggaacactgtctgaagccctgctgcaactccagttcgatgacgaagatctgggcgccctgctgggaaactctacagatcctgccgtgtttaccgatctggccagcgtggacaacagcgagtttcagcagctcctgaaccagggcatcccagtggctcctcacaccacagagcctatgctgatggaataccccgaggccatcaccagactggtcaccggcgcacaaagaccacctgatcctgctccagcaccgcttggagcacctggactgcctaatggactgctgtctggcgacgaggacttcagttctatcgctgatatggatttctctgcccttctgtctcagattagtagccagctgtaa

(19) United States
(12) Patent Application Publication
(10) Pub. No.: US 2023/0011052 A1
(43) Pub. Date: Jan. 12, 2023

(54) METHOD AND COMPOSITIONS FOR REGULATED ARMORING OF CELLS

(71) Applicant: Senti Biosciences, Inc., South San Francisco, CA (US)

(72) Inventors: Marcela Guzman Ayala, San Francisco, CA (US); Russell Morrison Gordley, San Francisco, CA (US); Michelle Elizabeth Hung, South San Francisco, CA (US); Gary Lee, Castro Valley, CA (US); Jack Tzu-Chiao Lin, Redwood City, CA (US); Timothy Kuan-Ta Lu, San Francisco, CA (US); Assen Boyanov Roguev, San Francisco, CA (US)

(21) Appl. No.: 17/838,118

(22) Filed: Jun. 10, 2022

Related U.S. Application Data
(63) Continuation of application No. PCT/US2020/064688, filed on Dec. 11, 2020.
(60) Provisional application No. 63/116,103, filed on Nov. 19, 2020.
(60) Provisional application No. 62/947,427, filed on Dec. 12, 2019.

(51) Int. Cl. A61K 48/00; C07K 14/005; A61P 35/00; C07K 14/725; C12N 15/63; C12N 15/86; A61K 45/06

(52) U.S. Cl. CPC A61K 48/0066; C07K 14/005; A61P 35/00; C07K 14/7051; C12N 15/635; C12N 15/86; A61K 45/06; A61K 2039/5156

(57) ABSTRACT

Provided herein are compositions and methods for regulating expression of effector molecules using regulatable transcription factors and/or activation inducible promoters.

### CROSS REFERENCE TO RELATED APPLICATIONS

This application is continuation of International Application No. PCT/US2020/064688, filed Dec. 11, 2020, which claims the benefit of U.S. Provisional Application Nos: 62/947,427 filed Dec. 12, 2019 and 63/116,103 filed Nov. 19, 2020, each of which is hereby incorporated in its entirety by reference for all purposes.

### SEQUENCE LISTING

The instant application contains a Sequence Listing which has been submitted via EFS-Web and is hereby incorporated by reference in its entirety. Said ASCII copy, created on Month XX, 20XX, is named US sequencelisting.txt, and is X,XXX,XXX bytes in size.

### BACKGROUND

Tumors employ a range of direct and indirect suppression strategies to avoid recognition and clearance by the immune system. These escape strategies can effectively shut down cell therapies. Combinatorial armoring, expression of combinations of effectors, can impact the entire cancer immunity cycle and boost the activity of cell therapies as unarmored therapies have poor efficacy in solid tumors. However, current cell and gene therapy products have no control. Uncontrolled armored therapies can have toxicity in subjects. Thus, additional methods of controlling and regulating the expression of combinations of effector molecules are required.

### SUMMARY

In one aspect, provided herein are engineered nucleic acids comprising: a first expression cassette comprising a first promoter and a first exogenous polynucleotide sequence encoding an activation-conditional control polypeptide (ACP), wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.

In another aspect, provided herein are engineered expression systems comprising: (a) a first expression cassette comprising a first promoter and a first exogenous polynucleotide sequence encoding an activation-conditional control polypeptide (ACP), wherein the first promoter is operably linked to the first exogenous polynucleotide; and (b) a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. In some embodiments, the first expression cassette and the second expression cassette are encoded by separate polynucleotide sequences. In some embodiments, the first expression cassette and the second expression cassette are encoded by the same polynucleotide sequence. In some embodiments, the first expression cassette and/or the second expression cassette further comprises an additional exogenous polynucleotide sequence encoding an antigen recognizing receptor. In some embodiments, the first expression cassette further comprises an additional exogenous polynucleotide sequence encoding an antigen recognizing receptor. In some embodiments, the second expression cassette further comprises an additional exogenous polynucleotide sequence encoding an antigen recognizing receptor. In some embodiments, the engineered expression system further comprises an additional expression cassette including an additional promoter and an additional exogenous polynucleotide sequence encoding an antigen recognizing receptor, wherein the additional promoter is operably linked to the additional exogenous polynucleotide. In some embodiments, the additional exogenous polynucleotide sequence is encoded by the same polynucleotide as the first expression cassette or the second expression cassette. In some embodiments, the additional exogenous polynucleotide sequence is encoded by the same polynucleotide as the first expression cassette. In some embodiments, the additional exogenous polynucleotide sequence is encoded by the same polynucleotide as the second expression cassette. In some embodiments, a first vector comprises the first expression cassette and the additional expression cassette if present, and a second vector comprises the second expression cassette. In some embodiments, a first vector comprises the first expression cassette, and a second vector comprises the second expression cassette and the the additional expression cassette if present. In some embodiments, a first vector comprises the first expression cassette and the second expression cassette, and a second vector comprises the additional expression cassette if present. In some embodiments, the engineered expression system comprises any of the aspects, features, or embodiments of the engineered nucleic acids described herein, including, but not limited to, any of the aspects, features, or embodiments described in enumerated embodiments 1-359.

In some embodiments, when the second expression cassette comprises two or more units of (L-E)_{X}, each linker polynucleotide sequence is operably associated with the translation of each molecule as a separate polypeptide.

In some embodiments, the linker polynucleotide sequence encodes a 2A ribosome skipping tag.

In some embodiments, the 2A ribosome skipping tag is selected from the group consisting of: P2A, T2A, E2A, and F2A.

In some embodiments, the linker polynucleotide sequence encodes an Internal Ribosome Entry Site (IRES).

In some embodiments, the linker polynucleotide sequence encodes a cleavable polypeptide.

In some embodiments, the cleavable polypeptide comprises a furin polypeptide sequence.

In some embodiments, the second expression cassette comprising one or more units of (L-E)_{X} further comprises a polynucleotide sequence encoding a secretion signal peptide.

In some embodiments, for each X the corresponding secretion signal peptide is operably associated with the effector molecule.

In some embodiments, each secretion signal peptide comprises a native secretion signal peptide native to the corresponding effector molecule.

In some embodiments, each secretion signal peptide comprises a non-native secretion signal peptide that is non-native to the corresponding effector molecule.

In some embodiments, the non-native secretion signal peptide is selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, the ACP-responsive promoter comprises an ACP-binding domain and a promoter sequence.

In some embodiments, the promoter sequence is derived from a promoter selected from the group consisting of: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.

In some embodiments, the ACP-responsive promoter is a synthetic promoter.

In some embodiments, the ACP-responsive promoter comprises a minimal promoter.

In some embodiments, the ACP-binding domain comprises one or more zinc finger binding sites.

In some embodiments, the first promoter is a constitutive promoter, an inducible promoter, or a synthetic promoter.

In some embodiments, the constitutive promoter is selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the chemokine is selected from the group consisting of: CCL21a, CXCL10, CXCL11, CXCL13, a CXCL10-CXCL11 fusion protein, CCL19, CXCL9, and XCL1.

In some embodiments, the homing molecule is selected from the group consisting of: anti-integrin alpha4,beta7; anti-MAdCAM; CCR9; CXCR4; SDF1; MMP-2; CXCR1; CXCR7; CCR2; CCR4; and GPR15.

In some embodiments, the growth factor is selected from the group consisting of: FLT3L and GM-CSF.

In some embodiments, the co-activation molecule is selected from the group consisting of: c-Jun, 4-1BBL and CD40L.

In some embodiments, the tumor microenvironment modifier is selected from the group consisting of: adenosine deaminase, TGFbeta inhibitors, immune checkpoint inhibitors, VEGF inhibitors, and HPGE2.

In some embodiments, the TGFbeta inhibitors are selected from the group consisting of: an anti-TGFbeta peptide, an anti-TGFbeta antibody, a TGFb-TRAP, and combinations thereof.

In some embodiments, the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

In some embodiments, the VEGF inhibitors comprise anti-VEGF antibodies, anti-VEGF peptides, or combinations thereof.

In some embodiments, each effector molecule is a human-derived effector molecule.

In some embodiments, the first exogenous polynucleotide sequence further encodes an antigen recognizing receptor.

In certain aspects, provided herein are engineered nucleic acids comprising: a first expression cassette comprising a first promoter and a first exogenous polynucleotide sequence encoding an activation-conditional control polypeptide (ACP) and an antigen recognizing receptor, wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.

In certain aspects, provided herein are engineered nucleic acids comprising: a first expression cassette comprising a first promoter and a first exogenous polynucleotide sequence encoding an antigen recognizing receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent.

In some embodiments, the ACP is capable of inducing expression of the second expression cassette by binding to the ACP-responsive promoter.

In some embodiments, the ACP is the antigen recognizing receptor and the ACP is capable of inducing expression of the second expression cassette following binding of the ACP to a cognate antigen. In some embodiments, the ACP-responsive promoter is an inducible promoter that is capable of being induced by the ACP binding to the cognate antigen. In some embodiments, the ACP-responsive promoter is derived from a promoter region of a gene upregulated following binding of the ACP to the cognate antigen.

In some embodiments, the ACP-responsive promoter is selected from the group consisting of a constitutive promoter, an inducible promoter, and a synthetic promoter.

In some embodiments, the ACP-responsive promoter comprises a minimal promoter.

In some embodiments, the ACP-binding domain comprises one or more zinc finger binding sites.

In some embodiments, further comprising a linker polynucleotide sequence localized between the first expression cassette and the second expression cassette.

In some embodiments, wherein the linker polynucleotide sequence is operably associated with the translation of the ACP and each effector molecule as separate polypeptides.

In some embodiments, the first exogenous polynucleotide sequence further comprises a linker polynucleotide sequence localized between the region of the first exogenous polynucleotide sequence encoding the ACP and the region of the first exogenous polynucleotide sequence encoding the antigen recognizing receptor. In some embodiments, the linker polynucleotide sequence is operably associated with the translation of the ACP and the antigen recognizing receptor as separate polypeptides.

In some embodiments, the engineered nucleic acids further comprise a linker polynucleotide sequence localized between the first expression cassette and the second expression cassette. In some embodiments, the linker polynucleotide sequence is operably associated with the translation of the antigen receptor and each effector molecule as separate polypeptides.

In some embodiments, the first promoter is operably linked to the first exogenous polynucleotide sequence encoding the ACP, linker polynucleotide sequence, and antigen recognizing receptor.

In some embodiments, the linker polynucleotide sequence encodes a 2A ribosome skipping tag. In some embodiments, the 2A ribosome skipping tag is selected from the group consisting of: P2A, T2A, E2A, and F2A. In some embodiments, the linker polynucleotide sequence encodes an Internal Ribosome Entry Site (IRES). In some embodiments, the linker polynucleotide sequence encodes a cleavable polypeptide. In some embodiments, the cleavable polypeptide comprises a furin polypeptide sequence.

In some embodiments, the antigen recognizing receptor recognizes an antigen selected from the group consisting of: 5T4, ADAMS, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1. In some embodiments, the antigen recognizing receptor recognizes GPC3. In some embodiments, the antigen recognizing receptor recognizes mesothelin (MSLN).

In some embodiments, the antigen recognizing receptor comprises an antigen-binding domain.

In some embodiments, the antigen-binding domain that binds to GPC3 comprises a heavy chain variable (VH) region and a light chain variable (VL) region, wherein the VH comprises: a heavy chain complementarity determining region 1 (CDR-H1) having the amino acid sequence of KNAMN (SEQ ID NO: 119), a heavy chain complementarity determining region 2 (CDR-H2) having the amino acid sequence of RIRNKTNNYATYYADSVKA (SEQ ID NO: 120), and a heavy chain complementarity determining region 3 (CDR-H3) having the amino acid sequence of GNSFAY (SEQ ID NO: 121), and wherein the VL comprises: a light chain complementarity determining region 1 (CDR-L1) having the amino acid sequence of KSSQSLLYSSNQKNYLA (SEQ ID NO: 122), a light chain complementarity determining region 2 (CDR-L2) having the amino acid sequence of WASSRES (SEQ ID NO: 123), and a light chain complementarity determining region 3 (CDR-L3) having the amino acid sequence of QQYYNYPLT (SEQ ID NO: 124).

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 125)
EVQLVETGGGMVQPEGSLKLSCAASGFTFNKNAMNWVRQAPGKGLEWVA
RIRNKTNNYATYYADSVKARFTISRDDSQSMLYLQMNNLKIEDTAMYYC
VAGNSFAYWGQGTLVTVSA
or
(SEQ ID NO: 126)
EVQLVESGGGLVQPGGSLRLSCAASGFTFNKNAMNWVRQAPGKGLEWVG
RIRNKTNNYATYYADSVKARFTISRDDSKNSLYLQMNSLKTEDTAVYYC
VAGNSFAYWGQGTLVTVSA.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 127)
DIVMSQSPSSLVVSIGEKVTMTCKSSQSLLYSSNQKNYLAWYQQKPGQS
PKLLIYWASSRESGVPDRFTGSGSGTDFTLTISSVKAEDLAVYYCQQYY
NYPLTFGAGTKLELK,
or
(SEQ ID NO: 128)
DIVMTQSPDSLAVSLGERATINCKSSQSLLYSSNQKNYLAWYQQKPGQP
PKLLIYWASSRESGVPDRFSGSGSGTDFTLTISSLQAEDVAVYYCQQYY
NYPLTFGQGTKLEIK.

In some embodiments, the antigen-binding domain that binds to MSLN comprises the three complementarity determining regions (CDRs) of a single-domain monoclonal antibody having the amino acid sequence of:

(SEQ ID NO: 129)
QVQLVESGGGTVQAGGSLKLACAASGLPRTYNVMGWFRQAPGKEREGVA
IIYTTTGATYYRDSVKGRATISQDNAKKSVSLQMNSLRPEDTAIYYCVA
RQPNSGPWEYWGQGTQVTVSS,
or
(SEQ ID NO: 130)
QVKLEESGGGSVQAGGSLRLSCTTSGYTNSYKWMGWFRQAPGQEREGVA
VIYTGNDRTYYSDSVKGRFTISRDNAKNMIYLDMTRLRPEDSAVYECAI
GHDGAWRYWGQGTQVTVSS.

In some embodiments, the antigen-binding domain comprises an antibody, an antigen-binding fragment of an antibody, a F(ab) fragment, a F(ab′) fragment, a single chain variable fragment (scFv), or a single-domain antibody (sdAb).

In some embodiments, the antigen-binding domain comprises a single chain variable fragment (scFv).

In some embodiments, the scFv comprises a heavy chain variable domain (VH) and a light chain variable domain (VL).

In some embodiments, the VH and VL are separated by a peptide linker.

In some embodiments, the scFv comprises the structure VH-L-VL or VL-L-VH, wherein VH is the heavy chain variable domain, L is the peptide linker, and VL is the light chain variable domain.

In some embodiments, the antigen recognizing receptor is a chimeric antigen receptor (CAR) or T cell receptor (TCR).

In some embodiments, the antigen recognizing receptor is a CAR.

In some embodiments, the CAR comprises one or more intracellular signaling domains, and each of the one or more intracellular signaling domains is selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

In some embodiments, the CAR comprises a transmembrane domain, and the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceRlg transmembrane domain, and an NKG2D.

In some embodiments, the CAR comprises a spacer region between the antigen-binding domain and the transmembrane domain.

In some embodiments, the ACP is a transcriptional modulator.

In some embodiments, the ACP is a transcriptional repressor.

In some embodiments, the ACP is a transcriptional activator.

In some embodiments, the ACP further comprises a repressible protease and one or more cognate cleavage sites of the repressible protease.

In some embodiments, the ACP further comprises a hormone-binding domain of estrogen receptor (ERT2 domain).

In some embodiments, the ACP is a transcription factor.

In some embodiments, the transcription factor is a zinc-finger-containing transcription factor.

In some embodiments, the ACP comprises a DNA-binding zinc finger protein domain (ZF protein domain) and a transcriptional effector domain.

In some embodiments, the ZF protein domain is modular in design and is composed of zinc finger arrays (ZFA).

In some embodiments, the ZF protein domain comprises one to ten ZFA.

In some embodiments, the effector domain is selected from the group consisting of: a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some embodiments, the one or more cognate cleavage sites of the repressible protease are localized between the ZF protein domain and the effector domain.

In some embodiments, the repressible protease is hepatitis C virus (HCV) nonstructural protein 3 (NS3).

In some embodiments, the cognate cleavage site comprises an NS3 protease cleavage site.

In some embodiments, the NS3 protease cleavage site comprises a NS3/NS4A, a NS4A/NS4B, a NS4B/NS5A, or a NS5A/NS5B junction cleavage site.

In some embodiments, the NS3 protease can be repressed by a protease inhibitor.

In some embodiments, the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir. In some embodiments, the protease inhibitor is grazoprevir. In some embodiments, the protease inhibitor is grazoprevir and and elbasvir. In some embodiments, wherein the grazoprevir and the elbasvir is co-formulated in a pharmaceutical composition. In some embodiments, the pharmaceutical composition is a tablet. In some embodiments, the grazoprevir and the elbasvir are at a 2 to 1 weight ratio. In some embodiments, the grazoprevir is 100 mg per unit dose and the elbasvir is 50 mg per unit dose.

In some embodiments, the ACP is capable of undergoing nuclear localization upon binding of the ERT2 domain to tamoxifen or a metabolite thereof.

In some embodiments, the tamoxifen metabolite is selected from the group consisting of: 4-hydroxytamoxifen, N-desmethyltamoxifen, tamoxifen-N-oxide, and endoxifen.

In some embodiments, the ACP further comprises a degron, and wherein the degron is operably linked to the ACP.

In some embodiments, the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

In some embodiments, the degron comprises a cereblon (CRBN) polypeptide substrate domain capable of binding CRBN in response to an immunomodulatory drug (IMiD) thereby promoting ubiquitin pathway-mediated degradation of the ACP.

In some embodiments, the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

In some embodiments, the CRBN polypeptide substrate domain is a chimeric fusion product of native CRBN polypeptide sequences.

In some embodiments, the CRBN polypeptide substrate domain is a IKZF3/ZFP91/IKZF3 chimeric fusion product having the amino acid sequence of

(SEQ ID NO: 131)
FNVLMVHKRSHTGERPLQCEICGFTCRQKGNLLRHIKLHTGEKPFKCHL
CNYACQRRDAL.

In some embodiments, the IMiD is an FDA-approved drug.

In some embodiments, the IMiD is selected from the group consisting of: thalidomide, lenalidomide, and pomalidomide.

In some embodiments, the degron is localized 5′ of the repressible protease, 3′ of the repressible protease, 5′ of the ZF protein domain, 3′ of the ZF protein domain, 5′ of the effector domain, or 3′ of the effector domain.

In some embodiments, the engineered nucleic acid further comprises an insulator.

In some embodiments, the insulator is localized between the first expression cassette and the second expression cassette.

In some embodiments, the first expression cassette is localized in the same orientation relative to the second expression cassette.

In some embodiments, the first expression cassette is localized in the opposite orientation relative to the second expression cassette.

In some embodiments, the engineered nucleic acid is selected from the group consisting of: a DNA, a cDNA, an RNA, an mRNA, and a naked plasmid.

In another aspect, provided herein are expression vectors comprising the engineered nucleic acid, the expression sysem, or the first expression cassette, the second expression cassette, and/or the additional expression cassettes disclosed herein.

In another aspect, provided herein are compositions comprising the engineered nucleic acid, the expression sysem, or the first expression cassette, the second expression cassette, and/or the additional expression cassette described herein, and a pharmaceutically acceptable carrier.

In another aspect, provided herein are isolated cells comprising the engineered nucleic acid, the expression sysem, or the first expression cassette, the second expression cassette, and/or the additional expression cassette described herein or the vector as described herein.

In some embodiments, the engineered nucleic acid is recombinantly expressed.

In some embodiments, the engineered nucleic acid is expressed from a vector or a selected locus from the genome of the cell.

In some embodiments, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the cell is a Natural Killer (NK) cell.

In some embodiments, the cell is autologous.

In some embodiments, the cell is allogeneic.

In some embodiments, the cell is a tumor cell selected from the group consisting of: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.

In some embodiments, the cell is engineered via transduction with an oncolytic virus.

In some embodiments, the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

In some embodiments, the oncolytic virus is a recombinant oncolytic virus comprising the first expression cassette and the second expression cassette.

In some embodiments, the cell is a bacterial cell selected from the group consisting of: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

In another aspect, provided herein are compositions comprising the isolated cell described herein, and a pharmaceutically acceptable carrier.

In another aspect, provided herein are methods of treating a subject in need thereof, the method comprising administering a therapeutically effective dose of any of the isolated cells or the compositions described herein.

In another aspect, provided herein are methods of stimulating a cell-mediated immune response to a tumor cell in a subject, the method comprising administering to a subject having a tumor a therapeutically effective dose of any of the isolated cells or the compositions described herein.

In another aspect, provided herein are methods of providing an anti-tumor immunity in a subject, the method comprising administering to a subject in need thereof a therapeutically effective dose of any of the isolated cells or the compositions described herein.

In another aspect, provided herein are methods of treating a subject having cancer, the method comprising administering a therapeutically effective dose of any of the isolated cells or the compositions described herein.

In another aspect, provided herein are methods of reducing tumor volume in a subject, the method comprising administering to a subject having a tumor a composition comprising any of the isolated cells or the compositions described herein.

In some embodiments, the administering comprises systemic administration.

In some embodiments, the administering comprises intratumoral administration.

In some embodiments, the isolated cell is derived from the subject.

In some embodiments, the isolated cell is allogeneic with reference to the subject.

In some embodiments, the method further comprises administering a checkpoint inhibitor.

In some embodiments, the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.

In some embodiments, the method further comprises administering an anti-CD40 antibody.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In another aspect, provided herein are lipid-based structures the engineered nucleic acid, the expression sysem, or the first expression cassette, the second expression cassette, and/or the additional expression cassette described herein.

In some embodiments, the lipid-based structure comprises a extracellular vesicle.

In some embodiments, the extracellular vesicle is selected from the group consisting of: a nanovesicle and an exosome.

In some embodiments, the lipid-based structure comprises a lipid nanoparticle or a micelle.

In some embodiments, the lipid-based structure comprises a liposome.

In another aspect, provided herein are compositions comprising the lipid-based structure described herein, and a pharmaceutically acceptable carrier.

In another aspect, provided herein are methods of treating a subject in need thereof, the method comprising administering a therapeutically effective dose of any of the lipid-based structures or compositions described herein.

In another aspect, provided herein are methods of stimulating a cell-mediated immune response to a tumor cell in a subject, the method comprising administering to a subject having a tumor a therapeutically effective dose of any of the lipid-based structures or compositions described herein.

In another aspect, provided herein are methods of providing an anti-tumor immunity in a subject, the method comprising administering to a subject in need thereof a therapeutically effective dose of any of the lipid-based structures or compositions described herein.

In another aspect, provided herein are methods of treating a subject having cancer, the method comprising administering a therapeutically effective dose of any of the lipid-based structures or compositions described herein.

In another aspect, provided herein are methods of reducing tumor volume in a subject, the method comprising administering to a subject having a tumor a composition comprising any of the lipid-based structures or compositions described herein.

In some embodiments, the administering comprises systemic administration.

In some embodiments, the administering comprises intratumoral administration.

In some embodiments, the lipid-based structure is capable of engineering a cell in the subject.

In some embodiments, the method further comprises administering a checkpoint inhibitor.

In some embodiments, the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.

In some embodiments, the method further comprises administering an anti-CD40 antibody.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In another aspect, provided herein are nanoparticles the engineered nucleic acid, the expression sysem, or the first expression cassette, the second expression cassette, and/or the additional expression cassette described herein.

In some embodiments, the nanoparticle comprises an inorganic material.

In another aspect, provided herein are compositions comprising the nanoparticles described herein.

In another aspect, provided herein are methods of treating a subject in need thereof, the method comprising administering a therapeutically effective dose of any of the nanoparticles or the compositions described herein

In another aspect, provided herein are methods of stimulating a cell-mediated immune response to a tumor cell in a subject, the method comprising administering to a subject having a tumor a therapeutically effective dose of any of the nanoparticles or the compositions described herein.

In another aspect, provided herein are methods of providing an anti-tumor immunity in a subject, the method comprising administering to a subject in need thereof a therapeutically effective dose of any of the nanoparticles or the compositions described herein.

In another aspect, provided herein are methods of treating a subject having cancer, the method comprising administering a therapeutically effective dose of any of the nanoparticles or the compositions described herein.

In another aspect, provided herein are methods of reducing tumor volume in a subject, the method comprising administering to a subject having a tumor a composition comprising any of the nanoparticles or the compositions described herein.

In some embodiments, the administering comprises systemic administration.

In some embodiments, the administering comprises intratumoral administration.

In some embodiments, the nanoparticle is capable of engineering a cell in the subject.

In some embodiments, the method further comprises administering a checkpoint inhibitor.

In some embodiments, the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.

In some embodiments, the method further comprises administering an anti-CD40 antibody.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In another aspect, provided herein are viruses engineered to comprise the engineered nucleic acid described herein.

In some embodiments, the virus is selected from the group consisting of: a lentivirus, a retrovirus, an oncolytic virus, an adenovirus, an adeno-associated virus (AAV), and a virus-like particle (VLP).

In some embodiments, the virus is an oncolytic virus.

In some embodiments, the first expression cassette and the second expression cassette are capable of being expressed in a tumor cell.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In some embodiments, the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

In another aspect, provided herein are compositions comprising the engineered virus or the compositions.

In another aspect, provided herein are methods of stimulating a cell-mediated immune response to a tumor cell in a subject, the method comprising administering to a subject having a tumor a therapeutically effective dose of any of the engineered viruses or the compositions.

In another aspect, provided herein are methods of providing an anti-tumor immunity in a subject, the method comprising administering to a subject in need thereof a therapeutically effective dose of any of the engineered viruses or the compositions.

In another aspect, provided herein are methods of treating a subject having cancer, the method comprising administering a therapeutically effective dose of any of the engineered viruses or the compositions.

In another aspect, provided herein are methods of reducing tumor volume in a subject, the method comprising administering to a subject having a tumor a composition comprising any of the engineered viruses or the compositions.

In some embodiments, the administering comprises systemic administration.

In some embodiments, the administering comprises intratumoral administration.

In some embodiments, the engineered virus infects a cell in the subject and expresses the first expression cassette and the second expression cassette.

In some embodiments, the method further comprises administering a checkpoint inhibitor.

In some embodiments, the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.

In some embodiments, the method further comprises administering an anti-CD40 antibody.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In another aspect, provided herein are engineered cells comprising: a first expression cassette comprising a first promoter and a first exogenous polynucleotide sequence encoding an activation-conditional control polypeptide (ACP), wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.

In some embodiments, the first expression cassette and the second expression cassette are encoded by separate polynucleotide sequences.

In some embodiments, the first expression cassette and the second expression cassette are encoded by a single polynucleotide sequence.

The engineered cell of any one of claims 153-155, wherein when the second expression cassette comprises two or more units of (L₁-E)_{X}, each L₁ linker polynucleotide sequence is operably associated with the translation of each effector molecule as a separate polypeptide.

In some embodiments, the engineered cell further comprises a second linker polynucleotide sequence, wherein the second linker polynucleotide links the first expression cassette to the second expression cassette.

In some embodiments, the second linker polynucleotide sequence is operably associated with the translation of each effector molecule and the ACP as separate polypeptides.

In some embodiments, each linker polynucleotide sequence encodes a 2A ribosome skipping tag.

In some embodiments, the 2A ribosome skipping tag is selected from the group consisting of: P2A, T2A, E2A, and F2A.

In some embodiments, each linker polynucleotide sequence encodes an Internal Ribosome Entry Site (IRES).

In some embodiments, the linker polynucleotide sequence encodes a cleavable polypeptide.

In some embodiments, the cleavable polypeptide comprises a furin polypeptide sequence.

In some embodiments, the second expression cassette comprising one or more units of (L₁-E)_{X} further comprises a polynucleotide sequence encoding a secretion signal peptide.

In some embodiments, for each X the corresponding secretion signal peptide is operably associated with the effector molecule.

In some embodiments, each secretion signal peptide comprises a native secretion signal peptide native to the corresponding effector molecule.

In some embodiments, each secretion signal peptide comprises a non-native secretion signal peptide that is non-native to the corresponding effector molecule.

In some embodiments, the non-native secretion signal peptide is selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, the ACP-responsive promoter comprises an ACP-binding domain and a promoter sequence.

In some embodiments, the promoter sequence is derived from a promoter selected from the group consisting of: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.

In some embodiments, the ACP-responsive promoter is a synthetic promoter.

In some embodiments, the ACP-responsive promoter comprises a minimal promoter.

In some embodiments, the ACP-binding domain comprises one or more zinc finger binding sites.

In some embodiments, the first promoter is a constitutive promoter, an inducible promoter, or a synthetic promoter.

In some embodiments, the constitutive promoter is selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the chemokine is selected from the group consisting of: CCL21a, CXCL10, CXCL11, CXCL13, a CXCL10-CXCL11 fusion protein, CCL19, CXCL9, and XCL1.

In some embodiments, the homing molecule is selected from the group consisting of: anti-integrin alpha4,beta7; anti-MAdCAM; CCR9; CXCR4; SDF1; MMP-2; CXCR1; CXCR7; CCR2; and GPR15.

In some embodiments, the growth factor is selected from the group consisting of: FLT3L and GM-CSF.

In some embodiments, the co-activation molecule is selected from the group consisting of: c-Jun, 4-1BBL, and CD40L.

In some embodiments, the tumor microenvironment modifier is selected from the group consisting of: adenosine deaminase, TGFbeta inhibitors, immune checkpoint inhibitors, VEGF inhibitors, and HPGE2.

In some embodiments, the TGFbeta inhibitors are selected from the group consisting of: an anti-TGFbeta peptide, an anti-TGFbeta antibody, a TGFb-TRAP, and combinations thereof.

In some embodiments, the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

In some embodiments, the VEGF inhibitors comprise anti-VEGF antibodies, anti-VEGF peptides, or combinations thereof.

In some embodiments, each effector molecule is a human-derived effector molecule.

In some embodiments, the cell further comprises a third expression cassette comprising a third promoter and a third exogenous polynucleotide sequence encoding an antigen recognizing receptor, wherein the third promoter is operably linked to the third exogenous polynucleotide.

In some embodiments, the first exogenous polynucleotide sequence further encodes an antigen recognizing receptor.

In another aspect, provided herein are engineered cells comprising: a first expression cassette comprising a first promoter and a first exogenous polynucleotide sequence encoding an activation-conditional control polypeptide (ACP) and an antigen recognizing receptor, wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.

In another aspect, provided herein are engineered cells comprising: a first expression cassette comprising a first promoter and a first exogenous polynucleotide sequence encoding an antigen recognizing receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent.

In some embodiments, the cell further comprises a third expression cassette comprising a third promoter and a third exogenous polynucleotide sequence encoding an activation-conditional control polypeptide (ACP), wherein the third promoter is operably linked to the third exogenous polynucleotide.

In some embodiments, the ACP is capable of inducing expression of the second expression cassette by binding to the ACP-responsive promoter.

In some embodiments, the ACP is the antigen recognizing receptor and the ACP is capable of inducing expression of the second expression cassette following binding of the ACP to a cognate antigen. In some embodiments, the ACP-responsive promoter is an inducible promoter that is capable of being induced by the ACP binding to the cognate antigen. In some embodiments, the ACP-responsive promoter is derived from a promoter region of a gene upregulated following binding of the ACP to the cognate antigen.

In some embodiments, the ACP is the antigen recognizing receptor and the ACP is capable of inducing expression of the second expression cassette by binding to its cognate antigen.

In some embodiments, the ACP-responsive promoter is an inducible promoter that is capable of being induced by the ACP binding to its cognate antigen.

In some embodiments, the ACP-responsive promoter is selected from the group consisting of a constitutive promoter, an inducible promoter, and a synthetic promoter.

In some embodiments, the ACP-responsive promoter comprises a minimal promoter.

In some embodiments, the ACP-binding domain comprises one or more zinc finger binding sites.

In some embodiments, the first exogenous polynucleotide sequence further comprises a third linker polynucleotide sequence localized between the region of the first exogenous polynucleotide sequence encoding the ACP and the region of the first exogenous polynucleotide sequence encoding the antigen recognizing receptor. In some embodiments, the third linker polynucleotide sequence is operably associated with the translation of the ACP and the antigen recognizing receptor as separate polypeptides. In some embodiments, the first promoter is operably linked to the first exogenous polynucleotide sequence encoding the ACP, third linker polynucleotide sequence, and antigen recognizing receptor.

In some embodiments, the cells further comprise a third linker polynucleotide sequence localized between the first expression cassette and the second expression cassette.

In some embodiments, the third linker polynucleotide sequence is operably associated with the translation of the antigen receptor and each effector molecule as separate polypeptides.

In some embodiments, the third linker polynucleotide sequence encodes a 2A ribosome skipping tag. In some embodiments, the 2A ribosome skipping tag is selected from the group consisting of: P2A, T2A, E2A, and F2A. In some embodiments, the third linker polynucleotide sequence encodes an Internal Ribosome Entry Site (IRES). In some embodiments, the third linker polynucleotide sequence encodes a cleavable polypeptide. In some embodiments, the cleavable polypeptide comprises a furin polypeptide sequence.

In some embodiments, the third linker polynucleotide sequence is operably associated with the translation of the ACP and the antigen recognizing receptor as separate polypeptides.

In some embodiments, the antigen recognizing receptor recognizes an antigen selected from the group consisting of: 5T4, ADAMS, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1. In some embodiments, the antigen recognizing receptor recognizes GPC3. In some embodiments, the antigen recognizing receptor recognizes mesothelin (MSLN).

In some embodiments, the antigen recognizing receptor comprises an antigen-binding domain.

In some embodiments, the antigen-binding domain that binds to GPC3 comprises a heavy chain variable (VH) region and a light chain variable (VL) region, wherein the VH comprises: a heavy chain complementarity determining region 1 (CDR-H1) having the amino acid sequence of KNAMN (SEQ ID NO: 119), a heavy chain complementarity determining region 2 (CDR-H2) having the amino acid sequence of RIRNKTNNYATYYADSVKA (SEQ ID NO: 120), and a heavy chain complementarity determining region 3 (CDR-H3) having the amino acid sequence of GNSFAY (SEQ ID NO: 121), and wherein the VL comprises: a light chain complementarity determining region 1 (CDR-L1) having the amino acid sequence of KSSQSLLYSSNQKNYLA (SEQ ID NO: 122), a light chain complementarity determining region 2 (CDR-L2) having the amino acid sequence of WASSRES (SEQ ID NO: 123), and a light chain complementarity determining region 3 (CDR-L3) having the amino acid sequence of QQYYNYPLT (SEQ ID NO: 124).

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 125)
EVQLVETGGGMVQPEGSLKLSCAASGFTFNKNAMNWVRQAPGKGLEWVA
RIRNKTNNYATYYADSVKARFTISRDDSQSMLYLQMNNLKIEDTAMYYC
VAGNSFAYWGQGTLVTVSA
or
(SEQ ID NO: 126)
EVQLVESGGGLVQPGGSLRLSCAASGFTFNKNAMNWVRQAPGKGLEWVG
RIRNKTNNYATYYADSVKARFTISRDDSKNSLYLQMNSLKTEDTAVYYC
VAGNSFAYWGQGTLVTVSA.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 127)
DIVMSQSPSSLVVS
IGEKVTMTCKSSQSLLYSSNQKNYLAWYQQKPGQS
PKLLIYWASSRESGVPDRFTGSGSGTDFTLTISSV
KAEDLAVYYCQQYYNYPLTFGAGTKLELK,
or
(SEQ ID NO: 128)
DIVMTQSPDSLAVSLGE
RATINCKSSQSLLYSSNQKNYLAWYQQKPGQPPKL
LIYWASSRESGVPDRFSGSGSGTDFTLTISSLQAE
DVAVYYCQQYYNYPLTFGQGTKLEIK

In some embodiments, the antigen-binding domain that binds to MSLN comprises the three complementarity determining regions (CDRs) of a single-domain monoclonal antibody having the amino acid sequence of:

(SEQ ID NO: 129)
QVQLVESGGGTVQAGGSLK
LACAASGLPRTYNVMGWFRQAPGKEREGVAIIYTT
TGATYYRDSVKGRATISQDNAKKSVSLQMNSLRPE
DTAIYYCVARQPNSGPWEYWGQGTQVTVSS,
or
(SEQ ID NO: 130)
QVKLEESGGGSVQAGG
SLRLSCTTSGYTNSYKWMGWFRQAPGQEREGVAVI
YTGNDRTYYSDSVKGRFTISRDNAKNMIYLDMTRL
RPEDSAVYECAIGHDGAWRYWGQGTQVTVSS.

In some embodiments, the antigen-binding domain comprises an antibody, an antigen-binding fragment of an antibody, a F(ab) fragment, a F(ab′) fragment, a single chain variable fragment (scFv), or a single-domain antibody (sdAb).

In some embodiments, the antigen-binding domain comprises a single chain variable fragment (scFv).

In some embodiments, the scFv comprises a heavy chain variable domain (VH) and a light chain variable domain (VL).

In some embodiments, the VH and VL are separated by a peptide linker.

In some embodiments, the scFv comprises the structure VH-L-VL or VL-L-VH, wherein VH is the heavy chain variable domain, L is the peptide linker, and VL is the light chain variable domain.

In some embodiments, the antigen recognizing receptor is a chimeric antigen receptor (CAR) or T cell receptor (TCR).

In some embodiments, the antigen recognizing receptor is a CAR.

In some embodiments, the CAR comprises one or more intracellular signaling domains, and the one or more intracellular signaling domains are selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain.

In some embodiments, the CAR comprises a transmembrane domain, and the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceRlg transmembrane domain, and an NKG2D transmembrane domain.

In some embodiments, the CAR comprises a spacer region between the antigen-binding domain and the transmembrane domain.

In some embodiments, the ACP is a transcriptional modulator.

In some embodiments, the ACP is a transcriptional repressor.

In some embodiments, the ACP is a transcriptional activator.

In some embodiments, the ACP further comprises a repressible protease and one or more cognate cleavage sites of the repressible protease.

In some embodiments, the ACP further comprises a hormone-binding domain of estrogen receptor (ERT2 domain).

In some embodiments, the ACP is a transcription factor.

In some embodiments, the ACP is a zinc-finger-containing transcription factor.

In some embodiments, the transcription factor comprises a DNA-binding zinc finger protein domain (ZF protein domain) and an effector domain.

In some embodiments, the ZF protein domain is modular in design and is composed of zinc finger arrays (ZFA).

In some embodiments, the ZF protein domain comprises one to ten ZFA.

In some embodiments, the effector domain is selected from the group consisting of: a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator consisting of the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some embodiments, the one or more cognate cleavage sites of the repressible protease are localized between the ZF protein domain and the effector domain.

In some embodiments, the repressible protease is a hepatitis C virus (HCV) nonstructural protein 3 (NS3).

In some embodiments, the cognate cleavage site comprises an NS3 protease cleavage site.

In some embodiments, the NS3 protease cleavage site comprises a NS3/NS4A, a NS4A/NS4B, a NS4B/NS5A, or a NS5A/NS5B junction cleavage site.

In some embodiments, the NS3 protease can be repressed by a protease inhibitor.

In some embodiments, the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir. In some embodiments, the protease inhibitor is grazoprevir. In some embodiments, the protease inhibitor is grazoprevir and and elbasvir. In some embodiments, wherein the grazoprevir and the elbasvir is co-formulated in a pharmaceutical composition. In some embodiments, the pharmaceutical composition is a tablet. In some embodiments, the grazoprevir and the elbasvir are at a 2 to 1 weight ratio. In some embodiments, the grazoprevir is 100 mg per unit dose and the elbasvir is 50 mg per unit dose.

In some embodiments, the ACP is capable of undergoing nuclear localization upon binding of the ERT2 domain to tamoxifen or a metabolite thereof.

In some embodiments, the tamoxifen metabolite is selected from the group consisting of: 4-hydroxytamoxifen, N-desmethyltamoxifen, tamoxifen-N-oxide, and endoxifen.

In some embodiments, the ACP further comprises a degron, and wherein the degron is operably linked to the ACP.

In some embodiments, the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

In some embodiments, the degron comprises a cereblon (CRBN) polypeptide substrate domain capable of binding CRBN in response to an immunomodulatory drug (IMiD) thereby promoting ubiquitin pathway-mediated degradation of the ACP.

In some embodiments, the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

In some embodiments, the CRBN polypeptide substrate domain is a chimeric fusion product of native CRBN polypeptide sequences.

In some embodiments, the CRBN polypeptide substrate domain is a IKZF3/ZFP91/IKZF3 chimeric fusion product having the amino acid sequence of

(SEQ ID NO: 131)
FNVLMVHKRSHTGERPLQCEICGFTCRQKGNLLRHIKL
HTGEKPFKCHLCNYACQRRDAL

In some embodiments, the IMiD is an FDA-approved drug.

In some embodiments, the IMiD is selected from the group consisting of: thalidomide, lenalidomide, and pomalidomide.

In some embodiments, the degron is localized 5′ of the repressible protease, 3′ of the repressible protease, 5′ of the ZF protein domain, 3′ of the ZF protein domain, 5′ of the effector domain, or 3′ of the effector domain.

In some embodiments, the engineered nucleic acid further comprises an insulator.

In some embodiments, the insulator is localized between the first expression cassette and the second expression cassette.

In some embodiments, the first expression cassette is localized in the same orientation relative to the second expression cassette.

In some embodiments, the first expression cassette is localized in the opposite orientation relative to the second expression cassette.

In some embodiments, the cell further comprises a third expression cassette comprising a third promoter and a third exogenous polynucleotide sequence having the formula: (L-E)_{X} wherein E comprises a polynucleotide sequence encoding an effector molecule, L comprises a linker polynucleotide sequence, X=1 to 20, wherein the third promoter is operably linked to the third exogenous polynucleotide, and wherein for the first iteration of the (L-E) unit, L is absent.

In some embodiments, when the third expression cassette comprises two or more units of (L-E)_{X}, each L linker polynucleotide sequence is operably associated with the translation of each effector molecule as a separate polypeptide.

In some embodiments, each linker polynucleotide sequence encodes a 2A ribosome skipping tag.

In some embodiments, the 2A ribosome skipping tag is selected from the group consisting of: P2A, T2A, E2A, and F2A.

In some embodiments, each linker polynucleotide sequence encodes an Internal Ribosome Entry Site (IRES).

In some embodiments, the linker polynucleotide sequence encodes a cleavable polypeptide.

In some embodiments, the cleavable polypeptide comprises a furin polypeptide sequence.

In some embodiments, the third expression cassette comprising one or more units of (L-E)_{X} further comprises a polynucleotide sequence encoding a secretion signal peptide.

In some embodiments, for each X the corresponding secretion signal peptide is operably associated with the effector molecule.

In some embodiments, each secretion signal peptide comprises a native secretion signal peptide native to the corresponding effector molecule.

In some embodiments, each secretion signal peptide comprises a non-native secretion signal peptide that is non-native to the corresponding effector molecule.

In some embodiments, the non-native secretion signal peptide is selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, the additional promoter is a constitutive promoter, an inducible promoter, or a synthetic promoter.

In some embodiments, the additional promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the chemokine is selected from the group consisting of: CCL21a, CXCL10, CXCL11, CXCL13, a CXCL10-CXCL11 fusion protein, CCL19, CXCL9, and XCL1.

In some embodiments, the homing molecule is selected from the group consisting of: anti-integrin alpha4,beta7; anti-MAdCAM; CCR9; CXCR4; SDF1; MMP-2; CXCR1; CXCR7; CCR2; and GPR15.

In some embodiments, the growth factor is selected from the group consisting of: FLT3L and GM-CSF.

In some embodiments, the co-activation molecule is selected from the group consisting of: c-Jun, 4-1BBL, and CD40L.

In some embodiments, the tumor microenvironment modifier is selected from the group consisting of: adenosine deaminase, TGFbeta inhibitors, immune checkpoint inhibitors, VEGF inhibitors, and HPGE2.

In some embodiments, the TGFbeta inhibitors are selected from the group consisting of: an anti-TGFbeta peptide, an anti-TGFbeta antibody, a TGFb-TRAP, and combinations thereof.

In some embodiments, the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

In some embodiments, the VEGF inhibitors comprise anti-VEGF antibodies, anti-VEGF peptides, or combinations thereof.

In some embodiments, each effector molecule is a human-derived effector molecule.

In some embodiments, the cell is selected from the group consisting of: a T cell, a CD8+ T cell, a CD4+ T cell, a gamma-delta T cell, a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the cell is a Natural Killer (NK) cell.

In some embodiments, the cell is autologous.

In some embodiments, the cell is allogeneic.

In some embodiments, the cell is a tumor cell selected from the group consisting of: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.

In some embodiments, the cell was engineered via transduction with an oncolytic virus.

In some embodiments, the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

In some embodiments, the oncolytic virus is a recombinant oncolytic virus comprising the first expression cassette and the second expression cassette.

In some embodiments, the cell is a bacterial cell selected from the group consisting of: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

In another aspect, provided herein are compositions comprising the engineered cells, and a pharmaceutically acceptable carrier.

In another aspect, provided herein are methods of treating a subject in need thereof, the method comprising administering a therapeutically effective dose of any of the engineered cells or the compositions.

In another aspect, provided herein are methods of stimulating a cell-mediated immune response to a tumor cell in a subject, the method comprising administering to a subject having a tumor a therapeutically effective dose of any of the engineered cells or the compositions.

In another aspect, provided herein are methods of providing an anti-tumor immunity in a subject, the method comprising administering to a subject in need thereof a therapeutically effective dose of any of the engineered cells or the compositions.

In another aspect, provided herein are methods of treating a subject having cancer, the method comprising administering a therapeutically effective dose of any of the engineered cells or the compositions.

In another aspect, provided herein are methods of reducing tumor volume in a subject, the method comprising administering to a subject having a tumor a composition comprising any of the engineered cells or the compositions.

In some embodiments, the administering comprises systemic administration.

In some embodiments, the administering comprises intratumoral administration.

In some embodiments, the engineered cell is derived from the subject.

In some embodiments, the engineered cell is allogeneic with reference to the subject.

In some embodiments, the method further comprises administering a checkpoint inhibitor.

In some embodiments, the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.

In some embodiments, the method further comprises administering an anti-CD40 antibody.

In some embodiments, the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

In some embodiments, the method further comprises administering a protease inhibitor. In some embodiments, the protease inhibitor is administered in a sufficient amount to repress a repressible protease. In some embodiments, the protease inhibitor is administered prior to, concurrently with, subsequent to administration of the engineered cells or the composition comprising the engineered cells. In some embodiments, the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir. In some embodiments, the protease inhibitor is grazoprevir. In some embodiments, the protease inhibitor is grazoprevir and and elbasvir. In some embodiments, wherein the grazoprevir and the elbasvir is co-formulated in a pharmaceutical composition. In some embodiments, the pharmaceutical composition is a tablet. In some embodiments, the grazoprevir and the elbasvir are at a 2 to 1 weight ratio. In some embodiments, the grazoprevir is 100 mg per unit dose and the elbasvir is 50 mg per unit dose.

In some embodiments, the method further comprises administering tamoxifen or a metabolite thereof. In some embodiments, the tamoxifen metabolite is selected from the group consisting of: 4-hydroxytamoxifen, N-desmethyltamoxifen, tamoxifen-N-oxide, and endoxifen.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1A provides a diagram of an exemplary regulatable TF protein and TF inducible gene with a minCMV promoter and an mCherry gene

FIG. 1B shows expression of the mCherry protein in cells expressing both the regulatable TF and the mCherry vector in the presence or absence of asunaprevir.

FIG. 1C provides a diagram of an exemplary regulatable TF protein and regulatable TF inducible gene with a minYB_TATA promoter and an mCherry gene.

FIG. 1D shows expression of the mCherry protein in cells expressing both the regulatable TF and the mCherry vector in the presence or absence of asunaprevir.

FIG. 2A provides a diagram of an exemplary single vector expressing a regulatable TF and a regulatable TF inducible gene with a minTK promoter and an IL-10 gene.

FIG. 2B shows IL-10 production in cells expressing the regulatable TF and IL-10 vector in the presence or absence of asunaprevir.

FIG. 3A provides a diagram of an exemplary single vector expressing a regulatable TF and a regulatable TF inducible gene with a minTK promoter and an IL-12 gene.

FIG. 3B shows IL-12 production in cells expressing the regulatable TF and IL-12 vector in the presence or absence of asunaprevir.

FIG. 4A provides a diagram of an exemplary single vector expressing a regulatable TF linked to a myc-tagged CAR gene and a regulatable TF inducible gene with a minYB_TATA promoter and an mCherry gene.

FIG. 4B shows expression of the CAR in cells expressing the regulatable TF and mCherry vector in the presence and absence of asunaprevir.

FIG. 4C shows expression of mCherry in cells expressing the regulatable TF and mCherry vector in the presence or absence of asunaprevir.

FIG. 5 provides additional exemplary vectors for regulatable TF and effector gene expression in a single vector system.

FIG. 6A shows a schematic of a GPC3 CAR construct (“1106”).

FIG. 6B shows the CAR transduction profile in combination with various constructs as assessed by flow cytometry.

FIG. 7 shows the transduction profile of various constructs as assessed YFP MFI (top panel) and percentage (bottom panel).

FIG. 8A shows IL-12 production of various constructs with (right) or without (left) co-expression of a CAR.

FIG. 8B shows IL-15 production of various constructs with (right) or without (left) co-expression of a CAR.

FIG. 8C shows IL-21 production of various constructs with (right) or without (left) co-expression of a CAR.

FIG. 9A shows IL-12 production of various constructs with (bottom) or without (top) co-culturing with target HepG2 cells.

FIG. 9B shows IL-15 production of various constructs with (bottom) or without (top) co-culturing with target HepG2 cells.

FIG. 9C shows IL-21 production of various constructs with (bottom) or without (top) co-culturing with target HepG2 cells.

FIG. 10A shows TNFa production of various constructs with (bottom) or without (top) co-culturing with target HepG2 cells.

FIG. 10B shows IFNg production of various constructs with (bottom) or without (top) co-culturing with target HepG2 cells.

FIG. 10C shows IL-2 production of various constructs with (bottom) or without (top) co-culturing with target HepG2 cells.

FIG. 11A shows a schematic of a GPC3 CAR construct (“1108”).

FIG. 11B shows the CAR transduction profile in combination with various constructs as assessed by flow cytometry.

FIG. 12 shows tumor sizes as assessed by BLI measurement on days 11, 14, 21, and 24 for mice treated with T cells transduced with various constructs. FIG. 13C—IL-15; FIG. 13D—IL-12; FIG. 13E—IL-21)

FIG. 13A shows individual mice treated T cells without virus (FIG. 13A—left panel) or GPC3-CAR T alone without a cytokine (FIG. 13A—right panel).

FIG. 13B shows individual mice treated with GPC3-CAR T engineered with an IL-12/IL-21 co-expression armoring.

FIG. 13C shows individual mice treated with GPC3-CAR T engineered with an IL-15 armoring.

FIG. 13D shows individual mice treated with GPC3-CAR T engineered with an IL-12 armoring.

FIG. 13E shows individual mice treated with GPC3-CAR T engineered with an IL-21 armoring.

FIG. 14A shows production of TNFa as assessed in plasma of mice treated with various constructs at Day 3 (top panel) and Day 13 (bottom panel) post treatment.

FIG. 14B shows production of IFNγ as assessed in plasma of mice treated with various constructs at Day 3 (top panel) and Day 13 (bottom panel) post treatment.

FIG. 14C shows production of IL-2 as assessed in plasma of mice treated with various constructs at Day 3 (top panel) and Day 13 (bottom panel) post treatment.

FIG. 15A shows production of IL-12 as assessed in plasma of mice treated with various constructs at Day 3 (top panel) and Day 10 (bottom panel) post treatment.

FIG. 15B shows production of IL-15 as assessed in plasma of mice treated with various constructs at Day 3 (top panel) and Day 10 (bottom panel) post treatment.

FIG. 15C shows production of IL-21 as assessed in plasma of mice treated with various constructs at Day 3 (top panel) and Day 10 (bottom panel) post treatment.

FIG. 16A shows tumor size (left panel) and human T cell persistence (right panel) for T cells engineered with various constructs at day 14 post tumor injection (Day 3 post T cell treatment).

FIG. 16B shows tumor size (left panel) and human T cell persistence (right panel) for T cells engineered with various constructs at day 21 post tumor injection (Day 13 post T cell treatment)

FIG. 17A shows schematics of various payload expression systems using a Tamoxifen-based regulatable TF expression system.

FIG. 17B shows reporter expression using various Tamoxifen-based regulatable TF expression systems following treatment with different amounts of 4-OHT.

FIG. 17C shows reporter expression using various Tamoxifen-based regulatable TF expression systems following treatment with different amounts of N-desmethyltamoxifen.

FIG. 17D shows reporter expression using various Tamoxifen-based regulatable TF expression systems following treatment with different amounts of endoxifen.

FIG. 17E shows a summary of reporter expression using various Tamoxifen-based regulatable TF expression systems following treatment.

FIG. 18 shows a summary of CAR expression using various Tamoxifen-based regulatable TF expression systems following treatment.

FIG. 19 shows flow cytometry plots of CAR expression using various Tamoxifen-based regulatable TF expression systems following treatment

FIG. 20 shows schematics of various payload expression systems using drug-inducible ACP-based regulatable TF expression systems.

FIG. 21 shows schematics of various payload expression systems using drug-inducible ACP-based regulatable TF expression systems.

FIG. 22 shows CAR expression using various drug-inducible ACP-based regulatable TF expression systems.

FIG. 23A shows reporter expression using a specific drug-inducible ACP-based regulatable TF expression system.

FIG. 23B shows CAR expression using a specific drug-inducible ACP-based regulatable TF expression system.

FIG. 24A shows reporter expression using a specific drug-inducible ACP-based regulatable TF expression system.

FIG. 24B shows CAR expression using a specific drug-inducible ACP-based regulatable TF expression system.

FIG. 25 shows a schematic of an ACP for drug-inducible formats (also referred to as “synTF”) using an NS3/NS4 protease cleavage site and a VPR transcriptional effector domain (Construct “1845”) as well as the expression cassette using a 4× BS minYB-TATA ACP-responsive promoter driving hIL-12 effector molecule payload.

FIG. 26 shows in vitro production of hIL-12 using an ACP-based regulatable expression system. Columns are no drug, 0.1 μM GRZ, and 0.5 μM GRZ from left to right, respectively.

FIG. 27 shows the experimental design used to assessed ACP-based regulatable expression systems in vivo.

FIG. 28 shows fold-expansion of T cells engineered with a constitutive hIL-12 expression system or an ACP-based regulatable expression system in vivo.

FIG. 29 shows the glucose profile of T cells engineered with a constitutive hIL-12 expression system or an ACP-based regulatable expression system in vivo.

FIG. 30 shows the percentage of circulating T cells engineered with a constitutive hIL-12 expression system or an ACP-based regulatable expression system in vivo.

FIG. 31 shows the production of hIL-12 in plasma produced by T cells engineered with a constitutive hIL-12 expression system or an ACP-based regulatable expression system in vivo.

FIG. 32 shows schematics of various payload expression systems using drug-inducible ACP-based regulatable TF expression systems.

FIG. 33 shows in vitro production of hIL-12 by T cells transduced with various ACP-based regulatable expression systems following treatment with various concentrations of grazoprevir.

FIG. 34 shows in vitro production of hIL-15 by T cells transduced with various ACP-based regulatable expression systems following treatment with various concentrations of grazoprevir.

FIG. 35 shows CAR expression in T cells transduced with various drug-inducible ACP-based regulatable TF expression systems.

FIG. 36 shows CAR activity using various drug-inducible ACP-based regulatable TF expression systems as assessed by target cell killing (LDH release).

FIG. 37 shows in vitro production of hIL-12 by NK cells transduced with various ACP-based regulatable expression systems following treatment with various concentrations of grazoprevir.

FIG. 38 shows schematics of various payload expression systems using drug-inducible ACP-based regulatable TF expression systems.

FIG. 39 shows in vitro production of hIL-12 by T cells transduced with various ACP-based regulatable expression systems following treatment with various concentrations of grazoprevir.

FIG. 40 shows in vitro production of hIL-15 by T cells transduced with various ACP-based regulatable expression systems following treatment with various concentrations of grazoprevir.

FIG. 41 shows in vitro production of hIL-15 by T cells transduced with various ACP-based regulatable expression systems following treatment with various concentrations of grazoprevir.

FIG. 42 shows the T cells in the blood (hCD3+:hCD45+ shown as % of live cells) engineered with a constitutive hIL-12 expression system or a drug-inducible ACP-based regulatable expression system in vivo over a time course (Day 4 top panel; Day 8 middle panel; Day 12 bottom panel) for various grazoprevir dosing regimens. *Samples of 2 groups were lost and not included in the analysis.

FIG. 43 shows the production of hIL-12 in plasma produced by T cells engineered with a constitutive hIL-12 expression system or a drug-inducible ACP-based regulatable expression system in vivo for various grazoprevir dosing regimens at Day 4.

FIG. 44 shows the production of hIL-12 in plasma produced by T cells engineered with a constitutive hIL-12 expression system or a drug-inducible ACP-based regulatable expression system in vivo for various grazoprevir dosing regimens at Days 8 (left panel) and 12 (right panel).

FIG. 45 shows an “on/off/on” Grazoprevir (Grz) dosing regimen.

FIG. 46 shows the T cells in the blood (hCD3+:hCD45+ shown as % of live cells) engineered with a constitutive hIL-12 expression system or a drug-inducible ACP-based regulatable expression system in vivo over a time course at the indicated days for an “on/off/on” Grazoprevir (Grz) dosing regimen.

FIG. 47 shows the production of hIL-12 in plasma produced by T cells engineered with a constitutive hIL-12 expression system or a drug-inducible ACP-based regulatable expression system in vivo over a time course at the indicated days for an “on/off/on” Grazoprevir (Grz) dosing regimen.

FIG. 48 shows body weight of mice administered T cells engineered with a constitutive hIL-12 expression system or a drug-inducible ACP-based regulatable expression system in vivo over a time course at the indicated days for an “on/off/on” Grazoprevir (Grz) dosing regimen.

FIG. 49 presents a workflow for a screen directed to assessing promoters that turn on transcription when CAR cells are activated by target cells.

FIG. 50 presents the constructs and candidate promoters assessed in a screen directed to promoters that turn on transcription when CAR cells are activated by target cells.

FIG. 51 shows quantified mKate expression by flow-cytometry for constructs and candidate promoters assessed in a screen directed to promoters that turn on transcription when CAR cells are activated by target cells at 24 hours following culturing with (right column) or without (left column) HepG2 target cells.

FIG. 52 shows quantified mKate expression by flow-cytometry for constructs and candidate promoters assessed in a screen directed to promoters that turn on transcription when CAR cells are activated by target cells at 48 hours following culturing with (right column) or without (left column) HepG2 target cells

FIG. 53 shows histograms of mKate expression by flow-cytometry for identified promoters that turn on transcription when CAR cells are activated by target cells at 24 hours (top panel) and 48 hours (bottom panel) following culturing with HepG2 target cells (“Promoter+Target”). Also shown is CAR only cultured without HepG2 targets (“CAR only”), CAR only cultured with HepG2 targets (“CAR+Target”), CAR+promoter cultured without HepG2 targets (“Promoter Only”).

FIG. 54 shows in vitro production of hIL-12 by T cells transduced with various ACP-based regulatable expression systems following treatment with various concentrations of grazoprevir with or without elbasvir.

### DETAILED DESCRIPTION

### Definitions

Terms used in the claims and specification are defined as set forth below unless otherwise specified.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Engineered Nucleic Acids and Polypeptides

Regulation of drug expression in cell therapies is required to hit therapeutic efficacy windows. Such methods are described herein using a regulatable transcription factor that can drive expression of any desirable effector molecule or combination of effector molecules. This system is versatile as it can regulate intracellular or membrane bound proteins by using, for example, a modular protease system that enables ON or OFF configurations. It can use protease switch drugs that are FDA approved and can be administered via oral delivery with a favorable pharmacokinetic profile. In addition, the methods and compositions described herein may be used, e.g., for regulated immunomodulatory effector expression in cell or gene therapies. The regulatable transcription factor can be used in conjunction with, e.g., CAR T cells, CAR NK cells, TCR T cells, TIL therapies, viral-specific T cells, or any other appropriate immune cell therapy.

In one aspect, provided herein are engineered nucleic acids comprising: a first expression cassette comprising a first promoter and a first exogenous polynucleotide sequence encoding an activation-conditional control polypeptide (ACP) and/or an antigen recognizing receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and optionally wherein the ACP is capable of inducing expression of the first expression cassette by binding to the ACP-responsive promoter. In some embodiments, the ACP includes a drug-inducible domain, such as a tetracycline responsive domain (e.g., a TetR domain) or a repressible protease domain (e.g., an NS3 protease). In some embodiments, the ACP is an antigen recognizing receptor and the receptor is capable of inducing expression of the second expression cassette following binding to its cognate antigen (“activation inducible system”), such as a CAR binding to a cognate antigen and the ACP-responsive promoter includes a promoter sequence capable of driving expression of the second expression cassette in response to CAR signaling.

In one aspect, provided herein are engineered nucleic acids comprising: a first expression cassette comprising: (a) a first promoter and a first exogenous polynucleotide sequence encoding an activation-conditional control polypeptide (ACP) and an antigen recognizing receptor, wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.

In one aspect, provided herein are engineered nucleic acids comprising: a first expression cassette comprising a first promoter and a first exogenous polynucleotide sequence encoding an antigen recognizing receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent. Expression of the second expression cassette can be induced by an ACP binding to the ACP-responsive promoter. An ACP can be a receptor, such as the antigen recognizing receptor, can induce expression of the second expression cassette upon ACP binding to a cognate ligand (e.g., a cognate antigen), such as downstream signaling following ligand binding inducing expression from an ACP-responsive promoter. In a non-limiting illustrative example, an ACP can be a chimeric antigen receptor (CAR), and upon CAR binding to a cognate receptor, downstream signaling (e.g., T cell or NK cell receptor signaling) can induce expression of a cytokine payload (e.g., cytokine armoring) from an ACP-responsive promoter that is specific to CAR binding of a target antigen. Examples of ACP-responsive promoters useful for in activation inducible systems are described below (see “Promoters”).

In some embodiments, when the second expression cassette comprises two or more units of (L-E)_{X}, each linker polynucleotide sequence is operably associated with the translation of each molecule as a separate polypeptide.

X can be 1, 2, 3, 4, 5, 6, 7, 8, 9, 10, 11, 12, 13, 14, 15, 16, 17, 18, 19, 20, or more.

In some embodiments, a single engineered nucleic acid comprises at least one, two, three four, five, or more expression cassettes. In general, each expression cassette refers to a promoter operably linked to a polynucleotide sequence encoding protein of interest. For example, each of an ACP, an effector molecule, and an antigen recognizing receptor can be encoded by a separate expression cassette on the same engineered nucleic acid (e.g., vector). The expression cassettes can be oriented in any direction relative to each other (e.g., the cassettes can be in the same orientation or the opposite orientation). In exemplary engineered nucleic acids with three or more expression cassettes the cassettes can be in the same orientation or a mixed orientation (e.g., the first and second cassette can be in the same orientation while the third cassette is in the opposite orientation). In some embodiments, the first expression cassette is localized in the same orientation relative to the second expression cassette. In some embodiments, the first expression cassette is localized in the opposite orientation relative to the second expression cassette.

In some embodiments, one or more engineered nucleic acids can comprise at least one, two, three four, five, or more expression cassettes. Strategies for regulated armoring including two or more engineered nucleic acids can be referred to as an “engineered expression system.” In one aspect, engineered expression systems are provided herein that include (a) a first expression cassette comprising a first promoter and a first exogenous polynucleotide sequence encoding an activation-conditional control polypeptide (ACP), wherein the first promoter is operably linked to the first exogenous polynucleotide; and (2) a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. In some embodiments of the expression system, the first expression cassette and the second expression cassette are encoded by separate polynucleotide sequences. In some embodiments of the expression system, the first expression cassette and the second expression cassette are encoded by the same polynucleotide sequence. In some embodiments of the expression system, the first expression cassette and/or the second expression cassette further includes an additional exogenous polynucleotide sequence encoding an antigen recognizing receptor. In some embodiments of the expression system, the first expression cassette further includes an additional exogenous polynucleotide sequence encoding an antigen recognizing receptor. In some embodiments of the expression system, the second expression cassette further includes an additional exogenous polynucleotide sequence encoding an antigen recognizing receptor. In some embodiments of the expression system, the engineered expression system further includes an additional expression cassette including an additional promoter and an additional exogenous polynucleotide sequence encoding an antigen recognizing receptor, wherein the additional promoter is operably linked to the additional exogenous polynucleotide. In some embodiments of the expression system, the additional exogenous polynucleotide sequence is encoded by the same polynucleotide as the first expression cassette or the second expression cassette. In some embodiments of the expression system, the additional exogenous polynucleotide sequence is encoded by the same polynucleotide as the first expression cassette. In some embodiments of the expression system, the additional exogenous polynucleotide sequence is encoded by the same polynucleotide as the second expression cassette. In some embodiments of the expression system, a first vector includes the first expression cassette and the additional expression cassette if present, and a second vector includes the second expression cassette. In some embodiments of the expression system, a first vector includes the first expression cassette, and a second vector includes the second expression cassette and the the additional expression cassette if present. In some embodiments of the expression system, a first vector includes the first expression cassette and the second expression cassette, and a second vector includes the additional expression cassette if present.

As illustrative non-limiting examples of expression systems, (1) an antigen recognizing receptor expression cassette and an effector molecule expression cassette can be encoded by a first engineered nucleic acid, and an ACP expression cassette can be encoded by a second engineered nucleic acid; (2) an ACP expression cassette and an effector molecule expression cassette can be encoded by a first engineered nucleic acid, and an antigen recognizing receptor expression cassette can be encoded by a second engineered nucleic acid; (3) an ACP expression cassette and an antigen recognizing receptor expression cassette can be encoded by a first engineered nucleic acid, and an effector molecule expression cassette can be encoded by a second engineered nucleic acid. In an additional illustrative non-limiting example, an effector molecule expression cassette can be encoded by a first engineered nucleic acid, and an ACP expression cassette can be encoded by a second engineered nucleic acid.

In some embodiments, expression cassettes can be multicistronic, i.e., more than one separate polypeptide (e.g., multiple exogenous polynucleotides or effector molecules) can be produced from a single mRNA transcript. For example, a multicistronic expression cassette can encode both an ACP and antigen recognizing receptor, e.g., both expressed from a single expression cassette driven by a constitutive promoter. In another example, a multicistronic expression cassette can encode both an effector molecule and an antigen recognizing receptor, e.g., both expressed from a single expression cassette driven by an ACP-responsive promoter. Expression cassettes can be multicistronic through the use of various linkers, e.g., a polynucleotide sequence encoding a first protein of interest can be linked to a nucleotide sequence encoding a second protein of interest, such as in a first gene:linker:second gene 5′ to 3′ orientation. Multicistronic features and options are described in the section “Multicistronic and Multiple Promoter Systems.”

In some embodiments, the engineered nucleic acid is selected from: a DNA, a cDNA, an RNA, an mRNA, and a naked plasmid. Also provided herein is an expression vector comprising the engineered nucleic acid.

In some embodiments, the engineered nucleic acid further comprises an insulator. The insulator can be localized between the first expression cassette and the second expression cassette. The insulator can be localized between the first expression cassette and the second expression cassette where both cassettes are in the same orientation relative to one another. The insulator can be localized between the first expression cassette and the second expression cassette where the cassettes are in the opposite orientation relative to one another. An insulator is a cis-regulatory element that has enhancer-blocking or barrier function. Enhancer-blocker insulators block enhancers from acting on the promoter of nearby genes. Barrier insulators prevent euchromatin silencing. An example of a suitable insulator of the present disclosure is the A2 insulator as described in Liu M, et al., Nat Biotechnol. 2015 February; 33(2):198-203. Additional insulators are described in West et al, Genes & Dev, 002. 16: 271-288, both of which are incorporated by reference in their entirety. Other examples of suitable insulators include, without limitation, an A1 insulator, a CTCF insulator, a gypsy insulator, an HS5 insulator, and a β-globin locus insulator, such as cHS4. In some embodiments, the insulator is an A2 insulator, an A1 insulator, a CTCF insulator, an HS5 insulator, a gypsy insulator, a β-globin locus insulator, or a cHS4 insulator. The insulator can be an A2 insulator.

Activation-Conditional Control Polypeptide (ACP)

In some embodiments, the ACP is a transcriptional modulator. In some embodiments, the ACP is a transcriptional repressor. In some embodiments, the ACP is a transcriptional activator. In some embodiments, the ACP is a transcription factor. In some embodiments, the ACP comprises a DNA-binding domain and a transcriptional effector domain. In some embodiments, the transcription factor is a zinc-finger-containing transcription factor. In some embodiments, the zinc-finger-containing transcription factor may be a synthetic transcription factor. In some embodiments, the ACP DNA-binding domain comprises a DNA-binding zinc finger protein domain (ZF protein domain) and an effector domain. In some embodiments, the DNA-binding domain comprises a tetracycline (or derivative thereof) repressor (TetR) domain. In some embodiments, the ACP is an antigen recognizing receptor of the present disclosure.

Zinc Finger Protein Domain

In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

An exemplary ZF protein domain is shown in the sequence

(SEQ ID NO: 88)
SRPGERPFQCRICMRNFSRRHGLDRHTRTHTGEK
PFQCRICMRNFSDHSSLKRHLRTHTGSQKPFQCRI
CMRNFSVRHNLTRHLRTHTGEKPFQCRICMRNFSD
HSNLSRHLKTHTGSQKPFQCRICMRNFSQRSSLVR
HLRTHTGEKPFQCRICMRNFSESGHLKRHLRTHLR
GS.

ACP Effector Domain

The ACP can also further comprise an effector domain, such as a transcriptional effector domain. For instance, a transcriptional effector domain can be the effector or activator domain of a transcription factor. Transcription factor activation domains are also known as transactivation domains, and act as scaffold domains for proteins such as transcription coregulators that act to activate or repress transcription of genes. Any suitable transcriptional effector domain can be used in the ACP including, but not limited to, a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, or any combination thereof.

In some embodiments, the effector domain is a transcription effector domain selected from: a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

Exemplary transcription effector domain protein sequences are shown in Table 8. Exemplary transcription effector domain nucleotide sequences are shown in Table 9.

TABLE 8
Transcriptional Effector Domain (Protein)

| | SEQ | |
|---|---|---|
| | ID | |
| Amino Acid Sequence | NO: | Description |
| RTLVTFKDVFVDFTREEWKL | 96 | KRAB |
| LDTAQQIVYRNVMLENYKNL | | |
| VSLGYQLTKPDVILRLEKGE | | |
| EPWLV | | |
| RTLVTFKDVFVDFTREEWKL | 97 | truncated KRAB |
| LDTAQQIVYRNVMLENYKNL | | (min KRAB) |
| VSLGY | | |
| EASGSGRADALDDFDLDMLG | 90 | VPR activation |
| SDALDDFDLDMLGSDALDDF | | domain |
| DLDMLGSDALDDFDLDMLIN | | |
| SRSSGSPKKKRKVGSQYLPD | | |
| TDDRHRIEEKRKRTYETFKS | | |
| IMKKSPFSGPTDPRPPPRRI | | |
| AVPSRSSASVPKPAPQPYPF | | |
| TSSLSTINYDEFPTMVFPSG | | |
| QISQASALAPAPPQVLPQAP | | |
| APAPAPAMVSALAQAPAPVP | | |
| VLAPGPPQAVAPPAPKPTQA | | |
| GEGTLSEALLQLQFDDEDLG | | |
| ALLGNSTDPAVFTDLASVDN | | |
| SEFQQLLNQGIPVAPHTTEP | | |
| MLMEYPEAITRLVTGAQRPP | | |
| DPAPAPLGAPGLPNGLLSGD | | |
| EDFSSIADMDFSALLGSGSG | | |
| SRDSREGMFLPKPEAGSAIS | | |
| DVFEGREVCQPKRIRPFHPP | | |
| GSPWANRPLPASLAPTPTGP | | |
| VHEPVGSLTPAPVPQPLDPA | | |
| PAVTPEASHLLEDPDEETSQ | | |
| AVKALREMADTVIPQKEEAA | | |
| ICGQMDLSHPPPRGHLDELT | | |
| TTLESMTEDLNLDSPLTPEL | | |
| NEILDTFLNDECLLHAMHIS | | |
| TGLSIFDTSLF | | |

TABLE 9
Transcriptional Effector Domain (Nucleotide)

| Nucleic Acid Sequence | SEQ ID NO: | Description |
|---|---|---|
| AGAACCCTGGTCACCTTCAA | 98 | KRAB |
| GGACGTGTTCGTGGACTTCA | | |
| CCCGGGAAGAGTGGAAGCTG | | |
| CTGGATACAGCCCAGCAGAT | | |
| CGTGTACCGGAACGTGATGC | | |
| TGGAAAACTACAAGAATCTG | | |
| GTGTCCCTGGGCTACCAGCT | | |
| GACCAAGCCTGACGTGATCC | | |
| TGCGGCTGGAAAAGGGCGAA | | |
| GAACCTTGGCTGGTG | | |
| AGAACCCTGGTCACCTTCAA | 99 | truncated KRAB |
| GGACGTGTTCGTGGACTTCA | | (min KRAB) |
| CCCGGGAAGAGTGGAAGCTG | | |
| CTGGATACAGCCCAGCAGAT | | |
| CGTGTACCGGAACGTGATGC | | |
| TGGAAAACTACAAGAATCTG | | |
| GTGTCCCTGGGCTAC | | |

Drug-Inducible Domains

In some embodiments, the ACP is a small molecule (e.g., drug) inducible polypeptide. For example, in some embodiments, the ACP may be induced by tetracycline (or derivative thereof), and comprises a TetR domain and a VP16 effector domain. In some embodiments, the ACP may be induced by tamoxifen, or a metabolite thereof, such as 4-hydroxy-tamoxifen (4-OHT), and comprises an estrogen receptor variant, such as ERT2. In some embodiments, the ACP is a small molecule (e.g., drug) inducible polypeptide that comprises a repressible protease and one or more cognate cleavage sites of the repressible protease.

The term “repressible protease” as used herein, refers to a protease that can be inactivated by the presence or absence of a specific agent (e.g., that binds to the protease). In some embodiments, a repressible protease is active (cleaves a cognate cleavage site) in the absence of the specific agent and is inactive (does not cleave a cognate cleavage site) in the presence of the specific agent. In some embodiments, the specific agent is a protease inhibitor. In some embodiments, the protease inhibitor specifically inhibits a given repressible protease of the present disclosure.

Non-limiting examples of repressible proteases include hepatitis C virus proteases (e.g., NS3 and NS2-3); signal peptidase; proprotein convertases of the subtilisin/kexin family (furin, PCI, PC2, PC4, PACE4, PC5, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N; insulin degrading enzyme; calpain; high molecular weight protease; and, caspases 1, 2, 3, 4, 5, 6, 7, 8, and 9. Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma; angiotensin converting enzyme secretase; TGF alpha secretase; T F alpha secretase; FAS ligand secretase; TNF receptor-I and -II secretases; CD30 secretase; KL1 and KL2 secretases; IL6 receptor secretase; CD43, CD44 secretase; CD 16-1 and CD 16-11 secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F, G, and H. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321: 265-279 (1997); Z. Werb, Cell 91: 439-442 (1997); T. G. Wolfsberg et al., J. Cell Biol. 131: 275-278 (1995); K. Murakami and J. D. Etlinger, Biochem. Biophys. Res. Comm. 146: 1249-1259 (1987); T. Berg et al., Biochem. J. 307: 313-326 (1995); M. J. Smyth and J. A. Trapani, Immunology Today 16: 202-206 (1995); R. V. Talanian et al., J. Biol. Chem. 272: 9677-9682 (1997); and N. A. Thomberry et al., J. Biol. Chem. 272: 17907-17911 (1997), the disclosures of which are incorporated herein.

The term “cognate cleavage site” as used herein, refers to a specific sequence or sequence motif recognized by and cleaved by the repressible protease. A cleavage site for a protease includes the specific amino acid sequence or motif recognized by the protease during proteolytic cleavage and typically includes the surrounding one to six amino acids on either side of the scissile bond, which bind to the active site of the protease and are used for recognition as a substrate.

Other proteases, including those listed above and in Table 1, can be used. When a protease is selected, its cognate cleavage site and protease inhibitors known in the art to bind and inhibit the protease can be used in a combination. Exemplary combinations for the use are provided below in Table 1. Representative sequences of the proteases are available from public database including UniProt through the uniprot.org website. UniProt accession numbers for the proteases are also provided below in Table 1.

TABLE 1

| Protease | | |
|---|---|---|
| (UniProt Accession Number or | | |
| SEQ ID NO.) | Cognate cleavage site | Protease inhibitors |
| HCVNS4A/4B | DEMEECSQHL | Simeprevir, Danoprevir, |
| | (SEQ ID NO: 81) | Asunaprevir, Ciluprevir, |
| | EDVVPCSMG | Boceprevir, Sovaprevir, |
| | (SEQ ID NO: 82) | Paritaprevir, Telaprevir, |
| | | Grazoprevir |
| HCVNS5A/5B | DEMEECSQHL | Simeprevir, Danoprevir, |
| | (SEQ ID NO: 81) | Asunaprevir, Ciluprevir, |
| | EDVVPCSMG | Boceprevir, Sovaprevir, |
| | (SEQ ID NO: 82) | Paritaprevir, Telaprevir, |
| | | Grazoprevir |
| HCVNS3 | DEMEECSQHL | Simeprevir, Danoprevir, |
| | (SEQ ID NO: 81) | Asunaprevir, Ciluprevir, |
| | EDVVPCSMG | Boceprevir, Sovaprevir, |
| | (SEQ ID NO: 82) | Paritaprevir, Telaprevir, |
| | | Grazoprevir |
| HCVNS2-3 | DEMEECSQHL | Simeprevir, Danoprevir, |
| | (SEQ ID NO: 81) | Asunaprevir, Ciluprevir, |
| | EDVVPCSMG | Boceprevir, Sovaprevir, |
| | (SEQ ID NO: 82) | Paritaprevir, Telaprevir, |
| | | Grazoprevir |
| HIV-1 protease | | Amprenavir, Atazanavir, |
| | | Darunavir, |
| | | Fosamprenavir, |
| | | Indinavir, Lopinavir, |
| | | Nelfinavir, Ritonavir, |
| | | Saquinavir, Tipranavir |
| Signal peptidase (P67812, | preference of eukaryotic signal peptidase for cleavage | |
| P15367, P00804, P0803) | after residue 20 (Xaa^{20↓}) of pre(Apro)apoA-II: Ala, Cys | |
| | > Gly > Ser, Thr > Pro > Asn, Val, Ile, Leu, Tyr, His, | |
| | Arg, Asp. | |
| proprotein convertases cleaving | (R/K)-X-(hydrophobic)-X↓, where X is any amino acid | |
| at hydrophobic residues (e.g., | | |
| Leu, Phe, Val, or Met) (Q16549, | | |
| Q8NBP7, Q92824, P29120, | | |
| Q6UW60, P29122, Q9QXV0) | | |
| proprotein convertases cleaving | (K/R)-(X)n-(K/R)↓, where n is 0, 2, 4 or 6 and X is any | |
| at small amino acid residues | amino acid | |
| such as Ala or Thr (Q16549, | | |
| Q8NBP7, Q92824, P29120, | | |
| Q6UW60, P29122) | | |
| proopiomelanocortin converting | Cleavage at paired basic residues in certain | |
| enzyme (PCE) (Q9UO77615, | prohormones, either between them, or on the carboxyl | |
| 0776133) | side | |
| chromaffin granule aspartic | tends to cleave dipeptide bonds that have hydrophobic | |
| protease (CGAP) | residues as well as a beta-methylene group | |
| prohormone thiol protease | | |
| (cathepsin L1) (P07154, P07711, | | |
| P06797, P25975, Q28944) | | |
| carboxypeptidases (e.g., | cleaves a peptide bond at the carboxy-terminal (C- | |
| carboxypeptidase E/H, | terminal) end of a protein or peptide | |
| carboxypeptidase D and | | |
| carboxypeptidase Z)(Q9M099, | | |
| P15169, Q04609, P08819, | | |
| P08818, 077564, P70627, | | |
| O35409, P07519, Q8VZU3, | | |
| P22792, P15087, P16870, | | |
| Q9JHH6, Q96IY4, Q7L8A9) | | |
| aminopeptidases (e.g., arginine | cleaves a peptide bond at the amino-terminal (N- | |
| aminopeptidase, lysine | terminal) end of a protein or peptide | |
| aminopeptidase, aminopeptidase | | |
| B) | | |
| prolyl endopeptidase (Q12884, | Hydrolysis of Pro-\|-Xaa >> Ala-\|-Xaa in oligopeptides. | |
| P48147, P97321, Q4J6C6) | Release of anN-terminal dipeptide, Xaa-Yaa-\|-Zaa-, | |
| | from a polypeptide, preferentially when Yaa is Pro, | |
| | provided Zaa is neither Pro nor hydroxyproline | |
| aminopeptidase N (P97449, | Release of anN-terminal amino acid, Xaa-\|-Yaa- from | |
| P15144, P15145, P15684) | a peptide, amide or arylamide. Xaa is preferably Ala, | |
| | but may be most amino acids including Pro (slow | |
| | action). When a terminal hydrophobic residue is | |
| | followed by a prolyl residue, the two may be released | |
| | as an intact Xaa-Pro dipeptide | |
| insulin degrading enzyme | Degradation of insulin, glucagon and other | |
| (P14735, P35559, Q9JHR7, | polypeptides. No action on proteins. | |
| P22817, Q24K02) | Cleaves multiple short polypeptides that vary | |
| | considerably in sequence | |
| Calpain (O08529, P17655, | No specific amino acid sequence is uniquely | |
| Q07009, Q27971, P20807, | recognized by calpains. Amongst protein substrates, | |
| P07384, O35350, O14815, | tertiary structure elements rather than primary amino | |
| P04632, Q9Y6Q1, O15484, | acid sequences appear to be responsible for directing | |
| Q9HC96, A6NHC0, Q9UMQ6) | cleavage to a specific substrate. Amongst peptide and | |
| | small-molecule substrates, the most consistently | |
| | reported specificity is for small, hydrophobic amino | |
| | acids (e.g., leucine, valine and isoleucine) at the P2 | |
| | position, and large hydrophobic amino acids (e.g., | |
| | phenylalanine and tyrosine) at the P1 position. One | |
| | fluorogenic calpain substrate is (EDANS)-Glu-Pro- | |
| | Leu-Phe═Ala-Glu-Arg-Lys-(DABCYL), with cleavage | |
| | occurring at the Phe=Ala bond. | |
| caspase 1 (P29466, P29452) | Strict requirement for an Asp residue at position P1 and | |
| | has a preferred cleavage sequence of Tyr-Val-Ala-Asp- | |
| | \|-. | |
| caspase 2 (P42575, P29594) | Strict requirement for an Asp residue at P1, with 3 16- | |
| | asp being essential for proteolytic activity and has a | |
| | preferred cleavage sequence of Val-Asp-Val-Ala-Asp- | |
| | \|-. | |
| caspase 3 (P42574, P70677) | Strict requirement for an Asp residue at positions Pl | |
| | and P4. It has a preferred cleavage sequence of Asp- | |
| | Xaa-Xaa-Asp-\|- with a hydrophobic amino-acid residue | |
| | at P2 and a hydrophilic amino-acid residue at P3, | |
| | although Val or Ala are also accepted at this position. | |
| caspase 4 (P70343, P49662) | Strict requirement for Asp at the P1 position. It has a | |
| | preferred cleavage sequence of Tyr-Val-Ala-Asp-\|- but | |
| | also cleaves at Asp-Glu-Val-Asp-\|- | |
| caspase 5 (P51878) | Strict requirement for Asp at the P1 position. It has a | |
| | preferred cleavage sequence of Tyr-Val-Ala-Asp-\|- but | |
| | also cleaves at Asp-Glu-Val-Asp-\|-. | |
| caspase 6 (P55212) | Strict requirement for Asp at position P1 and has a | |
| | preferred cleavage sequence of Val-Glu-His-Asp-\|-. | |
| caspase 7 (P97864, P55210) | Strict requirement for an Asp residue at position P1 and | |
| | has a preferred cleavage sequence of Asp-Glu-Val- | |
| | Asp-\|-. | |
| caspase 8 (Q8IRY7, O89110, | Strict requirement for Asp at position P1 and has a | |
| Q14790) | preferred cleavage sequence of (Leu/Asp/Val)-Glu- | |
| | Thr-Asp-\|-(Gly/Ser/Ala). | |
| caspase 9 (P55211, Q8C3Q9, | Strict requirement for an Asp residue at position P1 and | |
| Q5IS54) | with a marked preference for His at position P2. It has | |
| | a preferred cleavage sequence of Leu-Gly-His-Asp-\|- | |
| | Xaa. | |
| caspase 10 (Q92851) | Strict requirement for Asp at position P1 and has a | |
| | preferred cleavage sequence of Leu-GIn-Thr-Asp-\|- | |
| | Gly. | |
| puromycin sensitive | Release of anN-terminal amino acid, preferentially | |
| aminopeptidase (P55786, | alanine, from a wide range of peptides, amides and | |
| Q11011) | arylamides. | |
| angiotensin converting enzyme | Release of a C-terminal dipeptide, oligopeptide-\|-Xaa- | Benazepril (Lotensin), |
| (ACE) (P12821, P09470, | Yaa, when Xaa is not Pro, and Yaa is neither Asp nor | Captopril, Enalapril |
| Q9BYF1) | Glu. | (Vasotec), Fosinopril, |
| | | Lisinopril (Prinivil, |
| | | Zestril), Moexipril, |
| | | Perindopril (Aceon), |
| | | Quinapril (Accupril), |
| | | Ramipril (Altace), |
| | | Trandolapril (Mavik), |
| | | Zofenopril |
| pyroglutamyl peptidase II | Release of the N-terminal pyroglutamyl group from | |
| (Q9NXJ5) | pGlu-His-Xaa tripeptides and pGlu-His-Xaa-Gly | |
| | tetrapeptides | |
| dipeptidyl peptidase IV (P27487, | Release of anN-terminal dipeptide, Xaa-Yaa-\|-Zaa-, | |
| P14740, P28843) | from a polypeptide, preferentially when Yaa is Pro, | |
| | provided Zaa is neither Pro nor hydroxyproline | |
| N-arginine dibasic convertase | Hydrolysis of polypeptides, preferably at -Xaa-\|-Arg- | |
| (O43847, Q8BHG1) | Lys-, and less commonly at -Arg-\|-Arg-Xaa-, in which | |
| | Xaa is not Arg or Lys | |
| endopeptidase 24.15 (thimet | Preferential cleavage of bonds with hydrophobic | |
| oligopeptidase) (P52888, | residues at P1, P2 and P3′ and a small residue at PT in | |
| P24155) | substrates of 5 to 15 residues | |
| endopeptidase 24.16 (neurolysin) | Preferential cleavage in neurotensin: 10-Pro-\|-Tyr-11 | |
| (Q9BYT8, Q91YP2) | | |
| amyloid precursor protein | Endopeptidase of broad specificity. | |
| secretase alpha (P05067, | | |
| P12023, Q9Y5Z0, P56817) | | |
| amyloid precursor protein | Broad endopeptidase specificity. Cleaves Glu-Val-Asn- | |
| secretase beta (P05067, P12023, | Leu-\|-Asp-Ala-Glu-Phe in the Swedish variant of | |
| Q9Y5Z0, P56817) | AlzhFeimer′s amyloid precursor protein | |
| amyloid precursor protein | intramembrane cleavage of | |
| secretase gamma (P05067, | integral membrane proteins | |
| P12023, Q9Y5Z0, P56817) | | |
| MMP 1 (P03956, Q9EPL5uy) | Cleavage of the triple helix of collagen at about three- | SB-3CT |
| | quarters of the length of the molecule from the N- | p-OH SB-3CT |
| | terminus, at 775-Gly-\|-Ile-776 in the alpha-l(I) chain. | O-phosphate SB-3CT |
| | Cleaves synthetic substrates and alpha-macroglobulins | RXP470.1 |
| | at bonds where PT is a hydrophobic residue. | |
| MMP 2 (P08253, P33434) | Cleavage of gelatin type I and collagen types IV, V, | SB-3CT |
| | VII, X. Cleaves the collagen-like sequence Pro-Gln- | p-OH SB-3CT |
| | Gly-\|-Ile-Ala-Gly-Gln. | O-phosphate SB-3CT |
| | | RXP470.1 |
| MMP 3 (P08254, P28862) | Preferential cleavage where PT, P2′ and P3′ are | SB-3CT |
| | hydrophobic residues. | p-OH SB-3CT |
| | | O-phosphate SB-3CT |
| | | RXP470.1 |
| MMP 7 (P09237, Q10738) | Cleavage of 14-Ala-\|-Leu-15 and 16-Tyr-\|-Leu-17 in B | SB-3CT |
| | chain of insulin. No action on collagen types I, II, IV, | p-OH SB-3CT |
| | V. Cleaves gelatin chain alpha-2(I) > alpha-1(I). | O-phosphate SB-3CT |
| | | RXP470.1 |
| MMP 8 (P22894, 070138) | Can degrade fibrillar type I, II, and III collagens. | SB-3CT |
| | Cleavage of interstitial collagens in the triple helical | p-OH SB-3CT |
| | domain. Unlike EC 3.4.24.7, this enzyme cleaves type | O-phosphate SB-3CT |
| | III collagen more slowly than type I. | RXP470.1 |
| MMP 9 (P14780, P41245) | Cleavage of gelatin types I and V and collagen types | SB-3CT |
| | IV and V. | p-OH SB-3CT |
| | Cleaves KiSSl at a Gly-\|-Leu bond. | O-phosphate SB-3CT |
| | Cleaves type IV and type V collagen into large C- | RXP470.1 |
| | terminal three quarter fragments and shorter N-terminal | |
| | one quarter fragments. Degrades fibronectin but not | |
| | laminin or Pz-peptide. | |
| MMP 10 (P09238, 055123) | Can degrade fibronectin, gelatins of type I, III, IV, and | SB-3CT |
| | V; weakly collagens III, IV, and V. | p-OH SB-3CT |
| | | O-phosphate SB-3CT |
| | | RXP470.1 |
| MMP 11 (P24347, Q02853) | A(A/Q)(N/A)↓(L/Y)(T/V/M/R)(R/K) | SB-3CT |
| | G(G/A)E↓LR | p-OH SB-3CT |
| | ↓ denotes the cleavage site | O-phosphate SB-3CT |
| | | RXP470.1 |
| MMP 12 (P39900, P34960) | Hydrolysis of soluble and insoluble elastin. Specific | SB-3CT |
| | cleavages are also produced at 14-Ala-\|-Leu-15 and 16- | p-OH SB-3CT |
| | Tyr-\|-Leu-17 in the B chain of insulin | O-phosphate SB-3CT |
| | Has significant elastolytic activity. Can accept large | RXP470.1 |
| | and small amino acids at the PT site, but has a | |
| | preference for leucine. Aromatic or hydrophobic | |
| | residues are preferred at the P1 site, with small | |
| | hydrophobic residues (preferably alanine) occupying | |
| | P3 | |
| MMP 13 (P45452, P33435) | Cleaves triple helical collagens, including type I, type | SB-3CT |
| | 11 and type III collagen, but has the highest activity | p-OH SB-3CT |
| | with soluble type II collagen. Can also degrade | O-phosphate SB-3CT |
| | collagen type IV, type XIV and type X | RXP470.1 |
| MMP 14 (P50281, P53690) | Activates progelatinase A by cleavage of the | SB-3CT |
| | propeptide at 37-Asn-\|-Leu-38. Other bonds | p-OH SB-3CT |
| | hydrolyzed include 35-Gly-\|-Ile-36 in the propeptide of | O-phosphate SB-3CT |
| | collagenase 3, and 341-Asn-\|-Phe-342, 441-Asp-\|-Leu- | RXP470.1 |
| | 442 and 354-Gln-\|-Thr-355 in the aggrecan | |
| | interglobular domain. | |
| urokinase plasminogen activator | Specific cleavage of Arg-\|-Val bond in plasminogen to | Plasminogen activator |
| (uPA) (P00749, P06869) | form plasmin. | inhibitors (PAI) |
| tissue plasminogen activator | Specific cleavage of Arg-\|-Val bond in plasminogen to | Plasminogen activator |
| (tPA) (P00750, P11214) | form plasmin. | inhibitors (PAI) |
| tissue plasminogen activator | Specific cleavage of Arg-\|-Val bond in plasminogen to | Plasminogen activator |
| (tPA) (P00750, P11214) | form plasmin. | inhibitors (PAI) |
| Plasmin (P00747, P20918) | Preferential cleavage: Lys-\|-Xaa > Arg-\|-Xaa, higher | α-2-antiplasmin (AP) |
| | selectivity than trypsin. Converts fibrin into soluble | |
| | products. | |
| Thrombin (P00734, P19221) | Cleaves bonds after Arg and Lys | |
| | Converts fibrinogen to fibrin and activates factors V, | |
| | VII, VIII, XIII, and, in complex with thrombomodulin, | |
| | protein C. | |
| BMP-1 (procollagen C- | Cleavage of the C-terminal propeptide at Ala-\|-Asp in | |
| peptidase) (P13497, P98063) | type I and II procollagens and at Arg-\|-Asp in type III. | |
| ADAM (Q9POK1, Q9UKQ2, | | SB-3CT |
| Q9JLN6, 014672, Q13444, | | p-OH SB-3CT |
| P78536, Q13443, 043184, | | O-phosphate SB-3CT |
| P78325, Q9UKF5, Q9BZ11, | | RXP470.1 |
| Q9H2U9, Q99965, 075077, | | |
| Q9HO13, O43506) | | |
| granzyme A (P12544, P11032) | Preferential cleavage: -Arg-\|-Xaa-, -Lys-\|-Xaa->>- | |
| | Phe-\|-Xaa- in small molecule substrates. | |
| granzyme B (P10144, P04187) | Preference for bulky and aromatic residues at the Pl | |
| | position and acidic residues at the P3′ and P4′ sites. | |
| granzyme M (P51124, Q03238) | Cleaves peptide substrates after methionine, leucine, | |
| | and norleucine. | |
| tobacco Etch virus (TEV) | E-Xaa-Xaa-Y-Xaa-Q-(G/S), with cleavage occurring | |
| protease (P04517, POCKO9) | between Q and G/S. The most common sequence is | |
| | ENLYFQS | |
| chymotrypsin-like serine | | -Thermobifida fusca |
| protease (P08217, Q9UNI1, | | Thermopin |
| Q91X79, P08861, P09093, | | -Pyrobaculum |
| P08218) | | aerophilum Aeropin |
| | | -Thermococcus |
| | | kodakaraensis Tk-serpin |
| | | -Alteromonas sp. |
| | | Marinostatin |
| | | -Streptomyces |
| | | misionensis SMTI |
| | | -Streptomyces sp. |
| | | chymostatin |
| alphavirus proteases (P08411, | | |
| P03317, P13886, Q8JUX6, | | |
| Q86924, Q4QXJ8, Q8QL53, | | |
| P27282, Q5XXP4) | | |
| chymotrypsin-like cysteine | | -Thermobifida fusca |
| proteases (Q86TL0, Q14790, | | Thermopin |
| Q99538, 015553) | | -Pyrobaculum |
| | | aerophilum Aeropin |
| | | -Thermococcus |
| | | kodakaraensis Tk-serpin |
| | | -Alteromonas sp. |
| | | Marinostatin |
| | | -Streptomyces |
| | | misionensis SMTI |
| papain-like cysteine proteases | | |
| (P25774, P53634, Q96K76) | | |
| picomavirus leader proteases | | |
| (P03305, P03311, P13899) | | |
| HIV proteases (P04585, P03367, | | |
| P04584, P03369, P12497, | | |
| P03366, P04587) | | |
| Herpesvirus proteases (P10220, | | |
| Q2HRB6, O40922, Q69527) | | |
| adenovirus proteases (P03252, | | |
| P24937, Q83906, P68985, | | |
| P09569, P11825, P10381) | | |
| Streptomyces griseus protease A | | |
| (SGPA) (P00776) | | |
| Streptomyces griseus protease B | | |
| (SGPB) (P00777) | | |
| alpha-lytic protease (P85142, | | |
| P00778) | | |
| serine proteases (P48740, | | |
| P98064, Q9UL52, P05981, | | |
| 060235) | | |
| cysteine proteases (Q86TL0, | | |
| Q14790, Q8WYN0, Q96DT6, | | |
| P55211) | | |
| aspartic proteases (Q9Y5Z0, | | |
| P56817, Q00663, Q53RT3, | | |
| POCY27) | | |
| threonine proteases (Q9UI38, | | |
| Q16512, Q9H6P5, Q8IWU2) | | |
| Mast cell (MC) chymase | Abz-HPFHL-Lys(Dnp)-NH2 | BAY 1142524 |
| (CMA1) (NM_001836) | | SUN13834 |
| Rat mast cell protease −1, −2, −3, | Abz-HPFHL-Lys(Dnp)-NH2 | TY-51469 |
| −4, −5 (NM_017145, NM_172044, | | |
| NM_001170466, NM_019321, | | |
| NM_013092) | | |
| Rat vascular chymase (RVCH) | Abz-HPFHL-Lys(Dnp)-NH2 | |
| (O70500) | | |
| DENV NS3pro (NS2B/NS3) | A strong preference for basic amino acid residues | Anthraquinone |
| | (Arg/Lys) at the P1 positions was observed, whereas | BP13944 |
| | the preferences for the P2-4 sites were in the order of | ZINC04321905 |
| | Arg > Thr > Gin/Asn/Lys for P2, Lys > Arg > Asn for | MB21 |
| | P3, and Nle > Leu > Lys > Xaa for P4. The prime site | Policresulen |
| | substrate specificity was for small and polar amino | SK-12 |
| | acids in P1 and P3. | NSC135618 |
| | | Biliverdin |
| NS3/NS4 protease cleavage site | EDWCCHSIY | Simeprevir, Danoprevir, |
| | (SEQ ID NO: 159) | Asunaprevir, Ciluprevir, |
| | LYQEFDEMEECSQH | Boceprevir, Sovaprevir, |
| | (SEQ ID NO: 160) | Paritaprevir, Telaprevir, |
| | | Grazoprevir |

In some embodiments, the one or more cognate cleavage sites of the repressible protease are localized between the DNA-binding domain and the effector domain of the ACP. In some embodiments, the repressible protease is hepatitis C virus (HCV) nonstructural protein 3 (NS3). In some embodiments, the cognate cleavage site comprises an NS3 protease cleavage site. In some embodiments, the NS3 protease cleavage site comprises a NS3/NS4A, a NS4A/NS4B, a NS4B/NS5A, or a NS5A/NS5B junction cleavage site.

In some embodiments, the NS3 protease can be repressed by a protease inhibitor. Any suitable protease inhibitor can be used, including, but not limited to, simeprevir, danoprevir, asunaprevir, ciluprevir, boceprevir, sovaprevir, paritaprevir, telaprevir, grazoprevir, glecaprevir, and voxiloprevir, or any combination thereof. In some embodiments, the protease inhibitor is selected from: simeprevir, danoprevir, asunaprevir, ciluprevir, boceprevir, sovaprevir, paritaprevir, telaprevir, grazoprevir, glecaprevir, and voxiloprevir. In some embodiments, the protease inhibitor is grazoprevir. In some embodiments, the protease inhibitor is a combination of grazoprevir and elbasvir (a NS5A inhibitor of the hepatitis C virus NS5A replication complex). Grazoprevir and elbasvir can be co-formulated as a pharmaceutical composition, such as in tablet form (e.g., the tablet available under the tradename Zepatier®). Grazoprevir and elbasvir can be co-formulated at a 2:1 weight ratio, respectively, such as at a unit dose of 100 mg grazoprevir 50 mg elbasvir (e.g., as in the tablet available under the tradename Zepatier®). Protease inhibitors that are structurally similar to grazoprevir can be used, such as any with the general formula (I) below

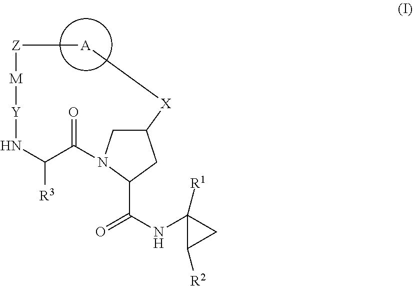

where

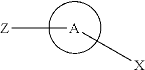

is one or more rings selected from the group consisting of:

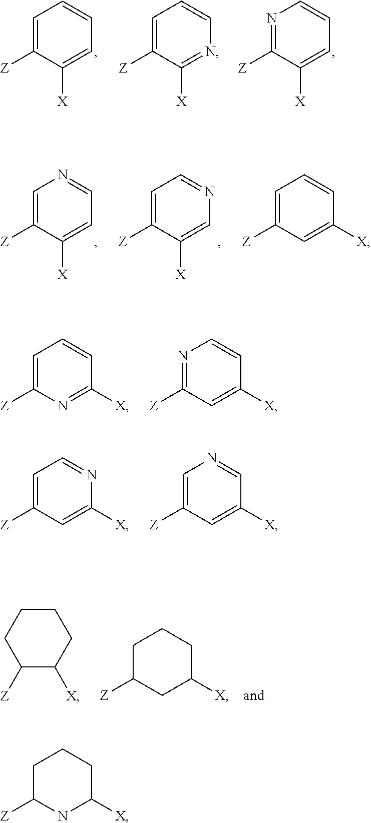

R¹ is selected from the group consisting of —CO₂R¹⁰ and —CONR¹⁰SO₂R⁶; R² is —CH═CH₂; R³ is C₁-C₆ alkyl; R⁶ is C₃ cycloalkyl; Y is selected from the group consisting of —OC(O)—; Z is a direct bond; M is selected from the group consisting of C₁-C₁₂ alkylenes and C₂-C₁₂ alkenylenes, M is substituted with 1 to 2 substituents F independently selected from the group consisting of C₁-C₈ alkyl and ═CH₂; X is selected from the group consisting of —(CH₂)₀₋₃O—, where ⊙ is attached to —(CH₂)₀₋₃ if present; and each R¹⁰ is independently H. Grazoprevir, elbasvir, and combinations thereof are described in U.S. Pat. Nos. 9,738,661; 7,973,040; and 8,871,759 and U.S. Pat. Pub No. US20160243128, each herein incorporated by reference for all purposes.

In some embodiments, an ACP of the present disclosure comprises a small molecule (e.g., drug) inducible hormone-binding domain of estrogen receptor (ERT2 domain). In some embodiments, the ERT2 domain is an estrogen receptor variant that binds to tamoxifen, and metabolites thereof, but not to estradiol. Non-limiting examples of tamoxifen metabolites may include 4-hydroxytamoxifen, N-desmethyltamoxifen, tamoxifen-N-oxide, and endoxifen. In some embodiments, when expressed in a cell and in the absence of the small molecule (e.g., tamoxifen or a metabolite thereof) the ACP comprising the ERT2 domain binds to HSP90 and is maintained in the cytoplasm of the cell. In some embodiments, upon introduction of the small molecule (e.g., tamoxifen or a metabolite thereof), the small molecule displaces HSP90 bound to the ERT2 domain, which allows the ACP comprising the ERT2 domain to translocate to the nucleus of the cell.

Accordingly, in some embodiments an ACP of the present disclosure comprising an ERT2 domain is capable of undergoing nuclear localization upon binding of the ERT2 domain to tamoxifen or a metabolite thereof. In some embodiments, the tamoxifen metabolite is selected from 4-hydroxy-tamoxifen (4-OHT), N-desmethyltamoxifen, tamoxifen-N-oxide, and endoxifen.

Degredation Sequences and Degrons

In some embodiments, the ACP further comprises a degron, wherein the degron is operably linked to the ACP. In some embodiments, the degron is localized 5′ of the repressible protease, 3′ of the repressible protease, 5′ of the DNA-binding domain, 3′ of the DNA-binding domain, 5′ of the effector domain, or 3′ of the effector domain.

The terms “degron” “degron domain,” as used herein, refers to a protein or a part thereof that is important in regulation of protein degradation rates. Various degrons known in the art, including but not limited to short amino acid sequences, structural motifs, and exposed amino acids, can be used in various embodiments of the present disclosure. Degrons identified from a variety of organisms can be used. Degrons and degron pathways are generally known, see, e.g., Varshazsky A., PNAS 2019 Jan. 8; 116(2):358-366, hereby incorporated by reference.

The term “degradation sequence” as used herein, refers to a sequence that promotes degradation of an attached protein through either the proteasome or autophagy-lysosome pathways. Degradation sequences known in the art can be used for various embodiments of the present disclosure. In some embodiments, a degradation sequence comprises a degron identified from an organism, or a modification thereof. In some embodiments, a degradation sequence is a polypeptide that destabilize a protein such that half-life of the protein is reduced at least two-fold, when fused to the protein. Many different degradation sequences/signals (e.g., of the ubiquitin-proteasome system) are known in the art, any of which may be used as provided herein. A degradation sequence may be operably linked to a cell receptor, but need not be contiguous with it as long as the degradation sequence still functions to direct degradation of the cell receptor. In some embodiments, the degradation sequence induces rapid degradation of the cell receptor. For a discussion of degradation sequences and their function in protein degradation, see, e.g., Kanemaki et al. (2013) Pflugers Arch. 465(3):419-425, Erales et al. (2014) Biochim Biophys Acta 1843(0:216-221, Schrader et al. (2009) Nat. Chem. Biol. 5(11): 815-822, Ravid et al. (2008) Nat. Rev. Mol. Cell. Biol. 9(9):679-690, Tasaki et al. (2007) Trends Biochem Sci. 32(11):520-528, Meinnel et al. (2006) Biol. Chem. 387(7):839-851, Kim et al. (2013) Autophagy 9(7): 1100-1103, Varshaysky (2012) Methods Mol. Biol. 832: 1-11, and Fayadat et al. (2003) Mol Biol Cell. 14(3): 1268-1278; herein incorporated by reference.

In some embodiments, the degron or degradation sequence is selected from: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

In some embodiments, the degron comprises a cereblon (CRBN) polypeptide substrate domain capable of binding CRBN in response to an immunomodulatory drug (IMiD) thereby promoting ubiquitin pathway-mediated degradation of the ACP. In some embodiments, the CRBN polypeptide substrate domain is selected from: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some embodiments, the CRBN polypeptide substrate domain is a chimeric fusion product of native CRBN polypeptide sequences. In some embodiments, the CRBN polypeptide substrate domain is a IKZF3/ZFP91/IKZF3 chimeric fusion product having the amino acid sequence of

(SEQ ID NO: 131)
FNVLMVHKRSHTGERPLQCEICGFTCRQKGNLLRH
IKLHTGEKPFKCHLCNYACQRRDAL

In some embodiments, the immunomodulatory drug (IMiD) is an FDA-approved drug. In some embodiments, the IMiD is selected from: thalidomide, lenalidomide, and pomalidomide.

Promoters

In some embodiments, an engineered nucleic acid of the present disclosure comprises a first expression cassette comprising a first promoter operably linked to an exogenous polynucleotide sequence encoding an activation-conditional control polypeptide (ACP). In some embodiments, an engineered nucleic acid of the present disclosure comprises a second expression cassette comprising an ACP-responsive promoter operably linked to a second exogenous polynucleotide sequence encoding one or more effector molecules. In some embodiments, the first expression cassette and second expression cassette are each encoded by a separate engineered nucleic acid of the present disclosure. In other embodiments, the first expression cassette and the second expression cassette are encoded by the same engineered nucleic acid of the present disclosure.

In some embodiments, an ACP-responsive promoter of the present disclosure comprises an ACP-binding domain and a promoter sequence. In some embodiments, the ACP-responsive promoter is operable linked to a nucleotide sequence encoding an effector molecule.

In some embodiments, an engineered nucleic acid comprises an ACP-responsive promoter operably linked to a nucleotide sequence encoding an effector molecule. In some embodiments, an engineered nucleic acid comprises an ACP-responsive promoter operably linked to a nucleotide sequence encoding at least 2 effector molecules. For example, the engineered nucleic acid may comprise an ACP-responsive promoter operably linked to a nucleotide sequence encoding at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 effector molecules. In some embodiments, an engineered nucleic acid comprises an ACP-responsive promoter operably linked to a nucleotide sequence encoding 1, 2, 3, 4, 5, 6, 7, 8, 9, 10, 11, 12, 13, 14, 15, 16, 17, 18, 19, 20, or more effector molecules.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid of the present disclosure may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 2, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Horner, M. & Weber, W. FEBS Letters 586 (2012) 20784-2096m, and references cited therein). Other non-limiting examples of inducible promoters include those shown in Table 3.

TABLE 2
Exemplary Inducible Promoters

| | Promoter and | Transcription | Inducer | Response to inducer | |
|---|---|---|---|---|---|
| System | operator | factor (TF) | molecule | TF | T |

Transcriptional activator-responsive promoters

| | | | | | |
|---|---|---|---|---|---|
| AIR | PAIR (OalcA- | AlcR | Acetaldehyde | n.d. | A |
| | PhCMVmin) | | | | |
| ART | PART (OARG- | ArgR-VP16 | l-Arginine | B | A |
| | PhCMVmin) | | | | |
| BIT | PBIT3 (OBirA3- | BIT (BirA-VP16) | Biotin | B | A |
| | PhCMVmin) | | | | |
| Cumate—activator | PCR5 (OCuO6- | cTA (CymR- | Cumate | D | DA |
| | PhCMVmin) | VP16) | | | |
| Cumate—reverse | PCR5 (OCuO6- | rcTA (rCymR- | Cumate | B | A |
| activator | PhCMVmin) | VP16) | | | |
| E-OFF | PETR(OETR- | ET (E-VP16) | Erythromycin | D | DA |
| | PhCMVmin) | | | | |
| NICE-OFF | PNIC (ONIC- | NT (HchioR-VP16) | 6-Hydroxy- | D | DA |
| | PhCMVmin) | | nicotine | | |
| PEACE | PTtgRl (OTtgR- | TtgAl (TtgR- | Phloretin | D | DA |
| | PhCMVmin) | VP16) | | | |
| PIP-OFF | PPIR (OPIR- | PIT (PIP-VP16) | Pristinamycinl | D | DA |
| | Phsp70min) | | | | |
| QuoRex | PSCA (OscbR- | SCA (ScbR-VP16) | SCB1 | D | DA |
| | PhCMVmin)PSPA | | | | |
| | (OpapRI-PhCMVmin) | | | | |
| Redox | PROP (OROP- | REDOX (REX- | NADH | D | DA |
| | PhCMVmin) | VP16) | | | |
| TET-OFF | PhCMV*-1 (OtetO7- | tTA (TetR-VP16) | Tetracycline | D | DA |
| | PhCMVmin) | | | | |
| TET-ON | PhCMV*-1 (OtetO7- | rtTA (rTetR-VP16) | Doxycycline | B | A |
| | PhCMVmin) | | | | |
| TIGR | PCTA (OrheO- | CTA (RheA-VP16) | Heat | D | DA |
| | PhCMVmin) | | | | |
| TraR | 07x(tra box)- | p65-TraR | 3-Oxo-C8- | B | A |
| | PhCMVmin | | HSL | | |
| VAC-OFF | PlVanO2 (0Van02- | VanAl (VanR- | Vanillic acid | D | DA |
| | PhCMVmin) | VP16) | | | |

Transcriptional repressor-responsive promoters

| Cumate—repressor | PCuO (PCMV5- | CymR | Cumate | D | DR |
|---|---|---|---|---|---|
| | OCuO) | | | | |
| E-ON | PETRON8 (PSV40- | E-KRAB | Erythromycin | D | DR |
| | OETR8) | | | | |
| NICE-ON | PNIC (PSV40- | NS (HdnoR- | 6-Hydroxy- | D | DR |
| | ONIC8) | KRAB) | nicotine | | |
| PIP-ON | PPIRON (PSV40- | PIT3 (PIP-KRAB) | Pristinamycin I | D | DR |
| | OPIR3) | | | | |
| Q-ON | PSCAON8 (PSV40- | SCS (ScbR- | SCBI | D | DR |
| | OscbR8) | KRAB) | | | |
| TET- | OtetO-PHPRT | tTS-H4 (TetR- | Doxycycline | D | DR |
| System | Promoter and | Transcription | Inducer | Response to inducer | |
| | operator | factor (TF) | molecule | | |
| ON<comma> | | HDAC4) | | | |
| repressor-based | | | | | |
| T-REX | PTetO (PhCMV- | TetR | Tetracycline | D | DR |
| | OtetO2) | | | | |
| UREX | PUREX8 (PSV40- | mUTS (KRAB- | Uric acid | D | DR |
| | OhucO8) | HueR) | | | |
| VAC-ON | PVanON8 (PhCMV- | VanA4 (VanR- | Vanillic acid | D | DR |
| | OVanO8) | KRAB) | | | |

Hybrid promoters

| | | | | | |
|---|---|---|---|---|---|
| QuoRexPIP- | OscbR8-OPIR3- | SCAPIT3 | SCBIPristina | DD | DADR |
| ON(NOT IF gate) | PhCMVmin | | mycin I | | |
| QuoRexE- | OscbR-OETR8- | SCAE-KRAB | SCBlErythro | DD | DADR |
| ON(NOT IF gate) | PhCMVmin | | mycin | | |
| TET-OFFE- | OtetO7-OETR8- | tTAE-KRAB | Tetracycline | DD | DADR |
| ON(NOT IF gate) | PhCMVmin | | Erythromycin | | |
| TET-OFFPIP- | OtetO7-OPIR3- | tTAPIT3E-KRAB | Tetracycline | DDD | DADRD |
| ONE-ON | OETR8-PhCMVmin | | Pristinamycin | | R |
| | | | IErythromycin | | |

TABLE 3
Exemplary Inducible Promoters

| Name | DNA SEQUENCE | Source |
|---|---|---|
| minimal promoter; minP | AGAGGGTATATAATGGAAGCTCGACTTC | EU581860.1 |
| | CAG (SEQ ID NO: 1) | (Promega) |
| NFkB response element | GGGAATTTCCGGGGACTTTCCGGGAATT | EU581860.1 |
| protein promoter; 5x | TCCGGGGACTTTCCGGGAATTTCC (SEQ | (Promega) |
| NFkB-RE | ID NO: 2) | |
| CREB response element | CACCAGACAGTGACGTCAGCTGCCAGAT | DQ904461.1 |
| protein promoter; 4x CRE | CCCATGGCCGTCATACTGTGACGTCTTT | (Promega) |
| | CAGACACCCCATTGACGTCAATGGGAG | |
| | AA (SEQ ID NO: 3) | |
| NF AT response element | GGAGGAAAAACTGTTTCATACAGAAGG | DQ904462.1 |
| protein promoter; | CGTGGAGGAAAAACTGTTTCATACAGA | (Promega) |
| 3x NF AT | AGGCGTGGAGGAAAAACTGTTTCATAC | |
| binding sites | AGAAGGCGT (SEQ ID NO: 4) | |
| SRF response element | AGGATGTCCATATTAGGACATCTAGGAT | FJ773212.1 |
| protein promoter; | GTCCATATTAGGACATCTAGGATGTCCA | (Promega) |
| 5x SRE | TATTAGGACATCTAGGATGTCCATATTA | |
| | GGACATCTAGGATGTCCATATTAGGACA | |
| | TCT (SEQ ID NO: 5) | |
| SRF response element | AGTATGTCCATATTAGGACATCTACCAT | FJ773213.1 |
| protein promoter 2; 5x | GTCCATATTAGGACATCTACTATGTCCA | (Promega) |
| SRF-RE | TATTAGGACATCTTGTATGTCCATATTA | |
| | GGACATCTAAAATGTCCATATTAGGACA | |
| | TCT (SEQ ID NO: 6) | |
| API response element | TGAGTCAGTGACTCAGTGAGTCAGTGAC | JQ858516.1 |
| protein promoter; | TCAGTGAGTCAGTGACTCAG (SEQ ID | (Promega) |
| 6x AP1-RE | NO: 7) | |
| TCF-LEF response element | AGATCAAAGGGTTTAAGATCAAAGGGC | JX099537.1 |
| promoter; | TTAAGATCAAAGGGTATAAGATCAAAG | (Promega) |
| 8x TCF-LEF-RE | GGCCTAAGATCAAAGGGACTAAGATCA | |
| | AAGGGTTTAAGATCAAAGGGCTTAAGA | |
| | TCAAAGGGCCTA (SEQ ID NO: 8) | |
| SBEx4 | GTCTAGACGTCTAGACGTCTAGACGTCT | Addgene Cat No: 16495 |
| | AGAC (SEQ ID NO: 9) | |
| SMAD2/3-CAGACA x4 | CAGACACAGACACAGACACAGACA | Jonk et al. |
| | (SEQ ID NO: 10) | (J Biol Chem. Aug. 14, |
| | | 1998 ; 273(33):21145-52. |
| STAT3 binding site | Ggatccggtactcgagatctgcgatcta | Addgene Sequencing |
| | agtaagcttggcattccggtactgttgg | Result #211335 |
| | taaagccac (SEQ ID NO: 11) | |
| 5x NF AT | GGGACTTTCCACTGGGGACTTTCCACTG | |
| | GGGACTTTCCACTGGGGACTTTCCACTG | |
| | GGGACTTTCC (SEQ ID NO: 152) | |
| min AdeP | AGACGCTAGCGGGGGGCTATAAAAGGG | |
| | GGTGGGGGCGTTCGTCCTCACTCT (SEQ | |
| | ID NO: 153) | |
| 5x NF AT minAdeP | GGGACTTTCCACTGGGGACTTTCCACTG | |
| | GGGACTTTCCACTGGGGACTTTCCACTG | |
| | GGGACTTTCCACTCCTGCAGGagctGGCG | |
| | CGCCAGACGCTAGCGGGGGGCTATAAA | |
| | AGGGGGTGGGGGCGTTCGTCCTCACTCT | |
| | (SEQ ID NO: 154) | |
| YB-TATA | TCTAGAGGGTATATAATGGGGGCCA | |
| | (SEQ ID NO: 155) | |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter. In some embodiments, the promoter is a constitutive promoter. Exemplary constitutive promoters are shown in Table 4.

TABLE 4
Exemplary Constitutive Promoters

| Name | DNA SEQUENCE |
|---|---|
| CMV | GTTGACATTGATTATTGACT |
| | AGTTATTAATAGTAATCAAT |
| | TACGGGGTCATTAGTTCATA |
| | GCCCATATATGGAGTTCCGC |
| | GTTACATAACTTACGGTAAA |
| | TGGCCCGCCTGGCTGACCGC |
| | CCAACGACCCCCGCCCATTG |
| | ACGTCAATAATGACGTATGT |
| | TCCCATAGTAACGCCAATAG |
| | GGACTTTCCATTGACGTCAA |
| | TGGGTGGAGTATTTACGGTA |
| | AACTGCCCACTTGGCAGTAC |
| | ATCAAGTGTATCATATGCCA |
| | AGTACGCCCCCTATTGACGT |
| | CAATGACGGTAAATGGCCCG |
| | CCTGGCATTATGCCCAGTAC |
| | ATGACCTTATGGGACTTTCC |
| | TACTTGGCAGTACATCTACG |
| | TATTAGTCATCGCTATTACC |
| | ATGGTGATGCGGTTTTGGCA |
| | GTACATCAATGGGCGTGGAT |
| | AGCGGTTTGACTCACGGGGA |
| | TTTCCAAGTCTCCACCCCAT |
| | TGACGTCAATGGGAGTTTGT |
| | TTTGGCACCAAAATCAACGG |
| | GACTTTCCAAAATGTCGTAA |
| | CAACTCCGCCCCATTGACGC |
| | AAATGGGCGGTAGGCGTGTA |
| | CGGTGGGAGGTCTATATAAG |
| | CAGAGCTC |
| | (SEQ ID NO: 12) |
| EF1a | GGCTCCGGTGCCCGTCAGTG |
| | GGCAGAGCGCACATCGCCCA |
| | CAGTCCCCGAGAAGTTGGGG |
| | GGAGGGGTCGGCAATTGAAC |
| | CGGTGCCTAGAGAAGGTGGC |
| | GCGGGGTAAACTGGGAAAGT |
| | GATGCCGTGTACTGGCTCCG |
| | CCTTTTTCCCGAGGGTGGGG |
| | GAGAACCGTATATAAGTGCA |
| | GTAGTCGCCGTGAACGTTCT |
| | TTTTCGCAACGGGTTTGCCG |
| | CCAGAACACAGGTAAGTGCC |
| | GTGTGTGGTTCCCGCGGGCC |
| | TGGCCTCTTTACGGGTTATG |
| | GCCCTTGCGTGCCTTGAATT |
| | ACTTCCACCTGGCTGCAGTA |
| | CGTGATTCTTGATCCCGAGC |
| | TTCGGGTTGGAAGTGGGTGG |
| | GAGAGTTCGAGGCCTTGCGC |
| | TTAAGGAGCCCCTTCGCCTC |
| | GTGCTTGAGTTGAGGCCTGG |
| | CCTGGGCGCTGGGGCCGCCG |
| | CGTGCGAATCTGGTGGCACC |
| | TTCGCGCCTGTCTCGCTGCT |
| | TTCGATAAGTCTCTAGCCAT |
| | TTAAAATTTTTGATGACCTG |
| | CTGCGACGCTTTTTTTCTGG |
| | CAAGATAGTCTTGTAAATGC |
| | GGGCCAAGATCTGCACACTG |
| | GTATTTCGGTTTTTGGGGCC |
| | GCGGGCGGCGACGGGGCCCG |
| | TGCGTCCCAGCGCACATGTT |
| | CGGCGAGGCGGGGCCTGCGA |
| | GCGCGACCACCGAGAATCGG |
| | ACGGGGGTAGTCTCAAGCTG |
| | GCCGGCCTGCTCTGGTGCCT |
| | GTCCTCGCGCCGCCGTGTAT |
| | CGCCCCGCCCCGGGCGGCAA |
| | GGCTGGCCCGGTCGGCACCA |
| | GTTGCGTGAGCGGAAAGATG |
| | GCCGCTTCCCGGTCCTGCTG |
| | CAGGGAGCTCAAAATGGAGG |
| | ACGCGGCGCTCGGGAGAGCG |
| | GGCGGGTGAGTCACCCACAC |
| | AAAGGAAAAGGGCCTTTCCG |
| | TCCTCAGCCGTCGCTTCATG |
| | TGACTCCACGGAGTACCGGG |
| | CGCCGTCCAGGCACCTCGAT |
| | TAGTTCTCGAGCTTTTGGAG |
| | TACGTCGTCTTTAGGTTGGG |
| | GGGAGGGGTTTTATGCGATG |
| | GAGTTTCCCCACACTGAGTG |
| | GGTGGAGACTGAAGTTAGGC |
| | CAGCTTGGCACTTGATGTAA |
| | TTCTCCTTGGAATTTGCCCT |
| | TTTTGAGTTTGGATCTTGGT |
| | TCATTCTCAAGCCTCAGACA |
| | GTGGTTCAAAGTTTTTTTCT |
| | TCCATTTCAGGTGTCGTGA |
| | (SEQ ID NO: 13) |
| EFS | GGATCTGCGATCGCTCCGGT |
| | GCCCGTCAGTGGGCAGAGCG |
| | CACATCGCCCACAGTCCCCG |
| | AGAAGTTGGGGGGAGGGGTC |
| | GGCAATTGAACCGGTGCCTA |
| | GAGAAGGTGGCGCGGGGTAA |
| | ACTGGGAAAGTGATGTCGTG |
| | TACTGGCTCCGCCTTTTTCC |
| | CGAGGGTGGGGGAGAACCGT |
| | ATATAAGTGCAGTAGTCGCC |
| | GTGAACGTTCTTTTTCGCAA |
| | CGGGTTTGCCGCCAGAACAC |
| | AGCTGAAGCTTCGAGGGGCT |
| | CGCATCTCTCCTTCACGCGC |
| | CCGCCGCCCTACCTGAGGCC |
| | GCCATCCACGCCGGTTGAGT |
| | CGCGTTCTGCCGCCTCCCGC |
| | CTGTGGTGCCTCCTGAACTG |
| | CGTCCGCCGTCTAGGTAAGT |
| | TTAAAGCTCAGGTCGAGACC |
| | GGGCCTTTGTCCGGCGCTCC |
| | CTTGGAGCCTACCTAGACTC |
| | AGCCGGCTCTCCACGCTTTG |
| | CCTGACCCTGCTTGCTCAAC |
| | TCTACGTCTTTGTTTCGTTT |
| | TCTGTTCTGCGCCGTTACAG |
| | ATCCAAGCTGTGACCGGCGC |
| | CTAC (SEQ ID NO: 14) |
| MND | TTTATTTAGTCTCCAGAAAA |
| | AGGGGGGAATGAAAGACCCC |
| | ACCTGTAGGTTTGGCAAGCT |
| | AGGATCAAGGTTAGGAACAG |
| | AGAGACAGCAGAATATGGGC |
| | CAAACAGGATATCTGTGGTA |
| | AGCAGTTCCTGCCCCGGCTC |
| | AGGGCCAAGAACAGTTGGAA |
| | CAGCAGAATATGGGCCAAAC |
| | AGGATATCTGTGGTAAGCAG |
| | TTCCTGCCCCGGCTCAGGGC |
| | CAAGAACAGATGGTCCCCAG |
| | ATGCGGTCCCGCCCTCAGCA |
| | GTTTCTAGAGAACCATCAGA |
| | TGTTTCCAGGGTGCCCCAAG |
| | GACCTGAAATGACCCTGTGC |
| | CTTATTTGAACTAACCAATC |
| | AGTTCGCTTCTCGCTTCTGT |
| | TCGCGCGCTTCTGCTCCCCG |
| | AGCTCAATAAAAGAGCCCA |
| | (SEQ ID NO: 15) |
| PGK | GGGGTTGGGGTTGCGCCTTT |
| | TCCAAGGCAGCCCTGGGTTT |
| | GCGCAGGGACGCGGCTGCTC |
| | TGGGCGTGGTTCCGGGAAAC |
| | GCAGCGGCGCCGACCCTGGG |
| | TCTCGCACATTCTTCACGTC |
| | CGTTCGCAGCGTCACCCGGA |
| | TCTTCGCCGCTACCCTTGTG |
| | GGCCCCCCGGCGACGCTTCC |
| | TGCTCCGCCCCTAAGTCGGG |
| | AAGGTTCCTTGCGGTTCGCG |
| | GCGTGCCGGACGTGACAAAC |
| | GGAAGCCGCACGTCTCACTA |
| | GTACCCTCGCAGACGGACAG |
| | CGCCAGGGAGCAATGGCAGC |
| | GCGCCGACCGCGATGGGCTG |
| | TGGCCAATAGCGGCTGCTCA |
| | GCGGGGCGCGCCGAGAGCAG |
| | CGGCCGGGAAGGGGCGGTGC |
| | GGGAGGCGGGGTGTGGGGCG |
| | GTAGTGTGGGCCCTGTTCCT |
| | GCCCGCGCGGTGTTCCGCAT |
| | TCTGCAAGCCTCCGGAGCGC |
| | ACGTCGGCAGTCGGCTCCCT |
| | CGTTGACCGAATCACCGACC |
| | TCTCTCCCCAG |
| | (SEQ ID NO: 16) |
| SFFV | GTAACGCCATTTTGCAAGGC |
| | ATGGAAAAATACCAAACCAA |
| | GAATAGAGAAGTTCAGATCA |
| | AGGGCGGGTACATGAAAATA |
| | GCTAACGTTGGGCCAAACAG |
| | GATATCTGCGGTGAGCAGTT |
| | TCGGCCCCGGCCCGGGGCCA |
| | AGAACAGATGGTCACCGCAG |
| | TTTCGGCCCCGGCCCGAGGC |
| | CAAGAACAGATGGTCCCCAG |
| | ATATGGCCCAACCCTCAGCA |
| | GTTTCTTAAGACCCATCAGA |
| | TGTTTCCAGGCTCCCCCAAG |
| | GACCTGAAATGACCCTGCGC |
| | CTTATTTGAATTAACCAATC |
| | AGCCTGCTTCTCGCTTCTGT |
| | TCGCGCGCTTCTGCTTCCCG |
| | AGCTCTATAAAAGAGCTCAC |
| | AACCCCTCACTCGGCGCGCC |
| | AGTCCTCCGACAGACTGAGT |
| | CGCCCGGG |
| | (SEQ ID NO: 17) |
| SV40 | CTGTGGAATGTGTGTCAGTT |
| | AGGGTGTGGAAAGTCCCCAG |
| | GCTCCCCAGCAGGCAGAAGT |
| | ATGCAAAGCATGCATCTCAA |
| | TTAGTCAGCAACCAGGTGTG |
| | GAAAGTCCCCAGGCTCCCCA |
| | GCAGGCAGAAGTATGCAAAG |
| | CATGCATCTCAATTAGTCAG |
| | CAACCATAGTCCCGCCCCTA |
| | ACTCCGCCCATCCCGCCCCT |
| | AACTCCGCCCAGTTCCGCCC |
| | ATTCTCCGCCCCATGGCTGA |
| | CTAATTTTTTTTATTTATGC |
| | AGAGGCCGAGGCCGCCTCTG |
| | CCTCTGAGCTATTCCAGAAG |
| | TAGTGAGGAGGCTTTTTTGG |
| | AGGCCTAGGCTTTTGCAAAA |
| | AGCT (SEQ ID NO: 18) |
| UbC | GCGCCGGGTTTTGGCGCCTC |
| | CCGCGGGCGCCCCCCTCCTC |
| | ACGGCGAGCGCTGCCACGTC |
| | AGACGAAGGGCGCAGGAGCG |
| | TTCCTGATCCTTCCGCCCGG |
| | ACGCTCAGGACAGCGGCCCG |
| | CTGCTCATAAGACTCGGCCT |
| | TAGAACCCCAGTATCAGCAG |
| | AAGGACATTTTAGGACGGGA |
| | CTTGGGTGACTCTAGGGCAC |
| | TGGTTTTCTTTCCAGAGAGC |
| | GGAACAGGCGAGGAAAAGTA |
| | GTCCCTTCTCGGCGATTCTG |
| | CGGAGGGATCTCCGTGGGGC |
| | GGTGAACGCCGATGATTATA |
| | TAAGGACGCGCCGGGTGTGG |
| | CACAGCTAGTTCCGTCGCAG |
| | CCGGGATTTGGGTCGCGGTT |
| | CTTGTTTGTGGATCGCTGTG |
| | ATCGTCACTTGGTGAGTTGC |
| | GGGCTGCTGGGCTGGCCGGG |
| | GCTTTCGTGGCCGCCGGGCC |
| | GCTCGGTGGGACGGAAGCGT |
| | GTGGAGAGACCGCCAAGGGC |
| | TGTAGTCTGGGTCCGCGAGC |
| | AAGGTTGCCCTGAACTGGGG |
| | GTTGGGGGGAGCGCACAAAA |
| | TGGCGGCTGTTCCCGAGTCT |
| | TGAATGGAAGACGCTTGTAA |
| | GGCGGGCTGTGAGGTCGTTG |
| | AAACAAGGTGGGGGGCATGG |
| | TGGGCGGCAAGAACCCAAGG |
| | TCTTGAGGCCTTCGCTAATG |
| | CGGGAAAGCTCTTATTCGGG |
| | TGAGATGGGCTGGGGCACCA |
| | TCTGGGGACCCTGACGTGAA |
| | GTTTGTCACTGACTGGAGAA |
| | CTCGGGTTTGTCGTCTGGTT |
| | GCGGGGGCGGCAGTTATGCG |
| | GTGCCGTTGGGCAGTGCACC |
| | CGTACCTTTGGGAGCGCGCG |
| | CCTCGTCGTGTCGTGACGTC |
| | ACCCGTTCTGTTGGCTTATA |
| | ATGCAGGGTGGGGCCACCTG |
| | CCGGTAGGTGTGCGGTAGGC |
| | TTTTCTCCGTCGCAGGACGC |
| | AGGGTTCGGGCCTAGGGTAG |
| | GCTCTCCTGAATCGACAGGC |
| | GCCGGACCTCTGGTGAGGGG |
| | AGGGATAAGTGAGGCGTCAG |
| | TTTCTTTGGTCGGTTTTATG |
| | TACCTATCTTCTTAAGTAGC |
| | TGAAGCTCCGGTTTTGAACT |
| | ATGCGCTCGGGGTTGGCGAG |
| | TGTGTTTTGTGAAGTTTTTT |
| | AGGCACCTTTTGAAATGTAA |
| | TCATTTGGGTCAATATGTAA |
| | TTTTCAGTGTTAGACTAGTA |
| | AAGCTTCTGCAGGTCGACTC |
| | TAGAAAATTGTCCGCTAAAT |
| | TCTGGCCGTTTTTGGCTTTT |
| | TTGTTAGAC |
| | (SEQ ID NO: 19) |
| hEF1aV1 | GGCTCCGGTGCCCGTCAGTG |
| | GGCAGAGCGCACATCGCCCA |
| | CAGTCCCCGAGAAGTTGGGG |
| | GGAGGGGTCGGCAATTGAAC |
| | CGGTGCCTAGAGAAGGTGGC |
| | GCGGGGTAAACTGGGAAAGT |
| | GATGTCGTGTACTGGCTCCG |
| | CCTTTTTCCCGAGGGTGGGG |
| | GAGAACCGTATATAAGTGCA |
| | GTAGTCGCCGTGAACGTTCT |
| | TTTTCGCAACGGGTTTGCCG |
| | CCAGAACACAGGTAAGTGCC |
| | GTGTGTGGTTCCCGCGGGCC |
| | TGGCCTCTTTACGGGTTATG |
| | GCCCTTGCGTGCCTTGAATT |
| | ACTTCCACCTGGCTGCAGTA |
| | CGTGATTCTTGATCCCGAGC |
| | TTCGGGTTGGAAGTGGGTGG |
| | GAGAGTTCGAGGCCTTGCGC |
| | TTAAGGAGCCCCTTCGCCTC |
| | GTGCTTGAGTTGAGGCCTGG |
| | CCTGGGCGCTGGGGCCGCCG |
| | CGTGCGAATCTGGTGGCACC |
| | TTCGCGCCTGTCTCGCTGCT |
| | TTCGATAAGTCTCTAGCCAT |
| | TTAAAATTTTTGATGACCTG |
| | CTGCGACGCTTTTTTTCTGG |
| | CAAGATAGTCTTGTAAATGC |
| | GGGCCAAGATCTGCACACTG |
| | GTATTTCGGTTTTTGGGGCC |
| | GCGGGCGGCGACGGGGCCCG |
| | TGCGTCCCAGCGCACATGTT |
| | CGGCGAGGCGGGGCCTGCGA |
| | GCGCGGCCACCGAGAATCGG |
| | ACGGGGGTAGTCTCAAGCTG |
| | GCCGGCCTGCTCTGGTGCCT |
| | GGTCTCGCGCCGCCGTGTAT |
| | CGCCCCGCCCTGGGCGGCAA |
| | GGCTGGCCCGGTCGGCACCA |
| | GTTGCGTGAGCGGAAAGATG |
| | GCCGCTTCCCGGCCCTGCTG |
| | CAGGGAGCTCAAAATGGAGG |
| | ACGCGGCGCTCGGGAGAGCG |
| | GGCGGGTGAGTCACCCACAC |
| | AAAGGAAAAGGGCCTTTCCG |
| | TCCTCAGCCGTCGCTTCATG |
| | TGACTCCACGGAGTACCGGG |
| | CGCCGTCCAGGCACCTCGAT |
| | TAGTTCTCGAGCTTTTGGAG |
| | TACGTCGTCTTTAGGTTGGG |
| | GGGAGGGGTTTTATGCGATG |
| | GAGTTTCCCCACACTGAGTG |
| | GGTGGAGACTGAAGTTAGGC |
| | CAGCTTGGCACTTGATGTAA |
| | TTCTCCTTGGAATTTGCCCT |
| | TTTTGAGTTTGGATCTTGGT |
| | TCATTCTCAAGCCTCAGACA |
| | GTGGTTCAAAGTTTTTTTCT |
| | TCCATTTCAGGTGTCGTGA |
| | (SEQ ID NO: 20) |
| hCAGG | ACTAGTTATTAATAGTAATC |
| | AATTACGGGGTCATTAGTTC |
| | ATAGCCCATATATGGAGTTC |
| | CGCGTTACATAACTTACGGT |
| | AAATGGCCCGCCTGGCTGAC |
| | CGCCCAACGACCCCCGCCCA |
| | TTGACGTCAATAATGACGTA |
| | TGTTCCCATAGTAACGCCAA |
| | TAGGGACTTTCCATTGACGT |
| | CAATGGGTGGAGTATTTACG |
| | GTAAACTGCCCACTTGGCAG |
| | TACATCAAGTGTATCATATG |
| | CCAAGTACGCCCCCTATTGA |
| | CGTCAATGACGGTAAATGGC |
| | CCGCCTGGCATTATGCCCAG |
| | TACATGACCTTATGGGACTT |
| | TCCTACTTGGCAGTACATCT |
| | ACGTATTAGTCATCGCTATT |
| | ACCATGGTCGAGGTGAGCCC |
| | CACGTTCTGCTTCACTCTCC |
| | CCATCTCCCCCCCCTCCCCA |
| | CCCCCAATTTTGTATTTATT |
| | TATTTTTTAATTATTTTGTG |
| | CAGCGATGGGGGCGGGGGGG |
| | GGGGGGGGGCGCGCGCCAGG |
| | CGGGGCGGGGCGGGGCGAGG |
| | GGCGGGGCGGGGCGAGGCGG |
| | AGAGGTGCGGCGGCAGCCAA |
| | TCAGAGCGGCGCGCTCCGAA |
| | AGTTTCCTTTTATGGCGAGG |
| | CGGCGGCGGCGGCGGCCCTA |
| | TAAAAAGCGAAGCGCGCGGC |
| | GGGCGGGGAGTCGCTGCGAC |
| | GCTGCCTTCGCCCCGTGCCC |
| | CGCTCCGCCGCCGCCTCGCG |
| | CCGCCCGCCCCGGCTCTGAC |
| | TGACCGCGTTACTCCCACAG |
| | GTGAGCGGGCGGGACGGCCC |
| | TTCTCCTCCGGGCTGTAATT |
| | AGCGCTTGGTTTAATGACGG |
| | CTTGTTTCTTTTCTGTGGCT |
| | GCGTGAAAGCCTTGAGGGGC |
| | TCCGGGAGGGCCCTTTGTGC |
| | GGGGGGAGCGGCTCGGGGGG |
| | TGCGTGCGTGTGTGTGTGCG |
| | TGGGGAGCGCCGCGTGCGGC |
| | TCCGCGCTGCCCGGCGGCTG |
| | TGAGCGCTGCGGGCGCGGCG |
| | CGGGGCTTTGTGCGCTCCGC |
| | AGTGTGCGCGAGGGGAGCGC |
| | GGCCGGGGGCGGTGCCCCGC |
| | GGTGCGGGGGGGGCTGCGAG |
| | GGGAACAAAGGCTGCGTGCG |
| | GGGTGTGTGCGTGGGGGGGT |
| | GAGCAGGGGGTGTGGGCGCG |
| | TCGGTCGGGCTGCAACCCCC |
| | CCTGCACCCCCCTCCCCGAG |
| | TTGCTGAGCACGGCCCGGCT |
| | TCGGGTGCGGGGCTCCGTAC |
| | GGGGCGTGGCGCGGGGCTCG |
| | CCGTGCCGGGCGGGGGGTGG |
| | CGGCAGGTGGGGGTGCCGGG |
| | CGGGGCGGGGCCGCCTCGGG |
| | CCGGGGAGGGCTCGGGGGAG |
| | GGGCGCGGCGGCCCCCGGAG |
| | CGCCGGCGGCTGTCGAGGCG |
| | CGGCGAGCCGCAGCCATTGC |
| | CTTTTATGGTAATCGTGCGA |
| | GAGGGCGCAGGGACTTCCTT |
| | TGTCCCAAATCTGTGCGGAG |
| | CCGAAATCTGGGAGGCGCCG |
| | CCGCACCCCCTCTAGCGGGC |
| | GCGGGGCGAAGCGGTGCGGC |
| | GCCGGCAGGAAGGAAATGGG |
| | CGGGGAGGGCCTTCGTGCGT |
| | CGCCGCGCCGCCGTCCCCTT |
| | CTCCCTCTCCAGCCTCGGGG |
| | CTGTCCGCGGGGGGACGGCT |
| | GCCTTCGGGGGGGACGGGGC |
| | AGGGCGGGGTTCGGCTTCTG |
| | GCGTGTGACCGGCGGCTCTA |
| | GAGCCTCTGCTAACCATGTT |
| | CATGCCTTCTTCTTTTTCCT |
| | ACAGCTCCTGGGCAACGTGC |
| | TGGTTATTGTGCTGTCTCAT |
| | CATTTTGGCAAAGAATTC |
| | (SEQ ID NO: 21) |
| hEF1aV2 | GGGCAGAGCGCACATCGCCC |
| | ACAGTCCCCGAGAAGTTGGG |
| | GGGAGGGGTCGGCAATTGAA |
| | CCGGTGCCTAGAGAAGGTGG |
| | CGCGGGGTAAACTGGGAAAG |
| | TGATGTCGTGTACTGGCTCC |
| | GCCTTTTTCCCGAGGGTGGG |
| | GGAGAACCGTATATAAGTGC |
| | AGTAGTCGCCGTGAACGTTC |
| | TTTTTCGCAACGGGTTTGCC |
| | GCCAGAACACAG (SEQ ID |
| | NO: 22) |
| hACTb | CCACTAGTTCCATGTCCTTA |
| | TATGGACTCATCTTTGCCTA |
| | TTGCGACACACACTCAATGA |
| | ACACCTACTACGCGCTGCAA |
| | AGAGCCCCGCAGGCCTGAGG |
| | TGCCCCCACCTCACCACTCT |
| | TCCTATTTTTGTGTAAAAAT |
| | CCAGCTTCTTGTCACCACCT |
| | CCAAGGAGGGGGAGGAGGAG |
| | GAAGGCAGGTTCCTCTAGGC |
| | TGAGCCGAATGCCCCTCTGT |
| | GGTCCCACGCCACTGATCGC |
| | TGCATGCCCACCACCTGGGT |
| | ACACACAGTCTGTGATTCCC |
| | GGAGCAGAACGGACCCTGCC |
| | CACCCGGTCTTGTGTGCTAC |
| | TCAGTGGACAGACCCAAGGC |
| | AAGAAAGGGTGACAAGGACA |
| | GGGTCTTCCCAGGCTGGCTT |
| | TGAGTTCCTAGCACCGCCCC |
| | GCCCCCAATCCTCTGTGGCA |
| | CATGGAGTCTTGGTCCCCAG |
| | AGTCCCCCAGCGGCCTCCAG |
| | ATGGTCTGGGAGGGCAGTTC |
| | AGCTGTGGCTGCGCATAGCA |
| | GACATACAACGGACGGTGGG |
| | CCCAGACCCAGGCTGTGTAG |
| | ACCCAGCCCCCCCGCCCCGC |
| | AGTGCCTAGGTCACCCACTA |
| | ACGCCCCAGGCCTGGTCTTG |
| | GCTGGGCGTGACTGTTACCC |
| | TCAAAAGCAGGCAGCTCCAG |
| | GGTAAAAGGTGCCCTGCCCT |
| | GTAGAGCCCACCTTCCTTCC |
| | CAGGGCTGCGGCTGGGTAGG |
| | TTTGTAGCCTTCATCACGGG |
| | CCACCTCCAGCCACTGGACC |
| | GCTGGCCCCTGCCCTGTCCT |
| | GGGGAGTGTGGTCCTGCGAC |
| | TTCTAAGTGGCCGCAAGCCA |
| | CCTGACTCCCCCAACACCAC |
| | ACTCTACCTCTCAAGCCCAG |
| | GTCTCTCCCTAGTGACCCAC |
| | CCAGCACATTTAGCTAGCTG |
| | AGCCCCACAGCCAGAGGTCC |
| | TCAGGCCCTGCTTTCAGGGC |
| | AGTTGCTCTGAAGTCGGCAA |
| | GGGGGAGTGACTGCCTGGCC |
| | ACTCCATGCCCTCCAAGAGC |
| | TCCTTCTGCAGGAGCGTACA |
| | GAACCCAGGGCCCTGGCACC |
| | CGTGCAGACCCTGGCCCACC |
| | CCACCTGGGCGCTCAGTGCC |
| | CAAGAGATGTCCACACCTAG |
| | GATGTCCCGCGGTGGGTGGG |
| | GGGCCCGAGAGACGGGCAGG |
| | CCGGGGGCAGGCCTGGCCAT |
| | GCGGGGCCGAACCGGGCACT |
| | GCCCAGCGTGGGGCGCGGGG |
| | GCCACGGCGCGCGCCCCCAG |
| | CCCCCGGGCCCAGCACCCCA |
| | AGGCGGCCAACGCCAAAACT |
| | CTCCCTCCTCCTCTTCCTCA |
| | ATCTCGCTCTCGCTCTTTTT |
| | TTTTTTCGCAAAAGGAGGGG |
| | AGAGGGGGTAAAAAAATGCT |
| | GCACTGTGCGGCGAAGCCGG |
| | TGAGTGAGCGGCGCGGGGCC |
| | AATCAGCGTGCGCCGTTCCG |
| | AAAGTTGCCTTTTATGGCTC |
| | GAGCGGCCGCGGCGGCGCCC |
| | TATAAAACCCAGCGGCGCGA |
| | CGCGCCACCACCGCCGAGAC |
| | CGCGTCCGCCCCGCGAGCAC |
| | AGAGCCTCGCCTTTGCCGAT |
| | CCGCCGCCCGTCCACACCCG |
| | CCGCCAGGTAAGCCCGGCCA |
| | GCCGACCGGGGCAGGCGGCT |
| | CACGGCCCGGCCGCAGGCGG |
| | CCGCGGCCCCTTCGCCCGTG |
| | CAGAGCCGCCGTCTGGGCCG |
| | CAGCGGGGGGCGCATGGGGG |
| | GGGAACCGGACCGCCGTGGG |
| | GGGCGCGGGAGAAGCCCCTG |
| | GGCCTCCGGAGATGGGGGAC |
| | ACCCCACGCCAGTTCGGAGG |
| | CGCGAGGCCGCGCTCGGGAG |
| | GCGCGCTCCGGGGGTGCCGC |
| | TCTCGGGGCGGGGGCAACCG |
| | GCGGGGTCTTTGTCTGAGCC |
| | GGGCTCTTGCCAATGGGGAT |
| | CGCAGGGTGGGCGCGGCGGA |
| | GCCCCCGCCAGGCCCGGTGG |
| | GGGCTGGGGCGCCATTGCGC |
| | GTGCGCGCTGGTCCTTTGGG |
| | CGCTAACTGCGTGCGCGCTG |
| | GGAATTGGCGCTAATTGCGC |
| | GTGCGCGCTGGGACTCAAGG |
| | CGCTAACTGCGCGTGCGTTC |
| | TGGGGCCCGGGGTGCCGCGG |
| | CCTGGGCTGGGGCGAAGGCG |
| | GGCTCGGCCGGAAGGGGTGG |
| | GGTCGCCGCGGCTCCCGGGC |
| | GCTTGCGCGCACTTCCTGCC |
| | CGAGCCGCTGGCCGCCCGAG |
| | GGTGTGGCCGCTGCGTGCGC |
| | GCGCGCCGACCCGGCGCTGT |
| | TTGAACCGGGCGGAGGCGGG |
| | GCTGGCGCCCGGTTGGGAGG |
| | GGGTTGGGGCCTGGCTTCCT |
| | GCCGCGCGCCGCGGGGACGC |
| | CTCCGACCAGTGTTTGCCTT |
| | TTATGGTAATAACGCGGCCG |
| | GCCCGGCTTCCTTTGTCCCC |
| | AATCTGGGCGCGCGCCGGCG |
| | CCCCCTGGCGGCCTAAGGAC |
| | TCGGCGCGCCGGAAGTGGCC |
| | AGGGCGGGGGCGACCTCGGC |
| | TCACAGCGCGCCCGGCTAT |
| | (SEQ ID NO: 23) |
| heIF4A1 | GTTGATTTCCTTCATCCCTG |
| | GCACACGTCCAGGCAGTGTC |
| | GAATCCATCTCTGCTACAGG |
| | GGAAAACAAATAACATTTGA |
| | GTCCAGTGGAGACCGGGAGC |
| | AGAAGTAAAGGGAAGTGATA |
| | ACCCCCAGAGCCCGGAAGCC |
| | TCTGGAGGCTGAGACCTCGC |
| | CCCCCTTGCGTGATAGGGCC |
| | TACGGAGCCACATGACCAAG |
| | GCACTGTCGCCTCCGCACGT |
| | GTGAGAGTGCAGGGCCCCAA |
| | GATGGCTGCCAGGCCTCGAG |
| | GCCTGACTCTTCTATGTCAC |
| | TTCCGTACCGGCGAGAAAGG |
| | CGGGCCCTCCAGCCAATGAG |
| | GCTGCGGGGCGGGCCTTCAC |
| | CTTGATAGGCACTCGAGTTA |
| | TCCAATGGTGCCTGCGGGCC |
| | GGAGCGACTAGGAACTAACG |
| | TCATGCCGAGTTGCTGAGCG |
| | CCGGCAGGCGGGGCCGGGGC |
| | GGCCAAACCAATGCGATGGC |
| | CGGGGCGGAGTCGGGCGCTC |
| | TATAAGTTGTCGATAGGCGG |
| | GCACTCCGCCCTAGTTTCTA |
| | AGGACCATG |
| | (SEQ ID NO: 24) |
| hGAPDH | AGTTCCCCAACTTTCCCGCC |
| | TCTCAGCCTTTGAAAGAAAG |
| | AAAGGGGAGGGGGCAGGCCG |
| | CGTGCAGTCGCGAGCGGTGC |
| | TGGGCTCCGGCTCCAATTCC |
| | CCATCTCAGTCGCTCCCAAA |
| | GTCCTTCTGTTTCATCCAAG |
| | CGTGTAAGGGTCCCCGTCCT |
| | TGACTCCCTAGTGTCCTGCT |
| | GCCCACAGTCCAGTCCTGGG |
| | AACCAGCACCGATCACCTCC |
| | CATCGGGCCAATCTCAGTCC |
| | CTTCCCCCCTACGTCGGGGC |
| | CCACACGCTCGGTGCGTGCC |
| | CAGTTGAACCAGGCGGCTGC |
| | GGAAAAAAAAAAGCGGGGAG |
| | AAAGTAGGGCCCGGCTACTA |
| | GCGGTTTTACGGGCGCACGT |
| | AGCTCAGGCCTCAAGACCTT |
| | GGGCTGGGACTGGCTGAGCC |
| | TGGCGGGAGGCGGGGTCCGA |
| | GTCACCGCCTGCCGCCGCGC |
| | CCCCGGTTTCTATAAATTGA |
| | GCCCGCAGCCTCCCGCTTCG |
| | CTCTCTGCTCCTCCTGTTCG |
| | ACAGTCAGCCGCATCTTCTT |
| | TTGCGTCGCCAGGTGAAGAC |
| | GGGCGGAGAGAAACCCGGGA |
| | GGCTAGGGACGGCCTGAAGG |
| | CGGCAGGGGCGGGCGCAGGC |
| | CGGATGTGTTCGCGCCGCTG |
| | CGGGGTGGGCCCGGGCGGCC |
| | TCCGCATTGCAGGGGCGGGC |
| | GGAGGACGTGATGCGGCGCG |
| | GGCTGGGCATGGAGGCCTGG |
| | TGGGGGAGGGGAGGGGAGGC |
| | GTGGGTGTCGGCCGGGGCCA |
| | CTAGGCGCTCACTGTTCTCT |
| | CCCTCCGCGCAGCCGAGCCA |
| | CATCGCTGAGACAC |
| | (SEQ ID NO: 25) |
| hGRP78 | AGTGCGGTTACCAGCGGAAA |
| | TGCCTCGGGGTCAGAAGTCG |
| | CAGGAGAGATAGACAGCTGC |
| | TGAACCAATGGGACCAGCGG |
| | ATGGGGCGGATGTTATCTAC |
| | CATTGGTGAACGTTAGAAAC |
| | GAATAGCAGCCAATGAATCA |
| | GCTGGGGGGGCGGAGCAGTG |
| | ACGTTTATTGCGGAGGGGGC |
| | CGCTTCGAATCGGCGGCGGC |
| | CAGCTTGGTGGCCTGGGCCA |
| | ATGAACGGCCTCCAACGAGC |
| | AGGGCCTTCACCAATCGGCG |
| | GCCTCCACGACGGGGCTGGG |
| | GGAGGGTATATAAGCCGAGT |
| | AGGCGACGGTGAGGTCGACG |
| | CCGGCCAAGACAGCACAGAC |
| | AGATTGACCTATTGGGGTGT |
| | TTCGCGAGTGTGAGAGGGAA |
| | GCGCCGCGGCCTGTATTTCT |
| | AGACCTGCCCTTCGCCTGGT |
| | TCGTGGCGCCTTGTGACCCC |
| | GGGCCCCTGCCGCCTGCAAG |
| | TCGGAAATTGCGCTGTGCTC |
| | CTGTGCTACGGCCTGTGGCT |
| | GGACTGCCTGCTGCTGCCCA |
| | ACTGGCTGGCAC |
| | (SEQ ID NO: 26) |
| hGRP94 | TAGTTTCATCACCACCGCCA |
| | CCCCCCCGCCCCCCCGCCAT |
| | CTGAAAGGGTTCTAGGGGAT |
| | TTGCAACCTCTCTCGTGTGT |
| | TTCTTCTTTCCGAGAAGCGC |
| | CGCCACACGAGAAAGCTGGC |
| | CGCGAAAGTCGTGCTGGAAT |
| | CACTTCCAACGAAACCCCAG |
| | GCATAGATGGGAAAGGGTGA |
| | AGAACACGTTGCCATGGCTA |
| | CCGTTTCCCCGGTCACGGAA |
| | TAAACGCTCTCTAGGATCCG |
| | GAAGTAGTTCCGCCGCGACC |
| | TCTCTAAAAGGATGGATGTG |
| | TTCTCTGCTTACATTCATTG |
| | GACGTTTTCCCTTAGAGGCC |
| | AAGGCCGCCCAGGCAAAGGG |
| | GCGGTCCCACGCGTGAGGGG |
| | CCCGCGGAGCCATTTGATTG |
| | GAGAAAAGCTGCAAACCCTG |
| | ACCAATCGGAAGGAGCCACG |
| | CTTCGGGCATCGGTCACCGC |
| | ACCTGGACAGCTCCGATTGG |
| | TGGACTTCCGCCCCCCCTCA |
| | CGAATCCTCATTGGGTGCCG |
| | TGGGTGCGTGGTGCGGCGCG |
| | ATTGGTGGGTTCATGTTTCC |
| | CGTCCCCCGCCCGCGAGAAG |
| | TGGGGGTGAAAAGCGGCCCG |
| | ACCTGCTTGGGGTGTAGTGG |
| | GCGGACCGCGCGGCTGGAGG |
| | TGTGAGGATCCGAACCCAGG |
| | GGTGGGGGGTGGAGGCGGCT |
| | CCTGCGATCGAAGGGGACTT |
| | GAGACTCACCGGCCGCACGT |
| | C (SEQ ID NO: 27) |
| hHSP70 | GGGCCGCCCACTCCCCCTTC |
| | CTCTCAGGGTCCCTGTCCCC |
| | TCCAGTGAATCCCAGAAGAC |
| | TCTGGAGAGTTCTGAGCAGG |
| | GGGCGGCACTCTGGCCTCTG |
| | ATTGGTCCAAGGAAGGCTGG |
| | GGGGCAGGACGGGAGGCGAA |
| | AACCCTGGAATATTCCCGAC |
| | CTGGCAGCCTCATCGAGCTC |
| | GGTGATTGGCTCAGAAGGGA |
| | AAAGGCGGGTCTCCGTGACG |
| | ACTTATAAAAGCCCAGGGGC |
| | AAGCGGTCCGGATAACGGCT |
| | AGCCTGAGGAGCTGCTGCGA |
| | CAGTCCACTACCTTTTTCGA |
| | GAGTGACTCCCGTTGTCCCA |
| | AGGCTTCCCAGAGCGAACCT |
| | GTGCGGCTGCAGGCACCGGC |
| | GCGTCGAGTTTCCGGCGTCC |
| | GGAAGGACCGAGCTCTTCTC |
| | GCGGATCCAGTGTTCCGTTT |
| | CCAGCCCCCAATCTCAGAGC |
| | GGAGCCGACAGAGAGCAGGG |
| | AACCC (SEQ ID NO: 28) |
| hKINb | GCCCCACCCCCGTCCGCGTT |
| | ACAACCGGGAGGCCCGCTGG |
| | GTCCTGCACCGTCACCCTCC |
| | TCCCTGTGACCGCCCACCTG |
| | ATACCCAAACAACTTTCTCG |
| | CCCCTCCAGTCCCCAGCTCG |
| | CCGAGCGCTTGCGGGGAGCC |
| | ACCCAGCCTCAGTTTCCCCA |
| | GCCCCGGGCGGGGCGAGGGG |
| | CGATGACGTCATGCCGGCGC |
| | GCGGCATTGTGGGGCGGGGC |
| | GAGGCGGGGCGCCGGGGGGA |
| | GCAACACTGAGACGCCATTT |
| | TCGGCGGCGGGAGCGGCGCA |
| | GGCGGCCGAGCGGGACTGGC |
| | TGGGTCGGCTGGGCTGCTGG |
| | TGCGAGGAGCCGCGGGGCTG |
| | TGCTCGGCGGCCAAGGGGAC |
| | AGCGCGTGGGTGGCCGAGGA |
| | TGCTGCGGGGCGGTAGCTCC |
| | GGCGCCCCTCGCTGGTGACT |
| | GCTGCGCCGTGCCTCACACA |
| | GCCGAGGCGGGCTCGGCGCA |
| | CAGTCGCTGCTCCGCGCTCG |
| | CGCCCGGCGGCGCTCCAGGT |
| | GCTGACAGCGCGAGAGAGCG |
| | CGGCCTCAGGAGCAACAC |
| | (SEQ ID NO:29) |
| hUBIb | TTCCAGAGCTTTCGAGGAAG |
| | GTTTCTTCAACTCAAATTCA |
| | TCCGCCTGATAATTTTCTTA |
| | TATTTTCCTAAAGAAGGAAG |
| | AGAAGCGCATAGAGGAGAAG |
| | GGAAATAATTTTTTAGGAGC |
| | CTTTCTTACGGCTATGAGGA |
| | ATTTGGGGCTCAGTTGAAAA |
| | GCCTAAACTGCCTCTCGGGA |
| | GGTTGGGCGCGGCGAACTAC |
| | TTTCAGCGGCGCACGGAGAC |
| | GGCGTCTACGTGAGGGGTGA |
| | TAAGTGACGCAACACTCGTT |
| | GCATAAATTTGCGCTCCGCC |
| | AGCCCGGAGCATTTAGGGGC |
| | GGTTGGCTTTGTTGGGTGAG |
| | CTTGTTTGTGTCCCTGTGGG |
| | TGGACGTGGTTGGTGATTGG |
| | CAGGATCCTGGTATCCGCTA |
| | ACAGGTACTGGCCCACAGCC |
| | GTAAAGACCTGCGGGGGCGT |
| | GAGAGGGGGGAATGGGTGAG |
| | GTCAAGCTGGAGGCTTCTTG |
| | GGGTTGGGTGGGCCGCTGAG |
| | GGGAGGGGAGGGCGAGGTGA |
| | CGCGACACCCGGCCTTTCTG |
| | GGAGAGTGGGCCTTGTTGAC |
| | CTAAGGGGGGCGAGGGCAGT |
| | TGGCACGCGCACGCGCCGAC |
| | AGAAACTAACAGACATTAAC |
| | CAACAGCGATTCCGTCGCGT |
| | TTACTTGGGAGGAAGGCGGA |
| | AAAGAGGTAGTTTGTGTGGC |
| | TTCTGGAAACCCTAAATTTG |
| | GAATCCCAGTATGAGAATGG |
| | TGTCCCTTCTTGTGTTTCAA |
| | TGGGATTTTTACTTCGCGAG |
| | TCTTGTGGGTTTGGTTTTGT |
| | TTTCAGTTTGCCTAACACCG |
| | TGCTTAGGTTTGAGGCAGAT |
| | TGGAGTTCGGTCGGGGGAGT |
| | TTGAATATCCGGAACAGTTA |
| | GTGGGGAAAGCTGTGGACGC |
| | TTGGTAAGAGAGCGCTCTGG |
| | ATTTTCCGCTGTTGACGTTG |
| | AAACCTTGAATGACGAATTT |
| | CGTATTAAGTGACTTAGCCT |
| | TGTAAAATTGAGGGGAGGCT |
| | TGCGGAATATTAACGTATTT |
| | AAGGCATTTTGAAGGAATAG |
| | TTGCTAATTTTGAAGAATAT |
| | TAGGTGTAAAAGCAAGAAAT |
| | ACAATGATCCTGAGGTGACA |
| | CGCTTATGTTTTACTTTTAA |
| | ACTAGGTCACC |
| | (SEQ ID NO: 30) |

In some embodiments, the promoter sequence is derived from a promoter selected from: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.

In some embodiments, the first promoter is a constitutive promoter, an inducible promoter, or a synthetic promoter. In some embodiments, the constitutive promoter is selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, the ACP-responsive promoter is a synthetic promoter. In some embodiments, the ACP-responsive promoter comprises a minimal promoter. In some embodiments, the ACP-binding domain comprises one or more zinc finger binding sites. The ACP-binding domain can comprise 1, 2, 3, 4, 5, 6 7, 8, 9, 10, or more zinc finger binding sites. In some embodiments, the ACP-binding domain comprises one zinc finger binding site. In some embodiments, the ACP-binding domain comprises two zinc finger binding sites. In some embodiments, the ACP-binding domain comprises three zinc finger binding sites. In some embodiments, the ACP-binding domain comprises four zinc finger binding sites. An exemplary ACP-binding domain comprising zinc finger binding sites is shown in the sequence

(SEQ ID NO: 92)
cgggtttcgtaacaatcgcatgaggattcgcaacgccttcGG
CGTAGCCGATGTCGCGctcccgtctcagtaaaggtcGGCGTA
GCCGATGTCGCGcaatcggactgccttcgtacGGCGTAGCCG
ATGTCGCGcgtatcagtcgcctcggaacGGCGTAGCCGATGT
CGCGcattcgtaagaggctcactctcccttacacggagtgga
taACTAGTTCTAGAGGGTATATAATGGGGGCCA.

In some embodiments, the ACP-responsive promoter comprises an enhancer that promotes transcription when an antigen recognizing receptor engages a cognate antigen, e.g., an antigen expressed on a target cell. Enhancers can include, but are not limited to, enhancers enriched in the ATAC-seq of activated T cells (Gate et al. Nat Genet. Author manuscript; available in PMC 2019 Jan. 9; herein incorporated by reference for all purposes) or enhancers associated with upregulated genes in single-cell RNA seq data (Xhangolli et al. Genomics Proteomics Bioinformatics. 2019 April; 17(2):129-139. doi: 10.1016/j.gpb.2019.03.002; herein incorporated by reference for all purposes). An enhancer can be a synthetic enhancer, such as a pair of transcription factors known or suspected to be upregulated in activated T cells or NK cells. Synthetic enhancers can include multiple iterations of transcription factor binding sites, such 4 iterations of two distinct transcription factor binding sites in an aaaabbbb or abababab organization. Illustrative non-limiting examples of genes from which enhancers can be derived include, but are not limited to, ATF2, ATF7, BACH1, BATF, Bcl-6, Blimp-1, BMI1, CBFB, CREB1, CREM, CTCF, E2F1, EBF1, EGR1, ETV6, FOS, FOXA1, FOXA2, GATA3, HIF1A, IKZF1, IKZF2, IRF4, JUN, JUNB, JUND, Lef1, NFAT, NFIA, NFIB, NFKB, NR2F1, Nur77, PU.1, RELA, RUNX3, SCRT1, SCRT2, SP1, STAT4, STAT5A, T-Bet, Tcf7, ZBED1, ZNF143, or ZNF217.

In some embodiments, the ACP-responsive promoter comprises a promoter that promotes transcription when a receptor engages a cognate ligand, such as in a activation inducible system. In some embodiments, the ACP-responsive promoter comprises a promoter that promotes transcription when an antigen recognizing receptor engages a cognate antigen, e.g., an antigen expressed on a target cell. For example, when the ACP is an antigen receptor (e.g., a CAR), the ACP-responsive promoter can include promoters that are induced by signal transduction following antigen receptor binding to a cognate antigen. ACP-responsive promoters can include promoters with increased transcriptional activity in activated T cells and/or NK cells. ACP-responsive promoters can include promoters derived from genes that are upregulated in activated cells, such as T cells and/or NK cells. ACP-responsive promoters can include promoters derived from genes that have increased transcription factor binding in activated cells, such T cells and/or NK cells. Derived promoters can include the genomic region 2 kb upstream of the gene. Derived promoters can include the genomic region −100 bp downstream of the transcription initiation site the gene. Derived promoters can include the genomic region 2 kb upstream of the gene to −100 bp downstream of the transcription initiation site the gene. Derived promoters can include the genomic region upstream of the translation initiation site the gene. Derived promoters can include the genomic region 2 kb upstream to the translation initiation site the gene. Derived promoters can include one or more enhancers identified in a promoter region. ACP-responsive promoters can include, but are not limited to, promoters derived from CCL3, CCL4, or MTA2 genes. ACP-responsive promoters can include, but are not limited to, a CCL3 promoter region (e.g., SEQ ID NO: 156), a CCL4 promoter region (e.g., SEQ ID NO: 157), and/or a MTA2 promoter region (e.g., SEQ ID NO: 158). ACP-responsive promoters can include enhancers present in a CCL3 promoter region (e.g., SEQ ID NO: 156), a CCL4 promoter region (e.g., SEQ ID NO: 157), and/or a MTA2 promoter region (e.g., SEQ ID NO: 158). ACP-responsive promoters can include synthetic promoters. For example, ACP-responsive promoters can include antigen induced enhancers or promoter sequences combined with other promoters, such as minimal promoters (e.g., min AdeP or YB-TATA). ACP-responsive promoters can include synthetic enhancers, such as promoters including multiple iterations of transcription factor binding sites. In an illustrative non-limiting example, ACP-responsive promoter including a synthetic promoter can include 5 iterations of NFAT transcription factor binding sites in combination with a minimal Ade promoter (5× NFAT_minAdeP).

Multicistronic and Multiple Promoter Systems

In some embodiments, engineered nucleic acids are configured to produce multiple effector molecules. For example, nucleic acids may be configured to produce 2-20 different effector molecules. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, nucleic acids are configured to produce 1, 2, 3, 4, 5, 6, 7, 8, 9, 10, 11, 12, 13, 14, 15, 16, 17, 18, 19, or 20 effector molecules.

In some embodiments, engineered nucleic acids can be multicistronic, i.e., more than one separate polypeptide (e.g., multiple exogenous polynucleotides or effector molecules) can be produced from a single mRNA transcript. Engineered nucleic acids can be multicistronic through the use of various linkers, e.g., a polynucleotide sequence encoding a first exogenous polynucleotide or effector molecule can be linked to a nucleotide sequence encoding a second exogenous polynucleotide or effector molecule, such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

In some embodiments, when the second expression cassette comprises two or more units of (L₁-E)_{X}, each L₁ linker polynucleotide sequence is operably associated with the translation of each effector molecule as a separate polypeptide.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

“Linkers,” as used herein can refer to polypeptides that link a first polypeptide sequence and a second polypeptide sequence or the multicistronic linkers described above.

Effector Molecules

Any suitable effector molecule known in the art can be encoded by the engineered nucleic acid or expressed by the engineered cell. Suitable effector molecules can be grouped into therapeutic classes based on structure similarity, sequence similarity, or function. Effector molecule therapeutic classes include, but are not limited to, cytokines, chemokines, homing molecules, growth factors, co-activation molecules, tumor microenvironment modifiers, receptors, ligands, antibodies, polynucleotides, peptides, and enzymes.

In some embodiments, each effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, a effector molecule is a chemokine. Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. Non-limiting examples of chemokines that may be encoded by the engineered nucleic acids of the present disclosure include: CCL21a, CXCL10, CXCL11, CXCL13, a CXCL10-CXCL11 fusion protein, CCL19, CXCL9, and XCL1, or any combination thereof. In some embodiments, the chemokine is selected from: CCL21a, CXCL10, CXCL11, CXCL13, a CXCL10-CXCL11 fusion protein, CCL19, CXCL9, and XCL1.

In some embodiments, a effector molecule is a cytokine. Non-limiting examples of cytokines that may be encoded by the engineered nucleic acids of the present disclosure include: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha, or any combination thereof. In some embodiments, the cytokine is selected from: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, engineered nucleic acids are configured to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, PDGFR, anti-integrin alpha4,beta7; anti-MAdCAM; CCR9; CXCR4; SDF1; MMP-2; CXCR1; CXCR7; CCR2; CCR4; and GPR15, or any combination thereof. In some embodiments, the homing molecule is selected from: anti-integrin alpha4,beta7; anti-MAdCAM; CCR9; CXCR4; SDF1; MMP-2; CXCR1; CXCR7; CCR2; CCR4; and GPR15.

In some embodiments, engineered nucleic acids are configured to produce at least one growth factor. Suitable growth factors for use as an effector molecule include, but are not limited to, FLT3L and GM-CSF, or any combination thereof. In some embodiments, the growth factor is selected from: FLT3L and GM-CSF.

In some embodiments, engineered nucleic acids are configured to produce at least one co-activation molecule. Suitable co-activation molecules for use as an effector molecule include, but are not limited to, c-Jun, 4-1BBL and CD40L, or any combination thereof. In some embodiments, the co-activation molecule is selected from: c-Jun, 4-1BBL and CD40L.

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007). Suitable tumor microenvironment modifiers for use as an effector molecule include, but are not limited to, adenosine deaminase, TGFbeta inhibitors, immune checkpoint inhibitors, VEGF inhibitors, and HPGE2, or any combination thereof. In some embodiments, the tumor microenvironment modifier is selected from: adenosine deaminase, TGFbeta inhibitors, immune checkpoint inhibitors, VEGF inhibitors, and HPGE2.

In some embodiments, engineered nucleic acids are configured to produce at least one TGFbeta inhibitor. Suitable TGFbeta inhibitors for use as an effector molecule include, but are not limited to, an anti-TGFbeta peptide, an anti-TGFbeta antibody, a TGFb-TRAP, or combinations thereof. In some embodiments, the TGFbeta inhibitors are selected from: an anti-TGFbeta peptide, an anti-TGFbeta antibody, a TGFb-TRAP, and combinations thereof.

In some embodiments, engineered nucleic acids are configured to produce at least one immune checkpoint inhibitor. Suitable immune checkpoint inhibitors for use as an effector molecule include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies, or any combination thereof. In some embodiments, the immune checkpoint inhibitors are selected from: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MEDI4736/Imfinzi®-Medimmune/AstraZeneca), atezolizumab (anti-PD-L1; Tecentriq®—Roche/Genentech), BMS-936559 (anti-PD-L1-BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca).

In some embodiments, engineered nucleic acids are configured to produce at least one VEGF inhibitor. Suitable VEGF inhibitors for use as an effector molecule include, but are not limited to, anti-VEGF antibodies, anti-VEGF peptides, or combinations thereof. In some embodiments, the VEGF inhibitors comprise anti-VEGF antibodies, anti-VEGF peptides, or combinations thereof.

In some embodiments, each effector molecule is a human-derived effector molecule.

Secrection Signals

In general, the one or more effector molecules comprise a secretion signal peptide (also referred to as a signal peptide or signal sequence) at the effector molecule's N-terminus that direct newly synthesized proteins destined for secretion or membrane insertion to the proper protein processing pathways. In embodiments with two or more effector molecules, each effector molecule can comprise a secretion signal (S). In embodiments with two or more effector molecules, each effector molecule can comprise a secretion signal such that each effector molecule is secreted from an engineered cell. In embodiments, the second expression cassette comprising one or more units of (L-E)_{X} further comprises a polynucleotide sequence encoding a secretion signal peptide (S). In embodiments, for each X the corresponding secretion signal peptide is operably associated with the effector molecule. In embodiments, the second expression cassette comprising an ACP-responsive promoter and a second exogenous polynucleotide sequence having the formula: (L-S-E)_{X}.

The secretion signal peptide operably associated with a effector molecule can be a native secretion signal peptide native secretion signal peptide (e.g., the secretion signal peptide generally endogenously associated with the given effector molecule). The secretion signal peptide operably associated with a effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 5.

TABLE 5
Exemplary Signal Secretion Peptides

| Name | Protein SEQUENCE | Source (Uniprot) | DNA SEQUENCE |
|---|---|---|---|
| IL-12 | MCHQQLVISWFSL | P29460 | ATGTGTCACCAGCAGCTCGTTAT |
| | VFLASPLVA (SEQ | | ATCCTGGTTTAGTTTGGTGTTTCT |
| | ID NO: 56) | | CGCTTCACCCCTGGTGGCA (SEQ |
| | | | ID NO: 31) |
| IL-12 (Codon | MCHQQLVISWFSL | — | ATGTGCCATCAGCAACTCGTCAT |
| Optimized) | VFLASPLVA (SEQ | | CTCCTGGTTCTCCCTTGTGTTCCT |
| | ID NO: 57) | | CGCTTCCCCTCTGGTCGCC (SEQ |
| | | | ID NO: 32) |
| IL-2 (Optimized) | MQLLSCIALILALV | — | ATGCAACTGCTGTCATGTATCGC |
| | (SEQ ID NO: 58) | | ACTCATCCTGGCGCTGGTA(SEQ |
| | | | ID NO: 33) |
| IL-2 (Native) | MYRMQLLSCIALSL | P60568 | ATGTATCGGATGCAACTTTTGAG |
| | ALVTNS (SEQ ID | | CTGCATCGCATTGTCTCTGGCGCT |
| | NO: 59) | | GGTGACAAATTCC (SEQ ID NO: |
| | | | 34) |
| Trypsinogen-2 | MNLLLILTFVAAAV | P07478 | ATGAATCTCTTGCTCATACTTACG |
| | A (SEQ ID NO: 60) | | TTTGTCGCTGCTGCCGTTGCG |
| | | | (SEQ ID NO: 35) |
| Gaussia | MGVKVLFALICIAV | — | ATGGGCGTGAAGGTCTTGTTTGC |
| Luciferase | AEA (SEQ ID NO: | | CCTTATCTGCATAGCTGTTGCGG |
| | 61) | | AGGCG (SEQ ID NO: 36) |
| CDS | MPMGSLQPLATLY | P06127 | ATGCCGATGGGGAGCCTTCAACC |
| | LLGMLVASCLG | | TTTGGCAACGCTTTATCTTCTGGG |
| | (SEQ ID NO: 62) | | GATGTTGGTTGCTAGTTGCCTTGG |
| | | | G (SEQ ID NO: 37) |
| IgKVII (mouse) | METDTLLLWVLLL | | ATGGAAACTGACACGTTGTTGCT |
| | WVPGSTGD (SEQ | | GTGGGTATTGCTCTTGTGGGTCCC |
| | ID NO: 63) | | AGGATCTACGGGCGAC (SEQ ID |
| | | | NO: 38) |
| IgKVII (human) | MDMRVPAQLLGLL | P01597 | ATGGATATGAGGGTTCCCGCCCA |
| | LLWLRGARC (SEQ | | GCTTTTGGGGCTGCTTTTGTTGTG |
| | ID NO: 64) | | GCTTCGAGGGGCTCGGTGT (SEQ |
| | | | ID NO: 39) |
| VSV-G | MKCLLYLAFLFIGV | — | ATGAAGTGTCTGTTGTACCTGGC |
| | NC (SEQ ID NO: 65) | | GTTTCTGTTCATTGGTGTAAACTG |
| | | | T (SEQ ID NO: 40) |
| Prolactin | MNIKGSPWKGSLL | P01236 | ATGAATATCAAAGGAAGTCCGTG |
| | LLLVSNLLLCQSVA | | GAAGGGTAGTCTCCTGCTGCTCC |
| | P (SEQ ID NO: 66) | | TCGTATCTAACCTTCTCCTTTGTC |
| | | | AATCCGTGGCACCC (SEQ ID NO: |
| | | | 41) |
| Serum albumin | MKWVTFISLLFLFS | P02768 | ATGAAATGGGTAACATTCATATC |
| preproprotein | SAYS (SEQ ID NO: | | ACTTCTCTTTCTGTTCAGCTCTGC |
| | 67) | | GTATTCT (SEQ ID NO: 42) |
| Azurocidin | MTRLTVLALLAGL | 20160 | ATGACAAGGCTTACTGTTTTGGC |
| preproprotein | LASSRA (SEQ ID | | TCTCCTCGCTGGACTCTTGGCTTC |
| | NO: 68) | | CTCCCGAGCA (SEQ ID NO: 43) |
| Osteonectin | MRAWIFFLLCLAG | P09486 | ATGAGGGCTTGGATTTTTTTTCTG |
| (BM40) | RALA (SEQ ID NO: | | CTCTGCCTTGCCGGTCGAGCCCT |
| | 69) | | GGCG (SEQ ID NO: 44) |
| CD33 | MPLLLLLPLLWAG | P20138 | ATGCCTCTTCTGCTTTTGCTTCCT |
| | ALA (SEQ ID NO: | | CTTTTGTGGGCAGGTGCCCTCGC |
| | 70) | | A (SEQ ID NO: 45) |
| IL-6 | MNSFSTSAFGPVAF | P05231 | ATGAACTCTTTCTCAACCTCTGCG |
| | SLGLLLVLPAAFPA | | TTTGGTCCGGTCGCTTTCTCCCTT |
| | P (SEQ ID NO: 71) | | GGGCTCCTGCTTGTCTTGCCAGC |
| | | | AGCGTTTCCTGCGCCA (SEQ ID |
| | | | NO: 46) |
| IL-8 | MTSKLAVALLAAF | P10145 | ATGACAAGTAAACTGGCGGTAGC |
| | LISAALC (SEQ ID | | CTTGCTCGCGGCCTTTTTGATTTC |
| | NO: 72) | | CGCAGCCCTTTGT |
| | | | (SEQ ID NO: 47) |
| CCL2 | MKVSAALLCLLLIA | P13500 | ATGAAGGTAAGTGCAGCGTTGCT |
| | ATFIPQGLA (SEQ | | TTGCCTTCTCCTCATTGCAGCGAC |
| | ID NO: 73) | | CTTTATTCCTCAAGGGCTGGCC |
| | | | (SEQ ID NO: 48) |
| TIMP2 | MGAAARTLRLALG | P16035 | ATGGGAGCGGCAGCTAGAACACT |
| | LLLLATLLRPADA | | TCGACTTGCCCTTGGGCTCTTGCT |
| | (SEQ ID NO: 74) | | CCTTGCAACCCTCCTTAGACCTGC |
| | | | CGACGCA (SEQ ID NO: 49) |
| VEGFB | MSPLLRRLLLAALL | P49765 | ATGTCACCGTTGTTGCGGAGATT |
| | QLAPAQA (SEQ ID | | GCTGTTGGCCGCACTTTTGCAACT |
| | NO: 75) | | GGCTCCTGCTCAAGCC (SEQ ID |
| | | | NO: 50) |
| Osteoprotegerin | MNNLLCCALVFLDI | O00300 | ATGAATAACCTGCTCTGTTGTGC |
| | SIKWTTQ (SEQ ID | | GCTCGTGTTCCTGGACATTTCTAT |
| | NO: 76) | | AAAATGGACAACGCAA (SEQ ID |
| | | | NO: 51) |
| Serpin E1 | MQMSPALTCLVLG | P05121 | ATGCAAATGTCTCCTGCCCTTACC |
| | LALVFGEGSA (SEQ | | TGTCTCGTACTTGGTCTTGCGCTC |
| | ID NO: 77) | | GTATTTGGAGAGGGATCAGCC |
| | | | (SEQ ID NO: 52) |
| GROalpha | MARAALSAAPSNP | P09341 | ATGGCAAGGGCTGCACTCAGTGC |
| | RLLRVALLLLLLVA | | TGCCCCGTCTAATCCCAGATTGCT |
| | AGRRAAG (SEQ ID | | TCGAGTTGCATTGCTTCTTCTGTT |
| | NO: 78) | | GCTGGTTGCAGCTGGTAGGAGAG |
| | | | CAGCGGGT (SEQ ID NO: 53) |
| CXCL12 | MNAKVVVVLVLV | P48061 | ATGAATGCAAAAGTCGTGGTCGT |
| | LTALCLSDG (SEQ | | GCTGGTTTTGGTTCTGACGGCGTT |
| | ID NO: 79) | | GTGTCTTAGTGATGGG (SEQ ID |
| | | | NO: 54) |
| IL-21 (Codon | MERIVICLMVIFLG | Q9HBE4 | ATGGAACGCATTGTGATCTGCCT |
| Optimized) | TLVHKSSS (SEQ ID | | GATGGTCATCTTCCTGGGCACCTT |
| | NO: 80) | | AGTGCACAAGTCGAGCAGC(SEQ |
| | | | ID NO: 55) |
| CD8a | MALPVTALLLPLAL | P01732 | ATGGCTCTCCCTGTAACTGCCCTG |
| | LLHAARP | | CTTCTTCCCCTTGCCTTGCTTCTC |
| | (SEQ ID NO: 83) | | CATGCCGCTAGACCC (SEQ ID NO: |
| | | | 84) |
| GMCSFR | MLLLVTSLLLCELP | P15509 | ATGCTGCTGCTGGTCACATCTCTG |
| | HPAFLLIP | | CTGCTGTGCGAGCTGCCCCATCC |
| | (SEQ ID NO: 85) | | TGCCTTTCTGCTGATCCCT (SEQ |
| | | | ID NO: 86) |
| | | | ATGCTGCTGCTGGTTACATCTCTG |
| | | | CTGCTGTGCGAGCTGCCCCATCC |
| | | | TGCCTTTCTGCTGATCCCT (SEQ |
| | | | ID NO: 87) |

Antigen Recognizing Receptors

Certain aspects of the present disclosure relate to an engineered nucleic comprising an antigen recognizing receptor. In some embodiments, an engineered nucleic acid of the present disclosure comprises a first expression cassette that further comprises an antigen recognizing receptor. In some embodiments, the first expression cassette comprises a polynucleotide sequence encoding the antigen recognizing receptor that is operably linked to the first exogenous polynucleotide sequence encoding the ACP and to the first promoter. Suitable antigen recognizing receptors for use as an effector molecule recognize antigens that include, but are not limited to, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin (MSLN), MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1, or any combination thereof.

In some embodiments, the antigen recognizing receptor recognizes an antigen selected from: 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.

In some embodiments, the antigen recognizing receptor recognizes GPC3. An antigen recognizing receptor that recognizes GPC3 can include an anti-binding domain that binds to GPC3. In some embodiments, the antigen-binding domain that binds to GPC3 includes a heavy chain variable (VH) region and a light chain variable (VL) region, wherein the VH includes: a heavy chain complementarity determining region 1 (CDR-H1) having the amino acid sequence of KNAMN (SEQ ID NO: 119), a heavy chain complementarity determining region 2 (CDR-H2) having the amino acid sequence of RIRNKTNNYATYYADSVKA (SEQ ID NO: 120), and a heavy chain complementarity determining region 3 (CDR-H3) having the amino acid sequence of GNSFAY (SEQ ID NO: 121), and wherein the VL includes: a light chain complementarity determining region 1 (CDR-L1) having the amino acid sequence of KSSQSLLYSSNQKNYLA (SEQ ID NO: 122), a light chain complementarity determining region 2 (CDR-L2) having the amino acid sequence of WASSRES (SEQ ID NO: 123), and a light chain complementarity determining region 3 (CDR-L3) having the amino acid sequence of QQYYNYPLT (SEQ ID NO: 124). In some embodiments, the antigen-binding domain that binds to GPC3 includes a heavy chain complementarity determining region 1 (CDR-H1) having the amino acid sequence of KNAMN (SEQ ID NO: 119). In some embodiments, the antigen-binding domain that binds to GPC3 includes a heavy chain complementarity determining region 2 (CDR-H2) having the amino acid sequence of RIRNKTNNYATYYADSVKA (SEQ ID NO: 120). In some embodiments, the antigen-binding domain that binds to GPC3 includes a heavy chain complementarity determining region 3 (CDR-H3) having the amino acid sequence of GNSFAY (SEQ ID NO: 121). In some embodiments, the antigen-binding domain that binds to GPC3 includes a light chain complementarity determining region 1 (CDR-L1) having the amino acid sequence of KSSQSLLYSSNQKNYLA (SEQ ID NO: 122). In some embodiments, the antigen-binding domain that binds to GPC3 includes a light chain complementarity determining region 2 (CDR-L2) having the amino acid sequence of WASSRES (SEQ ID NO: 123). In some embodiments, the antigen-binding domain that binds to GPC3 includes a light chain complementarity determining region 3 (CDR-L3) having the amino acid sequence of QQYYNYPLT (SEQ ID NO: 124).

In some embodiments, the antigen-binding domain that binds to GPC3 includes a VH region having an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 125)
EVQLVETGGGMVQPEGSLKLSCAASGFTFNKNAMN
WVRQAPGKGLEWVARIRNKTNNYATYYADSVKARF
TISRDDSQSMLYLQMNNLKIEDTAMYYCVAGNSFA
YWGQGTLVTVSA
or
(SEQ ID NO: 126)
EVQLVESGGGLVQPGGSLRLSCAASGFTFNKNAMN
WVRQAPGKGLEWVGRIRNKTNNYATYYADSVKARF
TISRDDSKNSLYLQMNSLKTEDTAVYYCVAGNSFA
YWGQGTLVTVSA.

In some embodiments, the antigen-binding domain that binds to GPC3 includes a VL region having an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 127)
DIVMSQSPSSLVVSIGEKVTMTCKSSQSLLYSSNQ
KNYLAWYQQKPGQSPKLLIYWASSRESGVPDRFTG
SGSGTDFTLTISSVKAEDLAVYYCQQYYNYPLTFG
AGTKLELK,
or
(SEQ ID NO: 128)
DIVMTQSPDSLAVSLGERATINCKSSQSLLYSSNQ
KNYLAWYQQKPGQPPKLLIYWASSRESGVPDRFSG
SGSGTDFTLTISSLQAEDVAVYYCQQYYNYPLTFG
QGTKLEIK

In some embodiments, the antigen recognizing receptor recognizes MSLN. An antigen recognizing receptor that recognizes MSLN can include an anti-binding domain that binds to MSLN. In some embodiments, the antigen-binding domain that binds to MSLN includes a single-domain binding domain having an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 129)
QVQLVESGGGTVQAGGSLKLACAASGLPRTYNVMG
WFRQAPGKEREGVAIIYTTTGATYYRDSVKGRATI
SQDNAKKSVSLQMNSLRPEDTAIYYCVARQPNSGP
WEYWGQGTQVTVSS,
or
(SEQ ID NO: 130)
QVKLEESGGGSVQAGGSLRLSCTTSGYTNSYKWMG
WFRQAPGQEREGVAVIYTGNDRTYYSDSVKGRFTI
SRDNAKNMIYLDMTRLRPEDSAVYECAIGHDGAWR
YWGQGTQVTVSS

In some embodiments, the antigen-binding domain that binds to MSLN includes each of the CDR sequences from a single-domain binding domain having an amino acid sequence

(SEQ ID NO: 129)
QVQLVESGGGTVQAGGSLKLACAASGLPRTYNVMGWFRQAPGKEREGVAI
IYTTTGATYYRDSVKGRATISQDNAKKSVSLQMNSLRPEDTAIYYCVARQ
PNSGPWEYWGQGTQVTVSS,
or
(SEQ ID NO: 130)
QVKLEESGGGSVQAGGSLRLSCTTSGYTNSYKWMGWFRQAPGQEREGVAV
IYTGNDRTYYSDSVKGRFTISRDNAKNMIYLDMTRLRPEDSAVYECAIGH
DGAWRYWGQGTQVTVSS

In some embodiments, the antigen-binding domain that binds to MSLN includes one or more CDR sequences from a single-domain binding domain having an amino acid sequence

(SEQ ID NO: 129)
QVQLVESGGGTVQAGGSLKLACAASGLPRTYNVMGWFRQAPGKEREGVAI
IYTTTGATYYRDSVKGRATISQDNAKKSVSLQMNSLRPEDTAIYYCVARQ
PNSGPWEYWGQGTQVTVSS,
or
(SEQ ID NO: 130)
QVKLEESGGGSVQAGGSLRLSCTTSGYTNSYKWMGWFRQAPGQEREGVAV
IYTGNDRTYYSDSVKGRFTISRDNAKNMIYLDMTRLRPEDSAVYECAIGH
DGAWRYWGQGTQVTVSS

In some embodiments, the first expression cassette further comprises a linker polynucleotide sequence localized between the ACP and the antigen recognizing receptor.

In some embodiments, the antigen recognizing receptor comprises an antigen-binding domain. In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, the scFv comprises a heavy chain variable domain (VH) and a light chain variable domain (VL). In some embodiments, the VH and VL are separated by a peptide linker.

An scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain. In some embodiments, the scFv comprises the structure VH-L-VL or VL-L-VH, wherein VH is the heavy chain variable domain, L is the peptide linker, and VL is the light chain variable domain.

An sdAb is a molecule in which one variable domain of an antibody specifically binds to an antigen without the presence of the other variable domain.

A F(ab) fragment contains the constant domain (CL) of the light chain and the first constant domain (CH1) of the heavy chain along with the variable domains VL and VH on the light and heavy chains respectively. F(ab′) fragments differ from Fab fragments by the addition of a few residues at the carboxy terminus of the heavy chain CH1 domain including one or more cysteines from the antibody hinge region. F(ab′)₂ fragments contain two Fab′ fragments joined, near the hinge region, by disulfide bonds.

In some embodiments, the antigen recognizing receptor is a chimeric antigen receptor (CAR) or T cell receptor (TCR). In some embodiments, the antigen recognizing receptor is a CAR. In some embodiments, the CAR comprises one or more intracellular signaling domains, and the one or more intracellular signaling domains are selected from: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain. In some embodiments, the CAR comprises a CD3zeta-chain intracellular signaling domain and one or more additional intracellular signaling domains (e.g., co-stimulatory domains) selected from a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, the CAR further comprises a transmembrane domain, and the transmembrane domain is selected from: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain.

In some embodiments, the CAR further comprises a spacer region (e.g., hinge domain) between the antigen-binding domain and the transmembrane domain. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domains may include, without limitation an IgG domain (such as an IgG1 hinge, an IgG2 hinge, an IgG3 hinge, or an IgG4 hinge), an IgD hinge domain, a CD8a hinge domain, and a CD28 hinge domain. In some embodiments, the spacer or hinge domain is an IgG domain, an IgD domain, a CD8a hinge domain, or a CD28 hinge domain.

Exemplary spacer or hinge domain protein sequences are shown in Table 6. Exemplary spacer or hinge domain nucleotide sequences are shown in Table 7.

TABLE 6

| | SEQ | |
|---|---|---|
| | ID | |
| Amino Acid Sequence | NO: | Description |
| AAAIEVMYPPPYLDNEKSNGT | 100 | CD28 hinge |
| IIHVKGKHLCPSPLFPGPSKP | | |
| ESKYGPPCPSCP | 101 | IgG4 minimal hinge |
| ESKYGPPAPSAP | 102 | IgG4 minimal hinge, |
| | | no disulfides |
| ESKYGPPCPPCP | 103 | IgG4 S228P minimal |
| | | hinge, enhanced |
| | | disulfide formation |
| EPKSCDKTHTCP | 104 | IgG1 minimal hinge |
| AAAFVPVFLPAKPTTTPAPRP | 105 | Extended CD8a hinge |
| PTPAPTIASQPLSLRPEACRP | | |
| AAGGAVHTRGLDFACDIYIWA | | |
| PLAGTCGVLLLSLVITLYCNH | | |
| RN | | |

TABLE 6

| | SEQ | |
|---|---|---|
| | ID | |
| Amino Acid Sequence | NO: | Description |
| TTTPAPRPPTPAPTIALQPLSLRPEACRP | 106 | CD8a hinge |
| AAGGAVHTRGLDFACD | | |
| ACPTGLYTHSGECCKACNLGEGVAQPCGA | 107 | LNGFR hinge |
| NQTVCEPCLDSVTFSDVVSATEPCKPCTE | | |
| CVGLQSMSAPCVEADDAVCRCAYGYYQDE | | |
| TTGRCEACRVCEAGSGLVFSCQDKQNTVC | | |
| EECPDGTYSDEADAEC | | |
| ACPTGLYTHSGECCKACNLGEGVAQPCGA | 108 | Truncated LNGFR |
| NQTVC | | hinge (TNFR- |
| | | Cys1) |
| AVGQDTQEVIVVPHSLPFKV | 109 | PDGFR-beta |
| | | extracellular |
| | | linker |

TABLE 7

| Nucleic Acid Sequence | SEQ ID NO: | Description |
|---|---|---|
| GCAGCAGCTATCGAGGTGATGTATCCTCCGC | 110 | CD28 hinge |
| CCTACCTGGATAATGAAAAGAGTAATGGGA | | |
| CTATCATTCATGTAAAAGGGAAGCATCTTTG | | |
| TCCTTCTCCCCTTTTCCCCGGTCCGTCTAAAC | | |
| CT | | |
| GAA AGC AAG TAC GGT CCA CCT TGC CCT | 111 | IgG4 minimal hinge |
| AGC TGT CCG | | |
| GAA TCC AAG TAC GGC CCC CCA GCG CCT | 112 | IgG4 minimal hinge, no |
| AGT GCC CCA | | disulfides |
| GAA TCT AAA TAT GGC CCG CCA TGC CCG | 113 | IgG4 S228P minimal hinge, |
| CCT TGC CCA | | enhanced disulfide formation |
| GAA CCG AAG TCT TGT GAT AAA ACT CAT | 114 | IgG1 minimal hinge |
| ACG TGC CCG | | |
| GCT GCT GCT TTC GTA CCC GTG TTC CTC | 115 | Extended CD8a hinge |
| CCT GCT AAG CCT ACG ACT ACC CCC GCA | | |
| CCG AGA CCA CCC ACG CCA GCA CCC ACG | | |
| ATTGCT AGC CAG CCC CTT AGT TTG CGA | | |
| CCA GAA GCT TGT CGG CCT GCT GCT GGT | | |
| GGC GCG GTA CAT ACC CGC GGC CTT GAT | | |
| TTT GCTTGC GAT ATA TAT ATC TGG GCG | | |
| CCT CTG GCC GGA ACA TGC GGG GTC CTC | | |
| CTC CTT TCT CTG GTT ATT ACT CTC TAC | | |
| TGT AAT CACAGG AAT | | |
| GCC TGC CCG ACC GGG CTC TAC ACT CAT | 116 | LNGFR hinge |
| AGC GGG GAA TGT TGT AAG GCA TGT AAC | | |
| TTG GGT GAG GGC GTC GCA CAG CCC TGC | | |
| GGAGCT AAC CAA ACA GTG TGC GAA CCC | | |
| TGC CTC GAT AGT GTG ACG TTC TCT GAT | | |
| GTT GTA TCA GCT ACA GAG CCT TGC AAA | | |
| CCA TGTACT GAG TGC GTT GGA CTT CAG | | |
| TCA ATG AGC GCT CCA TGT GTG GAG GCA | | |
| GAT GAT GCG GTC TGT CGA TGT GCT TAC | | |
| GGA TAC TACCAA GAC GAG ACA ACA GGG | | |
| CGG TGC GAG GCC TGT AGA GTT TGT GAG | | |
| GCG GGC TCC GGG CTG GTG TTT TCA TGT | | |
| CAA GAC AAG CAAAAT ACG GTC TGT GAA | | |
| GAG TGC CCT GAT GGC ACC TAC TCA GAC | | |
| GAA GCA GAT GCA GAA TGC | | |
| GCC TGC CCT ACA GGA CTC TAC ACG CAT | 117 | Truncated LNGFR hinge (TNFR- |
| AGC GGT GAG TGT TGT AAA GCA TGC AAC | | Cys1) |
| CTC GGG GAA GGT GTA GCC CAG CCA TGC | | |
| GGG GCT AAC CAA ACC GTT TGC | | |
| GCTGTGGGCCAGGACACGCAGGAGGTCATC | 118 | PDGFR-beta extracellular linker |
| GTGGTGCCACACTCCTTGCCCTTTAAGGTG | | |

Suitable transmembrane domains, spacer or hinge domains, and intracellular domains for use in a CAR are generally described in Stoiber et al, Cells 2019, 8(5), 472; Guedan et al, Mol Therapy: Met & Clinic Dev, 2019 12:145-156; and Sadelain et al, Cancer Discov; 2013, 3(4); 388-98, each of which are hereby incorporated by reference in their entirety.

In some embodiments, the CAR further comprises a secretion signal peptide. Any suitable secretion signal peptide of the present disclosure may be used.

Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

Engineered Cells

Also provided herein are cells, and methods of producing cells, that comprise one or more engineered nucleic acids of the present disclosure. These cells are referred to herein as “engineered cells.” These cells, which typically contain one or more engineered nucleic acids, do not occur in nature. In some embodiments, the cells are isolated cells that recombinantly express the one or more engineered nucleic acids. In some embodiments, the engineered one or more nucleic acids are expressed from one or more vectors or a selected locus from the genome of the cell. In some embodiments, the cells are engineered to include a first nucleic acid comprising a promoter operable linked to a nucleotide sequence encoding an activation-conditional control polypeptide (ACP), such as a transcription factor, and/or antigen recognizing receptor. In some embodiments, the transcription factor comprises a repressible protease and a cognate cleavage site. In some embodiments, the transcription factor comprises a degron. In some embodiments, the ACP is the antigen recognizing receptor.

In some embodiments, the engineered cells comprise a first expression cassette comprising a first promoter and a first exogenous polynucleotide sequence encoding an activation-conditional control polypeptide (ACP) and/or an antigen recognizing receptor, wherein the first promoter is operably linked to the first exogenous polynucleotide; and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. ACP is the antigen recognizing receptor and the receptor is capable of inducing expression of the second expression cassette by binding to its cognate antigen.

In some embodiments, X can be 1, 2, 3, 4, 5, 6, 7, 8, 9, 10, 11, 12, 13, 14, 15, 16, 17, 18, 19, 20, or more.

In some embodiments, the first expression cassette and the second expression cassette are encoded by separate polynucleotide sequences in engineered cells. For example, in some embodiments, the engineered cell comprises two engineered nucleic acids; a first engineered nucleic acid comprising a polynucleotide sequence encoding the first expression cassette and a second engineered nucleic acid comprising a polynucleotide sequence encoding the second expression cassette. In an illustrative example, an effector molecule expression cassette can be encoded by a first engineered nucleic acid in an engineered cell, and an ACP expression cassette can be encoded by a second engineered nucleic acid in the engineered cell. In another illustrative example, an effector molecule expression cassette can be encoded by a first engineered nucleic acid, an ACP expression cassette can be encoded by a second engineered nucleic acid, and an antigen recognizing receptor expression cassette can be encoded by a third engineered nucleic acid in an engineered cell.

In some embodiments, the first expression cassette and the second expression cassette are encoded by a single polynucleotide sequence in engineered cells. For example, in some embodiments, the engineered cells comprises a single engineered nucleic acid comprising a polynucleotide sequence encoding both the first expression cassette and the second expression cassette. Other illustrative examples include, but are not limited to, (1) an antigen recognizing receptor expression cassette and an effector molecule expression cassette can be encoded by a first engineered nucleic acid, and an ACP expression cassette can be encoded by a second engineered nucleic acid; (2) an ACP expression cassette and an effector molecule expression cassette can be encoded by a first engineered nucleic acid, and an antigen recognizing receptor expression cassette can be encoded by a second engineered nucleic acid; (3) an ACP expression cassette and an antigen recognizing receptor expression cassette can be encoded by a first engineered nucleic acid, and an effector molecule expression cassette can be encoded by a second engineered nucleic acid.

In some embodiments, expression cassettes of polynucleotide sequences in engineered cells can be multicistronic, i.e., more than one separate polypeptide (e.g., multiple exogenous polynucleotides or effector molecules) can be produced from a single mRNA transcript. For example, a multicistronic expression cassette can encode both an ACP and antigen recognizing receptor, e.g., both expressed from a single expression cassette driven by a constitutive promoter. In another example, a multicistronic expression cassette can encode both an effector molecule and an antigen recognizing receptor, e.g., both expressed from a single expression cassette driven by an ACP-responsive promoter. Expression cassettes can be multicistronic through the use of various linkers, e.g., a polynucleotide sequence encoding a first protein of interest can be linked to a nucleotide sequence encoding a second protein of interest, such as in a first gene:linker:second gene 5′ to 3′ orientation. Multicistronic features and options are described in the section “Multicistronic and Multiple Promoter Systems.”

In some embodiments, the second expression cassette comprises two or more units of (L-E)_{X}, each L linker polynucleotide sequence is operably associated with the translation of each effector molecule as a separate polypeptide. In some embodiments, the second expression cassette comprising one or more units of (L-E)_{X} further comprises a polynucleotide sequence encoding a secretion signal peptide. In some embodiments, for each X the corresponding secretion signal peptide is operably associated with the effector molecule. In some embodiments, each secretion signal peptide comprises a native secretion signal peptide native to the corresponding effector molecule. In some embodiments, each secretion signal peptide comprises a non-native secretion signal peptide that is non-native to the corresponding effector molecule. In some embodiments, the non-native secretion signal peptide is selected from: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, the cells are engineered to include an additional expression cassette comprising an additional promoter operably linked to an additional exogenous nucleotide sequence encoding an additional effector molecule, for example, one that stimulates an immune response. In some embodiments, the engineered cell further comprises an additional expression cassette comprising an additional promoter and an additional exogenous polynucleotide sequence having the formula: (L-E)_{X} wherein E comprises a polynucleotide sequence encoding an effector molecule, L comprises a linker polynucleotide sequence, X=1 to 20, wherein the additional promoter is operably linked to the additional exogenous polynucleotide, and wherein for the first iteration of the (L-E) unit, L is absent.

X can be 1, 2, 3, 4, 5, 6, 7, 8, 9, 10, 11, 12, 13, 14, 15, 16, 17, 18, 19, 20, or more.

In some embodiments, the additional expression cassette comprises two or more units of (L-E)_{X}, each L linker polynucleotide sequence is operably associated with the translation of each effector molecule as a separate polypeptide. In some embodiments, the additional expression cassette comprises one or more units of (L-E)_{X} further comprises a polynucleotide sequence encoding a secretion signal peptide. In some embodiments, for each X the corresponding secretion signal peptide is operably associated with the effector molecule. In some embodiments, each secretion signal peptide comprises a native secretion signal peptide native to the corresponding effector molecule. In some embodiments, each secretion signal peptide comprises a non-native secretion signal peptide that is non-native to the corresponding effector molecule. In some embodiments, the non-native secretion signal peptide is selected from the group consisting of: IL12, IL2, optimized IL2, trypsiongen-2, Gaussia luciferase, CD5, CD8, human IgKVIICD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, the first promoter and/or the additional promoter is a constitutive promoter, an inducible promoter, or a synthetic promoter. In some embodiments, the first promoter and/or the additional promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

An engineered cell of the present disclosure can comprise an engineered nucleic acid integrated into the cell's genome. An engineered cell can comprise an engineered nucleic acid capable of expression without integrating into the cell's genome, for example, engineered with a transient expression system such as a plasmid or mRNA.

The present disclosure also encompasses additivity and synergy between an effector molecule(s) and the engineered cell from which they are produced. In some embodiments, cells are engineered to produce one or more (e.g., 1, 2, 3, 4, 5, 6, 7, 8, 9, 10, 11, 12, 13, 14, 15, 16, 17, 18, 19, 20 or more) effector molecules, each of which may modulate a different tumor-mediated immunosuppressive mechanism. In other embodiments, cells are engineered to produce at least one effector molecule that is not natively produced by the cells. Such an effector molecule may, for example, complement the function of effector molecules natively produced by the cells.

In some embodiments, a cell (e.g., a tumor cell, an erythrocyte, a platelet cell, or a bacterial cell) is engineered to produce one or more effector molecules. For example, cells may be engineered to produce 1-20 different effector molecules. In some embodiments, cells engineered to produce 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, cells are engineered to produce 1, 2, 3, 4, 5, 6, 7, 8, 9, 10, 11, 12, 13, 14, 15, 16, 17, 18, 19, or 20 effector molecules.

In some embodiments, engineered cells comprise one or more engineered nucleic acids comprising a first expression cassette comprising a first promoter and a first exogenous polynucleotide sequence encoding an activation-conditional control polypeptide (ACP), wherein the first promoter is operably linked to the first exogenous polynucleotide and a second expression cassette comprising an ACP-responsive promoter and a second exogenous polynucleotide sequence having the formula: (L-E)_{X} wherein E comprises a polynucleotide sequence encoding an effector molecule, L comprises a linker polynucleotide sequence, X=1 to 20 wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. In some embodiments, cells are engineered to include a plurality of engineered nucleic acids, e.g., at least two engineered nucleic acids, each encoding a first expression cassette comprising a first promoter and a first exogenous polynucleotide sequence encoding an ACP and a second exogenous polynucleotide sequence having the formula: (L-E)_{X} wherein E comprises a polynucleotide sequence encoding an effector molecule, L comprises a linker polynucleotide sequence, X=1 to 20. In some embodiments, the second exogenous polynucleotide sequence encodes at least one (e.g., 1, 2 or 3) effector molecule. The second exogenous polynucleotide sequence can encode at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more effector molecules. For example, cells may be engineered to comprise at least 2, at least 3, at least 4, at least 5, at least 6, at least 7, at least 8, at least 8, at least 9, at least 10, or more, engineered nucleic acids, each encoding a first expression cassette comprising a promoter operably linked to an ACP polynucleotide sequence, and a second expression cassette comprising an ACP-responsive promoter and an exogenous nucleotide sequence encoding at least one (e.g., 1, 2, 3, or more) effector molecules. In some embodiments, the cells are engineered to comprise 2, 3, 4, 5, 6, 7, 8, 9, 10, or more engineered nucleic acids, each encoding a first expression cassette comprising a promoter operably linked to an ACP polynucleotide sequence, and a second expression cassette comprising an ACP-responsive promoter and an exogenous nucleotide sequence encoding at least one (e.g., 1, 2, 3, or more) effector molecules.

In some embodiments, the engineered cells further comprise a third expression cassette comprising a third promoter and a third exogenous polynucleotide sequence encoding an antigen recognizing receptor, wherein the third promoter is operably linked to the third exogenous polynucleotide. In some embodiments, the first exogenous polynucleotide sequence further encodes an antigen recognizing receptor.

In some embodiments, engineered cells comprise one or more engineered nucleic acids comprising a first expression cassette comprising a first promoter and a first exogenous polynucleotide sequence encoding an activation-conditional control polypeptide (ACP) and/or an antigen recognizing receptor, wherein the first promoter is operably linked to the first exogenous polynucleotide and a second expression cassette comprising an activation-conditional control polypeptide-responsive (ACP-responsive) promoter and a second exogenous polynucleotide sequence having the formula: (L-E)_{X} wherein E comprises a polynucleotide sequence encoding an effector molecule, L comprises a linker polynucleotide sequence, X=1 to 20 wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. In embodiments where the first exogenous polynucleotide sequence encodes an antigen recognizing receptor, the engineered cells may further comprise a third expression cassette comprising a third promoter and a third exogenous polynucleotide sequence encoding an activation-conditional control polypeptide (ACP), wherein the third promoter is operably linked to the third exogenous polynucleotide. Exemplary antigen recognizing receptors include chimeric antigen receptors (CARs) or T cell receptors (TCRs). In some embodiments, the ACP is capable of inducing expression of the second expression cassette by binding to the ACP-responsive promoter. In some embodiments, the ACP is the antigen recognizing receptor and the ACP is capable of inducing expression of the second expression cassette by binding to its cognate antigen. In some embodiments, the ACP-responsive promoter is an inducible promoter that is capable of being induced by the ACP binding to its cognate antigen.

Engineered cells can comprise an engineered nucleic acid encoding at least one of the linkers described above, such as polypeptides that link a first polypeptide sequence and a second polypeptide sequence, one or more multicistronic linker described above, one or more additional promoters operably linked to additional ORFs, or a combination thereof. In some embodiments, the first expression cassette and the second expression cassette are encoded by separate polynucleotide sequences. In some embodiments, the first expression cassette and the second expression cassette are encoded by a single polynucleotide sequence. In some embodiments, when the second expression cassette comprises two or more units of (L₁-E)_{X}, each L₁ linker polynucleotide sequence is operably associated with the translation of each effector molecule as a separate polypeptide. In some embodiments, the engineered cell further comprises a second linker polynucleotide sequence, wherein the second linker polynucleotide links the first expression cassette to the second expression cassette. In some embodiments, the second linker polynucleotide sequence is operably associated with the translation of each effector molecule and the ACP as separate polypeptides.

In some embodiments, a cell (e.g., a T cell, an immune cell, a stem cell, a tumor cell, an erythrocyte, or a platelet cell) is engineered to produce an effector molecule independently selected from a therapeutic class, wherein the therapeutic class is selected from: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, a cell of the present disclosure (e.g., a T cell, an immune cell, a stem cell, a tumor cell, an erythrocyte, or a platelet cell) is engineered to produce a chemokine. In some embodiments, the chemokine is selected from: CCL21a, CXCL10, CXCL11, CXCL13, a CXCL10-CXCL11 fusion protein, CCL19, CXCL9, and XCL1.

In some embodiments, a cell of the present disclosure (e.g., a T cell, an immune cell, a stem cell, a tumor cell, an erythrocyte, or a platelet cell) is engineered to produce a cytokine. In some embodiments, the cytokine is selected from: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, a cell of the present disclosure (e.g., a T cell, an immune cell, a stem cell, a tumor cell, an erythrocyte, or a platelet cell) is engineered to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. In some embodiments, the homing molecule is selected from: anti-integrin alpha4,beta7; anti-MAdCAM; CCR9; CXCR4; SDF1; MMP-2; CXCR1; CXCR7; CCR2; CCR4; and GPR15.

In some embodiments, a cell of the present disclosure (e.g., a T cell, an immune cell, a stem cell, a tumor cell, an erythrocyte, or a platelet cell) is engineered to produce at least one growth factor. In some embodiments, the growth factor is selected from: FLT3L and GM-CSF.

In some embodiments, a cell of the present disclosure (e.g., a T cell, an immune cell, a stem cell, a tumor cell, an erythrocyte, or a platelet cell) is engineered to produce at least one co-activation molecule. In some embodiments, the co-activation molecule is selected from: c-Jun, 4-1BBL and CD40L.

In some embodiments, a cell of the present disclosure (e.g., a T cell, an immune cell, a stem cell, a tumor cell, an erythrocyte, or a platelet cell) is engineered to produce at least one TGFbeta inhibitor. In some embodiments, the TGFbeta inhibitors are selected from: an anti-TGFbeta peptide, an anti-TGFbeta antibody, a TGFb-TRAP, and combinations thereof.

In some embodiments, a cell of the present disclosure (e.g., a T cell, an immune cell, a stem cell, a tumor cell, an erythrocyte, or a platelet cell) is engineered to produce at least one immune checkpoint inhibitor. In some embodiments, the immune checkpoint inhibitors are selected from: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011— Teva/CureTech), AMP224 (anti-PD-1; NCI), avelumab (anti-PD-L1; Bavencio®—Pfizer), durvalumab (anti-PD-L1; MEDI4736/Imfinzi®-Medimmune/AstraZeneca), atezolizumab (anti-PD-L1; Tecentriq®—Roche/Genentech), BMS-936559 (anti-PD-L1—BMS), tremelimumab (anti-CTLA-4; Medimmune/AstraZeneca), ipilimumab (anti-CTLA-4; Yervoy®—BMS), lirilumab (anti-MR; BMS), monalizumab (anti-NKG2A; Innate Pharma/AstraZeneca).

In some embodiments, a cell of the present disclosure (e.g., a tumor cell, an erythrocyte, a platelet cell, or a bacterial cell) is engineered to produce at least one VEGF inhibitor. In some embodiments, the VEGF inhibitors comprise anti-VEGF antibodies, anti-VEGF peptides, or combinations thereof.

In some embodiments, each effector molecule is a human-derived effector molecule.

Engineered Cell Types

An engineered cell or isolated cell of the present disclosure can be a human cell. An engineered cell or isolated cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer cell. An engineered primary cell can be any somatic cell. An engineered primary cell can be a MSC. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered or isolated cell of the present disclosure is selected from: a T cell, a CD8+ T cell, a CD4+ T cell, a gamma-delta T cell, a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the engineered cell is a Natural Killer (NK) cell. In some embodiments, an engineered cell is autologous. In some embodiments, an engineered cell is allogeneic.

In some embodiments, an engineered cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.

In some embodiments, an engineered cell of the present disclosure is a bacterial cell selected from: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Methods of Engineering Cells

Also provided herein are compositions and methods for engineering cells to produce the activation conditional control polypeptide (ACP) and one or more effectors molecules encoded by any engineered nucleic acid comprising the first and second expression cassettes as described herein, or.

In general, cells are engineered to produce ACPs and effector molecules through introduction (i.e., delivery) of one or more polynucleotides of the present disclosure comprising the first promoter and the exogenous polynucleotide sequence encoding the ACP and the second expression cassette comprising an ACP-responsive promoter and the second exogenous sequence encoding one or more effector molecules into the cell's cytosol and/or nucleus. For example, the polynucleotide expression cassettes encoding the ACP polypeptide and the one or more effector molecules can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

In some embodiments, the engineered cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some embodiments, the oncolytic virus is a recombinant oncolytic virus comprising the first expression cassette and the second expression cassette. In some embodiments, the oncolytic virus further comprises the third expression cassette.

The virus, including any of the oncolytic viruses described herein, can be a recombinant virus that encodes one more transgenes encoding one or more effector molecules, such as any of the engineered nucleic acids described herein. The virus, including any of the oncolytic viruses described herein, can be a recombinant virus that encodes one more transgenes encoding one or more of the two or more effector molecules, such as any of the engineered nucleic acids described herein. In some embodiments, the cell is engineered via transduction with an oncolytic virus.

Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more effector molecules. The transgenes encoding the one or more effector molecules can be configured to express the one or more effector molecules.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat & amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Lipid Structure Delivery Systems

Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

Genomic Editing Systems

A genomic editing systems can be used to engineer a host genome to encode an engineered nucleic acid, such as an engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

Methods of Use

Methods for treatment of diseases are also encompassed by this disclosure. Said methods include administering a therapeutically effective amount of an engineered nucleic acid, engineered cell, or isolated cell as described above. In some aspects, provided herein are methods of treating a subject in need thereof, the method comprising administering a therapeutically effective dose of any of the engineered cells, isolated cells, or compositions disclosed herein.

In some aspects, provided herein are methods of stimulating a cell-mediated immune response to a tumor cell in a subject, the method comprising administering to a subject having a tumor a therapeutically effective dose of any of the engineered cells, isolated cells, or compositions disclosed herein.

In some aspects, provided herein are methods of providing an anti-tumor immunity in a subject, the method comprising administering to a subject in need thereof a therapeutically effective dose of any of the engineered cells, isolated cells, or compositions disclosed herein.

In some aspects, provided herein are methods of treating a subject having cancer, the method comprising administering a therapeutically effective dose of any of the engineered cells, isolated cells, or compositions disclosed herein.

In some aspects, provided herein are methods of reducing tumor volume in a subject, the method comprising administering to a subject having a tumor a composition comprising any of the engineered cells, isolated cells, or compositions disclosed herein.

In some embodiments, the administering comprises systemic administration. In some embodiments, the administering comprises intratumoral administration. In some embodiments, the isolated cell is derived from the subject. In some embodiments, the isolated cell is allogeneic with reference to the subject.

In some embodiments, the method further comprises administering a checkpoint inhibitor. the checkpoint inhibitor is selected from: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody. In some embodiments, the method further comprises administering an anti-CD40 antibody.

In some embodiments, the tumor is selected from: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

Some methods comprise selecting a subject (or patient population) having a tumor (or cancer) and treating that subject with engineered cells or delivery vehicles that modulate tumor-mediated immunosuppressive mechanisms.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells.

The methods provided herein also include administering a drug or pharmaceutical composition in combination with a therapeutically effective dose of any of the engineered cells, isolated cells, or compositions disclosed herein such that the ACP is induced and/or that a repressible protease is repressed. For example, tamoxifen or a metabolite thereof (e.g., 4-hydroxytamoxifen, N-desmethyltamoxifen, tamoxifen-N-oxide, or endoxifen) can be administered to induce the ACP. The drug or pharmaceutical can be administered prior to, concurrently with, simultaneously with, and/or subsequent to administration of any of the engineered cells, isolated cells, or compositions disclosed herein. The drug or pharmaceutical can be administered serially. The drug or pharmaceutical can be administered concurrently or simultaneously with administration of any of the engineered cells, isolated cells, or compositions disclosed herein. The drug or pharmaceutical can be administered at separate intervals than (e.g., prior to or subsequent to) administration of any of the engineered cells, isolated cells, or compositions disclosed herein. The drug or pharmaceutical can be administered both concurrently/simultaneously as well as at separate intervals than any of the engineered cells, isolated cells, or compositions disclosed herein. The drug or pharmaceutical composition and the engineered cells, isolated cells, or compositions can be administered via different routes, e.g., the drug or pharmaceutical composition can be administered orally and the engineered cells, isolated cells, or compositions can be administered intraperitoneally, intravenously, subcutaneously, or any other route appropriate for administration, as will be appreciated by one skilled in the art.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The methods provided herein include administering a protease inhibitor. In some embodiments, the NS3 protease can be repressed by a protease inhibitor. Any suitable protease inhibitor can be used, including, but not limited to, simeprevir, danoprevir, asunaprevir, ciluprevir, boceprevir, sovaprevir, paritaprevir, telaprevir, grazoprevir, glecaprevir, and voxiloprevir, or any combination thereof. In some embodiments, the protease inhibitor is selected from: simeprevir, danoprevir, asunaprevir, ciluprevir, boceprevir, sovaprevir, paritaprevir, telaprevir, grazoprevir, glecaprevir, and voxiloprevir.

In some embodiments, the protease inhibitor is grazoprevir. In some embodiments, the protease inhibitor is a combination of grazoprevir and elbasvir (a NSSA inhibitor of the hepatitis C virus NSSA replication complex). Grazoprevir and elbasvir can be co-formulated as a pharmaceutical composition, such as in tablet form (e.g., the tablet available under the tradename Zepatier®). Grazoprevir and elbasvir can be co-formulated at a 2:1 weight ratio, respectively, such as at a unit dose of 100 mg grazoprevir 50 mg elbasvir (e.g., as in the tablet available under the tradename Zepatier®). The protease inhibitor can be administered at a dose capable of repressing a repressible protease domain of an ACP. The protease inhibitor can be administered at an approved dose for another indication. As an illustrative non-limiting example, Zepatier can be administered at its approved dose for treatment of HCV.

Grazoprevir, including in combination with elbasvir, can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, grazoprevir, including in combination with elbasvir, can be provided in the form of tablets or capsules containing 1.0 to 500 mg of the active ingredient, particularly 1, 5, 10, 15, 20, 25, 50, 75, 100, 150, 200, 250, 300, 400, 500, and 750 mg of the active ingredient for the symptomatic adjustment of the dosage to the patient to be treated. Generally, a total daily dosage of grazoprevir, including in combination with elbasvir, can range from about 1 to about 2500 mg per day, although variations will necessarily occur depending on the target of therapy, the patient and the route of administration. In one embodiment, the dosage of grazoprevir, including in combination with elbasvir, is from about 10 to about 1000 mg/day, administered in a single dose or in 2-4 divided doses. In another embodiment, the dosage of grazoprevir, including in combination with elbasvir, is from about 1 to about 500 mg/day, administered in a single dose or in 2-4 divided doses. In still another embodiment, the dosage of grazoprevir, including in combination with elbasvir, is from about 1 to about 100 mg/day, administered in a single dose or in 2-4 divided doses. In yet another embodiment, the dosage of grazoprevir, including in combination with elbasvir, is from about 1 to about 50 mg/day, administered in a single dose or in 2-4 divided doses. In another embodiment, the dosage of grazoprevir, including in combination with elbasvir, is from about 500 to about 1500 mg/day, administered in a single dose or in 2-4 divided doses. In still another embodiment, the dosage of grazoprevir, including in combination with elbasvir, is from about 500 to about 1000 mg/day, administered in a single dose or in 2-4 divided doses. In yet another embodiment, the dosage of grazoprevir, including in combination with elbasvir, is from about 100 to about 500 mg/day, administered in a single dose or in 2-4 divided doses.

In vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the effector molecules described herein. The methods provided herein also include delivering a composition in vivo capable of producing two or more of the effector molecules described herein. Compositions capable of in vivo production of effector molecules include, but are not limited to, any of the engineered nucleic acids described herein. Compositions capable of in vivo production of effector molecules can be a naked mRNA or a naked plasmid.

Pharmaceutical Compositions

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Whether it is a polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### ADDITIONAL EMBODIMENTS

Provided below are enumerated embodiments describing specific embodiments of the invention:

Embodiment 1: An engineered nucleic acid comprising:
a) a first expression cassette comprising a first promoter and a first exogenous polynucleotide sequence encoding an activation-conditional control polypeptide (ACP), wherein the first promoter is operably linked to the first exogenous polynucleotide; and
b) a second expression cassette comprising an ACP-responsive promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.
Embodiment 2: The engineered nucleic acid of embodiment 1, wherein when the second expression cassette comprises two or more units of (L-E)_{X}, each linker polynucleotide sequence is operably associated with the translation of each effector molecule as a separate polypeptide.
Embodiment 3: The engineered nucleic acid of embodiment 1 or embodiment 2, wherein the linker polynucleotide sequence encodes a 2A ribosome skipping tag.
Embodiment 4: The engineered nucleic acid of embodiment 3, wherein the 2A ribosome skipping tag is selected from the group consisting of: P2A, T2A, E2A, and F2A.
Embodiment 5: The engineered nucleic acid of any one of embodiments 1-2, the linker polynucleotide sequence encodes an Internal Ribosome Entry Site (IRES).
Embodiment 6: The engineered nucleic acid of any one of embodiments 1-5, wherein the linker polynucleotide sequence encodes a cleavable polypeptide.
Embodiment 7: The engineered nucleic acid of embodiment 6, wherein the cleavable polypeptide comprises a furin polypeptide sequence.
Embodiment 8: The engineered nucleic acid of any one of embodiments 1-7, wherein the second expression cassette comprising one or more units of (L-E)_{X} further comprises a polynucleotide sequence encoding a secretion signal peptide for each X.
Embodiment 9: The engineered nucleic acid of embodiment 8, wherein for each X the corresponding secretion signal peptide is operably associated with the effector molecule.
Embodiment 10: The engineered nucleic acid of embodiment 8 or embodiment 9, wherein each secretion signal peptide comprises a native secretion signal peptide native to the corresponding effector molecule.
Embodiment 11: The engineered nucleic acid of any one of embodiments 8-10, wherein each secretion signal peptide comprises a non-native secretion signal peptide that is non-native to the corresponding effector molecule.
Embodiment 12: The engineered nucleic acid of embodiment 11, wherein the non-native secretion signal peptide is a secretion signal peptide of a molecule selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.
Embodiment 13: The engineered nucleic acid of any one of embodiments 1-12, wherein the ACP-responsive promoter comprises an ACP-binding domain sequence and a promoter sequence.
Embodiment 14: The engineered nucleic acid of embodiment 13, wherein the promoter sequence is derived from a promoter selected from the group consisting of: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.
Embodiment 15: The engineered nucleic acid of any one of embodiments 1-14, wherein the ACP-responsive promoter comprises a synthetic promoter.
Embodiment 16: The engineered nucleic acid of any one of embodiments 1-15, wherein the ACP-responsive promoter comprises a minimal promoter.
Embodiment 17: The engineered nucleic acid of any one of embodiments 12-16, wherein the ACP-binding domain comprises one or more zinc finger binding sites.
Embodiment 18: The engineered nucleic acid of any one of embodiments 1-17, wherein the first promoter comprises a constitutive promoter, an inducible promoter, or a synthetic promoter.
Embodiment 19: The engineered nucleic acid of embodiment 18, wherein the constitutive promoter is selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 20: The engineered nucleic acid of any one of embodiments 1-19, wherein each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.
Embodiment 21: The engineered nucleic acid of embodiment 20, wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
Embodiment 22: The engineered nucleic acid of embodiment 20, wherein the chemokine is selected from the group consisting of: CCL21a, CXCL10, CXCL11, CXCL13, a CXCL10-CXCL11 fusion protein, CCL19, CXCL9, and XCL1.
Embodiment 23: The engineered nucleic acid of embodiment 20, wherein the homing molecule is selected from the group consisting of: anti-integrin alpha4,beta7; anti-MAdCAM; CCR9; CXCR4; SDF1; MMP-2; CXCR1; CXCR7; CCR2; CCR4; and GPR15.
Embodiment 24: The engineered nucleic acid of embodiment 20, wherein the growth factor is selected from the group consisting of: FLT3L and GM-CSF.
Embodiment 25: The engineered nucleic acid of embodiment 20, wherein the co-activation molecule is selected from the group consisting of: c-Jun, 4-1BBL and CD40L.
Embodiment 26: The engineered nucleic acid of embodiment 20, wherein the tumor microenvironment modifier is selected from the group consisting of: adenosine deaminase, TGFbeta inhibitors, immune checkpoint inhibitors, VEGF inhibitors, and HPGE2.
Embodiment 27: The engineered nucleic acid of embodiment 26, wherein the TGFbeta inhibitors are selected from the group consisting of: an anti-TGFbeta peptide, an anti-TGFbeta antibody, a TGFb-TRAP, and combinations thereof.
Embodiment 28: The engineered nucleic acid of embodiment 26, wherein the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 29: The engineered nucleic acid of embodiment 26, wherein the VEGF inhibitors comprise anti-VEGF antibodies, anti-VEGF peptides, or combinations thereof.
Embodiment 30: The engineered nucleic acid of any one of embodiments 1-29, wherein each effector molecule is a human-derived effector molecule.
Embodiment 31: The engineered nucleic acid of any one of embodiments 1-30, wherein the first exogenous polynucleotide sequence further encodes an antigen recognizing receptor.
Embodiment 32: An engineered nucleic acid comprising:
a) a first expression cassette comprising a first promoter and a first exogenous polynucleotide sequence encoding an activation-conditional control polypeptide (ACP) and an antigen recognizing receptor, wherein the first promoter is operably linked to the first exogenous polynucleotide; and
b) a second expression cassette comprising an ACP-responsive promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.
Embodiment 33: An engineered nucleic acid comprising:
a) a first expression cassette comprising a first promoter and a first exogenous polynucleotide sequence encoding an antigen recognizing receptor, wherein the first promoter is operably linked to the first exogenous polynucleotide; and
b) a second expression cassette comprising an activation-conditional control polypeptide-responsive (ACP-responsive) promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent.
Embodiment 34: The engineered nucleic acid of embodiment 33, wherein the ACP is capable of inducing expression of the second expression cassette by binding to the ACP-responsive promoter.
Embodiment 35: The engineered nucleic acid of embodiment 33, wherein the ACP is the antigen recognizing receptor and the ACP is capable of inducing expression of the second expression cassette following binding of the ACP to a cognate antigen.
Embodiment 36: The engineered nucleic acid of embodiment 35, wherein the ACP-responsive promoter is an inducible promoter that is capable of being induced by the ACP binding to the cognate antigen.
Embodiment 37: The engineered nucleic acid of embodiment 36, wherein the ACP-responsive promoter is derived from a promoter region of a gene upregulated following binding of the ACP to the cognate antigen.
Embodiment 38: The engineered nucleic acid of any one of embodiments 32-34, wherein the ACP-responsive promoter is selected from the group consisting of a constitutive promoter, an inducible promoter, and a synthetic promoter.
Embodiment 39: The engineered nucleic acid of any one of embodiments 32-38, wherein the ACP-responsive promoter comprises a minimal promoter.
Embodiment 40: The engineered nucleic acid of any one of embodiments 32-39, wherein the ACP-binding domain comprises one or more zinc finger binding sites.
Embodiment 41: The engineered nucleic acid of any one of embodiments 1-30, further comprising a linker polynucleotide sequence localized between the first expression cassette and the second expression cassette.
Embodiment 42: The engineered nucleic acid of embodiment 41, wherein the linker polynucleotide sequence is operably associated with the translation of the ACP and each effector molecule as separate polypeptides.
Embodiment 43: The engineered nucleic acid of embodiment 31 or embodiment 32, wherein the first exogenous polynucleotide sequence further comprises a linker polynucleotide sequence localized between the region of the first exogenous polynucleotide sequence encoding the ACP and the region of the first exogenous polynucleotide sequence encoding the antigen recognizing receptor.
Embodiment 44: The engineered nucleic acid of embodiment 43, wherein the linker polynucleotide sequence is operably associated with the translation of the ACP and the antigen recognizing receptor as separate polypeptides.
Embodiment 45: The engineered nucleic acid of any one of embodiments 33-36, further comprising a linker polynucleotide sequence localized between the first expression cassette and the second expression cassette.
Embodiment 46: The engineered nucleic acid of embodiment 45, wherein the linker polynucleotide sequence is operably associated with the translation of the antigen receptor and each effector molecule as separate polypeptides.
Embodiment 47: The engineered nucleic acid of any one of embodiments 41-46, wherein the linker polynucleotide sequence encodes a 2A ribosome skipping tag.
Embodiment 48: The engineered nucleic acid of embodiment 47, wherein the 2A ribosome skipping tag is selected from the group consisting of: P2A, T2A, E2A, and F2A.
Embodiment 49: The engineered nucleic acid of any one of embodiments 41-46, wherein the linker polynucleotide sequence encodes an Internal Ribosome Entry Site (IRES).
Embodiment 50: The engineered nucleic acid of any one of embodiments 41-49, wherein the linker polynucleotide sequence encodes a cleavable polypeptide.
Embodiment 51: The engineered nucleic acid of embodiment 50, wherein the cleavable polypeptide comprises a furin polypeptide sequence.
Embodiment 52: The engineered nucleic acid of any one of embodiments 31-51, wherein the antigen recognizing receptor recognizes an antigen selected from the group consisting of: 5T4, ADAMS, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
Embodiment 53: The engineered nucleic acid of any one of embodiments 31-52, wherein the antigen recognizing receptor recognizes GPC3.
Embodiment 54: The engineered nucleic acid of any one of embodiments 31-52, wherein the antigen recognizing receptor recognizes mesothelin (MSLN).
Embodiment 55: The engineered nucleic acid of any one of embodiments 31-54, wherein the antigen recognizing receptor comprises an antigen-binding domain.
Embodiment 56: The engineered nucleic acid of embodiment 53 or embodiment 55, wherein the antigen-binding domain that binds to GPC3 comprises a heavy chain variable (VH) region and a light chain variable (VL) region, wherein the VH comprises:
a heavy chain complementarity determining region 1 (CDR-H1) having the amino acid sequence of KNAMN (SEQ ID NO: 119), a heavy chain complementarity determining region 2 (CDR-H2) having the amino acid sequence of RIRNKTNNYATYYADSVKA (SEQ ID NO: 120), and a heavy chain complementarity determining region 3 (CDR-H3) having the amino acid sequence of GNSFAY (SEQ ID NO: 121), and wherein the VL comprises:
a light chain complementarity determining region 1 (CDR-L1) having the amino acid sequence of KSSQSLLYSSNQKNYLA (SEQ ID NO: 122), a light chain complementarity determining region 2 (CDR-L2) having the amino acid sequence of WASSRES (SEQ ID NO: 123), and a light chain complementarity determining region 3 (CDR-L3) having the amino acid sequence of QQYYNYPLT (SEQ ID NO: 124).
   Embodiment 57: The engineered nucleic acid of embodiment 56, wherein the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 125)
EVQLVETGGGMVQPEGSLKLSCAASGFTFNKNAMNWVRQAPGKGLEWVAR
IRNKTNNYATYYADSVKARFTISRDDSQSMLYLQMNNLKIEDTAMYYCVA
GNSFAYWGQGTLVTVSA
or
(SEQ ID NO: 126)
EVQLVESGGGLVQPGGSLRLSCAASGFTFNKNAMNWVRQAPGKGLEWVGR
IRNKTNNYATYYADSVKARFTISRDDSKNSLYLQMNSLKTEDTAVYYCVA
GNSFAYWGQGTLVTVSA.

Embodiment 58: The engineered nucleic acid of embodiment 56 or embodiment 57, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 127)
DIVMSQSPSSLVVSIGEKVTMTCKSSQSLLYSSNQKNYLAWYQQKPGQSP
KLLIYWASSRESGVPDRFTGSGSGTDFTLTISSVKAEDLAVYYCQQYYNY
PLTFGAGTKLELK,
or
(SEQ ID NO: 128)
DIVMTQSPDSLAVSLGERATINCKSSQSLLYSSNQKNYLAWYQQKPGQPP
KLLIYWASSRESGVPDRFSGSGSGTDFTLTISSLQAEDVAVYYCQQYYNY
PLTFGQGTKLEIK

Embodiment 59: The engineered nucleic acid of embodiment 54 or embodiment 55, wherein the antigen-binding domain that binds to MSLN comprises the three complementarity determining regions (CDRs) of a single-domain monoclonal antibody having the amino acid sequence of:

(SEQ ID NO: 129)
QVQLVESGGGTVQAGGSLKLACAASGLPRTYNVMGWFRQAPGKEREGVAI
IYTTTGATYYRDSVKGRATISQDNAKKSVSLQMNSLRPEDTAIYYCVARQ
PNSGPWEYWGQGTQVTVSS,
or
(SEQ ID NO: 130)
QVKLEESGGGSVQAGGSLRLSCTTSGYTNSYKWMGWFRQAPGQEREGVAV
IYTGNDRTYYSDSVKGRFTISRDNAKNMIYLDMTRLRPEDSAVYECAIGH
DGAWRYWGQGTQVTVSS.

Embodiment 60: The engineered nucleic acid of any one of embodiments 55-59, wherein the antigen-binding domain comprises an antibody, an antigen-binding fragment of an antibody, a F(ab) fragment, a F(ab′) fragment, a single chain variable fragment (scFv), or a single-domain antibody (sdAb).
Embodiment 61: The engineered nucleic acid of any one of embodiments 55-59, wherein the antigen-binding domain comprises a single chain variable fragment (scFv).
Embodiment 62: The engineered nucleic acid of embodiment 61, wherein the scFv comprises a heavy chain variable domain (VH) and a light chain variable domain (VL).
Embodiment 63: The engineered nucleic acid of embodiment 62, wherein the VH and VL are separated by a peptide linker.
Embodiment 64: The engineered nucleic acid of embodiment 63, wherein the scFv comprises the structure VH-L-VL or VL-L-VH, wherein VH is the heavy chain variable domain, L is the peptide linker, and VL is the light chain variable domain.
Embodiment 65: The engineered nucleic acid of any one of embodiments 31-64, wherein the antigen recognizing receptor is a chimeric antigen receptor (CAR) or T cell receptor (TCR).
Embodiment 66: The engineered nucleic acid of any one of embodiments 31-65, wherein the antigen recognizing receptor is a CAR.
Embodiment 67: The engineered nucleic acid of embodiment 66, wherein the CAR comprises one or more intracellular signaling domains, and each of the one or more intracellular signaling domains is selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.
Embodiment 68: The engineered nucleic acid of embodiment 66 or embodiment 67, wherein the CAR comprises a transmembrane domain, and the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain.
Embodiment 69: The engineered nucleic acid of any one of embodiments 66-68, wherein the CAR comprises a spacer region between the antigen-binding domain and the transmembrane domain.
Embodiment 70: The engineered nucleic acid of any one of embodiments 1-69, wherein the ACP is a transcriptional modulator.
Embodiment 71: The engineered nucleic acid of any one of embodiments 1-70, wherein the ACP is a transcriptional repressor.
Embodiment 72: The engineered nucleic acid of any one of embodiments 1-70, wherein the ACP is a transcriptional activator.
Embodiment 73: The engineered nucleic acid of any one of embodiments 1-72, wherein the ACP further comprises a repressible protease and one or more cognate cleavage sites of the repressible protease.
Embodiment 74: The engineered nucleic acid of any one of embodiments 1-73, wherein the ACP further comprises a hormone-binding domain of estrogen receptor (ERT2 domain).
Embodiment 75: The engineered nucleic acid of any one of embodiments 72-74, wherein the ACP is a transcription factor.
Embodiment 76: The engineered nucleic acid of embodiment 74, wherein the transcription factor is a zinc-finger-containing transcription factor.
Embodiment 77: The engineered nucleic acid of any one of embodiments 1-76, wherein the ACP comprises a DNA-binding zinc finger protein domain (ZF protein domain) and a transcriptional effector domain.
Embodiment 78: The engineered nucleic acid of embodiment 77, wherein the ZF protein domain is modular in design and is composed of zinc finger arrays (ZFA).
Embodiment 79: The engineered nucleic acid of embodiment 78, wherein the ZF protein domain comprises one to ten ZFA.
Embodiment 80: The engineered nucleic acid of any one of embodiments 77-79, wherein the effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a histone acetyltransferase (HAT) core domain of the human E1A-associated protein p300 (p300 HAT core activation domain); a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.
Embodiment 81: The engineered nucleic acid of any one of embodiments 77-80, wherein the one or more cognate cleavage sites of the repressible protease are localized between the ZF protein domain and the effector domain.
Embodiment 82: The engineered nucleic acid of any one of embodiments 73-81, wherein the repressible protease is hepatitis C virus (HCV) nonstructural protein 3 (NS3).
Embodiment 83: The engineered nucleic acid of embodiment 82, wherein the cognate cleavage site comprises an NS3 protease cleavage site.
Embodiment 84: The engineered nucleic acid of embodiment 83, wherein the NS3 protease cleavage site comprises a NS3/NS4A, a NS4A/NS4B, a NS4B/NSSA, or a NS5A/NS5B junction cleavage site.
Embodiment 85: The engineered nucleic acid of any one of embodiments 82-84, wherein the NS3 protease can be repressed by a protease inhibitor.
Embodiment 86: The engineered nucleic acid of embodiment 85, wherein the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir.
Embodiment 87: The engineered nucleic acid of embodiment 85, wherein the protease inhibitor is grazoprevir.
Embodiment 88: The engineered nucleic acid of embodiment 85, wherein the protease inhibitor comprises grazoprevir and elbasvir.
Embodiment 89: The engineered nucleic acid of embodiment 88, wherein the grazoprevir and the elbasvir is co-formulated in a pharmaceutical composition.
Embodiment 90: The engineered nucleic acid of embodiment 89, wherein the pharmaceutical composition is a tablet.
Embodiment 91: The engineered nucleic acid of embodiment 89 or 90, wherein the grazoprevir and the elbasvir are at a 2 to 1 weight ratio.
Embodiment 92: The engineered nucleic acid of embodiment 91, wherein the grazoprevir is 100 mg per unit dose and the elbasvir is 50 mg per unit dose.
Embodiment 93: The engineered nucleic acid of any one of embodiments 74-92, wherein the ACP is capable of undergoing nuclear localization upon binding of the ERT2 domain to tamoxifen or a metabolite thereof.
Embodiment 94: The engineered nucleic acid of embodiment 93, wherein the tamoxifen metabolite is selected from the group consisting of: 4-hydroxytamoxifen, N-desmethyltamoxifen, tamoxifen-N-oxide, and endoxifen.
Embodiment 95: The engineered nucleic acid of any one of embodiments 1-92, wherein the ACP further comprises a degron, and wherein the degron is operably linked to the ACP.
Embodiment 96: The engineered nucleic acid of embodiment 95, wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.
Embodiment 97: The engineered nucleic acid of embodiment 93, wherein the degron comprises a cereblon (CRBN) polypeptide substrate domain capable of binding CRBN in response to an immunomodulatory drug (IMiD) thereby promoting ubiquitin pathway-mediated degradation of the ACP.
Embodiment 98: The engineered nucleic acid of embodiment 97, wherein the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.
Embodiment 99: The engineered nucleic acid of embodiment 97, wherein the CRBN polypeptide substrate domain is a chimeric fusion product of native CRBN polypeptide sequences.
Embodiment 100: The engineered nucleic acid of embodiment 97, wherein the CRBN polypeptide substrate domain is a IKZF3/ZFP91/IKZF3 chimeric fusion product having the amino acid sequence of

(SEQ ID NO: 131)
FNVLMVHKRSHTGERPLQCEICGFTCRQKGNLLRHIKLHTGEKPFKCHLC
NYACQRRDAL

Embodiment 101: The engineered nucleic acid of any one of embodiments 97-100, wherein the IMiD is an FDA-approved drug.
Embodiment 102: The engineered nucleic acid of any one of embodiments 97-101, wherein the IMiD is selected from the group consisting of: thalidomide, lenalidomide, and pomalidomide.
Embodiment 103: The engineered nucleic acid of any one of embodiments 93-102, wherein the degron is localized 5′ of the repressible protease, 3′ of the repressible protease, 5′ of the ZF protein domain, 3′ of the ZF protein domain, 5′ of the effector domain, or 3′ of the effector domain.
Embodiment 104: The engineered nucleic acid of any one of embodiments 1-103, wherein the engineered nucleic acid further comprises an insulator.
Embodiment 105: The engineered nucleic acid of embodiment 104, wherein the insulator is localized between the first expression cassette and the second expression cassette.
Embodiment 106: The engineered nucleic acid of any one of embodiments 1-105, wherein the first expression cassette is localized in the same orientation relative to the second expression cassette.
Embodiment 107: The engineered nucleic acid of any one of embodiments 1-106, wherein the first expression cassette is localized in the opposite orientation relative to the second expression cassette.
Embodiment 108: The engineered nucleic acid of any one of embodiments 1-107, wherein the engineered nucleic acid is selected from the group consisting of: a DNA, a cDNA, an RNA, an mRNA, and a naked plasmid.
Embodiment 109: An expression vector comprising the engineered nucleic acid of any one of embodiments 1-108.
Embodiment 110: A composition comprising the engineered nucleic acid of any one of embodiments 1-108, and a pharmaceutically acceptable carrier.
Embodiment 111: An isolated cell comprising the engineered nucleic acid of any one of embodiments 1-108 or the vector of embodiment 109.
Embodiment 112: The isolated cell of embodiment 111, wherein the engineered nucleic acid is recombinantly expressed.
Embodiment 113: The isolated cell of embodiment 111 or embodiment 112, wherein the engineered nucleic acid is expressed from a vector or a selected locus from the genome of the cell.
Embodiment 114: The isolated cell of any one of embodiments 111-113, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
Embodiment 115: The isolated cell of any one of embodiments 111-114, wherein the cell is a Natural Killer (NK) cell.
Embodiment 116: The isolated cell of any one of embodiments 111-115, wherein the cell is autologous.
Embodiment 117: The isolated cell of any one of embodiments 111-115, wherein the cell is allogeneic.
Embodiment 118: The isolated cell of any one of embodiments 111-113, wherein the cell is a tumor cell selected from the group consisting of: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.
Embodiment 119: The isolated cell of embodiment 118, wherein the cell was engineered via transduction with an oncolytic virus.
Embodiment 120: The isolated cell of embodiment 119, wherein the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.
Embodiment 121: The isolated cell of embodiment 119 or embodiment 120 wherein the oncolytic virus is a recombinant oncolytic virus comprising the first expression cassette and the second expression cassette.
Embodiment 122: The isolated cell of any one of embodiments 111-113, wherein the cell is a bacterial cell selected from the group consisting of: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.
Embodiment 123: A composition comprising the isolated cell of any one of embodiments 111-122, and a pharmaceutically acceptable carrier.
Embodiment 124: A method of treating a subject in need thereof, the method comprising administering a therapeutically effective dose of any of the isolated cells of any one of embodiments 111-122 or the composition of embodiment 123.
Embodiment 125: A method of stimulating a cell-mediated immune response to a tumor cell in a subject, the method comprising administering to a subject having a tumor a therapeutically effective dose of any of the isolated cells of any one of embodiments 111-122 or the composition of embodiment 123.
Embodiment 126: A method of providing an anti-tumor immunity in a subject, the method comprising administering to a subject in need thereof a therapeutically effective dose of any of the isolated cells of any one of embodiments 111-122 or the composition of embodiment 123.
Embodiment 127: A method of treating a subject having cancer, the method comprising administering a therapeutically effective dose of any of the isolated cells of any one of embodiments 111-122 or the composition of embodiment 123.
Embodiment 128: A method of reducing tumor volume in a subject, the method comprising administering to a subject having a tumor a composition comprising any of the isolated cells of any one of embodiments 111-122 or the composition of embodiment 123.
Embodiment 129: The method of any one of embodiments 124-128, wherein the administering comprises systemic administration.
Embodiment 130: The method of any one of embodiments 124-128, wherein the administering comprises intratumoral administration.
Embodiment 131: The method of any one of embodiments 124-130, wherein the isolated cell is derived from the subject.
Embodiment 132: The method of any one of embodiments 124-130, wherein the isolated cell is allogeneic with reference to the subject.
Embodiment 133: The method of any one of embodiments 124-132, wherein the method further comprises administering a checkpoint inhibitor.
Embodiment 134: The method of embodiment 133, wherein the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 135: The method of any one of embodiments 124-134, wherein the method further comprises administering an anti-CD40 antibody.
Embodiment 136: The method of any one of embodiments 125-135, wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 137: A lipid-based structure comprising the engineered nucleic acid of any one of embodiments 1-108.
Embodiment 138: The lipid-based structure of embodiment 137, wherein the lipid-based structure comprises a extracellular vesicle.
Embodiment 139: The lipid-based structure of embodiment 138, wherein the extracellular vesicle is selected from the group consisting of: a nanovesicle and an exosome.
Embodiment 140: The lipid-based structure of embodiment 137, wherein the lipid-based structure comprises a lipid nanoparticle or a micelle.
Embodiment 141: The lipid-based structure of embodiment 137, wherein the lipid-based structure comprises a liposome.
Embodiment 142: A composition comprising the lipid-based structure of any one of embodiments 137-141, and a pharmaceutically acceptable carrier.
Embodiment 143: A method of treating a subject in need thereof, the method comprising administering a therapeutically effective dose of any of the lipid-based structures of any one of embodiments 137-141 or the composition of embodiment 142.
Embodiment 144: A method of stimulating a cell-mediated immune response to a tumor cell in a subject, the method comprising administering to a subject having a tumor a therapeutically effective dose of any of the lipid-based structures of any one of embodiments 137-141 or the composition of embodiment 142.
Embodiment 145: A method of providing an anti-tumor immunity in a subject, the method comprising administering to a subject in need thereof a therapeutically effective dose of any of the lipid-based structures of any one of embodiments 137-141 or the composition of embodiment 142.
Embodiment 146: A method of treating a subject having cancer, the method comprising administering a therapeutically effective dose of any of the lipid-based structures of any one of embodiments 137-141 or the composition of embodiment 142.
Embodiment 147: A method of reducing tumor volume in a subject, the method comprising administering to a subject having a tumor a composition comprising any of the lipid-based structures of any one of embodiments 137-141 or the composition of embodiment 142.
Embodiment 148: The method of any one of embodiments 143-147, wherein the administering comprises systemic administration.
Embodiment 149: The method of any one of embodiments 144-147, wherein the administering comprises intratumoral administration.
Embodiment 150: The method of any one of embodiments 143-149, the lipid-based structure is capable of engineering a cell in the subject.
Embodiment 151: The method of any one of embodiments 143-150, wherein the method further comprises administering a checkpoint inhibitor.
Embodiment 152: The method of embodiment 151, wherein the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 153: The method of any one of embodiments 143-152, wherein the method further comprises administering an anti-CD40 antibody.
Embodiment 154: The method of any one of embodiments 144-153, wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 155: A nanoparticle comprising the engineered nucleic acid of any one of embodiments 1-108.
Embodiment 156: The nanoparticle of embodiment 155, wherein the nanoparticle comprises an inorganic material.
Embodiment 157: A composition comprising the nanoparticle of embodiment 155 or embodiment 156.
Embodiment 158: A method of treating a subject in need thereof, the method comprising administering a therapeutically effective dose of any of the nanoparticles of embodiment 155 or embodiment 156, or the composition of embodiment 157.
Embodiment 159: A method of stimulating a cell-mediated immune response to a tumor cell in a subject, the method comprising administering to a subject having a tumor a therapeutically effective dose of any of the nanoparticles of embodiment 155 or embodiment 156, or the composition of embodiment 157.
Embodiment 160: A method of providing an anti-tumor immunity in a subject, the method comprising administering to a subject in need thereof a therapeutically effective dose of any of the nanoparticles of embodiment 155 or embodiment 156, or the composition of embodiment 157.
Embodiment 161: A method of treating a subject having cancer, the method comprising administering a therapeutically effective dose of any of the nanoparticles of embodiment 155 or embodiment 156, or the composition of embodiment 157.
Embodiment 162: A method of reducing tumor volume in a subject, the method comprising administering to a subject having a tumor a composition comprising any of the nanoparticles of embodiment 155 or embodiment 156, or the composition of embodiment 157.
Embodiment 163: The method of any one of embodiments 158-162, wherein the administering comprises systemic administration.
Embodiment 164: The method of any one of embodiments 159-162, wherein the administering comprises intratumoral administration.
Embodiment 165: The method of any one of embodiments 158-164, the nanoparticle is capable of engineering a cell in the subject.
Embodiment 166: The method of any one of embodiments 158-165, wherein the method further comprises administering a checkpoint inhibitor.
Embodiment 167: The method of embodiment 166, wherein the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 168: The method of any one of embodiments 158-167, wherein the method further comprises administering an anti-CD40 antibody.
Embodiment 169: The method of any one of embodiments 159-168, wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 170: A virus engineered to comprise the engineered nucleic acid of any one of embodiments 1-108.
Embodiment 171: The engineered virus of embodiment 170, wherein the virus is selected from the group consisting of: a lentivirus, a retrovirus, an oncolytic virus, an adenovirus, an adeno-associated virus (AAV), and a virus-like particle (VLP).
Embodiment 172: The engineered virus of embodiment 170, wherein the virus is an oncolytic virus.
Embodiment 173: The engineered virus of embodiment 172, wherein the first expression cassette and the second expression cassette are capable of being expressed in a tumor cell.
Embodiment 174: The engineered virus of embodiment 173, wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 175: The engineered virus of any one of embodiments 171-174, wherein the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.
Embodiment 176: A composition comprising the engineered virus of any one of embodiments 170-175, and a pharmaceutically acceptable carrier.
Embodiment 177: A method of stimulating a cell-mediated immune response to a tumor cell in a subject, the method comprising administering to a subject having a tumor a therapeutically effective dose of any of the engineered viruses of any one of embodiments 170-175 or the composition of embodiment 176.
Embodiment 178: A method of providing an anti-tumor immunity in a subject, the method comprising administering to a subject in need thereof a therapeutically effective dose of any of the engineered viruses of any one of embodiments 170-175 or the composition of embodiment 176.
Embodiment 179: A method of treating a subject having cancer, the method comprising administering a therapeutically effective dose of any of the engineered viruses of any one of embodiments 170-175 or the composition of embodiment 176.
Embodiment 180: A method of reducing tumor volume in a subject, the method comprising administering to a subject having a tumor a composition comprising any of the engineered viruses of any one of embodiments 170-175 or the composition of embodiment 176.
Embodiment 181: The method of any one of embodiments 177-180, wherein the administering comprises systemic administration.
Embodiment 182: The method of any one of embodiments 177-180, wherein the administering comprises intratumoral administration.
Embodiment 183: The method of any one of embodiments 177-182, the engineered virus infects a cell in the subject and expresses the first expression cassette and the second expression cassette.
Embodiment 184: The method of any one of embodiments 177-183, wherein the method further comprises administering a checkpoint inhibitor.
Embodiment 185: The method of embodiment 184, wherein the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 186: The method of any one of embodiments 177-185, wherein the method further comprises administering an anti-CD40 antibody.
Embodiment 187: The method of any one of embodiments 177-186 wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 188: An engineered cell comprising:
a) a first expression cassette comprising a first promoter and a first exogenous polynucleotide sequence encoding an activation-conditional control polypeptide (ACP), wherein the first promoter is operably linked to the first exogenous polynucleotide; and
b) a second expression cassette comprising an ACP-responsive promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.
Embodiment 189: The engineered cell of embodiment 188, wherein the first expression cassette and the second expression cassette are encoded by separate polynucleotide sequences.
Embodiment 190: The engineered cell of embodiment 188, wherein the first expression cassette and the second expression cassette are encoded by a single polynucleotide sequence.
Embodiment 191: The engineered cell of any one of embodiments 188-190, wherein when the second expression cassette comprises two or more units of (L₁-E)_{X}, each Li linker polynucleotide sequence is operably associated with the translation of each effector molecule as a separate polypeptide.
Embodiment 192: The engineered cell of embodiment 190 or embodiment 191, wherein the engineered cell further comprises a second linker polynucleotide sequence, wherein the second linker polynucleotide links the first expression cassette to the second expression cassette.
Embodiment 193: The engineered cell of embodiment 192, wherein the second linker polynucleotide sequence is operably associated with the translation of each effector molecule and the ACP as separate polypeptides.
Embodiment 194: The engineered cell of any one of embodiments 188-193, wherein each linker polynucleotide sequence encodes a 2A ribosome skipping tag.
Embodiment 195: The engineered cell of embodiment 194, wherein the 2A ribosome skipping tag is selected from the group consisting of: P2A, T2A, E2A, and F2A.
Embodiment 196: The engineered cell of any one of embodiments 188-193, wherein each linker polynucleotide sequence encodes an Internal Ribosome Entry Site (IRES).
Embodiment 197: The engineered cell of any one of embodiments 188-196, wherein the linker polynucleotide sequence encodes a cleavable polypeptide.
Embodiment 198: The engineered cell of embodiment 197, wherein the cleavable polypeptide comprises a furin polypeptide sequence.
Embodiment 199: The engineered cell of any one of embodiments 188-198, wherein the second expression cassette comprising one or more units of (L₁-E)_{X} further comprises a polynucleotide sequence encoding a secretion signal peptide for each X.
Embodiment 200: The engineered cell of embodiment 199, wherein for each X the corresponding secretion signal peptide is operably associated with the effector molecule.
Embodiment 201: The engineered cell of embodiment 199 or embodiment 200, wherein each secretion signal peptide comprises a native secretion signal peptide native to the corresponding effector molecule.
Embodiment 202: The engineered cell of any one of embodiments 199-201, wherein each secretion signal peptide comprises a non-native secretion signal peptide that is non-native to the corresponding effector molecule.
Embodiment 203: The engineered cell of embodiment 202, wherein the non-native secretion signal peptide is a secretion signal peptide of a molecule selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.
Embodiment 204: The engineered cell of any one of embodiments 188-203, wherein the ACP-responsive promoter comprises an ACP-binding domain and a promoter sequence.
Embodiment 205: The engineered cell embodiment 204, wherein the promoter sequence is derived from a promoter selected from the group consisting of: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.
Embodiment 206: The engineered cell of any one of embodiments 188-205, wherein the ACP-responsive promoter is a synthetic promoter.
Embodiment 207: The engineered cell of any one of embodiments 188-206, wherein the ACP-responsive promoter comprises a minimal promoter.
Embodiment 208: The engineered cell of any one of embodiments 204-207, wherein the ACP-binding domain comprises one or more zinc finger binding sites.
Embodiment 209: The engineered cell of any one of embodiments 188-208, wherein the first promoter is a constitutive promoter, an inducible promoter, or a synthetic promoter.
Embodiment 210: The engineered cell of embodiment 209, wherein the constitutive promoter is selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 211: The engineered cell of any one of embodiments 188-210, wherein each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.
Embodiment 212: The engineered cell of embodiment 211, wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
Embodiment 213: The engineered cell of embodiment 212, wherein the chemokine is selected from the group consisting of: CCL21a, CXCL10, CXCL11, CXCL13, a CXCL10-CXCL11 fusion protein, CCL19, CXCL9, and XCL1.
Embodiment 214: The engineered cell of embodiment 211, wherein the homing molecule is selected from the group consisting of: anti-integrin alpha4,beta7; anti-MAdCAM; CCR9; CXCR4; SDF1; MMP-2; CXCR1; CXCR7; CCR2; and GPR15.
Embodiment 215: The engineered cell of embodiment 211, wherein the growth factor is selected from the group consisting of: FLT3L and GM-CSF.
Embodiment 216: The engineered cell of embodiment 211, wherein the co-activation molecule is selected from the group consisting of: c-Jun, 4-1BBL, and CD40L.
Embodiment 217: The engineered cell of embodiment 211, wherein the tumor microenvironment modifier is selected from the group consisting of: adenosine deaminase, TGFbeta inhibitors, immune checkpoint inhibitors, VEGF inhibitors, and HPGE2.
Embodiment 218: The engineered cell of embodiment 217, wherein the TGFbeta inhibitors are selected from the group consisting of: an anti-TGFbeta peptide, an anti-TGFbeta antibody, a TGFb-TRAP, and combinations thereof.
Embodiment 219: The engineered cell of embodiment 217, wherein the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 220: The engineered cell of embodiment 217, wherein the VEGF inhibitors comprise anti-VEGF antibodies, anti-VEGF peptides, or combinations thereof.
Embodiment 221: The engineered cell of any one of embodiments 188-217, wherein each effector molecule is a human-derived effector molecule.
Embodiment 222: The engineered cell of any one of embodiments 188-221, wherein the cell further comprises a third expression cassette comprising a third promoter and a third exogenous polynucleotide sequence encoding an antigen recognizing receptor, wherein the third promoter is operably linked to the third exogenous polynucleotide.
Embodiment 223: The engineered cell of any one of embodiments 188-221, wherein the first exogenous polynucleotide sequence further encodes an antigen recognizing receptor.
Embodiment 224: An engineered cell comprising:
a) a first expression cassette comprising a first promoter and a first exogenous polynucleotide sequence encoding an activation-conditional control polypeptide (ACP) and an antigen recognizing receptor, wherein the first promoter is operably linked to the first exogenous polynucleotide; and
b) a second expression cassette comprising an ACP-responsive promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter.
Embodiment 225: An engineered cell comprising:
a) a first expression cassette comprising a first promoter and a first exogenous polynucleotide sequence encoding an antigen recognizing receptor, wherein the first promoter is operably linked to the first exogenous polynucleotide; and
b) a second expression cassette comprising an activation-conditional control polypeptide-responsive (ACP-responsive) promoter and a second exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the ACP-responsive promoter is operably linked to the second exogenous polynucleotide, wherein for the first iteration of the (L-E) unit, L is absent.
Embodiment 226: The engineered cell of embodiment 225, wherein the cell further comprises a third expression cassette comprising a third promoter and a third exogenous polynucleotide sequence encoding an activation-conditional control polypeptide (ACP), wherein the third promoter is operably linked to the third exogenous polynucleotide.
Embodiment 227: The engineered cell of embodiment 226, wherein the ACP is capable of inducing expression of the second expression cassette by binding to the ACP-responsive promoter.
Embodiment 228: The engineered cell of embodiment 225, wherein the ACP is the antigen recognizing receptor and the ACP is capable of inducing expression of the second expression cassette following binding of the ACP to a cognate antigen.
Embodiment 229: The engineered cell of embodiment 228, wherein the ACP-responsive promoter is an inducible promoter that is capable of being induced by the ACP binding to the cognate antigen.
Embodiment 230: The engineered cell of embodiment 229, wherein the ACP-responsive promoter is derived from a promoter region of a gene upregulated following binding of the ACP to the cognate antigen.
Embodiment 231: The engineered cell of any one of embodiments 224-227, wherein the ACP-responsive promoter is selected from the group consisting of a constitutive promoter, an inducible promoter, and a synthetic promoter.
Embodiment 232: The engineered cell of any one of embodiments 224-231, wherein the first expression cassette and the second expression cassette are encoded by separate polynucleotide sequences.
Embodiment 233: The engineered cell of any one of embodiments 224-232, wherein the ACP-responsive promoter comprises a minimal promoter.
Embodiment 234: The engineered cell of any one of embodiments 224-233, wherein the ACP-binding domain comprises one or more zinc finger binding sites.
Embodiment 235: The engineered cell of embodiment 224 or embodiment 225, wherein the first exogenous polynucleotide sequence further comprises a third linker polynucleotide sequence localized between the region of the first exogenous polynucleotide sequence encoding the ACP and the region of the first exogenous polynucleotide sequence encoding the antigen recognizing receptor.
Embodiment 236: The engineered cell of embodiment 235, wherein the third linker polynucleotide sequence is operably associated with the translation of the ACP and the antigen recognizing receptor as separate polypeptides.
Embodiment 237: The engineered cell of any one of embodiments 225-234, further comprising a third linker polynucleotide sequence localized between the first expression cassette and the second expression cassette.
Embodiment 238: The engineered cell of embodiment 237, wherein the third linker polynucleotide sequence is operably associated with the translation of the antigen receptor and each effector molecule as separate polypeptides.
Embodiment 239: The engineered cell of any one of embodiments 235-238, wherein the third linker polynucleotide sequence encodes a 2A ribosome skipping tag.
Embodiment 240: The engineered nucleic acid of embodiment 239, wherein the 2A ribosome skipping tag is selected from the group consisting of: P2A, T2A, E2A, and F2A.
Embodiment 241: The engineered cell of any one of embodiments 235-238, the third linker polynucleotide sequence encodes an Internal Ribosome Entry Site (IRES).
Embodiment 242: The engineered nucleic acid of any one of embodiments 235-241, wherein the third linker polynucleotide sequence encodes a cleavable polypeptide.
Embodiment 243: The engineered nucleic acid of embodiment 242, wherein the cleavable polypeptide comprises a furin polypeptide sequence.
Embodiment 244: The engineered cell of any one of embodiments 222-243, wherein the antigen recognizing receptor recognizes an antigen selected from the group consisting of: 5T4, ADAMS, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
Embodiment 245: The engineered cell of any one of embodiments 222-244, wherein the antigen recognizing receptor recognizes GPC3.
Embodiment 246: The engineered cell of any one of embodiments 222-244, wherein the antigen recognizing receptor recognizes mesothelin.
Embodiment 247: The engineered cell of any one of embodiments 222-246, wherein the antigen recognizing receptor comprises an antigen-binding domain.
Embodiment 248: The engineered cell of embodiment 245 or embodiment 247, wherein the antigen-binding domain that binds to GPC3 comprises a heavy chain variable (VH) region and a light chain variable (VL) region, wherein the VH comprises:
a heavy chain complementarity determining region 1 (CDR-H1) having the amino acid sequence of KNAMN (SEQ ID NO: 119),
a heavy chain complementarity determining region 2 (CDR-H2) having the amino acid sequence of RIRNKTNNYATYYADSVKA (SEQ ID NO: 120), and
a heavy chain complementarity determining region 3 (CDR-H3) having the amino acid sequence of GNSFAY (SEQ ID NO: 121), and
wherein the VL comprises:
a light chain complementarity determining region 1 (CDR-L1) having the amino acid sequence of KSSQSLLYSSNQKNYLA (SEQ ID NO: 122),
a light chain complementarity determining region 2 (CDR-L2) having the amino acid sequence of WASSRES (SEQ ID NO: 123), and
a light chain complementarity determining region 3 (CDR-L3) having the amino acid sequence of QQYYNYPLT (SEQ ID NO: 124).
Embodiment 249: The engineered cell of embodiment 248, wherein the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 125)
EVQLVETGGGMVQPEGSLKLSCAASGFTFNKNAMNWVRQAPGKGLEWVAR
IRNKTNNYATYYADSVKARFTISRDDSQSMLYLQMNNLKIEDTAMYYCVA
GNSFAYWGQGTLVTVSA
or
(SEQ ID NO: 126)
EVQLVESGGGLVQPGGSLRLSCAASGFTFNKNAMNWVRQAPGKGLEWVGR
IRNKTNNYATYYADSVKARFTISRDDSKNSLYLQMNSLKTEDTAVYYCVA
GNSFAYWGQGTLVTVSA.

Embodiment 250: The engineered cell of embodiment 248 or embodiment 249, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of

(SEQ ID NO: 127)
DIVMSQSPSSLVVSIGEKVTMTCKSSQSLLYSSNQKNYLAWYQQKPGQSP
KLLIYWASSRESGVPDRFTGSGSGTDFTLTISSVKAEDLAVYYCQQYYNY
PLTFGAGTKLELK,
or
(SEQ ID NO: 128)
DIVMTQSPDSLAVSLGERATINCKSSQSLLYSSNQKNYLAWYQQKPGQPP
KLLIYWASSRESGVPDRFSGSGSGTDFTLTISSLQAEDVAVYYCQQYYNY
PLTFGQGTKLEIK

Embodiment 251: The engineered cell of embodiment 246 or embodiment 247, wherein the antigen-binding domain that binds to MSLN comprises the three complementarity determining regions (CDRs) of a single-domain monoclonal antibody having the amino acid sequence of:

(SEQ ID NO: 129)
QVQLVESGGGTVQAGGSLKLACAASGLPRTYNVMGWFRQAPGKEREGVAI
IYTTTGATYYRDSVKGRATISQDNAKKSVSLQMNSLRPEDTAIYYCVARQ
PNSGPWEYWGQGTQVTVSS,
or
(SEQ ID NO: 130)
QVKLEESGGGSVQAGGSLRLSCTTSGYTNSYKWMGWFRQAPGQEREGVAV
IYTGNDRTYYSDSVKGRFTISRDNAKNMIYLDMTRLRPEDSAVYECAIGH
DGAWRYWGQGTQVTVSS.

Embodiment 252: The engineered cell of any one of embodiments 247-251, wherein the antigen-binding domain comprises an antibody, an antigen-binding fragment of an antibody, a F(ab) fragment, a F(ab′) fragment, a single chain variable fragment (scFv), or a single-domain antibody (sdAb).
Embodiment 253: The engineered cell of any one of embodiments 247-251, wherein the antigen-binding domain comprises a single chain variable fragment (scFv).
Embodiment 254: The engineered cell of embodiment 253, wherein the scFv comprises a heavy chain variable domain (VH) and a light chain variable domain (VL).
Embodiment 255: The engineered cell of embodiment 254, wherein the VH and VL are separated by a peptide linker.
Embodiment 256: The engineered cell of embodiment 254 or embodiment 255, wherein the scFv comprises the structure VH-L-VL or VL-L-VH, wherein VH is the heavy chain variable domain, L is the peptide linker, and VL is the light chain variable domain.
Embodiment 257: The engineered cell of any one of embodiments 222-256, wherein the antigen recognizing receptor is a chimeric antigen receptor (CAR) or T cell receptor (TCR).
Embodiment 258: The engineered cell of embodiment 257, wherein the antigen recognizing receptor is a CAR.
Embodiment 259: The engineered cell of embodiment 258, wherein the CAR comprises one or more intracellular signaling domains, and each of the one or more intracellular signaling domains is selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.
Embodiment 260: The engineered cell of embodiment 258 or embodiment 259, wherein the CAR comprises a transmembrane domain, and the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain.
Embodiment 261: The engineered cell of any one of embodiments 258-260, wherein the CAR comprises a spacer region between the antigen-binding domain and the transmembrane domain.
Embodiment 262: The engineered cell of any one of embodiments 188-261, wherein the ACP is a transcriptional modulator.
Embodiment 263: The engineered cell of any one of embodiments 188-261, wherein the ACP is a transcriptional repressor.
Embodiment 264: The engineered cell of any one of embodiments 188-261, wherein the ACP is a transcriptional activator.
Embodiment 265: The engineered cell of any one of embodiments 188-264, wherein the ACP further comprises a repressible protease and one or more cognate cleavage sites of the repressible protease.
Embodiment 266: The engineered cell of any one of embodiments 188-264, wherein the ACP further comprises a hormone-binding domain of estrogen receptor (ERT2 domain).
Embodiment 267: The engineered cell of any one of embodiments 264-266, wherein the ACP is a transcription factor.
Embodiment 268: The engineered cell of embodiment 237, wherein the ACP is a zinc-finger-containing transcription factor.
Embodiment 269: The engineered cell of embodiment 268, wherein the zinc finger-containing transcription factor comprises a DNA-binding zinc finger protein domain (ZF protein domain) and an effector domain.
Embodiment 270: The engineered cell of embodiment 269, wherein the ZF protein domain is modular in design and is composed of zinc finger arrays (ZFA).
Embodiment 271: The engineered cell of embodiment 270, wherein the ZF protein domain comprises one to ten ZFA.
Embodiment 272: The engineered cell of any one of embodiments 269-271, wherein the effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a histone acetyltransferase (HAT) core domain of the human E1A-associated protein p300 (p300 HAT core activation domain); a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.
Embodiment 273: The engineered cell of any one of embodiments 269-272, wherein the one or more cognate cleavage sites of the repressible protease are localized between the ZF protein domain and the effector domain.
Embodiment 274: The engineered cell of any one of embodiments 265-273, wherein the repressible protease is a hepatitis C virus (HCV) nonstructural protein 3 (NS3).
Embodiment 275: The engineered cell of embodiment 274, wherein the cognate cleavage site comprises an NS3 protease cleavage site.
Embodiment 276: The engineered cell of embodiment 275, wherein the NS3 protease cleavage site comprises a NS3/NS4A, a NS4A/NS4B, a NS4B/NS5A, or a NS5A/NS5B junction cleavage site.
Embodiment 277: The engineered cell of any one of embodiments 274-276, wherein the NS3 protease can be repressed by a protease inhibitor.
Embodiment 278: The engineered cell of embodiment 277, wherein the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir.
Embodiment 279: The engineered cell of embodiment 277, wherein the protease inhibitor is grazoprevir.
Embodiment 280: The engineered cell of embodiment 277, wherein the protease inhibitor is grazoprevir and elbasvir.
Embodiment 281: The engineered cell of embodiment 280, wherein the grazoprevir and the elbasvir is co-formulated in a pharmaceutical composition.
Embodiment 282: The engineered cell of embodiment 281, wherein the pharmaceutical composition is a tablet.
Embodiment 283: The engineered cell of embodiment 281 or 282, wherein the grazoprevir and the elbasvir are at a 2 to 1 weight ratio.
Embodiment 284: The engineered cell of embodiment 283, wherein the grazoprevir is 100 mg per unit dose and the elbasvir is 50 mg per unit dose.
Embodiment 285: The engineered cell of any one of embodiments 266-284, wherein the ACP is capable of undergoing nuclear localization upon binding of the ERT2 domain to tamoxifen or a metabolite thereof.
Embodiment 286: The engineered cell of embodiment 285, wherein the tamoxifen metabolite is selected from the group consisting of: 4-hydroxytamoxifen, N-desmethyltamoxifen, tamoxifen-N-oxide, and endoxifen.
Embodiment 287: The engineered cell of any one of embodiments 188-286, wherein the ACP further comprises a degron, and wherein the degron is operably linked to the ACP.
Embodiment 288: The engineered cell of embodiment 287, wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.
Embodiment 289: The engineered cell of embodiment 287, wherein the degron comprises a cereblon (CRBN) polypeptide substrate domain capable of binding CRBN in response to an immunomodulatory drug (IMiD) thereby promoting ubiquitin pathway-mediated degradation of the ACP.
Embodiment 290: The engineered cell of embodiment 289, wherein the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.
Embodiment 291: The engineered cell of embodiment 289, wherein the CRBN polypeptide substrate domain is a chimeric fusion product of native CRBN polypeptide sequences.
Embodiment 292: The engineered cell of embodiment 289, wherein the CRBN polypeptide substrate domain is a IKZF3/ZFP91/IKZF3 chimeric fusion product having the amino acid sequence of

(SEQ ID NO: 131)
FNVLMVHKRSHTGERPLQCEICGFTCRQKGNLLRHIKLHTGEKPFKCHLC
NYACQRRDAL

Embodiment 293: The engineered cell of any one of embodiments 289-292, wherein the IMiD is an FDA-approved drug.
Embodiment 294: The engineered cell of any one of embodiments 289-293, wherein the IMiD is selected from the group consisting of: thalidomide, lenalidomide, and pomalidomide.
Embodiment 295: The engineered cell of any one of embodiments 285-294, wherein the degron is localized 5′ of the repressible protease, 3′ of the repressible protease, 5′ of the ZF protein domain, 3′ of the ZF protein domain, 5′ of the effector domain, or 3′ of the effector domain.
Embodiment 296: The engineered cell of any one of embodiments 190-295, wherein the engineered nucleic acid further comprises an insulator.
Embodiment 297: The engineered cell of embodiment 296, wherein the insulator is localized between the first expression cassette and the second expression cassette.
Embodiment 298: The engineered cell of any one of embodiments 190-297, wherein the first expression cassette is localized in the same orientation relative to the second expression cassette.
Embodiment 299: The engineered cell of any one of embodiments 190-297, wherein the first expression cassette is localized in the opposite orientation relative to the second expression cassette.
Embodiment 300: The engineered cell of any one of embodiments 188-299, wherein the cell further comprises an additional expression cassette comprising an additional promoter and an additional exogenous polynucleotide sequence having the formula:

(L-E)_{X}

wherein
E comprises a polynucleotide sequence encoding an effector molecule,
L comprises a linker polynucleotide sequence,

### X=1 to 20,

wherein the additional promoter is operably linked to the additional exogenous polynucleotide, and wherein for the first iteration of the (L-E) unit, L is absent.
Embodiment 301: The engineered cell of embodiment 300, wherein when the additional expression cassette comprises two or more units of (L-E)_{X}, each L linker polynucleotide sequence is operably associated with the translation of each effector molecule as a separate polypeptide.
Embodiment 302: The engineered cell of embodiment 300 or embodiment 301, wherein each linker polynucleotide sequence encodes a 2A ribosome skipping tag.
Embodiment 303: The engineered cell of embodiment 302, wherein the 2A ribosome skipping tag is selected from the group consisting of: P2A, T2A, E2A, and F2A.
Embodiment 304: The engineered cell of embodiment 300 or embodiment 301, wherein each linker polynucleotide sequence encodes an Internal Ribosome Entry Site (IRES).
Embodiment 305: The engineered cell of any one of embodiments 300-304, wherein the linker polynucleotide sequence encodes a cleavable polypeptide.
Embodiment 306: The engineered cell of embodiment 305, wherein the cleavable polypeptide comprises a furin polypeptide sequence.
Embodiment 307: The engineered cell of any one of embodiments 300-306, wherein the additional expression cassette comprising one or more units of (L-E)_{X} further comprises a polynucleotide sequence encoding a secretion signal peptide for each X.
Embodiment 308: The engineered cell of embodiment 307, wherein for each X the corresponding secretion signal peptide is operably associated with the effector molecule.
Embodiment 309: The engineered cell of embodiment 307 or embodiment 308, wherein each secretion signal peptide comprises a native secretion signal peptide native to the corresponding effector molecule.
Embodiment 310: The engineered cell of any one of embodiments 307-309, wherein each secretion signal peptide comprises a non-native secretion signal peptide that is non-native to the corresponding effector molecule.
Embodiment 311: The engineered cell of embodiment 310 wherein the non-native secretion signal peptide is a secretion signal peptide of a molecule selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.
Embodiment 312: The engineered cell of any one of embodiments 300-311, wherein the additional promoter is a constitutive promoter, an inducible promoter, or a synthetic promoter.
Embodiment 313: The engineered cell of any one of embodiments 300-311, wherein the additional promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 314: The engineered cell of any one of embodiments 300-313, wherein each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme.
Embodiment 315: The engineered cell of embodiment 314, wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
Embodiment 316: The engineered cell of embodiment 314, wherein the chemokine is selected from the group consisting of: CCL21a, CXCL10, CXCL11, CXCL13, a CXCL10-CXCL11 fusion protein, CCL19, CXCL9, and XCL1.
Embodiment 317: The engineered cell of embodiment 314, wherein the homing molecule is selected from the group consisting of: anti-integrin alpha4,beta7; anti-MAdCAM; CCR9; CXCR4; SDF1; MMP-2; CXCR1; CXCR7; CCR2; and GPR15.
Embodiment 318: The engineered cell of embodiment 314, wherein the growth factor is selected from the group consisting of: FLT3L and GM-CSF.
Embodiment 319: The engineered cell of embodiment 314, wherein the co-activation molecule is selected from the group consisting of: c-Jun, 4-1BBL, and CD40L.
Embodiment 320: The engineered cell of embodiment 314, wherein the tumor microenvironment modifier is selected from the group consisting of: adenosine deaminase, TGFbeta inhibitors, immune checkpoint inhibitors, VEGF inhibitors, and HPGE2.
Embodiment 321: The engineered cell of embodiment 320, wherein the TGFbeta inhibitors are selected from the group consisting of: an anti-TGFbeta peptide, an anti-TGFbeta antibody, a TGFb-TRAP, and combinations thereof.
Embodiment 322: The engineered cell of embodiment 320, wherein the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-MR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 323: The engineered cell of embodiment 320, wherein the VEGF inhibitors comprise anti-VEGF antibodies, anti-VEGF peptides, or combinations thereof.
Embodiment 324: The engineered cell of any one of embodiments 300-320, wherein each effector molecule is a human-derived effector molecule.
Embodiment 325: The engineered cell of any one of embodiments 188-324, wherein the cell is selected from the group consisting of: a T cell, a CD8+ T cell, a CD4+ T cell, a gamma-delta T cell, a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
Embodiment 326: The engineered cell of any one of embodiments 188-325, wherein the cell is a Natural Killer (NK) cell.
Embodiment 327: The engineered cell of any one of embodiments 188-326, wherein the cell is autologous.
Embodiment 328: The engineered cell of any one of embodiments 188-326 wherein the cell is allogeneic.
Embodiment 329: The engineered cell of any one of embodiments 188-324, wherein the cell is a tumor cell selected from the group consisting of: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.
Embodiment 330: The engineered cell of embodiment 329, wherein the cell was engineered via transduction with an oncolytic virus.
Embodiment 331: The engineered cell of embodiment 330, wherein the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.
Embodiment 332: The engineered cell of embodiment 330 or embodiment 331, wherein the oncolytic virus is a recombinant oncolytic virus comprising the first expression cassette and the second expression cassette.
Embodiment 333: The engineered cell of any one of embodiments 188-324, wherein the cell is a bacterial cell selected from the group consisting of: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.
Embodiment 334: A composition comprising the engineered cell of any one of embodiments 188-333, and a pharmaceutically acceptable carrier.
Embodiment 335: A method of treating a subject in need thereof, the method comprising administering a therapeutically effective dose of any of the engineered cells of any one of embodiments 188-333 or the composition of embodiment 334.
Embodiment 336: A method of stimulating a cell-mediated immune response to a tumor cell in a subject, the method comprising administering to a subject having a tumor a therapeutically effective dose of any of the engineered cells of any one of embodiments 188-333 or the composition of embodiment 334.
Embodiment 337: A method of providing an anti-tumor immunity in a subject, the method comprising administering to a subject in need thereof a therapeutically effective dose of any of the engineered cells of any one of embodiments 188-333 or the composition of embodiment 334.
Embodiment 338: A method of treating a subject having cancer, the method comprising administering a therapeutically effective dose of any of the engineered cells of any one of embodiments 188-333 or the composition of embodiment 334.
Embodiment 339: A method of reducing tumor volume in a subject, the method comprising administering to a subject having a tumor a composition comprising any of the engineered cells of any one of embodiments 188-333 or the composition of embodiment 334.
Embodiment 340: The method of any one of embodiments 335-339, wherein the administering comprises systemic administration.
Embodiment 341: The method of any one of embodiments 336-339, wherein the administering comprises intratumoral administration.
Embodiment 342: The method of any one of embodiments 335-341, wherein the engineered cell is derived from the subject.
Embodiment 343: The method of any one of embodiments 335-341, wherein the engineered cell is allogeneic with reference to the subject.
Embodiment 344: The method of any one of embodiments 335-343, wherein the method further comprises administering a checkpoint inhibitor.
Embodiment 345: The method of embodiment 344, wherein the checkpoint inhibitor is selected from the group consisting of: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-MR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 346: The method of any one of embodiments 335-345, wherein the method further comprises administering an anti-CD40 antibody.
Embodiment 347: The method of any one of embodiments 336-346, wherein the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.
Embodiment 348: The method of any one of embodiments 335-347, wherein the method further comprises administering a protease inhibitor.
Embodiment 349: The method of embodiment 348, wherein the protease inhibitor is administered in a sufficient amount to repress a repressible protease.
Embodiment 350: The method of embodiment 348 or 349, wherein the protease inhibitor is administered prior to, concurrently with, subsequent to administration of the engineered cells or the composition comprising the engineered cells.
Embodiment 351: The method of embodiment 350, wherein the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir
Embodiment 352: The method of embodiment 350, wherein the protease inhibitor is grazoprevir.
Embodiment 353: The method of embodiment 350, wherein the protease inhibitor comprises grazoprevir and elbasvir.
Embodiment 354: The method of embodiment 353, wherein the grazoprevir and the elbasvir is co-formulated in a pharmaceutical composition.
Embodiment 355: The method of embodiment 354, wherein the pharmaceutical composition is a tablet.
Embodiment 356: The method of embodiment 354 or 355, wherein the grazoprevir and the elbasvir are at a 2 to 1 weight ratio.
Embodiment 357: The engineered nucleic acid of embodiment 356, wherein the grazoprevir is 100 mg per unit dose and the elbasvir is 50 mg per unit dose.
Embodiment 358: The method of any one of embodiments 335-347, wherein the method further comprises administering tamoxifen or a metabolite thereof.
Embodiment 359: The method of embodiment 358, wherein the tamoxifen metabolite is selected from the group consisting of: 4-hydroxytamoxifen, N-desmethyltamoxifen, tamoxifen-N-oxide, and endoxifen.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Regulation of Effector Gene Expression Via Drug-Inducible Regulatable Transcription Factors

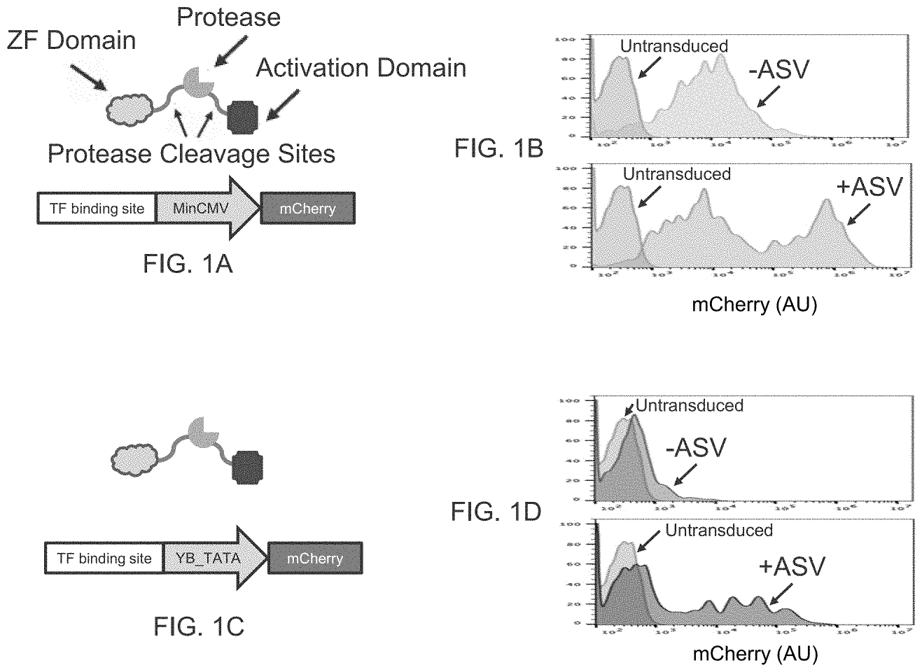

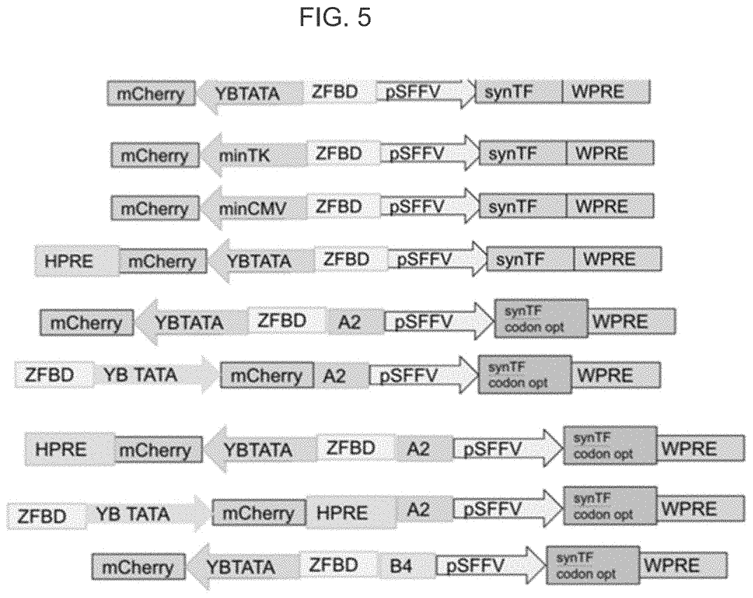

An exemplary zinc finger transcription factor with a repressible protease and protease cleavage site (regulatable TF) to drive effector gene expression was constructed. The regulatable TF is a zinc-finger (ZF) DNA binding domain linked to an NS3 protease, NS3 cleavage site, and the activation domain of a transcription factor (FIG. 1A, the ZF domain is the circle shown on the left of the diagram in the top panel, the NS3 protease is the partial circle shown in the middle of the diagram in the top panel, and the activation domain is the square shown on the right of the diagram in the top panel). The full construct described in FIG. 1A is an example of activation-conditional control polypeptide (ACP). Expression of the regulatable TF is driven via the constitutive SFFV promoter. In untreated cells (e.g., in the absence of a protease inhibitor) the regulatable TF protein is made, but the NS3 protease self cleaves the activation domain from the ZF DNA binding domain of the regulatable TF protein, which inhibits the TF protein from inducing expression of an effector gene. However, in the presence of a protease inhibitor, such as asunaprevir (ASV), the NS3 protease is inhibited and the regulatable TF protein is stabilized. The regulatable TF then binds via the ZF DNA binding domain to a ZF-specific promoter region and drives expression of the effector gene. The ZF-specific DNA sequence is titled the BD (binding domain) in FIGS. 1, 2, 3, 4, and 5.

In this example, a regulatable TF dual vector system was synthesized. In the dual vector system, the regulatable TF gene is in one expression vector, while the ZF-BD and effector gene are on a separate expression vector. In this example, two different ZF-BD and effector gene expression cassettes and vectors were made, one with a minCMV promoter (FIG. 1A) and one with a minYB_TATA promoter (FIG. 1C). FIG. 1, panel A shows an exemplary schematic of a dual vector regulatable TF system with a minCMV promoter.

Materials and Methods

T cells were thawed and rested in X-Vivo 10™ (X Lonza) media with 10 U/mL hrIL-2 (Day 0). On Day 1, T cells were activated with CD3/CD28 Dynabeads (Dynabeads T-activator) at a 3:1 ratio and supplemented with 100 U/mL hrIL-2. On Day 2, T cells were co-transduced with the ACP TF vector and the ACP TF-inducible mCherry vector using lentiviral vectors at 1×10⁵ GVs (GoStix value) units for each lentivirus. Dynabeads were removed on Day 3 and T cells were diluted to 1×10⁶ cell/ml in fresh media 100 U/mL hrIL-2. On Day 6, T cells were passaged into fresh media X-vivo 10 with 100 U/mL hrIL-2 in the presence or absence of 2.5 μM asunaprevir (ASV). T cells were incubated for a further two days and then harvested on Day 8 for flow cytometry to assess mCherry expression.

### Results

Both dual vector systems showed induction of mCherry expression in T cells upon asunaprevir addition (FIGS. 1B and 1D). The cells expressing high levels of mCherry in each case likely represent the co-transduced cells that received both the regulatable TF and the mCherry vector. The minCMV dual vector system likely showed leaky expression of mCherry as the transduced but non-asunaprevir treated cells showed mCherry expression above the untransduced cells (FIG. 1B). In contrast, the minYB_TATA dual vector system shows minimal leaky expression as the transduced but non-asunaprevir treated cells showed little mCherry expression above the untransduced cells (FIG. 1D). mCherry was expressed in the transduced cells once asunaprevir was added to inhibit degradation of the regulatable TF, indicating that the mCherry expression was largely due to regulatable TF-induced transcription.

The minCMV dual vector system showed a higher mCherry expression in the induced state (asunaprevir treated cells) than the minYB_TATA system. Thus, altering the minimal promoter allowed for control over the dynamic range and baseline expression in cells expressing payloads regulated by the regulatable TF.

### Example 2: Single Vector Regulatable Transcription Factor Regulation of IL-10 and IL-12 Expression

A single vector TF and effector gene expression system was assessed.

Materials and Methods

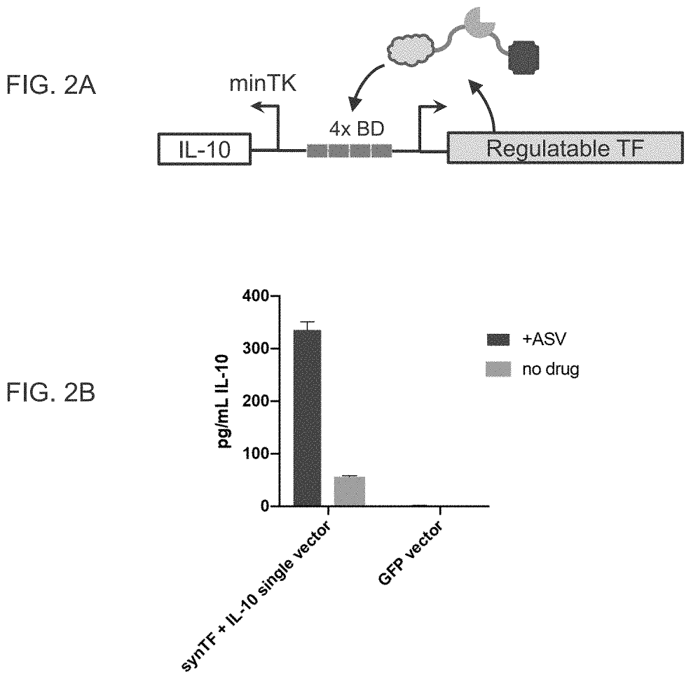

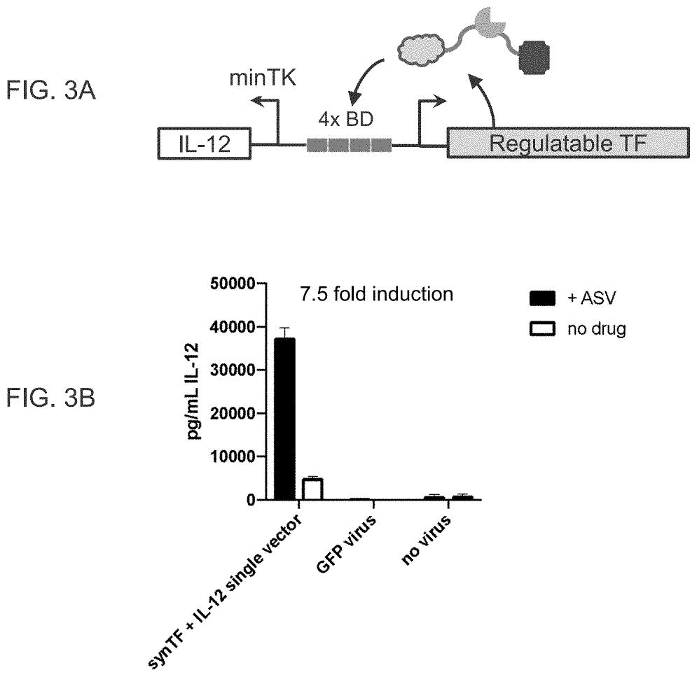

Two regulatable TF and effector gene expression cassettes were constructed as shown in FIG. 2A and FIG. 3A. In FIG. 2A, an IL-10 effector gene with a minTK promoter and a 4× ZF binding sequence (ZF-BD) was arranged in a 3′ to 5′ orientation upstream of the 5′ end of a drug inducible promoter and the regulatable TF. In FIG. 3A, an IL-12 effector gene with a minTK promoter and a 4× ZF binding sequence (ZF-BD) was arranged in a 3′ to 5′ orientation upstream of the 5′ end of a drug inducible promoter and the regulatable TF.

T cells were thawed and rested in X-Vivo 10 media with 10 U/mL hrIL-2 (Day 0). On Day 1, T cells were activated with a 3:1 incubation of CD3/CD28 Dynabeads and supplemented with 100 U/mL hr IL-2. On Day 2, T cells were transduced with 1×10⁵ GV units of a lentiviral vector containing the synTF (also referred to as Pro-Dial)-inducible IL-10 or IL-12 vector. A GFP lentiviral vector was used as a negative control. CD3/CD28 Dynabeads were removed on Day 4 and T cells were passaged. On Day 6, T cells were passaged into wells in the presence or absence of 2.5 μM asunaprevir in X-Vivo 10 media with 100 U/mL hrIL-2. Transduced T cells were incubated for a further two days and the supernatant collected via centrifugation for IL-10 or IL-12 quantification.

For IL-10, supernatants were diluted 1/100 in PBS/BSA and analyzed using the human IL-10 Quantikine ELISA kit (R&D Systems, cat #D1000B).

For IL-12, supernatants were diluted 1/100 in PBS/BSA and analyzed using the human IL-12 p70 Quantikine ELISA kit (R&D Systems, cat #D1200B).

### Results

T cells transduced with the regulatable TF inducible IL-10 single vector showed a 6-fold increase in secreted IL-10 levels after addition of asunaprevir to inhibit the NS3 protease activity as compared to transduced T cells in the absence of asunaprevir (FIG. 2B). The control cells transduced with GFP vector produced no IL-10. Thus, the single vector regulatable TF system is capable of drug-regulated cytokine production.

In addition, T cells transduced with the regulatable TF inducible IL-12 single vector showed a 7.5 fold increase in secreted IL-12 levels after addition of asunaprevir to inhibit the NS3 protease activity as compared to transduced T cells in the absence of asunaprevir (FIG. 3B). The control cells transduced with GFP vector produced no IL-12. Thus, the single vector regulatable TF system is capable of drug-regulated cytokine production of different cytokines.

### Example 3: Co-Expression of Single Vector Regulatable Transcription Factor and Additional Proteins

A single vector regulatable TF and effector gene expression system with co-expression of an additional protein was assessed.

Materials and Methods

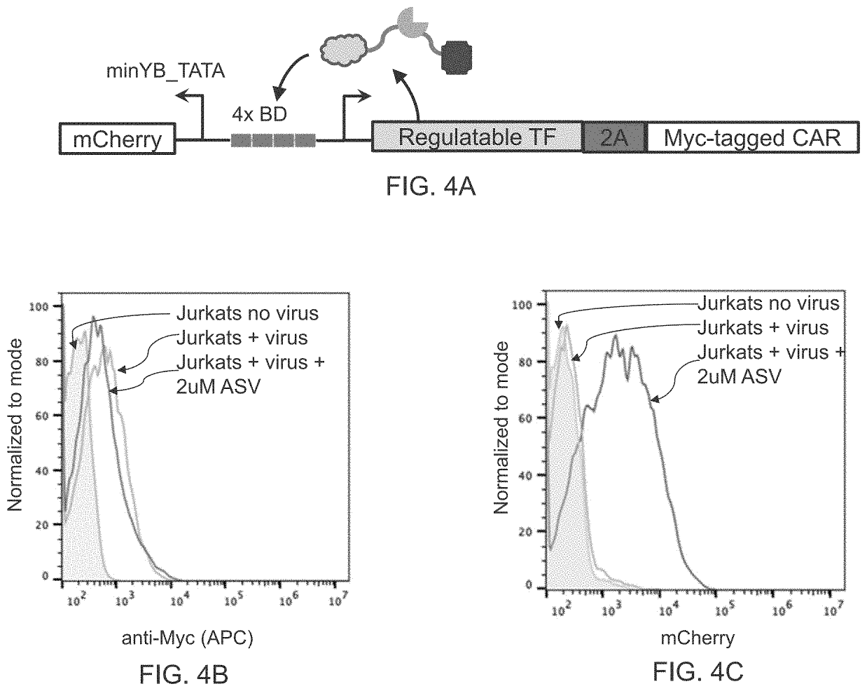

A regulatable TF and reporter gene expression cassette was constructed as shown in FIG. 4A. The regulatable TF gene was linked to a myc-tagged chimeric antigen receptor (CAR) via a 2A linker at the 3′ end of the regulatable TF gene. An mCherry reporter gene with a minYB_TATA promoter and a 4× ZF binding sequence (ZF-BD) was arranged in a 3′ to 5′ orientation upstream of the 5′ end of a drug inducible promoter and the linked regulatable TF-CAR genes.

On Day 0, Jurkat cells were plated in RPMI (RPMI media) at 1×10⁶ cells/ml in a 24 well plate (1 ml/well). The Jurkat cells were transduced with 3×10⁵ GVs units of virus/well. On Day 2, Jurkat cells were passaged into wells in the presence or absence of 2 μM asunaprevir. On Day 6, Jurkats were harvested for flow cytometry. Cells were stained with a-Myc-APC antibody (R&D Systems, cat #IC3696A), to assess the expression of the CAR. mCherry expression was also assessed.

### Results

As shown in FIG. 4B, the myc-tagged CAR was expressed at similar levels in both the asunaprevir-treated cells and untreated cells, indicating that the regulatable TF can be co-expressed with a CAR in Jurkat cells. The presence and activity of the protease in the regulatable TF did not affect the expression or stability of the CAR protein linked to the regulatable TF. This indicates that the regulatable TF system can function in CAR-T cells.

As shown in FIG. 4C, the addition of asunaprevir to inhibit the protease resulted in regulatable TF-induced expression of the mCherry reporter gene. Thus, the regulatable TF expression system can function correctly in the presence of an additional co-expressed protein to induce expression of an effector protein.

### Example 4: CAR and Payload Expression

CAR and payload/cytokine expression was assessed in cells co-transduced with a CAR construct and effector gene expression systems for “armoring” cells. Payload/cytokine expression was assessed in cells transduced with an effector gene expression system with or without a CAR construct.

### Materials and Methods

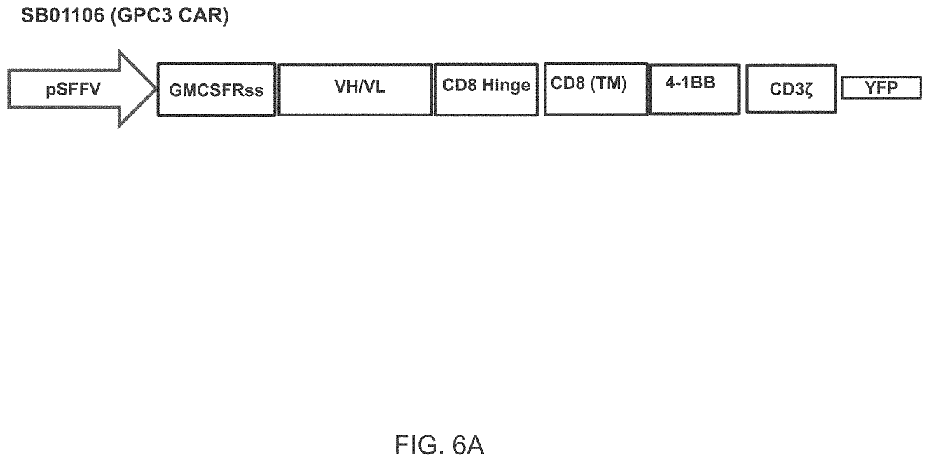

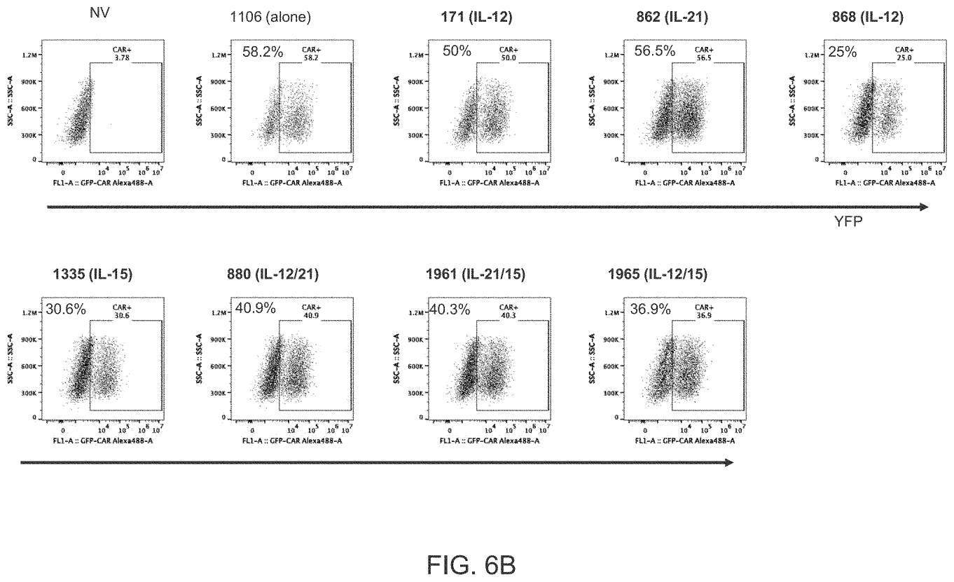

Effector gene (e.g., cytokine payload of interest) expression cassettes were constructed and cloned into a viral vector then used to produce payload virus, see Table 10. A CAR construct targeting GPC3, a protein highly and specifically expressed in liver cancer [Sun et al, Medical Science Monitor, 2017], was constructed as shown in FIG. 6A (construct “1106”) and cloned into a viral vector and used to produce GPC3-CAR virus. The GPC3-CAR was fused to a YFP reporter to monitor expression.

On Day 0, CD4/CD8 T cells (FL00711) were thawed and activated with Dynabeads (3:1) in complete T cell media (Optimizer+Supplements—Gibco+5% human serum)+IL-2 (100 Units/ml). On Day 1, cells were transduced with 1×10⁵ GV of payload virus+/−1×10⁵ GV of GPC3-CAR (construct 1106) virus. On Day 4, CAR transduction efficiency was assessed by flow cytometry (Cytoflex). Florescent median intensity (MFI) and percentage of cells expressing GPC3-CAR was analyzed by FlowJo. The cells were then transferred to Grex 24 (Wilson Wolf) in full T cell media+IL2 for further expansion.

On day 8, cells were harvested from Grex plate and counted then 1×10⁶ cells were spin down and resuspended in 1 mL of Full T cells media without IL-2. Cells were seeded in a 24-well plate for 48 hours. On day 10, supernatant was harvested and cells were counted from the 24-well plate to determine their viability. Cells were spun down and supernatant transferred to −80C for further processing by Luminex using Luminex 3plex (IL-12/21/15) kit (LXSAHM-03; R&D) for payload expression.

For co-culturing experiments with target cells, 2.5×10⁴HepG2 cells/well were seeded for 5 hrs in full EMEM. Following which, EMEM was removed and 1×10⁶ cells total transduced T cells were added in T cell media without IL-2 (4:1, E:T). Supernatant was harvested 48 hours later. Payload expression and T cell activation was assessed using Luminex-6plex kit (LXSAHM-06; R&D) (IL-12/21/15/2/TNFa/IFNg).

TABLE 10
Effector gene constructs

| SB# | Promoter | Insert |
|---|---|---|
| SB00171 | SSFV | IL12 |
| SB00862 | SSFV | SS12-IL12 |
| SB00880 | SSFV | ssIL12-T2A-ssIL21 |
| SB00868 | SSFV | IL21ss-IL21(ATUM) |
| SB01335 | SSFV | ssIL-15 |
| SB01961 | SSFV | SS.IL21—Furin T2a—ss.mIgGk-IL15 |
| SB01965 | SSFV | IL12-Furin E2A-T2A- IL 15 |

### Results

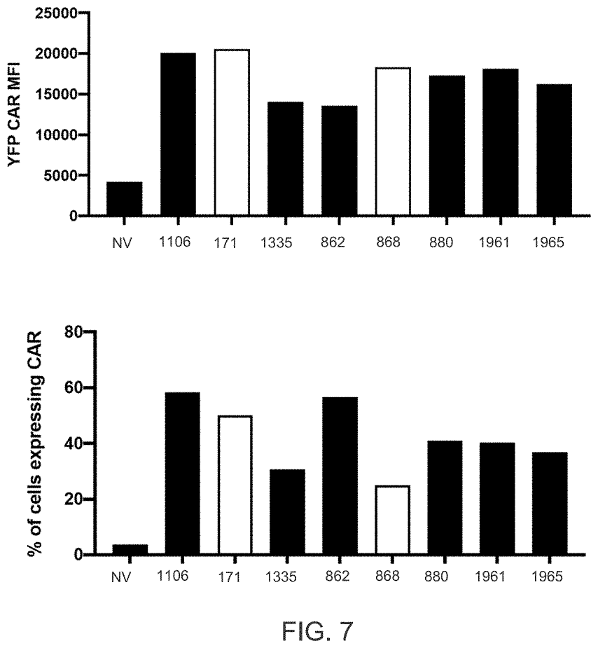

CAR expression was assessed in cells also engineered to express an effector molecule (e.g., cytokine) payload. As shown in FIG. 6B and FIG. 7 (bottom panel), the GPC3-CAR was expressed in 25.0-58.2% of transduced cells 4 days after transduction. As shown in FIG. 7 (top panel), GPC3-CAR expression level as assessed by YFP MFI was generally comparable between transduction with GPC3-CAR virus only (“1106”) and the different payload constructs examined. As shown in Table 11, viability was also generally comparable between transduction with GPC3-CAR virus only (“1106”) and the different payload constructs examined. Accordingly, CAR expression and viability was not significantly altered by engineering with an armoring effector molecule payload.

TABLE 11
Cell line cytokine payloads and viability

| | Viability | |
|---|---|---|
| Cell line | (%) | Cytokine |
| NV | 85 | |
| 1106 | 83 | |
| 171 + 1106 | 85 | IL-12 |
| 862 + 1106 | 83 | IL-21 |
| 868 + 1106 | 83 | IL-12 |
| 1335 + 1106 | 75 | IL-15 |
| 880 + 1106 | 76 | IL-12/21 |
| 1961 + 1106 | 82 | IL-21/15 |
| 1965 + 1106 | 81 | IL-12/15 |

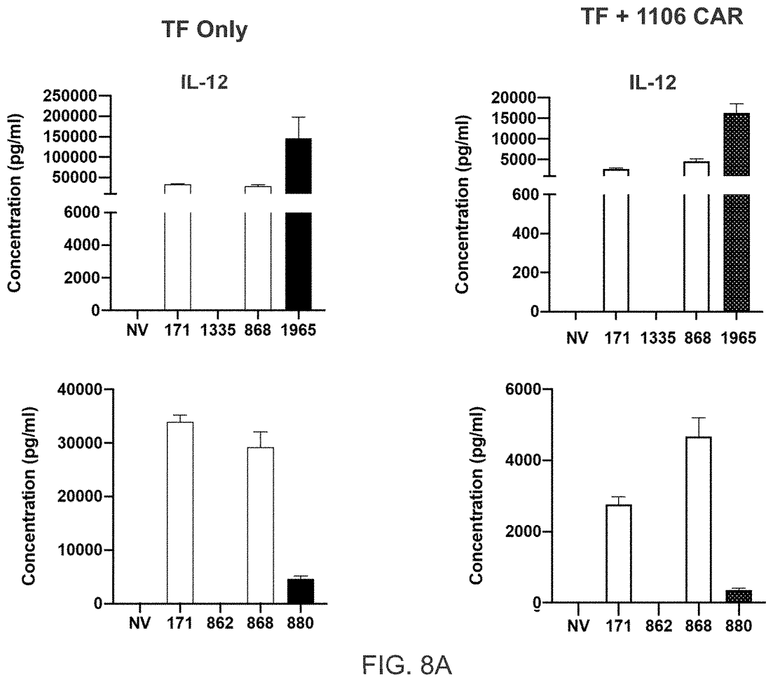

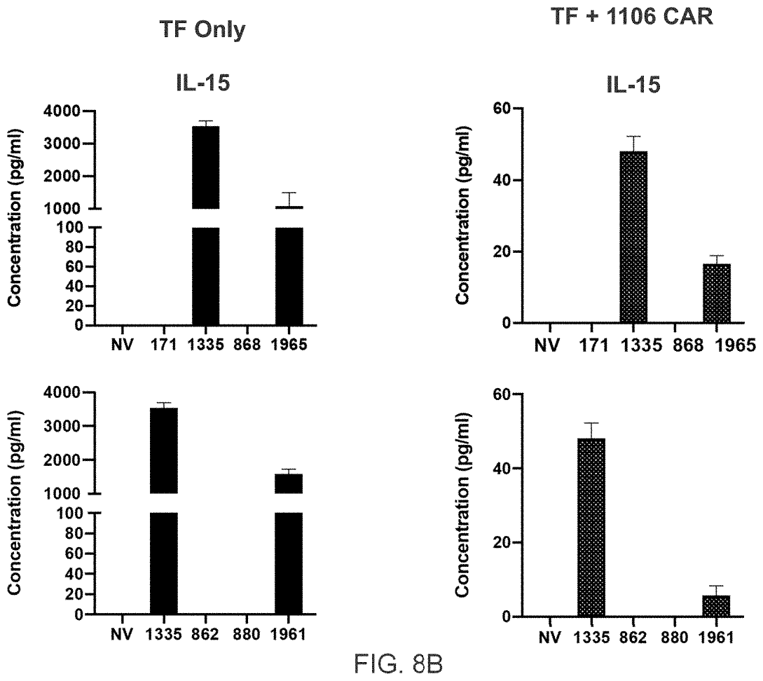

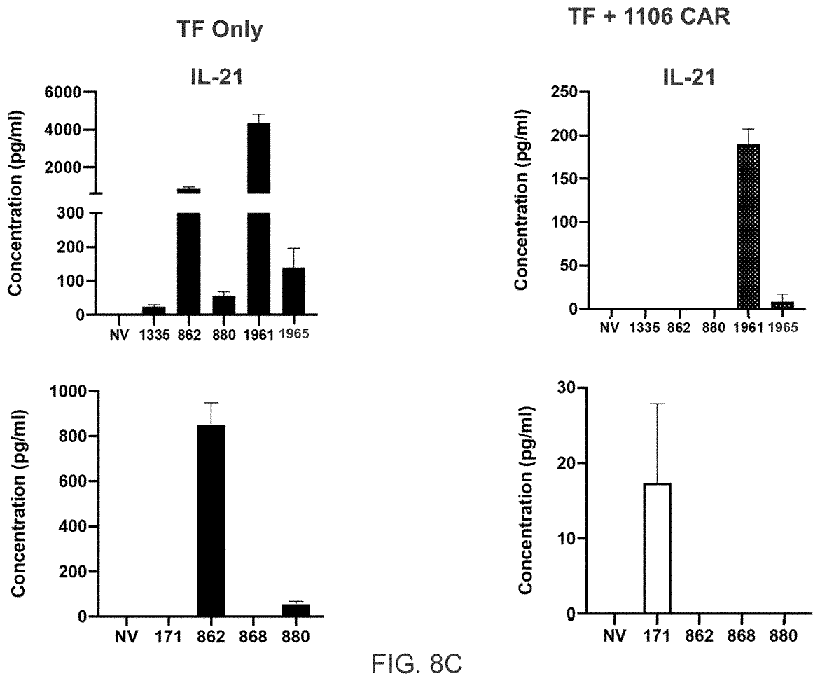

Payload expression of cytokines was assessed in cells transduced with an armoring effector molecule expression system with or without cells also engineered to express a CAR construct. As shown in FIG. 8, payload cytokine expression was detected in all constructs encoding the cytokine payload of interest only (left panels: FIG. 8A—IL-12;

FIG. 8B—IL-15; FIG. 8C—IL-21). Expression of cytokines was also generally better in constructs engineered to encode a single cytokine (left panels vs right panels). Notably, when co-transduced with the GPC3-CAR, cytokine levels were reduced generally by a log-fold or undetected (right panels: FIG. 8A—IL-12; FIG. 8B—IL-15; FIG. 8C—IL-21).

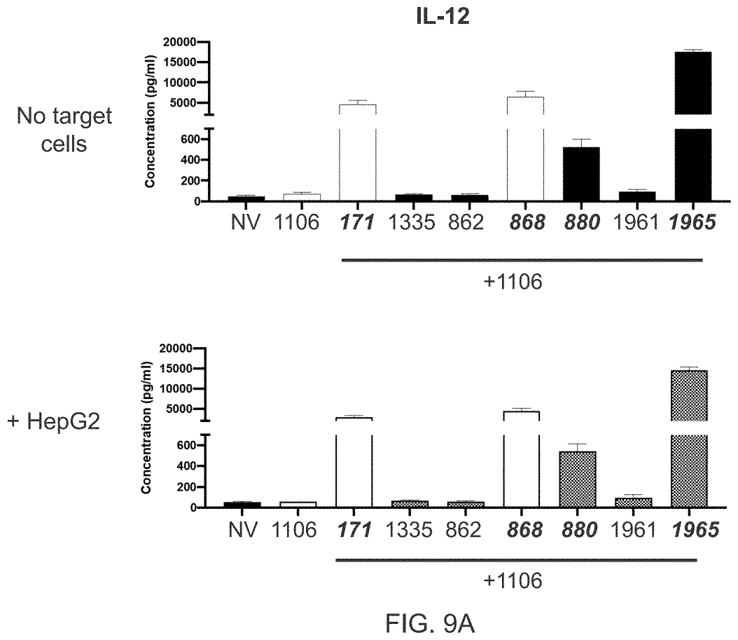

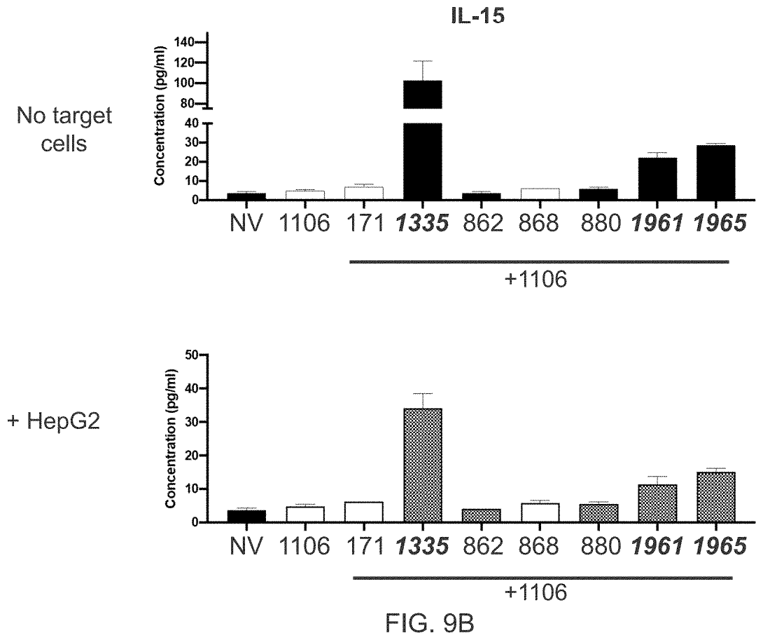

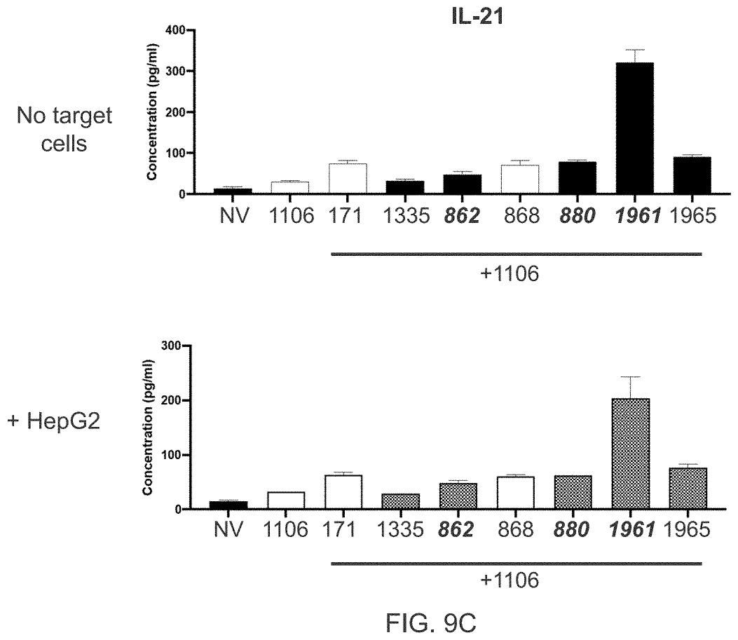

Payload expression of cytokines was next assessed when CAR T cells were co-cultured with target cells. As shown in FIG. 9, no significant change of payload cytokine expression for IL-12 or IL-21 was observed in the presence of HepG2 cells, while expression of IL-15 was reduced by approximately 50% (top panels vs bottom panels: FIG. 9A—IL-12;

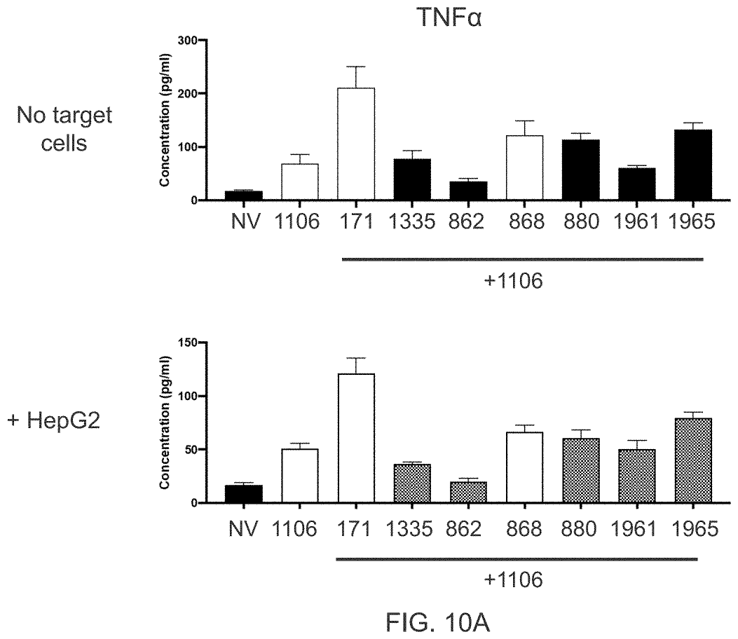

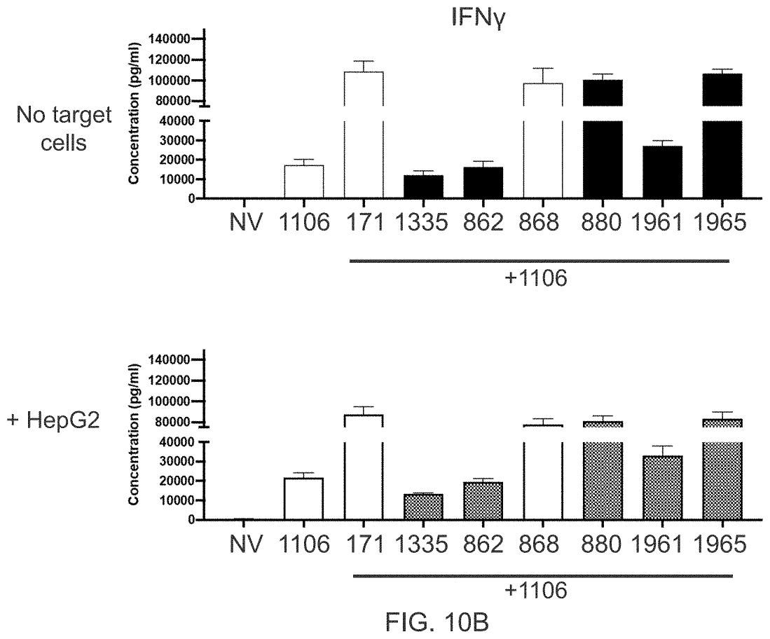

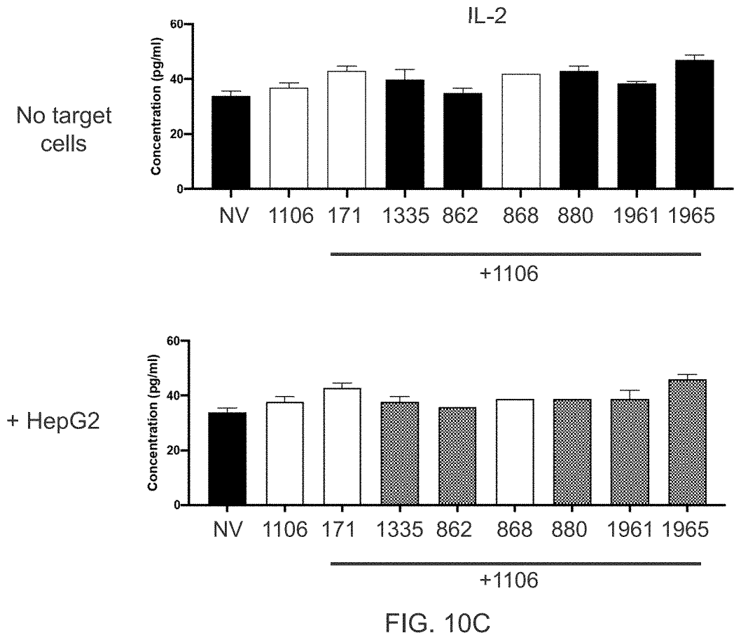

FIG. 9B—IL-15; FIG. 9C—IL-21). In addition to payload cytokine expression, production of additional T cell activation cytokines was assessed. As shown in FIG. 10, no significant change of TNFα, IFNγ, or IL-2 expression was observed in the presence of HepG2 cells target cells (top panels vs bottom panels: FIG. 10A—TNFα; FIG. 10B—IFNγ; FIG. 10C—IL-2).

### Example 5: In Vivo CAR Activity in Co-Expression Systems

CAR expression, payload/cytokine expression, and anti-tumor activity was assessed in cells co-transduced with a CAR construct and effector gene expression systems for “armoring” cells.

### Materials and Methods

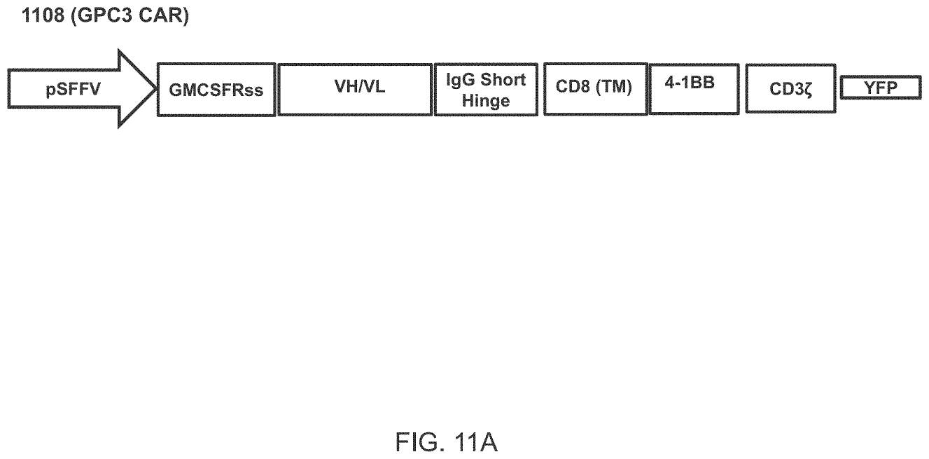

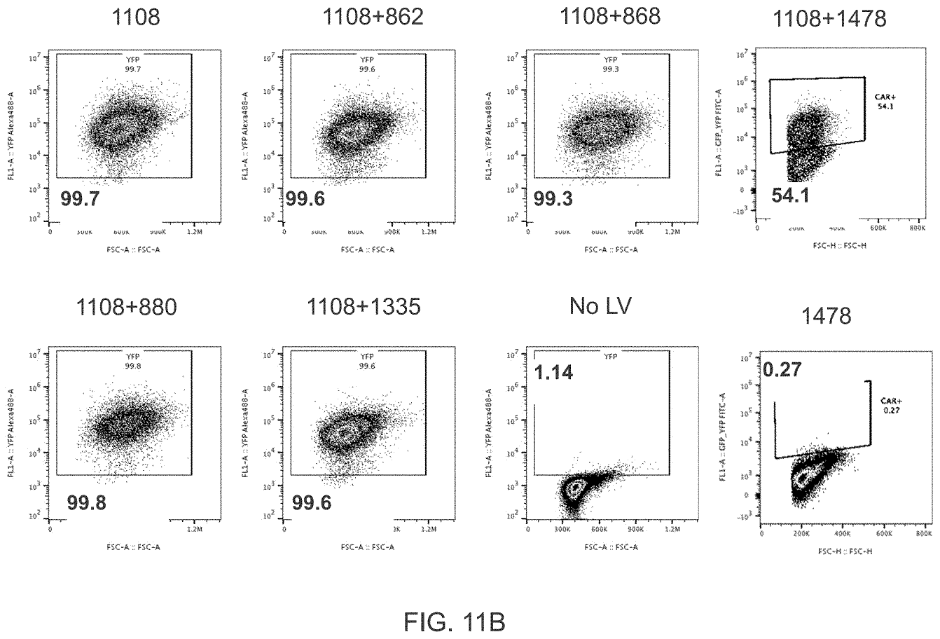

Effector gene (e.g., cytokine payload of interest) expression cassettes were constructed and cloned into a viral vector then used to produce payload virus, see Table 12. A CAR construct targeting GPC3, a protein highly and specifically expressed in liver cancer (Sun et al, Medical Science Monitor, 2017), was constructed as shown in FIG. 11A (construct “1108”) and cloned into a viral vector and used to produce GPC3-CAR virus. The GPC3-CAR was fused to a YFP reporter to monitor expression.

On day 0, 6×10⁶ HepG2 fLuc cells were implanted (IP cavity) in NGS female mice. On day 7, body weight (BW) was measured and mice were randomized. On day 11, 5×10⁶ CAR-T cells were injected IV. Mice were observed for overall health condition, BLI measurement, and body weight twice a week. Mice were also bled once a week into EDTA tubes and spun down to separate cells from plasma. Plasma frozen at −80C until Luminex analysis using a Luminex-6plex kit (LXSAHM-06; R&D) (IL-12/21/15/2/TNFa/IFNg) and the cell pellet was resuspended and processed for flow cytometry. Mice were sacrificed when body weight drops more than 15% of original weight. On day 45, all mice were sacrificed with tumors, lung, liver, and spleen were collected and fixed as well as tumors weighed.

On Day 0, CD4/CD8 T cells (FL00711) were thawed and activated with Dynabeads (3:1) in complete T cell media (Optimizer+Supplements—Gibco+5% human serum)+IL-2 (100 Units/ml). On Day 1, cells were transduced with 1×10⁵ GV of payload virus+/−1×10⁵ GV of GPC3-CAR virus (construct “1108”). On Day 4, CAR transduction efficiency was assessed by flow cytometry (Cytoflex). Florescent median intensity (MFI) and percentage of cells expressing GPC3-CAR was analyzed by FlowJo. The cells were then transferred to Grex 24 (Wilson Wolf) in full T cell media+IL2 for further expansion.

On day 9, cells were harvested from Grex plate and counted then 1×10⁶ cells were spin down and resuspended in 1 mL of Full T cells media without IL-2. Cells were seeded in a 24-well plate for 48 hours. On day 11, supernatant was harvested and cells were counted from the 24-well plate to determine their viability. Cells were spun down and supernatant transferred to −80C for further processing by Luminex using Luminex 3plex (IL-12/21/15) kit (LXSAHM-03; R&D) for payload expression.

For ex vivo cell analysis, 1 ml PBS was added to blood samples, mixed and transferred to 15 ml conical tubes then 2 ml/tube lysis buffer added. Tubes were inverted 2× to mix the incubate at RT for 5 min. Next, ˜14 ml of FACS buffer wash was added and repeated 2×. At last spin, cells were resuspended in ˜250-400 ul and transferred to v-bottom plate, then washed with 400 ul/well PBS. Next, 100 ul/well of viability dye (zombieUV; Biolegend) was added and incubated at RT dark for 15 min. Wells then received 200 ul FACS buffer, spun at 400 g 5 min, decanted, then 200 ul/well antibody mix added and incubated at RT for 45 min. Cells were washed with 200 ul FACS buffer, spun at 400 g 5 min, decanted, and was repeated 2×. Cells were resuspended in 150 ul/well FACS buffer, transferred to u-bottom plate and read. Antibodies were purchased from Biolegend and included: mCD45 clone 30-F11; hCD45 clone H130; hCD3 clone OKT3; hCD8 clone SK1.

TABLE 12
Effector gene constructs

| SB # | Promoter | Insert |
|---|---|---|
| SB00880 | SSFV | ssIL12-T2A-ssIL21 |
| SB00862 | SSFV | SS12-IL12 |
| SB00868 | SSFV | IL21ss-IL21(ATUM) |
| SB01335 | SSFV | ssIL-15 |
| SB01478 | SSFV | ssIL21-T2 A-IL15_ss + propeptideIL15_mature |

### Results

CAR expression was assessed in T cells also engineered to express an armoring cytokine payload. As shown in FIG. 11B, the GPC3-CAR was expressed in greater than 54% of all constructs examined, greater than 99% of 5 out of 6 constructs, 4 days after transduction. Payload expression of cytokines was next assessed. As shown in Table 13, the engineered CAR T cells expressed their respective payload cytokine.

TABLE 13
Cytokine expression (pg/ml)

| | IL12 | IL21 | IL15 |
|---|---|---|---|
| NV | 56.54 | 19.34 | 6.382 |
| 1108 | 48.77 | 17.66 | 4.55 |
| (CAR) | | | |
| CAR + 862 | 48.77 | 152.5 | 6.945 |
| CAR + 868 | 3348 | 70.14 | 6.94 |
| CAR + 880 | 163.5 | 46.27 | 6.946 |
| CAR +1335 | 84.85 | 32.30 | 3165 |
| CAR + 1874 | n/a | n/a | n/a |
| 1478 | — | 456.1 | 70.64 |

*Bold denotes constructs engineered to express the indicated cytokine

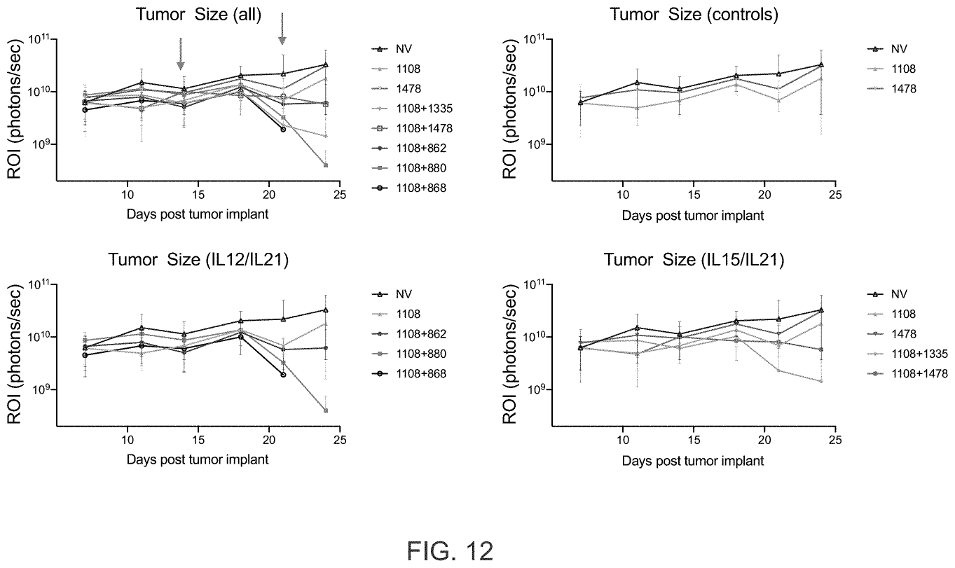

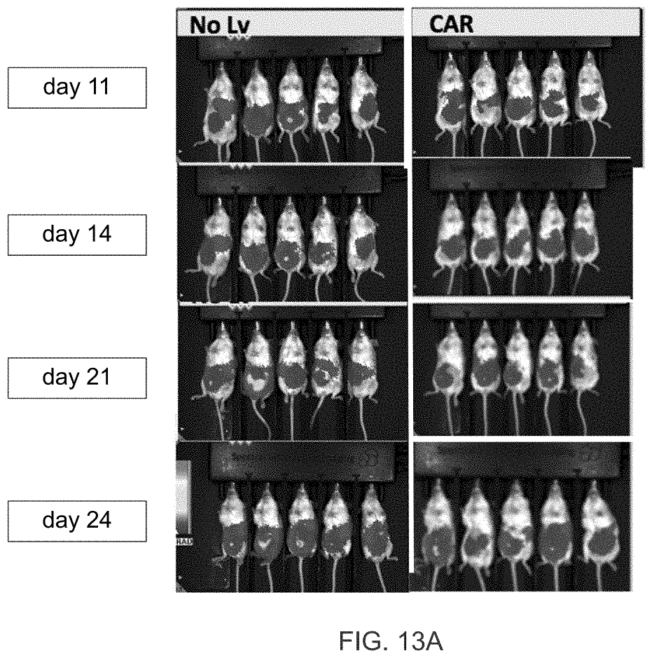

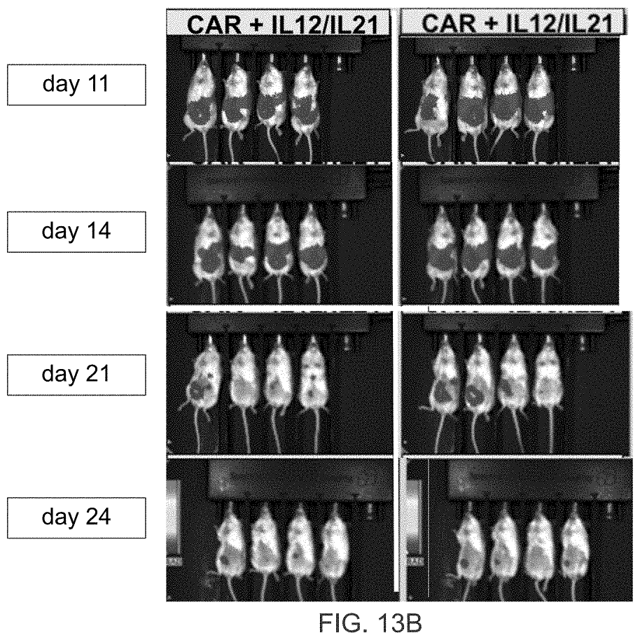

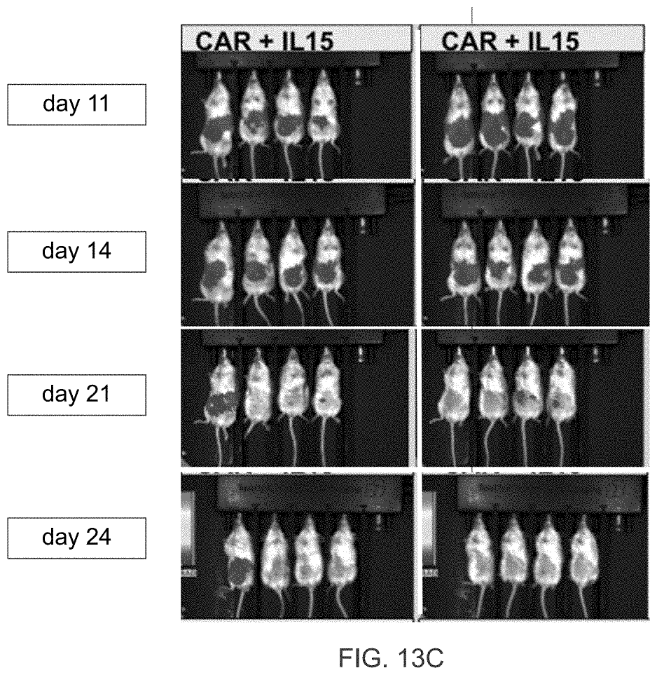

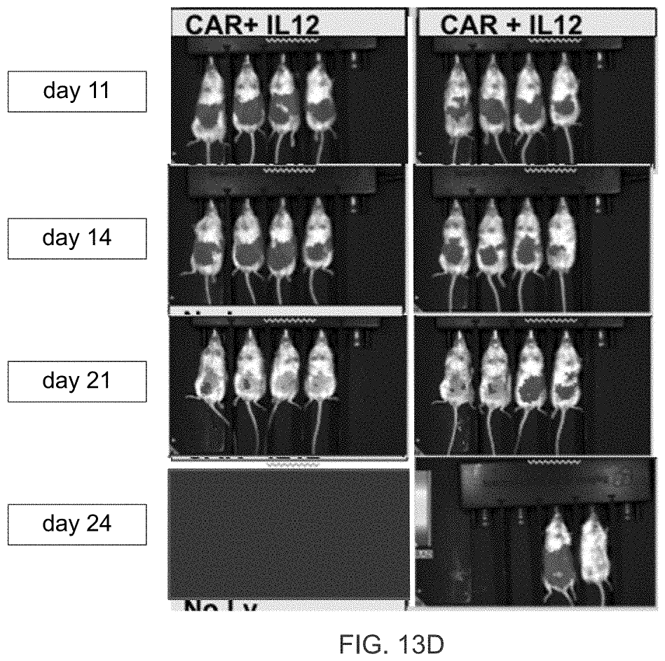

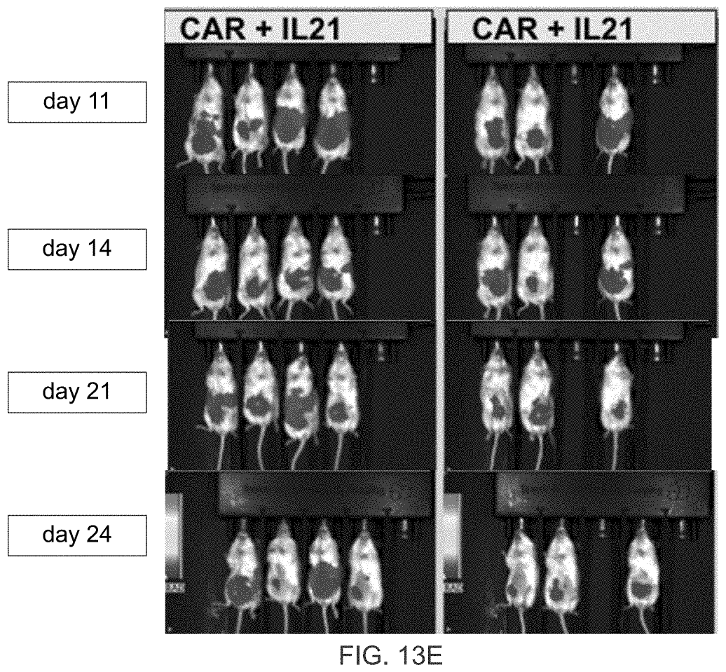

Efficacy of the CAR T cells engineered with a armoring cytokine payload was then assessed. Mice treated with CAR-T cells+IL-12 (868) were sacrificed at day 21 and mice treated with CAR-T cells+IL-15/IL-21 (1478) were sacrificed at day 24 due to poor health conditions. As shown and summarized in FIG. 12, tumor sizes were assessed by BLI measurement on days 11, 14, 21, and 24 for mice treated with T cells transduced without virus (FIG. 13A—left panel), GPC3-CAR T alone without a cytokine (FIG. 13A—right panel), or GPC3-CAR T engineered with an armoring cytokine (FIG. 13B—IL-12/IL-21 co-expression; FIG. 13C—IL-15; FIG. 13D—IL-12; FIG. 13E—IL-21). At low dose (5e6 cell/ml), T cells transduced without virus or with the GPC3-CAR alone demonstrated increased tumor burden. In contrast, T cells engineered to co-express the CAR and IL-12/IL-21 (“880” FIG. 12; FIG. 13B) or IL-15 (“1335” FIG. 12; FIG. 13C) demonstrated tumor burden reduction by day 24. T cells engineered to co-express the CAR and IL-12 demonstrated initial tumor burden reduction (“868” FIG. 12; FIG. 13D), but the combination demonstrated high toxicity as mice were sacrificed due to poor health. T cells engineered to co-express the CAR and IL-21 demonstrated overall tumor size maintenance (“862” FIG. 12) with tumor burden reduction detected in individual mice (FIG. 13E), potentially due to the low levels of the cytokine. Accordingly, the results indicate CAR T cells co-expressing select cytokines and cytokine combinations demonstrated efficacy in reducing tumor burden, but potentially present high toxicity suggesting that armoring cytokine expression may benefit from regulation.

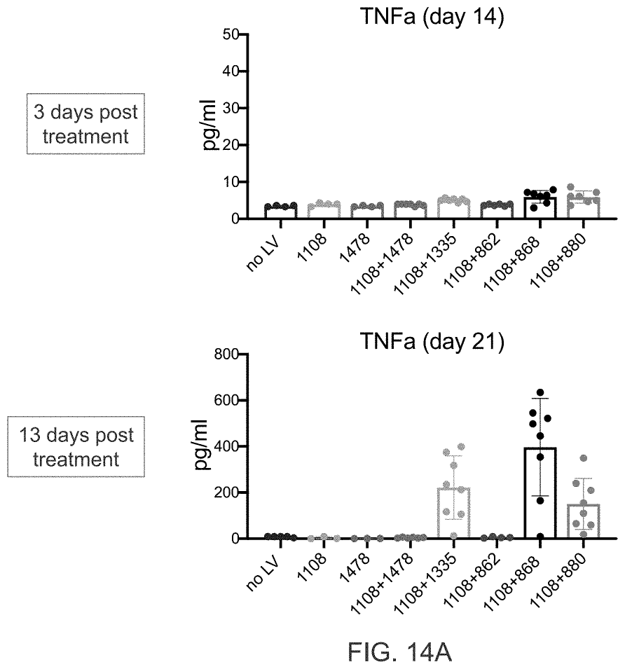

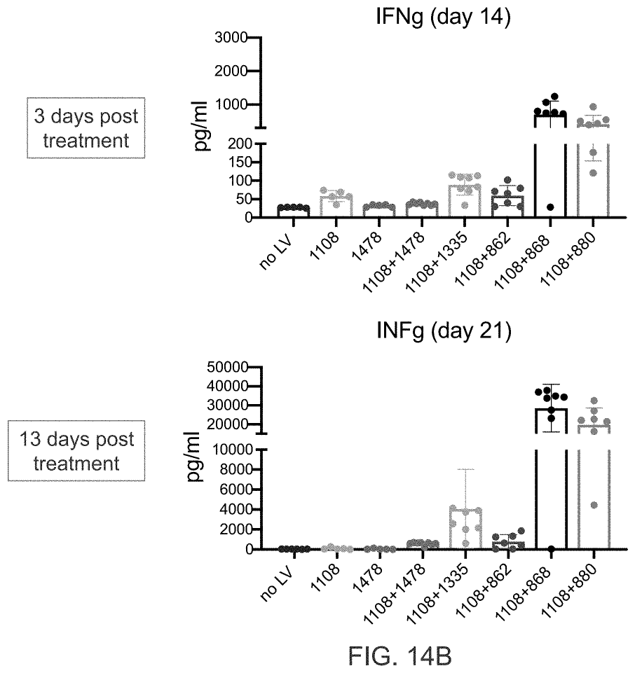

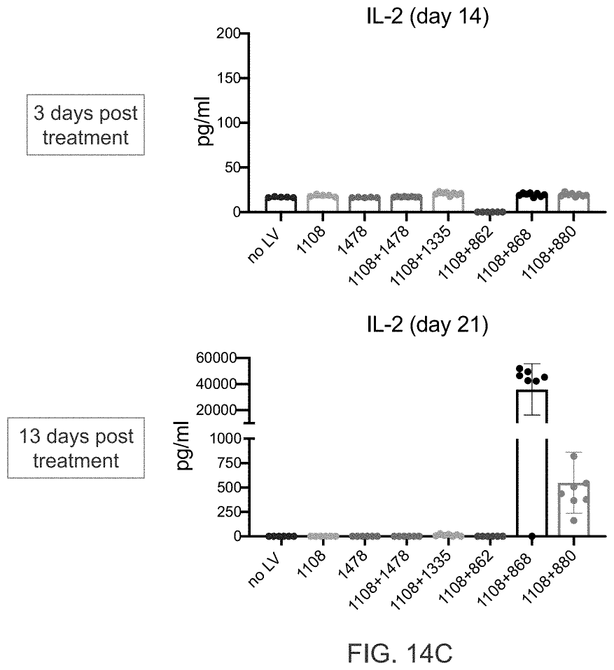

T cell activation cytokines was assessed in plasma of treated mice. As shown in FIG. 14, no significant detection of TNFa (FIG. 14A) or IL-2 (FIG. 14C) was observed on Day 14, 3 days after treatment (top panel). IFNγ (FIG. 14B) was observed in treatment groups expressing IL-12 alone (“880”) or co-expressing IL-12/21 (“880”) on Day 14 (FIG. 14B—top panel). In contrast, TNFa (FIG. 14A—bottom panel) was observed in treatment groups that correlated with tumor reduction (“868”/“880”/“1335”; see FIG. 12 and FIG. 13) on Day 21, 10 days after treatment (bottom panel). IFNγ (FIG. 14B—bottom panel) was also observed in treatment groups that correlated with tumor reduction at Day 21, though notably higher detection (˜10×) in IL-12 and IL-12/21 systems. IL-2 (FIG. 14C—bottom panel) was also observed was in IL-12 and IL-12/21 systems at Day 21, with notably higher detection in the IL-12 alone system. Accordingly, the results indicate CAR T cells engineered to express select armoring cytokines and armoring cytokine combinations demonstrated production of T cell activation cytokines.

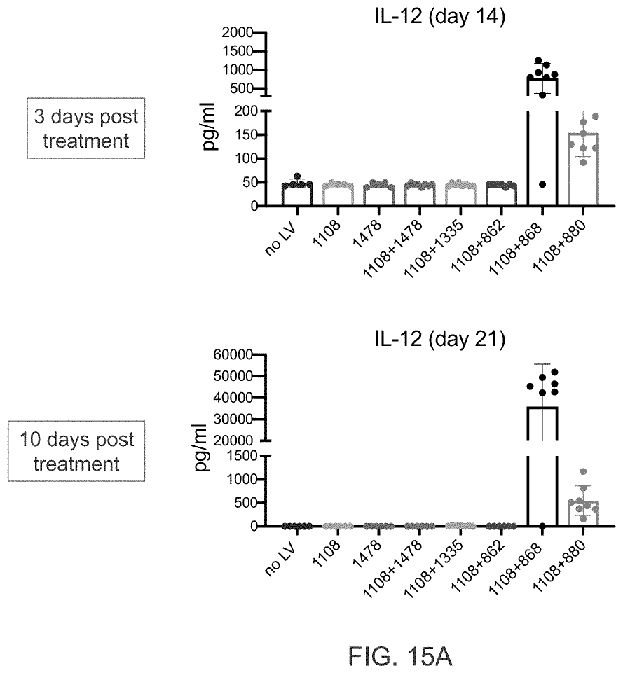

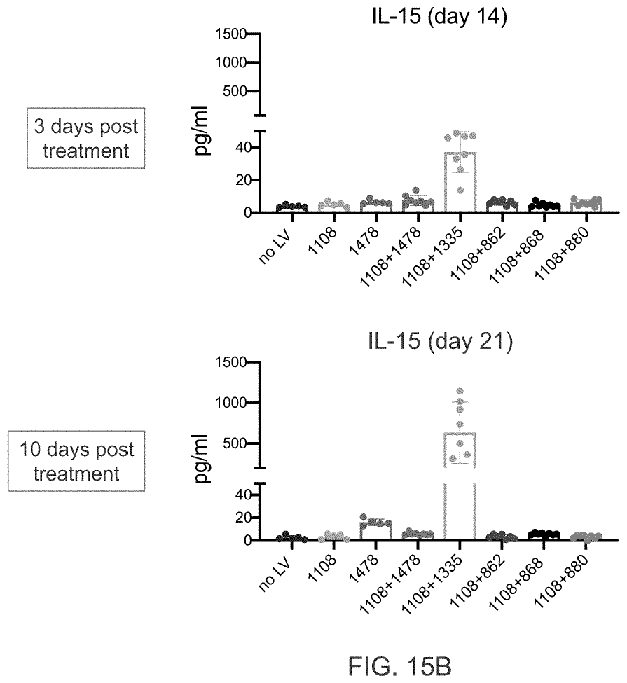

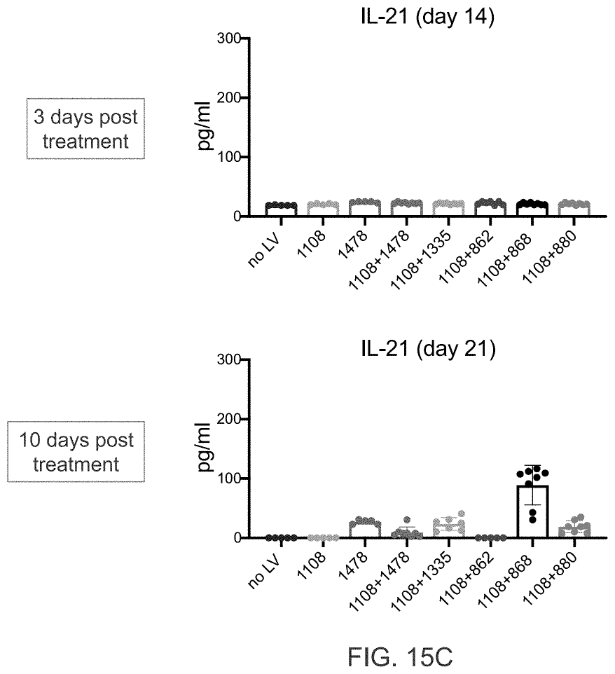

Payload expression of cytokines was also assessed in the plasma of treated mice. As shown in FIG. 15A, IL-12 was observed on Day 14, 3 days after treatment (top panel) and on Day 21, 10 days after treatment (bottom panel) in IL-12 and IL-12/21 systems, though notably higher detection (˜10×) in IL-12 alone. IL-12 expression also increased as the percentage of human CD45+ cells increased in mice (see below). As shown in FIG. 15B, IL-15 was observed on Day 14, 3 days after treatment (top panel) and on Day 21, 10 days after treatment (bottom panel) in the IL-15 system. IL-15 expression also increased as the percentage of human CD45+ cells increased in mice (see below), though more moderately than IL-12. Unexpectedly, IL-21 was only detected on Day 21, 10 days after treatment (bottom panel) in the IL-12 system (“868”) and not in IL-21 alone or IL-12/IL-21 co-expression systems (FIG. 15C). Accordingly, the results indicate CAR T cells engineered to express armoring cytokines demonstrated production of payload cytokines in select systems.

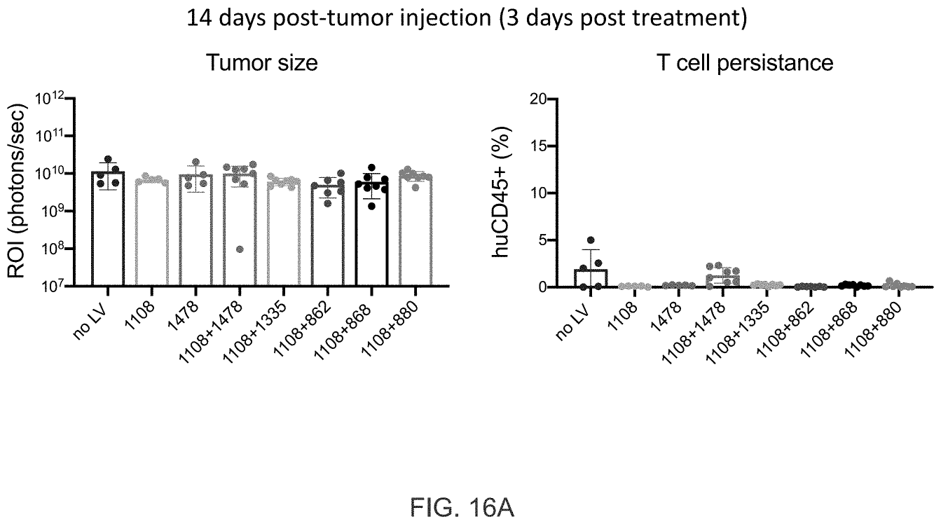

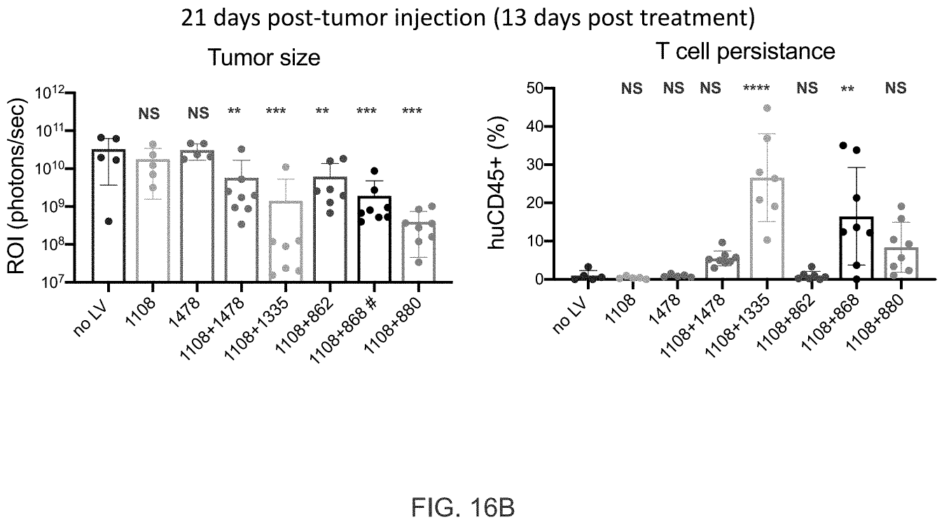

Ex vivo analysis of T cell persistence was assessed. As shown in FIG. 16A, no or minimal human CD45+ CAR T cells were detected on Day 14, 3 days after treatment (right panel) correlating with no observed reduction in tumor size (left panel). In contrast, as shown in FIG. 16B, CD45+ CAR T cells were detected in the armored CAR systems (“868”/“880”/“1335”; right panel) that demonstrated tumor reduction (left panel), though notably all armored groups present high degree of toxicity, potentially due to GvHD.

### Example 6: Tamoxifen Regulatable Transcription Factor Systems

Payload expression was assessed using a regulatable TF expression system.

### Materials and Methods

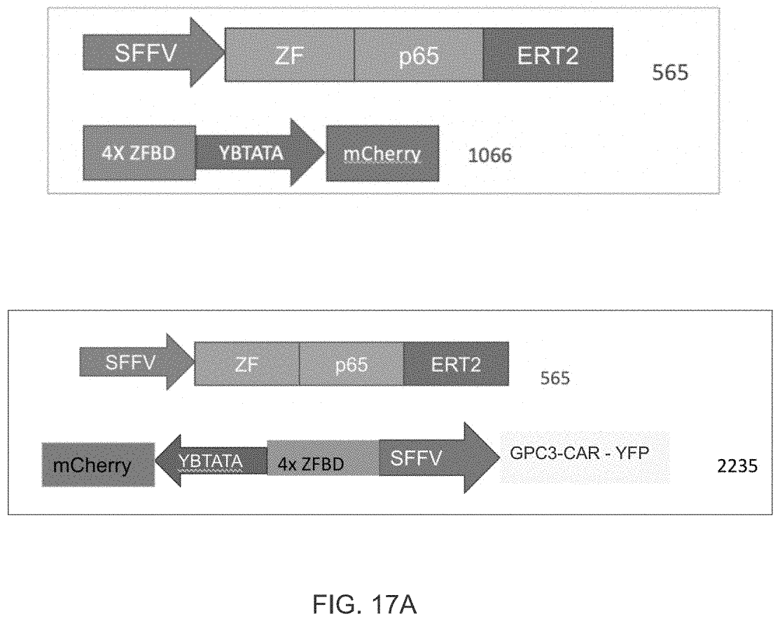

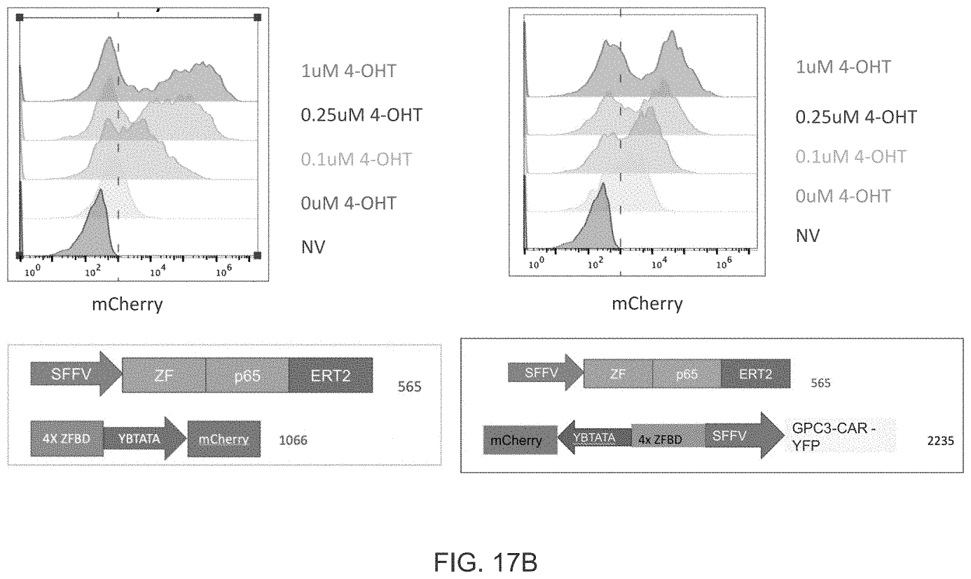

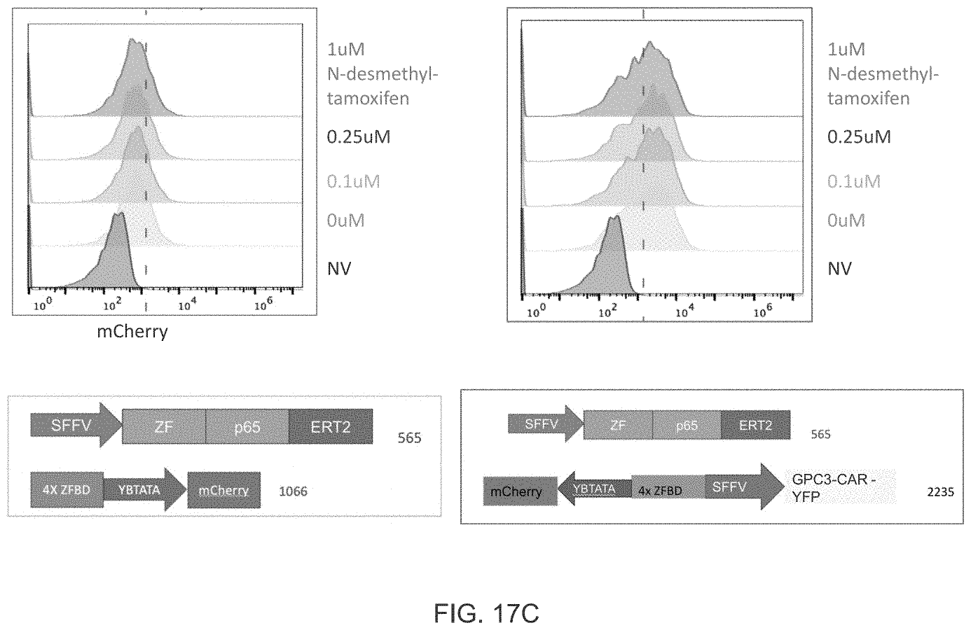

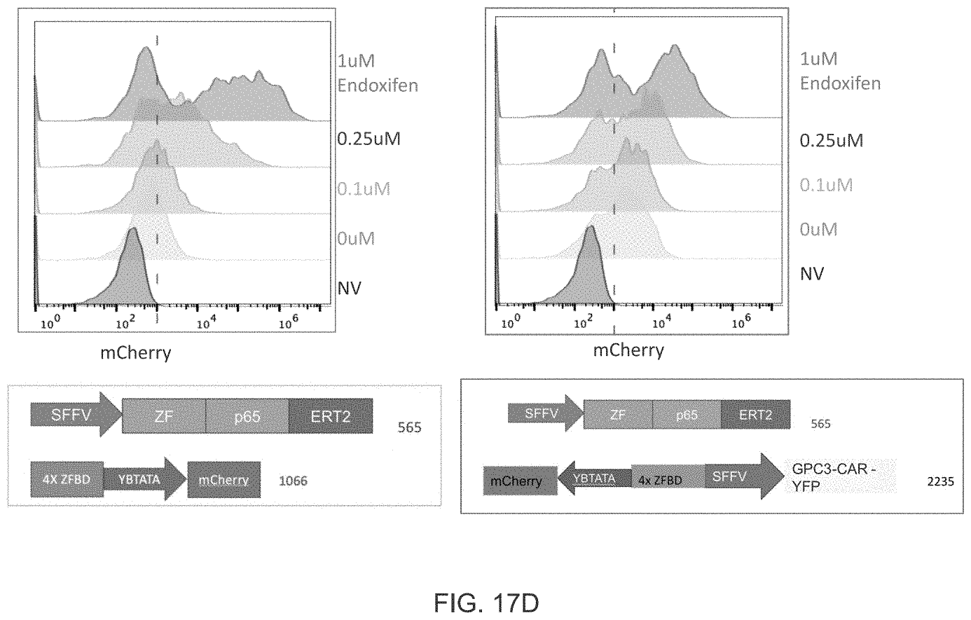

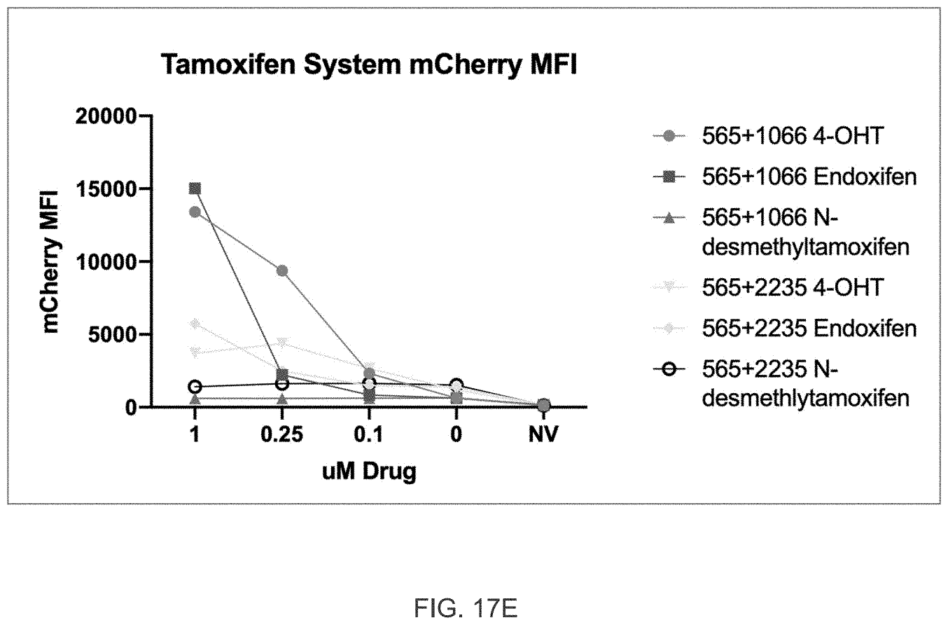

Various regulatable TF (also referred to as an activation-conditional control polypeptide (ACP) for drug-inducible formats or “synTF”) and expression cassettes were constructed as shown in FIG. 17A. Regulation of transcription factor activity was constructed through fusion of the zinc-finger with P65 activation motif and an ERT2 sequence that allows tamoxifen-controlled nuclear localization of the regulatable TF (“565”). A reporter payload sequence was constructed under the inducible control (mCherry driven by YB-TATA promoter and a 4× ZF binding sequence “ZFBD”) on a separate construct from the regulatable TF, with (“2235”) or without (“1066”) a GPC3-CAR-YFP construct expressed by an SFFV promoter in an opposite orientation. The cassettes were cloned into a viral vector and used to produce virus.

On Day 0, CD4/CD8 T cells (FL00711) were thawed and activated with Dynabeads (3:1) in complete T cell media (Optimizer+Supplements—Gibco+5% human serum)+IL-2 (100 Units/ml). On Day 2, cells were transduced with 1×10⁵ GV of virus. On Day 7, 4-hydroxytamoxifen (4-OHT), N-desmethyltamoxifen, or endoxifen were added to each well at 1 uM, 0.25 uM, 0.1 uM, or with no drug. Florescent median intensity (MFI) was assessed of Day 9.

### Results

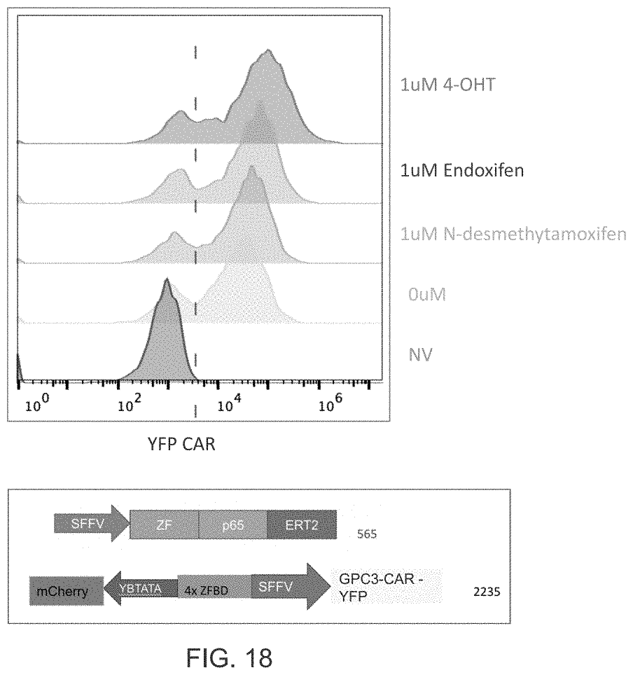

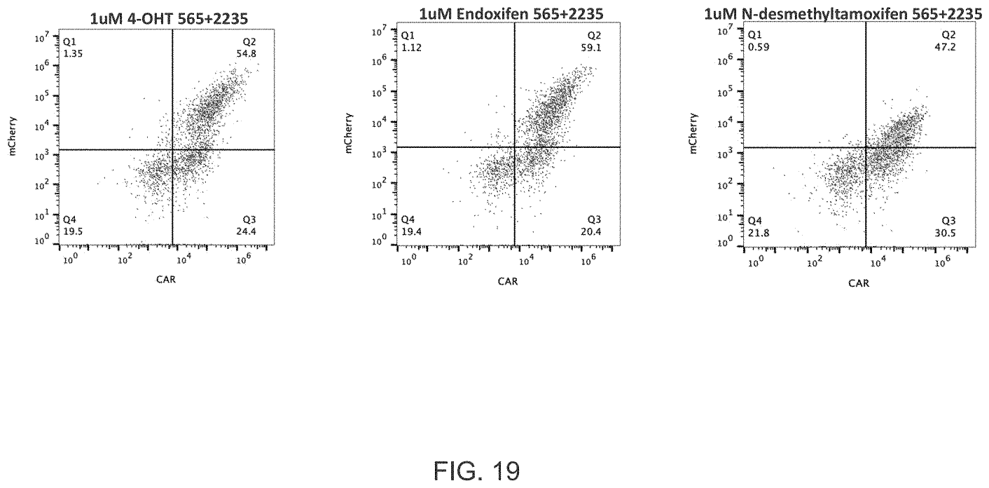

ERT2 regulatable TF systems were assessed using 4-hydroxytamoxifen (4-OHT), N-desmethyltamoxifen, or endoxifen. As shown in FIG. 17B, 4-OHT treatment resulted in regulatable and titratable expression of the reporter either expressed alone (left panel) or expression from a construct also encoding a CAR construct (right panel). As shown in FIG. 17C, N-desmethyltamoxifen treatment did not result expression of the reporter relative to the no-drug control, both expressed alone (left panel) and from a construct also encoding a CAR construct (right panel). As shown in FIG. 17D, endoxifen treatment resulted in regulatable and titratable expression of the reporter either expressed alone (left panel) or expression from a construct also encoding a CAR construct (right panel). A summary of the results is presented in FIG. 17E. As shown in FIG. 18 and FIG. 19, expression of the CAR was confirmed and comparable across the untreated and highest concentration drug treatments. Accordingly, the results indicate T cells can be engineered with an ERT2 regulatable TF system to produce regulatable and titratable expression of a payload using 4-OHT or endoxifen, including in a CAR T system.

### Example 7: Regulatable Transcription Factor Systems in Combination with CAR Expression

Various strategies for the dual-engineering of CAR and regulatable TF expression systems was assessed.

### Materials and Methods

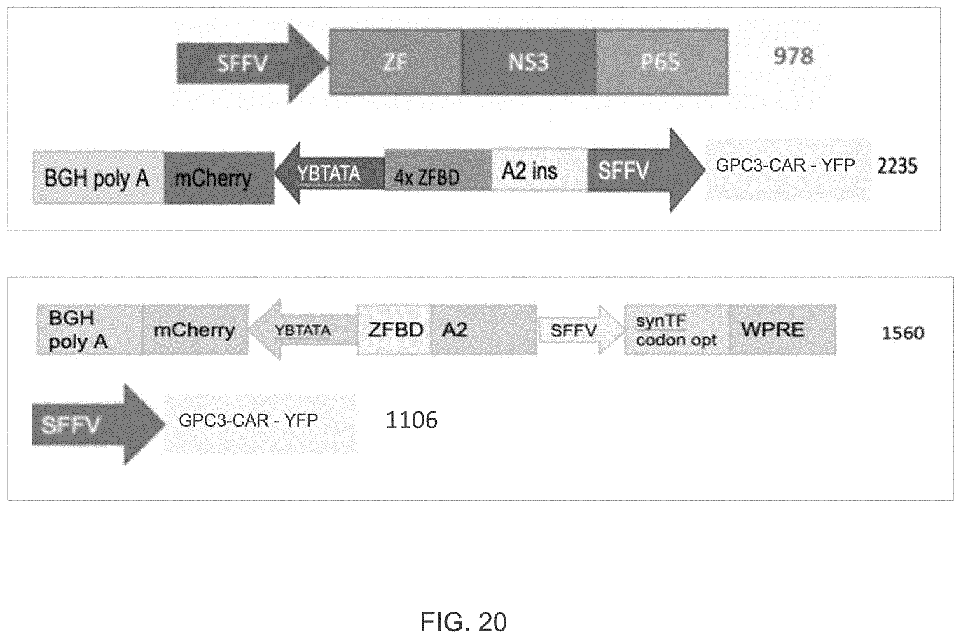

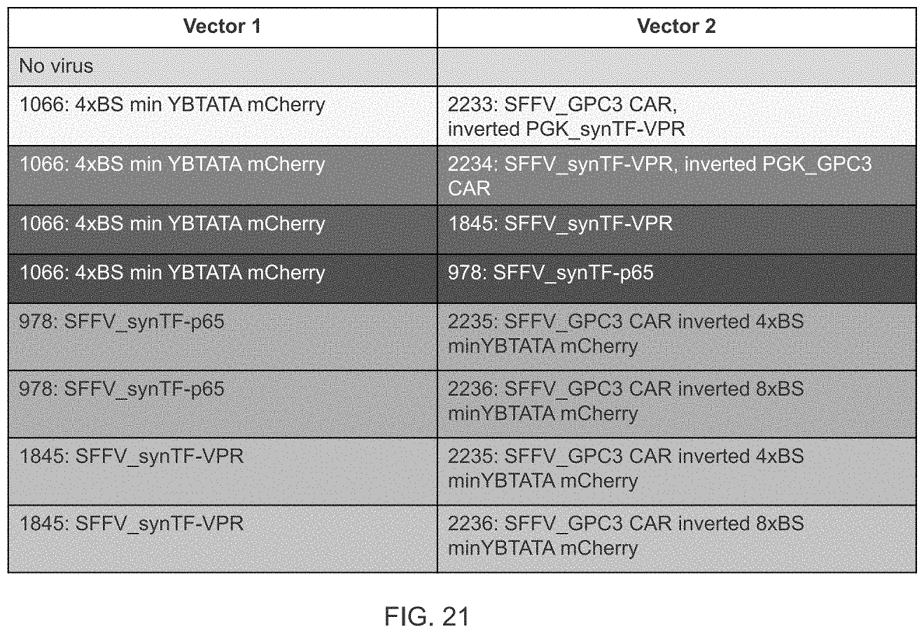

Various strategies for dual-engineering of CAR and regulatable TF expression systems are shown in FIG. 20 and FIG. 21 describing the various orientations, components, and two vector constructions examined.

On Day 0, CD4/CD8 T cells (FL00711) were thawed and activated with Dynabeads (3:1) in complete T cell media (Optimizer+Supplements—Gibco+5% human serum)+IL-2 (100 Units/ml). On Day 2, cells were transduced with 1×10⁵ GV of virus. On Day 7, 2 uM Grazoprevir or no drug was added. Florescent median intensity (MFI) was assessed of Day 9.

### Results

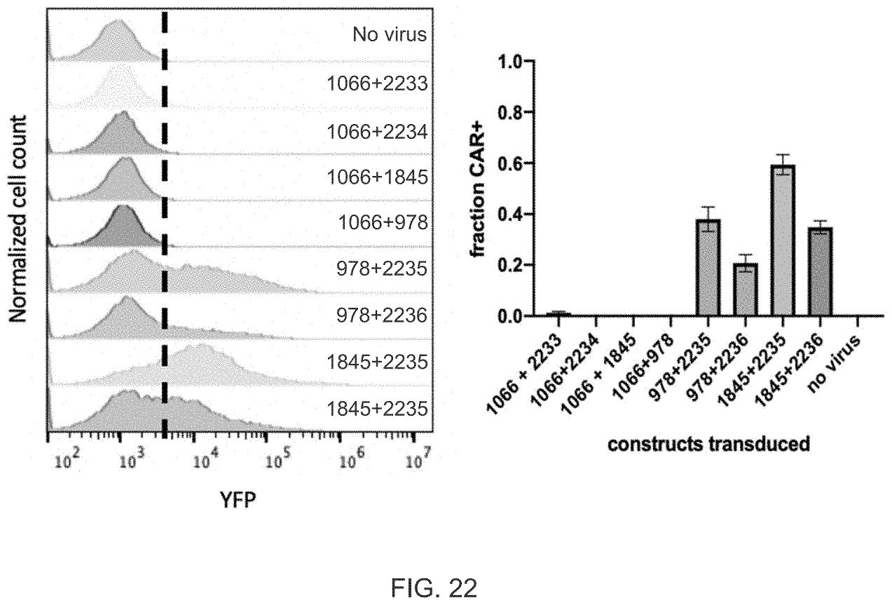

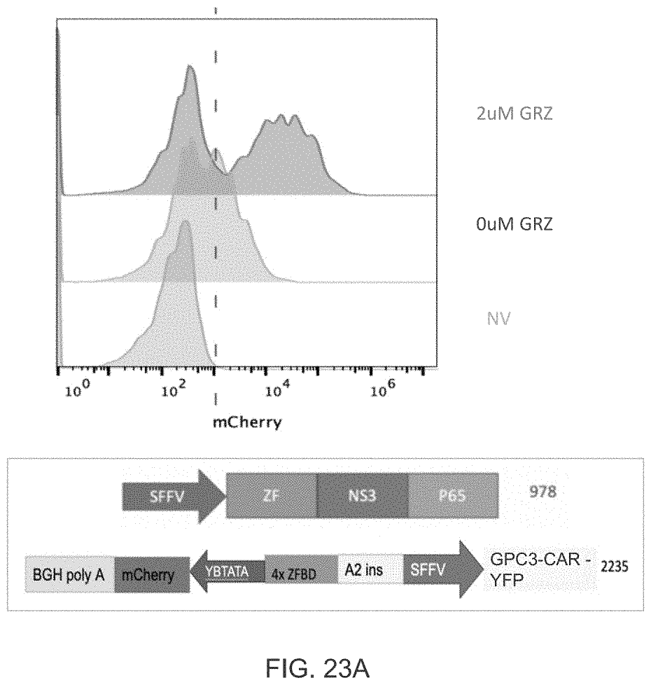

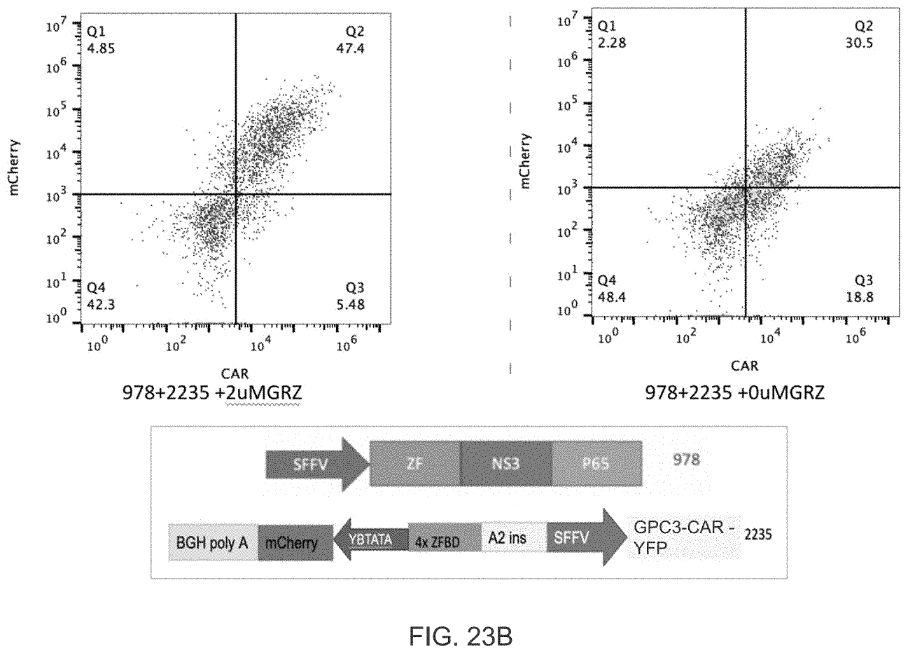

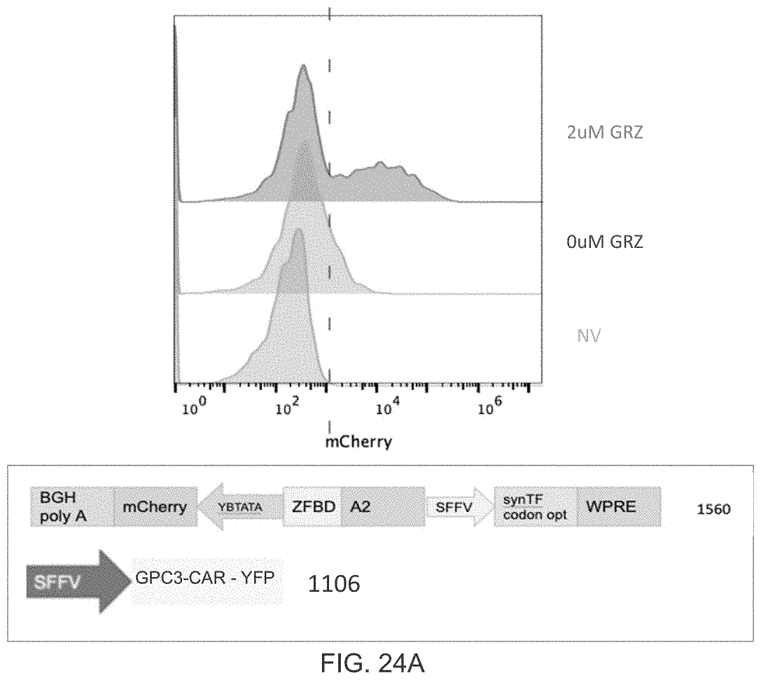

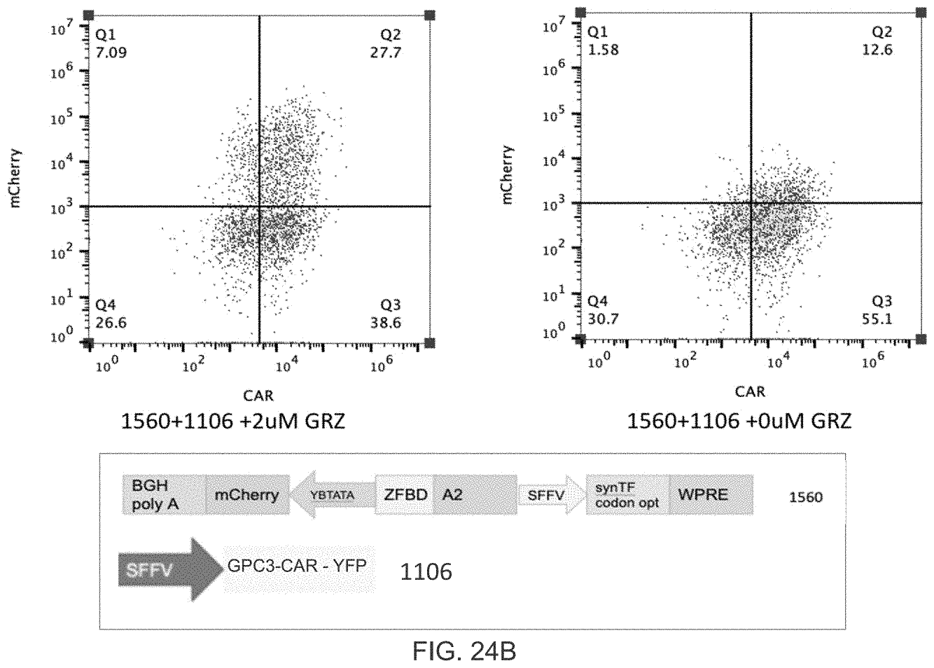

Various orientations, components, and two vector constructions were examined to assess expression of a CAR, and in particular if CAR expression is improved when encoded on the payload vector, regulated TF vector, or alone. As shown in FIG. 22, assessment of the various strategies described in FIG. 21 demonstrated CAR expression was only detected when encoded on the vector with the reporter payload, and not when on vector encoding the regulatable TF (ACP). As shown in FIG. 23A and FIG. 23B, a strategy encoding the payload and CAR on one vector and the regulatable TF on a separate vector resulted in about 50% of transduced cells expressing the CAR and reporter, with a large proportion co-expressing both. As shown in FIG. 24A and FIG. 24B, a strategy encoding the payload and regulatable TF on one vector and the CAR on a separate vector resulted in about 65% of transduced cells that express the CAR co-expressing the reporter, and about 35% of transduced cells expressing the reporter overall after induction. Accordingly, the results indicate (1) encoding CAR on the same vector as the payload and the regulatable TF on a separate vector produced more cells in an induced (payload/reporter positive) state; and (2) encoding the payload and regulatable TF on one vector and the CAR on a separate vector produced more CAR-expressing cells.

### Example 8: Regulatable Transcription Factor Systems Demonstrate Regulated Expression In Vitro and In Vivo

Regulatable TF expression systems were assessed both in vitro and in vivo.

### Materials and Methods

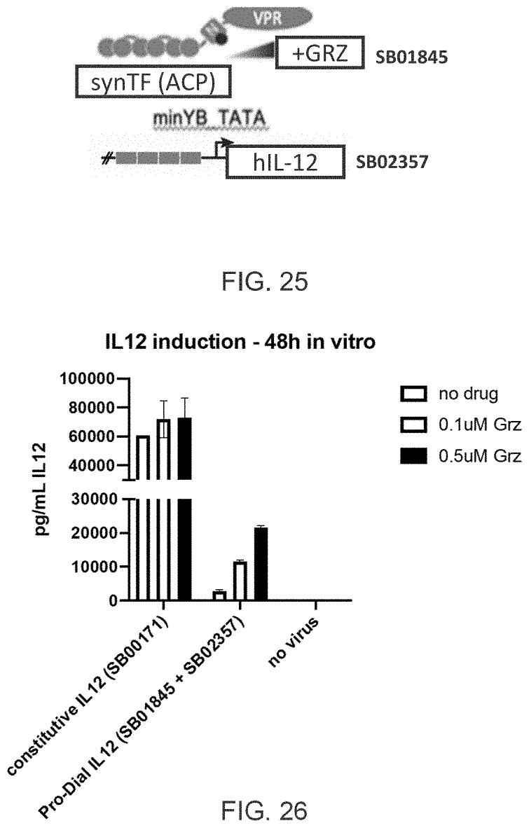

Schematics of the ACP for drug-inducible formats (also referred to as “synTF”) using an NS3/NS4 protease cleavage site and a VPR transcriptional effector domain (Construct “1845”) as well as the expression cassette using a 4× BS minYB-TATA ACP-responsive promoter driving hIL-12 effector molecule payload are shown in FIG. 25. A construct with hIL-12 driven by a constitutive SFFV promoter (Construct “171”) was also assessed. Relevant sequences of the constructs are provided in Table 14.

TABLE 14
ACP Sequences

| SB# | Domain | Sequence |
|---|---|---|
| SB01845 | Zinc | SRPGERPFQCRICMRNFSRRHGLDRHTRTHTGEKPFQCRICMR |
| | Finger | NFSDHSSLKRHLRTHTGSQKPFQCRICMRNFSVRHNLTRHLRT |
| | | HTGEKPFQCRICMRNFSDHSNLSRHLKTHTGSQKPFQCRICMR |
| | | NFSQRSSLVRHLRTHTGEKPFQCRICMRNFSESGHLKRHLRTH |
| | | LRGS (SEQ ID NO: 88) |
| SB01845 | Spacer- | TCRDYKDHDGDYKDHDIDYKDDDDKMAPKKKRKVGIHGVPG |
| | Cleavage | GLEGGGGSGGTEDVVCCHSIYGKKKGDIDTYRYIGSSGTGCVVI |
| | Site-linker- | VGRIVLSGSGTSAPITAYAQQTRGLLGCIITSLTGRDKNQVE |
| | Protease- | GEVQIVSTATQTFLATCINGVCWAVYHGAGTRTIASPKGPV |
| | linker- | IQMYTNVDQDLVGWPAPQGSRSLTPCTCGSSDLYLVTRHA |
| | Cleavage | DVIPVRRRGDSRGSLLSPRPISYLKGSSGGPLLCPAGHAVGL |
| | Site-spacer | FRAAVCTRGVAKAVDFIPVENLETTMRSPVFTDNSSPPAVTL |
| | | THPITKIDREVLYQEFDEMEECSQHYPYDVPDYAGGGGSGGT |
| | | (SEQ ID NO: 89) |
| SB01845 | VPR | See SEQ ID NO: 90 |
| SB01845 | Full | atgTCTAGACCCGGAGAGCGCCCATTCCAGTGTCGGATTTGC |
| | Construct | ATGCGGAACTTTTCGAGAAGACACGGCCTGGACAGACATAC |
| | (DNA) | CCGTACTCATACAGGTGAAAAACCCTTTCAGTGTCGGATCT |
| | | GTATGCGAAATTTCTCCGACCACAGCAGCCTGAAGAGACAT |
| | | CTACGTACCCACACCGGCAGCCAGAAGCCATTTCAGTGTCG |
| | | GATCTGTATGCGGAACTTCTCCGTGAGACACAACCTGACCA |
| | | GACATCTACGTACGCACACCGGAGAGAAGCCATTCCAATGC |
| | | CGAATATGCATGCGCAACTTCAGTGACCACAGCAACCTGAG |
| | | CAGACACCTAAAAACCCACACCGGTTCCCAGAAGCCATTTC |
| | | AGTGTCGGATCTGTATGCGGAACTTCTCCCAGCGCAGCAGC |
| | | CTGGTGAGACATCTACGTACGCACACCGGAGAGAAGCCATT |
| | | CCAATGCCGAATATGCATGCGCAACTTCAGTGAGAGCGGCC |
| | | ACCTGAAGAGACACCTGCGTACGCACCTGAGGGGATCCaCC |
| | | TGCAGGgactacaaagaccatgacggtgattataaagatcatgacatcgattacaaggatgac |
| | | gatgacaagatggcccccaagaaaaagaggaaggtgggcattcacggggtgccgggtggactc |
| | | gagggaggcggtggaagcggcggtaccGAGGACGTGGTGTGCTGCCACTC |
| | | AATCTACGGCAAGAAGAAGGGTGATATCGACACCTACCGAT |
| | | ACATAGGCTCTTCCGGGACAGGCTGCGTGGTCATAGTGGGC |
| | | AGGATCGTCTTGTCCGGATCCGGCACTAGTGCGCCCATCAC |
| | | GGCGTACGCCCAGCAGACGAGAGGCCTCCTAGGGTGTATAA |
| | | TCACCAGCCTGACTGGCCGGGACAAAAACCAAGTGGAGGG |
| | | TGAGGTCCAGATCGTGTCAACTGCTACCCAAACCTTCCTGG |
| | | CAACGTGCATCAATGGGGTATGCTGGGCAGTCTACCACGGG |
| | | GCCGGAACGAGGACCATCGCATCACCCAAGGGTCCTGTCAT |
| | | CCAGATGTATACCAATGTGGACCAAGACCTTGTGGGCTGGC |
| | | CCGCTCCTCAAGGTTCCCGCTCATTGACACCCTGTACCTGCG |
| | | GCTCCTCGGACCTTTACCTGGTCACGAGGCACGCCGATGTC |
| | | ATTCCCGTGCGCCGGCGAGGTGATAGTAGAGGCTCTCTGCT |
| | | GAGCCCCAGACCTATCAGCTACCTGAAGGGCTCTAGCGGCG |
| | | GACCTCTGCTTTGTCCTGCTGGACATGCCGTGGGCCTGTTTA |
| | | GAGCCGCCGTGTGTACAAGAGGCGTGGCCAAAGCCGTGGA |
| | | CTTCATCCCCGTGGAAAACCTGGAAACCACCATGCGGAGCC |
| | | CCGTGTTCACCGACAATTCTAGCCCTCCAGCCGTGACACTG |
| | | ACACACCCCATCACCAAGATCGACAGAGAGGTGCTGTACCA |
| | | AGAGTTCGACGAGATGGAAGAGTGCAGCCAGCACTACCCCT |
| | | ACGACGTGCCAGATTATGCTGGCGGCGGAGGATCTGGCGGA |
| | | ACAGAAGCCTCTGGAAGCGGCAGAGCTGACGCCCTGGATG |
| | | ACTTCGACCTGGATATGCTGGGCAGCGACGCTCTGGACGAT |
| | | TTTGACCTCGACATGCTGGGATCTGATGCACTCGACGATTTC |
| | | GATTTGGACATGCTCGGCAGTGATGCCTTGGACGACTTTGA |
| | | TCTTGATATGCTCATCAACAGCCGGTCCAGCGGCAGCCCCA |
| | | AGAAAAAAAGAAAAGTGGGCTCCCAGTACCTGCCTGACAC |
| | | CGACGACAGACACCGGATCGAGGAAAAGCGGAAGCGGACC |
| | | TACGAGACATTCAAGAGCATCATGAAGAAGTCCCCATTCAG |
| | | CGGCCCCACCGATCCTAGACCTCCACCTAGAAGAATCGCCG |
| | | TGCCTAGCAGATCTAGCGCCTCCGTGCCTAAACCTGCTCCTC |
| | | AGCCTTATCCTTTCACCAGCAGCCTGAGCACCATCAACTAC |
| | | GACGAGTTCCCTACCATGGTGTTCCCCAGCGGCCAGATCTCT |
| | | CAGGCTTCTGCTCTTGCTCCAGCTCCTCCTCAGGTTCTGCCT |
| | | CAAGCTCCTGCACCAGCACCGGCTCCAGCTATGGTTTCTGCT |
| | | TTGGCTCAGGCCCCTGCTCCTGTGCCTGTTCTTGCTCCTGGA |
| | | CCACCTCAGGCTGTTGCTCCTCCTGCTCCAAAACCTACACAG |
| | | GCCGGCGAAGGCACACTGTCTGAAGCTCTGCTGCAGCTCCA |
| | | GTTCGATGACGAAGATCTGGGCGCCCTGCTGGGCAATTCTA |
| | | CAGATCCTGCCGTGTTTACCGATCTGGCCAGCGTGGACAAC |
| | | AGCGAGTTTCAGCAGCTCCTGAATCAGGGCATCCCTGTGGC |
| | | TCCTCACACCACCGAACCTATGCTGATGGAATACCCCGAGG |
| | | CCATCACCAGACTGGTCACCGGTGCTCAAAGACCACCTGAT |
| | | CCAGCTCCAGCACCACTGGGAGCACCTGGACTGCCTAATGG |
| | | ACTGCTGTCTGGCGACGAGGACTTCAGCTCTATCGCCGACA |
| | | TGGATTTCTCTGCCCTGCTCGGCTCTGGCAGCGGCTCTAGAG |
| | | ATAGCAGAGAAGGCATGTTCCTGCCTAAGCCTGAGGCCGGC |
| | | TCTGCCATCTCCGATGTGTTCGAGGGAAGAGAAGTGTGCCA |
| | | GCCTAAGCGGATCCGGCCTTTTCACCCTCCTGGAAGCCCTTG |
| | | GGCCAACAGACCTCTGCCTGCTTCTCTGGCCCCTACACCAAC |
| | | AGGACCTGTGCACGAACCTGTGGGCAGTCTGACCCCAGCTC |
| | | CTGTTCCTCAACCTCTGGATCCCGCTCCTGCTGTGACACCTG |
| | | AAGCCTCTCATCTGCTGGAAGATCCCGACGAAGAGACAAGC |
| | | CAGGCCGTGAAGGCCCTGAGAGAAATGGCCGACACAGTGA |
| | | TCCCTCAGAAAGAGGAAGCCGCCATCTGCGGACAGATGGAC |
| | | CTGTCTCATCCTCCACCAAGAGGCCACCTGGACGAGCTGAC |
| | | AACCACACTGGAATCCATGACCGAGGACCTGAACCTGGACA |
| | | GCCCTCTGACACCCGAGCTGAACGAGATCCTGGACACCTTC |
| | | CTGAACGACGAGTGTCTGCTGCACGCCATGCACATCTCTAC |
| | | CGGCCTGAGCATCTTCGACACCAGCCTGTTTTGA (SEQ ID |
| | | NO: 91) |
| SB02357 | ACP- | cgggtttcgtaacaatcgcatgaggattcgcaacgccttcGGCGTAGCCGATGTCGC |
| | responsive | GctcccgtctcagtaaaggtcGGCGTAGCCGATGTCGCGcaatcggactgccttc |
| | promoter | gtacGGCGTAGCCGATGTCGCGcgtatcagtcgcctcggaacGGCGTAGC |
| | | CGATGTCGCGcattcgtaagaggctcactctcccttacacggagtggataACTAGTT |
| | | CTAGAGGGTATATAATGGGGGCCA (SEQ ID NO: 92) |
| SB02357 | inducible | cgggtttcgtaacaatcgcatgaggattcgcaacgccttcGGCGTAGCCGATGTCGC |
| | promoter | GctcccgtctcagtaaaggtcGGCGTAGCCGATGTCGCGcaatcggactgccttc |
| | IL12 | gtacGGCGTAGCCGATGTCGCGcgtatcagtcgcctcggaacGGCGTAGC |
| | (4x ZF BS | CGATGTCGCGcattcgtaagaggctcactctcccttacacggagtggataACTAGTT |
| | YB-TATA | CTAGAGGGTATATAATGGGGGCCAACGCGTGCCGCCACCAT |
| | IL12) | GTGCCATCAGCAACTCGTCATCTCCTGGTTCTCCCTTGTGTT |
| | | CCTCGCTTCCCCTCTGGTCGCCATTTGGGAACTGAAGAAGG |
| | | ACGTCTACGTGGTCGAGCTGGATTGGTACCCGGACGCCCCT |
| | | GGAGAAATGGTCGTGCTGACTTGCGATACGCCAGAAGAGG |
| | | ACGGCATAACCTGGACCCTGGATCAGAGCTCCGAGGTGCTC |
| | | GGAAGCGGAAAGACCCTGACCATTCAAGTCAAGGAGTTCG |
| | | GCGACGCGGGCCAGTACACTTGCCACAAGGGTGGCGAAGT |
| | | GCTGTCCCACTCCCTGCTGCTGCTGCACAAGAAAGAGGATG |
| | | GAATCTGGTCCACTGACATCCTCAAGGACCAAAAAGAACCG |
| | | AAGAACAAGACCTTCCTCCGCTGCGAAGCCAAGAACTACAG |
| | | CGGTCGGTTCACCTGTTGGTGGCTGACGACAATCTCCACCG |
| | | ACCTGACTTTCTCCGTGAAGTCGTCACGGGGATCAAGCGAT |
| | | CCTCAGGGCGTGACCTGTGGAGCCGCCACTCTGTCCGCCGA |
| | | GAGAGTCAGGGGAGACAACAAGGAATATGAGTACTCCGTG |
| | | GAATGCCAGGAGGACAGCGCCTGCCCTGCCGCGGAAGAGT |
| | | CCCTGCCTATCGAGGTCATGGTCGATGCCGTGCATAAGCTG |
| | | AAATACGAGAACTACACTTCCTCCTTCTTTATCCGCGACATC |
| | | ATCAAGCCTGACCCCCCCAAGAACTTGCAGCTGAAGCCACT |
| | | CAAGAACTCCCGCCAAGTGGAAGTGTCTTGGGAATATCCAG |
| | | ACACTTGGAGCACCCCGCACTCATACTTCTCGCTCACTTTCT |
| | | GTGTGCAAGTGCAGGGAAAGTCCAAACGGGAGAAGAAAGA |
| | | CCGGGTGTTCACCGACAAAACCTCCGCCACTGTGATTTGTC |
| | | GGAAGAACGCGTCAATCAGCGTCCGGGCGCAGGATAGATA |
| | | CTACTCGTCCTCCTGGAGCGAATGGGCCAGCGTGCCTTGTTC |
| | | CGGTGGCGGATCAGGCGGAGGTTCAGGAGGAGGCTCCGGA |
| | | GGAGGTTCCCGGAACCTCCCTGTGGCAACCCCCGACCCTGG |
| | | AATGTTCCCGTGCCTACACCACTCCCAAAACCTCCTGAGGG |
| | | CTGTGTCGAACATGTTGCAGAAGGCCCGCCAGACCCTTGAG |
| | | TTCTACCCCTGCACCTCGGAAGAAATTGATCACGAGGACAT |
| | | CACCAAGGACAAGACCTCGACCGTGGAAGCCTGCCTGCCGC |
| | | TGGAACTGACCAAGAACGAATCGTGTCTGAACTCCCGCGAG |
| | | ACAAGCTTTATCACTAACGGCAGCTGCCTGGCGTCGAGAAA |
| | | GACCTCATTCATGATGGCGCTCTGTCTTTCCTCGATCTACGA |
| | | AGATCTGAAGATGTATCAGGTCGAGTTCAAGACCATGAACG |
| | | CCAAGCTGCTCATGGACCCGAAGCGGCAGATCTTCCTGGAC |
| | | CAGAATATGCTCGCCGTGATTGATGAACTGATGCAGGCCCT |
| | | GAATTTCAACTCCGAGACTGTGCCTCAAAAGTCCAGCCTGG |
| | | AAGAACCGGACTTCTACAAGACCAAGATCAAGCTGTGCATC |
| | | CTGTTGCACGCTTTCCGCATTCGAGCCGTGACCATTGACCGC |
| | | GTGATGTCCTACCTGAACGCCAGTtaa (SEQ ID NO: 150) |
| SB02357 | hIL-12 | MCHQQLVISWFSLVFLASPLVAIWELKKDVYVVELDWYPDAP |
| and | protein | GEMVVLTCDTPEEDGITWTLDQSSEVLGSGKTLTIQVKEFGDA |
| SB00171 | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGGSGGGSGGGSGGG |
| | | SRNLPVATPDPGMFPCLHHSQNLLRAVSNMLQKARQTLEFYP |
| | | CTSEEIDHEDITKDKTSTVEACLPLELTKNESCLNSRETSFITNG |
| | | SCLASRKTSFMMALCLSSIYEDLKMYQVEFKTMNAKLLMDPK |
| | | RQIFLDQNMLAVIDELMQALNFNSETVPQKSSLEEPDFYKTKIK |
| | | LCILLHAFRIRAVTIDRVMSYLNAS (SEQ ID NO: 93) |
| SB02357 | hIL-12 | ATGTGCCATCAGCAACTCGTCATCTCCTGGTTCTCCCTTGTG |
| | DNA | TTCCTCGCTTCCCCTCTGGTCGCCATTTGGGAACTGAAGAAG |
| | (codon | GACGTCTACGTGGTCGAGCTGGATTGGTACCCGGACGCCCC |
| | optimized) | TGGAGAAATGGTCGTGCTGACTTGCGATACGCCAGAAGAGG |
| | | ACGGCATAACCTGGACCCTGGATCAGAGCTCCGAGGTGCTC |
| | | GGAAGCGGAAAGACCCTGACCATTCAAGTCAAGGAGTTCG |
| | | GCGACGCGGGCCAGTACACTTGCCACAAGGGTGGCGAAGT |
| | | GCTGTCCCACTCCCTGCTGCTGCTGCACAAGAAAGAGGATG |
| | | GAATCTGGTCCACTGACATCCTCAAGGACCAAAAAGAACCG |
| | | AAGAACAAGACCTTCCTCCGCTGCGAAGCCAAGAACTACAG |
| | | CGGTCGGTTCACCTGTTGGTGGCTGACGACAATCTCCACCG |
| | | ACCTGACTTTCTCCGTGAAGTCGTCACGGGGATCAAGCGAT |
| | | CCTCAGGGCGTGACCTGTGGAGCCGCCACTCTGTCCGCCGA |
| | | GAGAGTCAGGGGAGACAACAAGGAATATGAGTACTCCGTG |
| | | GAATGCCAGGAGGACAGCGCCTGCCCTGCCGCGGAAGAGT |
| | | CCCTGCCTATCGAGGTCATGGTCGATGCCGTGCATAAGCTG |
| | | AAATACGAGAACTACACTTCCTCCTTCTTTATCCGCGACATC |
| | | ATCAAGCCTGACCCCCCCAAGAACTTGCAGCTGAAGCCACT |
| | | CAAGAACTCCCGCCAAGTGGAAGTGTCTTGGGAATATCCAG |
| | | ACACTTGGAGCACCCCGCACTCATACTTCTCGCTCACTTTCT |
| | | GTGTGCAAGTGCAGGGAAAGTCCAAACGGGAGAAGAAAGA |
| | | CCGGGTGTTCACCGACAAAACCTCCGCCACTGTGATTTGTC |
| | | GGAAGAACGCGTCAATCAGCGTCCGGGCGCAGGATAGATA |
| | | CTACTCGTCCTCCTGGAGCGAATGGGCCAGCGTGCCTTGTTC |
| | | CGGTGGCGGATCAGGCGGAGGTTCAGGAGGAGGCTCCGGA |
| | | GGAGGTTCCCGGAACCTCCCTGTGGCAACCCCCGACCCTGG |
| | | AATGTTCCCGTGCCTACACCACTCCCAAAACCTCCTGAGGG |
| | | CTGTGTCGAACATGTTGCAGAAGGCCCGCCAGACCCTTGAG |
| | | TTCTACCCCTGCACCTCGGAAGAAATTGATCACGAGGACAT |
| | | CACCAAGGACAAGACCTCGACCGTGGAAGCCTGCCTGCCGC |
| | | TGGAACTGACCAAGAACGAATCGTGTCTGAACTCCCGCGAG |
| | | ACAAGCTTTATCACTAACGGCAGCTGCCTGGCGTCGAGAAA |
| | | GACCTCATTCATGATGGCGCTCTGTCTTTCCTCGATCTACGA |
| | | AGATCTGAAGATGTATCAGGTCGAGTTCAAGACCATGAACG |
| | | CCAAGCTGCTCATGGACCCGAAGCGGCAGATCTTCCTGGAC |
| | | CAGAATATGCTCGCCGTGATTGATGAACTGATGCAGGCCCT |
| | | GAATTTCAACTCCGAGACTGTGCCTCAAAAGTCCAGCCTGG |
| | | AAGAACCGGACTTCTACAAGACCAAGATCAAGCTGTGCATC |
| | | CTGTTGCACGCTTTCCGCATTCGAGCCGTGACCATTGACCGC |
| | | GTGATGTCCTACCTGAACGCCAGTTAA (SEQ ID NO: 94) |
| SB00171 | hIL-12 | ATGTGCCATCAGCAGCTTGTCATATCTTGGTTTTCACTTGTA |
| | DNA | TTCCTGGCCAGCCCTTTGGTTGCGATCTGGGAGCTCAAGAA |
| | | GGATGTGTACGTTGTAGAGCTGGACTGGTACCCCGATGCTC |
| | | CCGGTGAGATGGTCGTTTTGACATGTGACACTCCAGAAGAG |
| | | GACGGTATTACGTGGACTCTGGACCAGTCCTCCGAAGTTCTT |
| | | GGTTCTGGTAAGACTCTGACTATCCAGGTGAAAGAATTTGG |
| | | GGATGCGGGACAATACACATGCCACAAGGGAGGCGAGGTG |
| | | TTGTCTCATAGTTTGCTGCTTCTCCACAAGAAAGAGGATGG |
| | | AATCTGGAGCACCGACATACTCAAGGATCAAAAGGAACCC |
| | | AAAAATAAGACATTTCTGCGATGTGAGGCTAAGAACTATAG |
| | | TGGCCGCTTCACTTGTTGGTGGCTGACTACCATCAGCACAG |
| | | ATCTCACGTTTTCAGTAAAAAGTAGTAGAGGTTCAAGTGAT |
| | | CCTCAAGGGGTAACGTGCGGTGCTGCAACACTGTCTGCTGA |
| | | ACGCGTAAGAGGAGATAATAAGGAGTACGAGTATTCCGTA |
| | | GAATGCCAAGAGGACAGTGCTTGTCCTGCGGCCGAGGAGTC |
| | | TCTCCCAATAGAAGTGATGGTGGACGCGGTGCATAAACTGA |
| | | AATATGAGAACTACACAAGCAGTTTTTTTATAAGAGATATC |
| | | ATCAAGCCCGATCCGCCGAAGAATTTGCAACTTAAACCGCT |
| | | TAAAAACTCACGCCAGGTTGAAGTATCCTGGGAGTATCCGG |
| | | ATACATGGTCAACACCACACAGCTATTTTTCCCTTACCTTCT |
| | | GTGTGCAGGTCCAAGGGAAGAGCAAAAGGGAGAAGAAGGA |
| | | CAGGGTATTCACTGATAAAACTTCCGCGACGGTCATCTGCC |
| | | GAAAAAACGCTAGTATATCTGTACGGGCGCAGGATAGGTAC |
| | | TATAGTTCTTCTTGGTCTGAGTGGGCCTCAGTTCCGTGCTCT |
| | | GGGGGAGGAAGTGGAGGAGGGTCCGGCGGTGGAAGCGGGG |
| | | GAGGGAGTCGCAACTTGCCAGTGGCTACACCAGATCCAGGC |
| | | ATGTTTCCATGTCTGCATCATTCCCAGAATCTCCTGAGAGCG |
| | | GTGTCAAATATGCTCCAAAAAGCGAGACAAACACTGGAATT |
| | | TTACCCGTGTACCAGTGAGGAGATTGATCACGAGGACATAA |
| | | CCAAGGACAAGACCTCAACTGTAGAAGCGTGTTTGCCGCTG |
| | | GAGTTGACTAAGAATGAGTCCTGCCTCAATTCCAGAGAAAC |
| | | TTCATTCATTACTAACGGCAGTTGTCTTGCATCCCGGAAAAC |
| | | GTCCTTTATGATGGCCCTTTGCCTTAGTTCAATTTACGAGGA |
| | | TCTTAAAATGTATCAAGTGGAGTTTAAAACCATGAATGCTA |
| | | AACTTCTTATGGACCCCAAACGACAAATTTTTCTGGATCAG |
| | | AATATGCTTGCCGTGATAGACGAACTCATGCAGGCGCTTAA |
| | | TTTTAACTCCGAAACAGTTCCACAAAAATCTAGCCTTGAAG |
| | | AACCTGATTTTTATAAAACGAAGATTAAACTGTGTATCCTGC |
| | | TGCATGCCTTTCGCATCCGAGCTGTCACAATCGATAGGGTTA |
| | | TGTCCTACCTTAACGCGAGCtaG (SEQ ID NO: 95) |

For in vitro assessment, on Day 0, CD4/CD8 T cells (FL00711) were thawed and activated with Dynabeads (3:1) in complete T cell media (Optimizer+Supplements—Gibco+5% human serum)+IL-2 (100 Units/nil). On Day 2, cells were transduced with 1×10⁵ GV of virus encoding the constructs described above. On Day 7, 0.1 uM Grazoprevir (GRZ), 0.5 uM Grazoprevir, or no drug was added. Transduced T cells were incubated for a further two days and the supernatant collected via centrifugation for IL-12 quantification. For IL-12, supernatants were diluted 1/100 in PBS/BSA and analyzed using the human IL-12 p70 Quantikine ELISA kit (R&D Systems, cat #D1200B).

For in vivo assessment, T cells were transduced with SB01845 & SB02357, each at an estimated MOI of 5 based on viral titering in HEK cells. Positive control T cells were transduced with 5 MOI of SB00171, encoding constitutive hIL-12 (driven by SFFV). Negative controls were untransduced T cells. The number of lentiviral genomes integrated into the T cells was analyzed in bulk by PCR (copy #assay). NSG mice were randomized on day −2 and vehicle or Grazoprevir (Grz) dosing began in the afternoon on day 1 (Vehicle: 2.5% DMSO, 30% PEG400, 67.5% PBS). Grazoprevir potassium salt was dissolved sequentially in DMSO, PEG400, and PBS to reach 10 mg/mL. Dosing: Drug treated mice received 25 mg/kg Grz at each dosing time point; vehicle treated mice received equivalent volume of vehicle; all Grz dosing administered IP. Dosing continued BID on days 2-4. On day 2, 20e6 T cells per mouse were injected by tail vein injection after the AM drug dosing. Mice were bled on day 4 and sacrificed/bled on day 5. Luminex assay was run to assess levels of hIL-12 in mouse plasma. Presence of human T cells in mouse blood was analyzed by flow cytometry. The treatment groups are presented in Table 15.

TABLE 15
Murine Regulated Armoring Treatment Groups

| Treatment | Drug | | Frequency of |
|---|---|---|---|
| (IV) | (IP) | Concentration | Dose |
| non-engineered T cells | Vehicle | — | BID |
| non-engineered T cells | Grazoprevir | 25 mg/kg | BID |
| constitutive IL12 T cells | Vehicle | — | BID |
| constitutive IL12 T cells | Grazoprevir | 25 mg/kg | BID |
| inducible IL12 T cells | Vehicle | — | BID |
| inducible IL12 T cells | Grazoprevir | 25 mg/kg | BID |

### Results

As shown in FIG. 26, a regulated expression system with an ACP for drug-inducible formats (also referred to as “synTF”) using an NS3/NS4 protease cleavage site and a VPR transcriptional effector domain (Construct “1845”) as well as the expression cassette using a 4× BS minYB-TATA ACP-responsive promoter driving hIL-12 effector molecule payload resulted in titratable hIL-12 secretion in vitro upon addition of increasing amounts of GRZ, with minimal hIL-12 detected in the absence of drug and no detectable hIL-12 in the absence of virus.

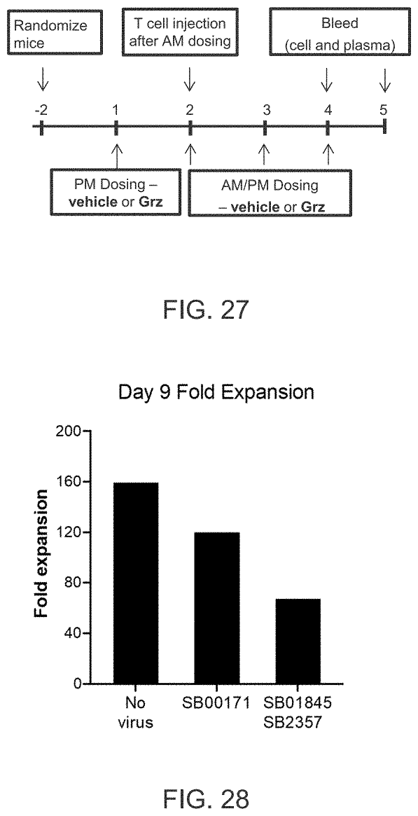

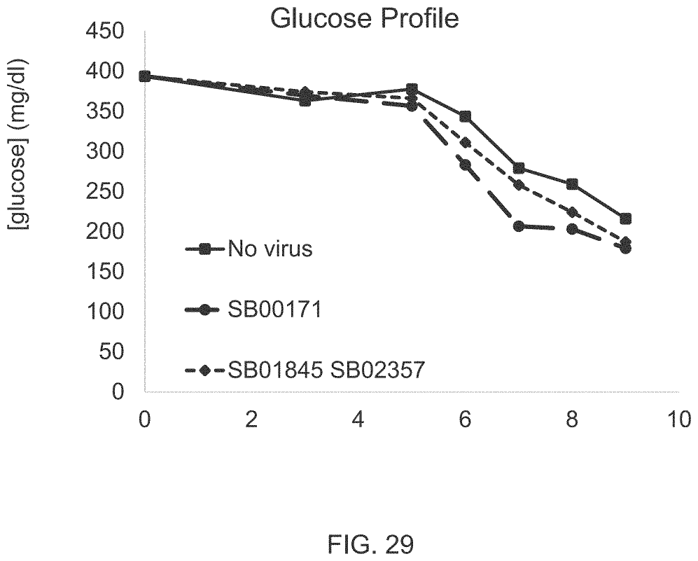

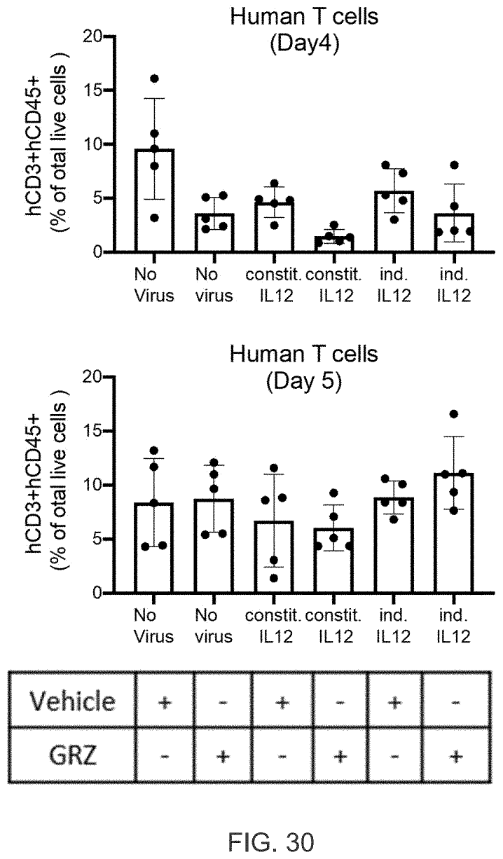

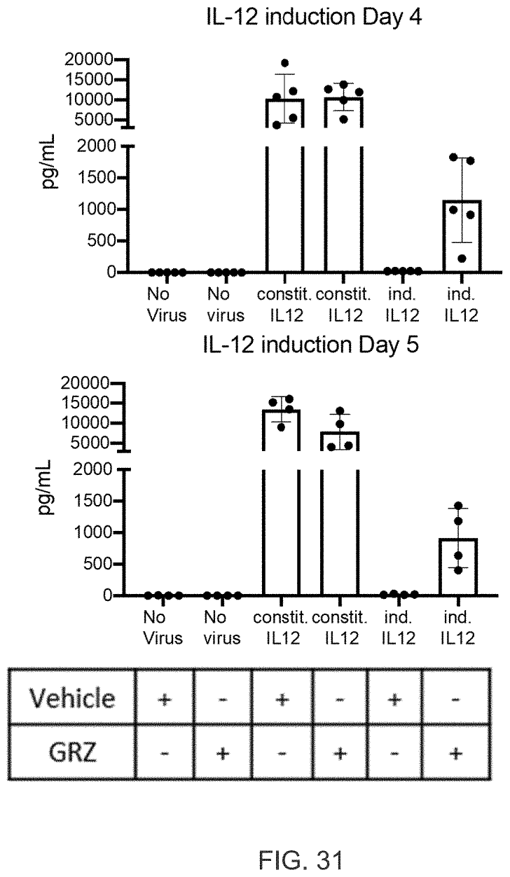

Next, the constructs were assessed in vivo. The experimental design is shown in FIG. 27. T cells were assessed following transduction and injection. As shown in Table 16, T cells with the regulatable system demonstrated ˜2.5 lower copy number viral copy number relative to the constitutive system, indicating lower transduction efficiency. As shown in FIG. 28, T cells at day 9 also demonstrated ˜2-fold expansion in the regulatable system compared to the no virus control, potentially due to transduction with two viruses. As shown in FIG. 29, T cells from each group demonstrated similar glucose profiles, indicating T cell growth and metabolism was generally equivalent across all groups. Next, production of hIL-12 in vivo was assessed. As shown in FIG. 30, T cells were equally present for each group demonstrating that the regulatable armoring system did not alter T cell longevity in vivo. As shown in FIG. 31, administration of GRZ lead to detectable hIL-12 in the plasma of mice (˜100-fold above vehicle) indicating the regulatable system provided a drug-inducible in vivo format for the regulated armoring of T cells.

TABLE 16
Viral Copy Number

| | Copy Number |
|---|---|
| Description | (copies/cell) |
| No virus (non-engineered) | UND |
| SB00171—constitutive IL12 T cells | 8.8 |
| SB01845 + 2357—inducible 1L12 T cells | 3.4 |

### Example 9: Activation Inducible System

Enhancers that turn on transcription when CAR-T are activated by target cells are assessed.

### Materials and Methods

Library Generation: A library of 15,000 enhancers linked to a minimal promoter (late Ade minimal promoter) was generated to screen for enhancers that turn on transcription when CAR-T are activated by target cells. Enhancers that were enriched in the ATAC-seq of activated T cells (Gate et al. Nat Genet. Author manuscript; available in PMC 2019 Jan. 9; herein incorporated by reference for all purposes) were chosen for the library (all enhancers <400 bp were used). Length of library members is 199 bp, thus for enhancers longer than 199 bp tiling was used to cover 2 regions of each enhancer with as much overlap as possible. Top upregulated genes in single-cell RNA seq data (Xhangolli et al. Genomics Proteomics Bioinformatics. 2019 April; 17(2):129-139. doi: 10.1016/j.gpb.2019.03.002; herein incorporated by reference for all purposes) were searched in the HACER database of human enhancers and enhancers from those genes were chosen. Synthetic enhancers were designed using pairs of transcription factors known from the literature to be upregulated in activated T cells. 4 binding sites for each TF were used to generate the synthetic enhancers (either aaaabbbb or abababab). All possible pairs from the following list of TFs were included: ATF2, ATF7, BACH1, BATF, Bcl-6, Blimp-1, BMI1, CBFB, CREB1, CREM, CTCF, E2F1, EBF1, EGR1, ETV6, FOS, FOXA1, FOXA2, GATA3, HIF1A, IKZF1, IKZF2, IRF4, JUN, JUNB, JUND, Lef1, NFAT, NFIA, NFIB, NFKB, NR2F1, Nur77, PU.1, RELA, RUNX3, SCRT1, SCRT2, SP1, STAT4, STAT5A, T-Bet, Tcf7, ZBED1, ZNF143, and ZNF217.

Candidate Selection: Using bioinformatic analysis of single-cell RNA-seq data and ATAC-seq data of activated and resting T cells, candidate enhancers were chosen to screen in parallel with the library. Using the single-cell RNA-seq data, transcription factors upregulated during CAR-T activation were identified. The ATAC-seq open enhancer regions were screened for binding sites of these upregulated transcription factors and a subset of these enhancers were chosen as candidates. Using the single-cell RNA-seq data, top genes upregulated in activated CAR-T relative to resting CAR-T were identified and the 2 kb region upstream of those genes in the genome were chosen as candidate promoters.

Screening: The library and selected candidates are screened by flow sorting (min promoter drives expression of a fluorescent mKate protein). Activated or not activated (resting) T cells are analyzed by flow cytometry, sorted for high/low expression, and compared by NGS which promoters are identified in the high bin in activated but not resting T cells.

Results

A library of enhancer and selected candidates are screened for enhancing transcription when CAR-T are activated by target cells. Enhancers that turn on transcription when CAR-T are activated by target cells are identified.

### Example 10: IL-12 and IL-15 Drug-inducible Expression Systems in T Cells

IL-12 and/or IL-15 payload expression was assessed in T cells for various regulatable TF expression system strategies. The strategies included co-expression of CAR constructs and CAR activity was assessed.

Materials and Methods

For in vitro assessment, on Day 0, CD4/CD8 T cells (donor derived) were thawed, seeded at 1e6 cells/mL/well in a 24 well-plate, and activated with anti-CD3/CD28 Dynabeads (3:1 bead to cell) in complete T cell media (Optimizer+Supplements—Gibco+5% human serum)+rhIL-2 (100 Units/ml; Peprotech). On Day 1, 0.5 ml media was removed and cells were transduced with 3e5 pg of virus for each the constructs indicated (relevant sequences of the constructs are provided in Table 17). On Day 2, 1.5 mL of Optimizer media+100U/mL rhIL-2 was added. Day 4 cells were counted and 1e6 cells/well were transferred to a 24 well Grex plate at a final volume of 8 mL. On Day 8, 6 mL of media was removed from the wells and cells counted then seeded at a concentration of 1e6 cells/mL in fresh media with grazoprevir (2, 1, 0.5, 0.1, 0.05, 0.01 μM and no drug). Two parallel plates of cells were seeded for determining payload induction and T-cell killing. On Day 8, cells were also assessed for CAR expression by flow cytometry. Transduced T cells were incubated for a further two days and the supernatant collected via centrifugation for IL-12 and IL-15 quantification. For IL-12 and IL-15, supernatants analyzed by Luminex (R&D IL12/IL15).

For T cell killing assays, on Day 0 T cells were seeded at 1e6/mL+/−2 μM of GRZ for 48 hours to induce payload. On Day 2, target cells were counted and 35K were seeded per well in a 96 well plate for 4-6 hrs in target cell media. Then target cell media was removed and T cells were counted and 35K CAR normalized T cells were seeded [E:T— 1:1] in 200u1 of Optimizer media overnight. On Day 3, cells were transferred to a V-bottom plate, spun down, and supernatant harvested to determine killing activity using an LDH assay (CyQUANT LDH Cytotoxicity Assay; Life Technologies).

TABLE 17
IL-12 and IL-15 Regulatable TF Expression Constructs

| SB# | Domain | Sequence |
|---|---|---|
| SB01845 | | See Table 14 above for various constructs sequences |
| SB02110 | 3x Flag | MDYKDDDDKDYKDDDDKDYKDDDDKPKKKRKVSRPGERPFQCRI |
| | NLS ZF | CMRNFSRRHGLDRHTRTHTGEKPFQCRICMRNFSDHSSLKRHLRTH |
| | | TGSQKPFQCRICMRNFSVRHNLTRHLRTHTGEKPFQCRICMRNFSD |
| | | HSNLSRHLKTHTGSQKPFQCRICMRNFSQRSSLVRHLRTHTGEKPF |
| | | QCRICMRNFSESGHLKRHLRTHLRGS (SEQ ID NO: 132) |
| SB02110 | NS3 cutsite | EDVVCCHSIYGKKKGDIDTYRYIGSSGTGCVVIVGRIVLSGSGTS |
| | and linker | (SEQ ID NO: 133) |
| SB02110 | NS3 | APITAYAQQTRGLLGCIITSLTGRDKNQVEGEVQIVSTATQTFLATCI |
| | protease | NGVCWAVYHGAGTRTIASPKGPVIQMYTNVDQDLVGWPAPQGSR |
| | | SLTPCTCGSSDLYLVTRHADVIPVRRRGDSRGSLLSPRPISYLKGSSG |
| | | GPLLCPAGHAVGLFRAAVCTRGVAKAVDFIPVENLETTMRSPVFTD |
| | | (SEQ ID NO: 134) |
| SB02110 | NS3 cutsite | NSSPPAVTLTHPITKIDREVLYQEFDEMEECSQH (SEQ ID NO: 135) |
| | and linker | |
| SB02110 | VPR | See SEQ ID NO: 90 |
| SB02110 | Full-length | ATGGACTACAAGGACGACGACGACAAGGATTACAAGGATGATG |
| | cassette | ATGATAAGGACTATAAGGACGATGATGACAAACCCAAGAAGAA |
| | (DNA) | GCGGAAGGTTTCCCGGCCTGGCGAGAGGCCTTTCCAGTGCAGAA |
| | | TCTGCATGCGGAACTTCAGCAGACGGCACGGCCTGGACAGACAC |
| | | ACCAGAACACACACAGGCGAGAAACCCTTCCAGTGCCGGATCT |
| | | GTATGAGAAATTTCAGCGACCACAGCAGCCTGAAGCGGCACCTG |
| | | AGAACCCATACCGGCAGCCAGAAACCATTTCAGTGTAGGATATG |
| | | CATGCGCAATTTCTCCGTGCGGCACAACCTGACCAGACACCTGA |
| | | GGACACACACCGGGGAGAAGCCTTTTCAATGTCGCATATGCATG |
| | | AGAAACTTCTCTGACCACTCCAACCTGAGCCGCCACCTCAAAAC |
| | | CCACACCGGCTCTCAAAAGCCCTTCCAATGTAGAATATGTATGA |
| | | GGAACTTTAGCCAGCGGAGCAGCCTCGTGCGCCATCTGAGAACT |
| | | CACACTGGCGAAAAGCCGTTTCAATGCCGTATCTGTATGCGCAA |
| | | CTTTAGCGAGAGCGGCCACCTGAAGAGACATCTGCGCACACACC |
| | | TGAGAGGCAGCGAGGATGTCGTGTGCTGCCACAGCATCTACGGA |
| | | AAGAAGAAGGGCGACATCGACACCTATCGGTACATCGGCAGCA |
| | | GCGGCACAGGCTGTGTTGTGATCGTGGGCAGAATCGTGCTGAGC |
| | | GGCTCTGGAACAAGCGCCCCTATCACAGCCTACGCTCAGCAGAC |
| | | AAGAGGCCTGCTGGGCTGCATCATCACAAGCCTGACCGGCAGA |
| | | GACAAGAACCAGGTGGAAGGCGAGGTGCAGATCGTGTCTACAG |
| | | CTACCCAGACCTTCCTGGCCACCTGTATCAATGGCGTGTGCTGG |
| | | GCCGTGTATCACGGCGCTGGCACAAGAACAATCGCCTCTCCAAA |
| | | GGGCCCCGTGATCCAGATGTACACCAACGTGGACCAGGACCTCG |
| | | TTGGCTGGCCTGCTCCTCAAGGCAGCAGAAGCCTGACACCTTGC |
| | | ACCTGTGGCTCCAGCGATCTGTACCTGGTCACCAGACACGCCGA |
| | | CGTGATCCCTGTCAGAAGAAGAGGGGATTCCAGAGGCAGCCTG |
| | | CTGAGCCCTAGACCTATCAGCTACCTGAAGGGCAGCTCTGGCGG |
| | | ACCTCTGCTTTGTCCTGCTGGACATGCCGTGGGCCTGTTTAGAGC |
| | | CGCCGTGTGTACAAGAGGCGTGGCCAAAGCCGTGGACTTCATCC |
| | | CCGTGGAAAACCTGGAAACCACCATGCGGAGCCCCGTGTTCACC |
| | | GACAATTCTAGCCCTCCAGCCGTGACACTGACACACCCCATCAC |
| | | CAAGATCGACAGAGAGGTGCTGTACCAAGAGTTCGACGAGATG |
| | | GAAGAGTGCAGCCAGCACGAGGCCTCTGGATCTGGTAGAGCCG |
| | | ACGCTCTGGACGACTTCGACCTGGATATGCTGGGCTCTGACGCC |
| | | CTGGATGATTTTGACCTCGACATGCTGGGAAGCGACGCCCTCGA |
| | | CGATTTCGATTTGGACATGCTCGGCAGTGATGCACTCGATGACT |
| | | TTGATCTTGATATGCTGATCAACAGCCGGTCCAGCGGCAGCCCT |
| | | AAGAAAAAACGGAAAGTGGGCAGCCAGTATCTGCCCGACACCG |
| | | ACGATCGGCACCGGATCGAGGAAAAGCGGAAGCGGACCTACGA |
| | | GACATTCAAGAGCATCATGAAGAAGTCCCCATTCAGCGGCCCCA |
| | | CCGATCCTAGACCTCCACCTAGAAGAATCGCCGTGCCTAGCAGA |
| | | TCTAGCGCCTCCGTGCCTAAACCTGCTCCACAGCCTTATCCTTTC |
| | | ACCTCTAGCCTGAGCACCATCAACTACGACGAGTTCCCTACCAT |
| | | GGTGTTCCCCAGCGGCCAGATCTCTCAGGCATCTGCTCTTGCTCC |
| | | AGCTCCACCTCAGGTTCTGCCTCAAGCTCCTGCTCCGGCTCCTGC |
| | | ACCAGCTATGGTGTCTGCACTTGCTCAGGCACCAGCTCCAGTGC |
| | | CTGTTCTTGCTCCTGGACCTCCTCAGGCTGTTGCTCCACCAGCAC |
| | | CTAAACCTACACAGGCCGGCGAGGGAACACTGTCTGAAGCCCTG |
| | | CTGCAACTCCAGTTCGATGACGAGGATCTGGGCGCCCTGCTGGG |
| | | AAATTCTACAGACCCTGCCGTGTTTACCGATCTGGCCAGCGTGG |
| | | ACAACAGCGAGTTTCAGCAGCTCCTGAACCAGGGCATCCCAGTG |
| | | GCTCCTCACACCACAGAGCCCATGCTGATGGAATACCCCGAGGC |
| | | CATCACCAGACTGGTCACCGGCGCACAAAGACCTCCAGATCCTG |
| | | CTCCAGCACCGCTTGGAGCACCTGGACTGCCTAATGGACTGCTG |
| | | TCTGGCGACGAGGACTTCAGCTCTATCGCCGACATGGATTTCTCT |
| | | GCCCTGCTCGGCTCTGGCAGCGGCTCTAGAGATAGCAGAGAAGG |
| | | CATGTTCCTGCCTAAGCCTGAGGCCGGCTCTGCCATCTCCGATGT |
| | | GTTCGAGGGAAGAGAAGTGTGCCAGCCTAAGCGGATCCGGCCTT |
| | | TTCACCCTCCTGGAAGCCCTTGGGCCAACAGACCTCTGCCTGCTT |
| | | CTCTGGCCCCTACACCAACAGGACCTGTGCACGAACCTGTGGGC |
| | | AGTCTTACCCCTGCTCCTGTTCCTCAGCCACTGGATCCCGCACCA |
| | | GCTGTGACACCTGAAGCCAGCCATCTGCTGGAAGATCCCGACGA |
| | | AGAAACCTCTCAGGCCGTGAAGGCCCTGAGAGAAATGGCCGAC |
| | | ACAGTGATCCCTCAGAAAGAGGAAGCCGCCATTTGCGGACAGA |
| | | TGGACCTGTCTCACCCTCCACCAAGAGGCCACCTGGACGAGCTG |
| | | ACCACCACACTGGAATCCATGACCGAGGACCTGAACCTGGACA |
| | | GCCCTCTGACACCCGAGCTGAACGAGATCCTGGACACCTTCCTG |
| | | AACGACGAGTGTCTGCTGCACGCCATGCACATCTCTACCGGCCT |
| | | GAGCATCTTCGACACCAGCCTGTTCTGA (SEQ ID NO: 136) |
| SB02661 | hIL12 | See SEQ ID NO: 93 |
| SB02661 | insulator + | tccccagcatgcctgctattctcttcccaatcctcccccttgctgtcctgccccaccccaccccccagaatag |
| | inducible | aatgacacctactcagacaatgcgatgcaatttcctcattttattaggaaaggacagtgggagtggcaccttc |
| | YB-TATA | cagggtcaaggaaggcacgggggaggggcaaacaacagatggctggcaactagaaggcacagttaA |
| | promoter | CTGGCGTTCAGGTAGGACATCACGCGGTCAATGGTCACGGCTCG |
| | hIL12 on | AATGCGGAAAGCGTGCAACAGGATGCACAGCTTGATCTTGGTCT |
| | negative | TGTAGAAGTCCGGTTCTTCCAGGCTGGACTTTTGAGGCACAGTC |
| | DNA | TCGGAGTTGAAATTCAGGGCCTGCATCAGTTCATCAATCACGGC |
| | strand | GAGCATATTCTGGTCCAGGAAGATCTGCCGCTTCGGGTCCATGA |
| | | GCAGCTTGGCGTTCATGGTCTTGAACTCGACCTGATACATCTTCA |
| | | GATCTTCGTAGATCGAGGAAAGACAGAGCGCCATCATGAATGA |
| | | GGTCTTTCTCGACGCCAGGCAGCTGCCGTTAGTGATAAAGCTTG |
| | | TCTCGCGGGAGTTCAGACACGATTCGTTCTTGGTCAGTTCCAGC |
| | | GGCAGGCAGGCTTCCACGGTCGAGGTCTTGTCCTTGGTGATGTC |
| | | CTCGTGATCAATTTCTTCCGAGGTGCAGGGGTAGAACTCAAGGG |
| | | TCTGGCGGGCCTTCTGCAACATGTTCGACACAGCCCTCAGGAGG |
| | | TTTTGGGAGTGGTGTAGGCACGGGAACATTCCAGGGTCGGGGGT |
| | | TGCCACAGGGAGGTTCCGGGAACCTCCTCCGGAGCCTCCTCCTG |
| | | AACCTCCGCCTGATCCGCCACCGGAACAAGGCACGCTGGCCCAT |
| | | TCGCTCCAGGAGGACGAGTAGTATCTATCCTGCGCCCGGACGCT |
| | | GATTGACGCGTTCTTCCGACAAATCACAGTGGCGGAGGTTTTGT |
| | | CGGTGAACACCCGGTCTTTCTTCTCCCGTTTGGACTTTCCCTGCA |
| | | CTTGCACACAGAAAGTGAGCGAGAAGTATGAGTGCGGGGTGCT |
| | | CCAAGTGTCTGGATATTCCCAAGACACTTCCACTTGGCGGGAGT |
| | | TCTTGAGTGGCTTCAGCTGCAAGTTCTTGGGGGGGTCAGGCTTG |
| | | ATGATGTCGCGGATAAAGAAGGAGGAAGTGTAGTTCTCGTATTT |
| | | CAGCTTATGCACGGCATCGACCATGACCTCGATAGGCAGGGACT |
| | | CTTCCGCGGCAGGGCAGGCGCTGTCCTCCTGGCATTCCACGGAG |
| | | TACTCATATTCCTTGTTGTCTCCCCTGACTCTCTCGGCGGACAGA |
| | | GTGGCGGCTCCACAGGTCACGCCCTGAGGATCGCTTGATCCCCG |
| | | TGACGACTTCACGGAGAAAGTCAGGTCGGTGGAGATTGTCGTCA |
| | | GCCACCAACAGGTGAACCGACCGCTGTAGTTCTTGGCTTCGCAG |
| | | CGGAGGAAGGTCTTGTTCTTCGGTTCTTTTTGGTCCTTGAGGATG |
| | | TCAGTGGACCAGATTCCATCCTCTTTCTTGTGCAGCAGCAGCAG |
| | | GGAGTGGGACAGCACTTCGCCACCCTTGTGGCAAGTGTACTGGC |
| | | CCGCGTCGCCGAACTCCTTGACTTGAATGGTCAGGGTCTTTCCGC |
| | | TTCCGAGCACCTCGGAGCTCTGATCCAGGGTCCAGGTTATGCCG |
| | | TCCTCTTCTGGCGTATCGCAAGTCAGCACGACCATTTCTCCAGGG |
| | | GCGTCCGGGTACCAATCCAGCTCGACCACGTAGACGTCCTTCTT |
| | | CAGTTCCCAAATGGCGACCAGAGGGGAAGCGAGGAACACAAGG |
| | | GAGAACCAGGAGATGACGAGTTGCTGATGGCACATGGTGGCGG |
| | | CACCGGTACGCGTTGGCCCCCATTATATACCCTCTAGAACTAGTt |
| | | atccactccgtgtaagggagagtgagcctcttacgaatgCGCGACATCGGCTACGCCgttc |
| | | cgaggcgactgatacgCGCGACATCGGCTACGCCgtacgaaggcagtccgattgCGC |
| | | GACATCGGCTACGCCgacctttactgagacgggagCGCGACATCGGCTACG |
| | | CCgaaggcgttgcgaatcctcatgcgattgttacgaaacccgTTAATTAAAGAGCGAGA |
| | | TTCCGTCTCAAAGAAAAAAAAAGTAATGAAATGAATAAAATGA |
| | | GTCCTAGAGCCAGTAAATGTCGTAAATGTCTCAGCTAGTCAGGT |
| | | AGTAAAAGGTCTCAACTAGGCAGTGGCAGAGCAGGATTCAAAT |
| | | TCAGGGCTGTTGTGATGCCTCCGCAGACTCTGAGCGCCACCTGG |
| | | TGGTAATTTGTCTGTGCCTCTTCTGACGTGGAAGAACAGCAACT |
| | | AACACACTAACACGGCATTTACTATGGGCCAGCCATTGT (SEQ ID |
| | | NO: 137) |
| SB02661 | GPC3 | MLLLVTSLLLCELPHPAFLLIPHMEVQLVESGGGLVQPGGSLRLSCA |
| | CAR | ASGFTFNKNAMNWVRQAPGKGLEWVGRIRNKTNNYATYYADSV |
| | | KARFTISRDDSKNSLYLQMNSLKTEDTAVYYCVAGNSFAYWGQGT |
| | | LVTVSAGGGGSGGGGSGGGGSDIVMTQSPDSLAVSLGERATINCKS |
| | | SQSLLYSSNQKNYLAWYQQKPGQPPKLLIYWASSRESGVPDRFSGS |
| | | GSGTDFTLTISSLQAEDVAVYYCQQYYNYPLTFGQGTKLEIKTTTP |
| | | APRPPTPAPTIASQPLSLRPEACRPAAGGAVHTRGLDFACDIYIWAP |
| | | LAGTCGVLLLSLVITKRGRKKLLYIFKQPFMRPVQTTQEEDGCSCRF |
| | | PEEEEGGCELRVKFSRSADAPAYQQGQNQLYNELNLGRREEYDVL |
| | | DKRRGRDPEMGGKPRRKNPQEGLYNELQKDKMAEAYSEIGMKGE |
| | | RRRGKGHDGLYQGLSTATKDTYDALHMQALPPRGMVSKGEELFT |
| | | GVVPILVELDGDVNGHKFSVSGEGEGDATYGKLTLKLICTTGKLPV |
| | | PWPTLVTTLGYGLQCFARYPDHMKQHDFFKSAMPEGYVQERTIFF |
| | | KDDGNYKTRAEVKFEGDTLVNRIELKGIDFKEDGNILGHKLEYNY |
| | | NSHNVYITADKQKNGIKANFKIRHNIEDGGVQLADHYQQNTPIGDG |
| | | PVLLPDNHYLSYQSALSKDPNEKRDHMVLLEFVTAAGITLGMDEL |
| | | YK (SEQ ID NO: 138) |
| SB02661 | SFFV | gtaacgccattttgcaaggcatggaaaaataccaaaccaagaatagagaagttcagatcaagggcgggta |
| | GPC3 | catgaaaatagctaacgttgggccaaacaggatatctgcggtgagcagtttcggccccggcccggggcca |
| | CAR | agaacagatggtcaccgcagtttcggccccggcccgaggccaagaacagatggtccccagatatggccc |
| | | aaccctcagcagtttcttaagacccatcagatgtttccaggctcccccaaggacctgaaatgaccctgcgcc |
| | | ttatttgaattaaccaatcagcctgcttctcgcttctgttcgcgcgcttctgcttcccgagctctataaaagagct |
| | | cacaacccctcactcggcgcgccagtcctccgacagactgagtcgcccgggGGATCcTACGTA |
| | | GCCGCCACCATGCTGCTGCTGGTCACATCTCTGCTGCTGTGCGA |
| | | GCTGCCCCATCCTGCCTTTCTGCTGATCCCTCACATGGAAGTGCA |
| | | GCTGGTGGAATCTGGCGGAGGACTGGTTCAACCTGGCGGCTCTC |
| | | TGAGACTGTCTTGTGCCGCCAGCGGCTTCACCTTCAACAAGAAC |
| | | GCCATGAACTGGGTCCGACAGGCCCCTGGCAAAGGCCTTGAATG |
| | | GGTCGGACGGATCAGAAACAAGACCAACAACTACGCCACCTAC |
| | | TACGCCGACAGCGTGAAGGCCAGGTTCACCATCTCCAGAGATGA |
| | | CAGCAAGAACAGCCTGTACCTCCAGATGAACTCCCTGAAAACCG |
| | | AGGACACCGCCGTGTACTATTGCGTGGCCGGCAATAGCTTTGCC |
| | | TACTGGGGACAGGGCACCCTGGTTACAGTTTCTGCTGGTGGTGG |
| | | AGGCTCAGGAGGGGGAGGTTCCGGAGGAGGCGGTTCGGACATC |
| | | GTGATGACACAGAGCCCTGACAGCCTGGCCGTGTCTCTGGGAGA |
| | | AAGAGCCACCATCAACTGCAAGAGCAGCCAGAGCCTGCTGTACT |
| | | CCAGCAACCAGAAGAACTACCTGGCCTGGTATCAGCAAAAGCC |
| | | CGGCCAGCCTCCTAAGCTGCTGATCTATTGGGCCAGCTCCAGAG |
| | | AAAGCGGCGTGCCCGATAGATTTTCTGGCTCTGGCAGCGGCACC |
| | | GACTTCACCCTGACAATTTCTAGCCTGCAAGCCGAGGACGTGGC |
| | | CGTGTATTACTGCCAGCAGTACTACAACTACCCTCTGACCTTCGG |
| | | CCAGGGCACCAAGCTGGAAATCAAGACCACCACACCAGCTCCTC |
| | | GGCCTCCAACTCCTGCTCCTACAATTGCCAGCCAGCCTCTGTCTC |
| | | TGAGGCCCGAAGCTTGTAGACCAGCTGCCGGCGGAGCTGTGCAT |
| | | ACAAGAGGACTGGATTTCGCCTGCGACATCTACATATGGGCCCC |
| | | CCTCGCCGGTACTTGCGGTGTTTTGCTTTTGTCACTGGTGATTAC |
| | | GAAGCGCGGTCGAAAAAAACTCCTCTACATCTTCAAACAACCTT |
| | | TCATGCGGCCTGTCCAAACAACTCAAGAAGAGGACGGGTGTTCA |
| | | TGCCGCTTTCCAGAGGAAGAGGAAGGTGGCTGTGAACTTCGGGT |
| | | GAAGTTCTCACGATCTGCGGACGCCCCCGCATACCAACAGGGAC |
| | | AGAACCAACTCTACAATGAACTGAACCTGGGGCGAAGAGAAGA |
| | | ATATGACGTTCTGGATAAGCGCAGAGGCCGCGACCCCGAAATG |
| | | GGAGGTAAGCCGAGAAGGAAAAATCCACAGGAAGGACTGTATA |
| | | ATGAGTTGCAAAAGGATAAGATGGCGGAAGCATATTCAGAGAT |
| | | TGGGATGAAAGGCGAAAGAAGAAGAGGCAAAGGACATGATGG |
| | | GCTGTATCAGGGCCTTTCCACGGCCACAAAAGATACGTATGACG |
| | | CGCTGCACATGCAAGCTCTTCCTCCCAGGGGAATGGTGTCCAAA |
| | | GGAGAAGAACTGTTCACCGGAGTGGTGCCGATTCTGGTGGAACT |
| | | GGACGGCGATGTCAACGGACACAAGTTCTCAGTCAGCGGAGAA |
| | | GGGGAAGGGGATGCTACGTACGGGAAGCTCACCCTGAAGCTCA |
| | | TCTGTACTACTGGGAAGCTGCCTGTGCCCTGGCCAACTCTGGTG |
| | | ACAACCCTCGGTTACGGGTTGCAGTGCTTCGCACGCTACCCTGA |
| | | CCACATGAAGCAGCACGATTTCTTCAAATCCGCCATGCCTGAGG |
| | | GTTACGTGCAAGAGCGGACCATCTTTTTCAAGGACGATGGCAAC |
| | | TACAAGACTCGGGCCGAAGTGAAATTCGAAGGGGACACTCTTGT |
| | | GAACCGCATCGAGTTGAAGGGCATTGACTTCAAGGAAGATGGC |
| | | AACATTTTGGGACACAAGCTGGAGTACAACTACAATTCCCATAA |
| | | CGTGTACATCACCGCCGACAAGCAGAAAAACGGGATCAAGGCC |
| | | AACTTCAAGATCCGCCACAATATCGAGGATGGCGGAGTGCAGCT |
| | | GGCTGACCACTACCAGCAGAACACCCCCATCGGTGATGGCCCAG |
| | | TTCTGCTCCCCGATAACCATTACCTGTCGTACCAATCGGCATTGA |
| | | GCAAGGACCCCAACGAGAAGCGCGATCACATGGTGTTGCTGGA |
| | | ATTTGTGACCGCTGCCGGCATTACCCTCGGGATGGACGAGCTCT |
| | | ACAAGTAA (SEQ ID NO: 139) |
| SB02662 | insulator + | tccccagcatgcctgctattctcttcccaatcctcccccttgctgtcctgccccaccccaccccccagaatag |
| | inducible | aatgacacctactcagacaatgcgatgcaatttcctcattttattaggaaaggacagtgggagtggcaccttc |
| | YB-TATA | cagggtcaaggaaggcacgggggaggggcaaacaacagatggctggcaactagaaggcacagTCA |
| | promoter | GCTGGTGTTGATGAACATCTGCACGATGTGCACGAAGCTCTGCA |
| | IL-12 | GGAACTCTTTGATATTCTTTTCTTCCAGTTCCTCGCATTCTTTGCA |
| | _2A_IL15 | GCCGGACTCGGTCACATTGCCGTTGGAGGACAGGCTGTTGTTGG |
| | on negative | CCAGGATGATCAGGTTTTCCACGGTATCGTGGATGGAGGCGTCG |
| | DNA | CCGCTTTCCAGGCTGATCACTTGCAGCTCGAGCAGAAAGCACTT |
| | strand | CATGGCGGTCACTTTACAGCTAGGGTGCACGTCGCTCTCGGTGT |
| | | ACAGTGTGGCGTCGATGTGCATGCTCTGGATCAGATCCTCGATC |
| | | TTCTTCAGATCGCTGATCACGTTGACCCAGTTGCCTGTAGAGCCG |
| | | GGCACCCAAAGAAGCAGCACCCACAGCAGCAGTGTGTCGGTTTC |
| | | CATAGGTCCAGGGTTTTCCTCCACGTCTCCACACGTCAGTAGACT |
| | | CCCGCGTCCCTCGCCAGATCCTCTCTTCCGTCTACTGGCGTTCAG |
| | | GTAGGACATCACGCGGTCAATGGTCACGGCTCGAATGCGGAAA |
| | | GCGTGCAACAGGATGCACAGCTTGATCTTGGTCTTGTAGAAGTC |
| | | CGGTTCTTCCAGGCTGGACTTTTGAGGCACAGTCTCGGAGTTGA |
| | | AATTCAGGGCCTGCATCAGTTCATCAATCACGGCGAGCATATTC |
| | | TGGTCCAGGAAGATCTGCCGCTTCGGGTCCATGAGCAGCTTGGC |
| | | GTTCATGGTCTTGAACTCGACCTGATACATCTTCAGATCTTCGTA |
| | | GATCGAGGAAAGACAGAGCGCCATCATGAATGAGGTCTTTCTCG |
| | | ACGCCAGGCAGCTGCCGTTAGTGATAAAGCTTGTCTCGCGGGAG |
| | | TTCAGACACGATTCGTTCTTGGTCAGTTCCAGCGGCAGGCAGGC |
| | | TTCCACGGTCGAGGTCTTGTCCTTGGTGATGTCCTCGTGATCAAT |
| | | TTCTTCCGAGGTGCAGGGGTAGAACTCAAGGGTCTGGCGGGCCT |
| | | TCTGCAACATGTTCGACACAGCCCTCAGGAGGTTTTGGGAGTGG |
| | | TGTAGGCACGGGAACATTCCAGGGTCGGGGGTTGCCACAGGGA |
| | | GGTTCCGGGAACCTCCTCCGGAGCCTCCTCCTGAACCTCCGCCT |
| | | GATCCGCCACCGGAACAAGGCACGCTGGCCCATTCGCTCCAGGA |
| | | GGACGAGTAGTATCTATCCTGCGCCCGGACGCTGATTGACGCGT |
| | | TCTTCCGACAAATCACAGTGGCGGAGGTTTTGTCGGTGAACACC |
| | | CGGTCTTTCTTCTCCCGTTTGGACTTTCCCTGCACTTGCACACAG |
| | | AAAGTGAGCGAGAAGTATGAGTGCGGGGTGCTCCAAGTGTCTG |
| | | GATATTCCCAAGACACTTCCACTTGGCGGGAGTTCTTGAGTGGC |
| | | TTCAGCTGCAAGTTCTTGGGGGGGTCAGGCTTGATGATGTCGCG |
| | | GATAAAGAAGGAGGAAGTGTAGTTCTCGTATTTCAGCTTATGCA |
| | | CGGCATCGACCATGACCTCGATAGGCAGGGACTCTTCCGCGGCA |
| | | GGGCAGGCGCTGTCCTCCTGGCATTCCACGGAGTACTCATATTC |
| | | CTTGTTGTCTCCCCTGACTCTCTCGGCGGACAGAGTGGCGGCTCC |
| | | ACAGGTCACGCCCTGAGGATCGCTTGATCCCCGTGACGACTTCA |
| | | CGGAGAAAGTCAGGTCGGTGGAGATTGTCGTCAGCCACCAACA |
| | | GGTGAACCGACCGCTGTAGTTCTTGGCTTCGCAGCGGAGGAAGG |
| | | TCTTGTTCTTCGGTTCTTTTTGGTCCTTGAGGATGTCAGTGGACC |
| | | AGATTCCATCCTCTTTCTTGTGCAGCAGCAGCAGGGAGTGGGAC |
| | | AGCACTTCGCCACCCTTGTGGCAAGTGTACTGGCCCGCGTCGCC |
| | | GAACTCCTTGACTTGAATGGTCAGGGTCTTTCCGCTTCCGAGCAC |
| | | CTCGGAGCTCTGATCCAGGGTCCAGGTTATGCCGTCCTCTTCTGG |
| | | CGTATCGCAAGTCAGCACGACCATTTCTCCAGGGGCGTCCGGGT |
| | | ACCAATCCAGCTCGACCACGTAGACGTCCTTCTTCAGTTCCCAA |
| | | ATGGCGACCAGAGGGGAAGCGAGGAACACAAGGGAGAACCAG |
| | | GAGATGACGAGTTGCTGATGGCACATGGTGGCGGCACCGGTAC |
| | | GCGTTGGCCCCCATTATATACCCTCTAGAACTAGTtatccactccgtgtaa |
| | | gggagagtgagcctcttacgaatgCGCGACATCGGCTACGCCgttccgaggcgactgata |
| | | cgCGCGACATCGGCTACGCCgtacgaaggcagtccgattgCGCGACATCGGC |
| | | TACGCCgacctttactgagacgggagCGCGACATCGGCTACGCCgaaggcgttgcg |
| | | aatcctcatgcgattgttacgaaacccgTTAATTAAAGAGCGAGATTCCGTCTCA |
| | | AAGAAAAAAAAAGTAATGAAATGAATAAAATGAGTCCTAGAGC |
| | | CAGTAAATGTCGTAAATGTCTCAGCTAGTCAGGTAGTAAAAGGT |
| | | CTCAACTAGGCAGTGGCAGAGCAGGATTCAAATTCAGGGCTGTT |
| | | GTGATGCCTCCGCAGACTCTGAGCGCCACCTGGTGGTAATTTGT |
| | | CTGTGCCTCTTCTGACGTGGAAGAACAGCAACTAACACACTAAC |
| | | ACGGCATTTACTATGGGCCAGCCATTGT (SEQ ID NO: 140) |
| SB02662 | CAR | See SB02661 sequences |
| SB02662 | IL-12 | See SEQ ID NO: 93 |
| SB02662 | IL-15 | METDTLLLWVLLLWVPGSTGNWVNVISDLKKIEDLIQSMHIDATLY |
| | | TESDVHPSCKVTAMKCFLLELQVISLESGDASIHDTVENLIILANNSL |
| | | SSNGNVTESGCKECEELEEKNIKEFLQSFVHIVQMFINTS (SEQ ID |
| | | NO: 141) |
| SB02662 | 2A peptide | RRKRGSGEGRGSLLTCGDVEENPGP (SEQ ID NO: 142) |
| SB02664 | insulator + | tccccagcatgcctgctattctcttcccaatcctcccccttgctgtcctgccccaccccaccccccagaatag |
| | inducible | aatgacacctactcagacaatgcgatgcaatttcctcattttattaggaaaggacagtgggagtggcaccttc |
| | minTK | cagggtcaaggaaggcacgggggaggggcaaacaacagatggctggcaactagaaggcacagttaA |
| | promoter | CTGGCGTTCAGGTAGGACATCACGCGGTCAATGGTCACGGCTCG |
| | hIL12 on | AATGCGGAAAGCGTGCAACAGGATGCACAGCTTGATCTTGGTCT |
| | negative | TGTAGAAGTCCGGTTCTTCCAGGCTGGACTTTTGAGGCACAGTC |
| | DNA | TCGGAGTTGAAATTCAGGGCCTGCATCAGTTCATCAATCACGGC |
| | strand | GAGCATATTCTGGTCCAGGAAGATCTGCCGCTTCGGGTCCATGA |
| | | GCAGCTTGGCGTTCATGGTCTTGAACTCGACCTGATACATCTTCA |
| | | GATCTTCGTAGATCGAGGAAAGACAGAGCGCCATCATGAATGA |
| | | GGTCTTTCTCGACGCCAGGCAGCTGCCGTTAGTGATAAAGCTTG |
| | | TCTCGCGGGAGTTCAGACACGATTCGTTCTTGGTCAGTTCCAGC |
| | | GGCAGGCAGGCTTCCACGGTCGAGGTCTTGTCCTTGGTGATGTC |
| | | CTCGTGATCAATTTCTTCCGAGGTGCAGGGGTAGAACTCAAGGG |
| | | TCTGGCGGGCCTTCTGCAACATGTTCGACACAGCCCTCAGGAGG |
| | | TTTTGGGAGTGGTGTAGGCACGGGAACATTCCAGGGTCGGGGGT |
| | | TGCCACAGGGAGGTTCCGGGAACCTCCTCCGGAGCCTCCTCCTG |
| | | AACCTCCGCCTGATCCGCCACCGGAACAAGGCACGCTGGCCCAT |
| | | TCGCTCCAGGAGGACGAGTAGTATCTATCCTGCGCCCGGACGCT |
| | | GATTGACGCGTTCTTCCGACAAATCACAGTGGCGGAGGTTTTGT |
| | | CGGTGAACACCCGGTCTTTCTTCTCCCGTTTGGACTTTCCCTGCA |
| | | CTTGCACACAGAAAGTGAGCGAGAAGTATGAGTGCGGGGTGCT |
| | | CCAAGTGTCTGGATATTCCCAAGACACTTCCACTTGGCGGGAGT |
| | | TCTTGAGTGGCTTCAGCTGCAAGTTCTTGGGGGGGTCAGGCTTG |
| | | ATGATGTCGCGGATAAAGAAGGAGGAAGTGTAGTTCTCGTATTT |
| | | CAGCTTATGCACGGCATCGACCATGACCTCGATAGGCAGGGACT |
| | | CTTCCGCGGCAGGGCAGGCGCTGTCCTCCTGGCATTCCACGGAG |
| | | TACTCATATTCCTTGTTGTCTCCCCTGACTCTCTCGGCGGACAGA |
| | | GTGGCGGCTCCACAGGTCACGCCCTGAGGATCGCTTGATCCCCG |
| | | TGACGACTTCACGGAGAAAGTCAGGTCGGTGGAGATTGTCGTCA |
| | | GCCACCAACAGGTGAACCGACCGCTGTAGTTCTTGGCTTCGCAG |
| | | CGGAGGAAGGTCTTGTTCTTCGGTTCTTTTTGGTCCTTGAGGATG |
| | | TCAGTGGACCAGATTCCATCCTCTTTCTTGTGCAGCAGCAGCAG |
| | | GGAGTGGGACAGCACTTCGCCACCCTTGTGGCAAGTGTACTGGC |
| | | CCGCGTCGCCGAACTCCTTGACTTGAATGGTCAGGGTCTTTCCGC |
| | | TTCCGAGCACCTCGGAGCTCTGATCCAGGGTCCAGGTTATGCCG |
| | | TCCTCTTCTGGCGTATCGCAAGTCAGCACGACCATTTCTCCAGGG |
| | | GCGTCCGGGTACCAATCCAGCTCGACCACGTAGACGTCCTTCTT |
| | | CAGTTCCCAAATGGCGACCAGAGGGGAAGCGAGGAACACAAGG |
| | | GAGAACCAGGAGATGACGAGTTGCTGATGGCACATGGTGGCGG |
| | | CACCGGTttaagcgggtcgctgcagggtcgctcggtgttcgaggccacacgcgtcaccttaatatgc |
| | | gaaACTAGTtatccactccgtgtaagggagagtgagcctcttacgaatgCGCGACATCGG |
| | | CTACGCCgttccgaggcgactgatacgCGCGACATCGGCTACGCCgtacgaaggc |
| | | agtccgattgCGCGACATCGGCTACGCCgacctttactgagacgggagCGCGACA |
| | | TCGGCTACGCCgaaggcgttgcgaatcctcatgcgattgttacgaaacccgTTAATTAA |
| | | AGAGCGAGATTCCGTCTCAAAGAAAAAAAAAGTAATGAAATGA |
| | | ATAAAATGAGTCCTAGAGCCAGTAAATGTCGTAAATGTCTCAGC |
| | | TAGTCAGGTAGTAAAAGGTCTCAACTAGGCAGTGGCAGAGCAG |
| | | GATTCAAATTCAGGGCTGTTGTGATGCCTCCGCAGACTCTGAGC |
| | | GCCACCTGGTGGTAATTTGTCTGTGCCTCTTCTGACGTGGAAGA |
| | | ACAGCAACTAACACACTAACACGGCATTTACTATGGGCCAGCCA |
| | | TTGT (SEQ ID NO: 143) |
| SB02664 | CAR | See SB02661 sequences |
| SB02664 | IL-12 | See SEQ ID NO: 93 |
| SB02665 | insulator + | tccccagcatgcctgctattctcttcccaatcctcccccttgctgtcctgccccaccccaccccccagaatag |
| | inducible | aatgacacctactcagacaatgcgatgcaatttcctcattttattaggaaaggacagtgggagtggcaccttc |
| | minTK | cagggtcaaggaaggcacgggggaggggcaaacaacagatggctggcaactagaaggcacagTCA |
| | promoter | GCTGGTGTTGATGAACATCTGCACGATGTGCACGAAGCTCTGCA |
| | IL-12 | GGAACTCTTTGATATTCTTTTCTTCCAGTTCCTCGCATTCTTTGCA |
| | _2A_IL15 | GCCGGACTCGGTCACATTGCCGTTGGAGGACAGGCTGTTGTTGG |
| | on negative | CCAGGATGATCAGGTTTTCCACGGTATCGTGGATGGAGGCGTCG |
| | DNA | CCGCTTTCCAGGCTGATCACTTGCAGCTCGAGCAGAAAGCACTT |
| | strand | CATGGCGGTCACTTTACAGCTAGGGTGCACGTCGCTCTCGGTGT |
| | | ACAGTGTGGCGTCGATGTGCATGCTCTGGATCAGATCCTCGATC |
| | | TTCTTCAGATCGCTGATCACGTTGACCCAGTTGCCTGTAGAGCCG |
| | | GGCACCCAAAGAAGCAGCACCCACAGCAGCAGTGTGTCGGTTTC |
| | | CATAGGTCCAGGGTTTTCCTCCACGTCTCCACACGTCAGTAGACT |
| | | CCCGCGTCCCTCGCCAGATCCTCTCTTCCGTCTACTGGCGTTCAG |
| | | GTAGGACATCACGCGGTCAATGGTCACGGCTCGAATGCGGAAA |
| | | GCGTGCAACAGGATGCACAGCTTGATCTTGGTCTTGTAGAAGTC |
| | | CGGTTCTTCCAGGCTGGACTTTTGAGGCACAGTCTCGGAGTTGA |
| | | AATTCAGGGCCTGCATCAGTTCATCAATCACGGCGAGCATATTC |
| | | TGGTCCAGGAAGATCTGCCGCTTCGGGTCCATGAGCAGCTTGGC |
| | | GTTCATGGTCTTGAACTCGACCTGATACATCTTCAGATCTTCGTA |
| | | GATCGAGGAAAGACAGAGCGCCATCATGAATGAGGTCTTTCTCG |
| | | ACGCCAGGCAGCTGCCGTTAGTGATAAAGCTTGTCTCGCGGGAG |
| | | TTCAGACACGATTCGTTCTTGGTCAGTTCCAGCGGCAGGCAGGC |
| | | TTCCACGGTCGAGGTCTTGTCCTTGGTGATGTCCTCGTGATCAAT |
| | | TTCTTCCGAGGTGCAGGGGTAGAACTCAAGGGTCTGGCGGGCCT |
| | | TCTGCAACATGTTCGACACAGCCCTCAGGAGGTTTTGGGAGTGG |
| | | TGTAGGCACGGGAACATTCCAGGGTCGGGGGTTGCCACAGGGA |
| | | GGTTCCGGGAACCTCCTCCGGAGCCTCCTCCTGAACCTCCGCCT |
| | | GATCCGCCACCGGAACAAGGCACGCTGGCCCATTCGCTCCAGGA |
| | | GGACGAGTAGTATCTATCCTGCGCCCGGACGCTGATTGACGCGT |
| | | TCTTCCGACAAATCACAGTGGCGGAGGTTTTGTCGGTGAACACC |
| | | CGGTCTTTCTTCTCCCGTTTGGACTTTCCCTGCACTTGCACACAG |
| | | AAAGTGAGCGAGAAGTATGAGTGCGGGGTGCTCCAAGTGTCTG |
| | | GATATTCCCAAGACACTTCCACTTGGCGGGAGTTCTTGAGTGGC |
| | | TTCAGCTGCAAGTTCTTGGGGGGGTCAGGCTTGATGATGTCGCG |
| | | GATAAAGAAGGAGGAAGTGTAGTTCTCGTATTTCAGCTTATGCA |
| | | CGGCATCGACCATGACCTCGATAGGCAGGGACTCTTCCGCGGCA |
| | | GGGCAGGCGCTGTCCTCCTGGCATTCCACGGAGTACTCATATTC |
| | | CTTGTTGTCTCCCCTGACTCTCTCGGCGGACAGAGTGGCGGCTCC |
| | | ACAGGTCACGCCCTGAGGATCGCTTGATCCCCGTGACGACTTCA |
| | | CGGAGAAAGTCAGGTCGGTGGAGATTGTCGTCAGCCACCAACA |
| | | GGTGAACCGACCGCTGTAGTTCTTGGCTTCGCAGCGGAGGAAGG |
| | | TCTTGTTCTTCGGTTCTTTTTGGTCCTTGAGGATGTCAGTGGACC |
| | | AGATTCCATCCTCTTTCTTGTGCAGCAGCAGCAGGGAGTGGGAC |
| | | AGCACTTCGCCACCCTTGTGGCAAGTGTACTGGCCCGCGTCGCC |
| | | GAACTCCTTGACTTGAATGGTCAGGGTCTTTCCGCTTCCGAGCAC |
| | | CTCGGAGCTCTGATCCAGGGTCCAGGTTATGCCGTCCTCTTCTGG |
| | | CGTATCGCAAGTCAGCACGACCATTTCTCCAGGGGCGTCCGGGT |
| | | ACCAATCCAGCTCGACCACGTAGACGTCCTTCTTCAGTTCCCAA |
| | | ATGGCGACCAGAGGGGAAGCGAGGAACACAAGGGAGAACCAG |
| | | GAGATGACGAGTTGCTGATGGCACATGGTGGCGGCACCGGTttaag |
| | | cgggtcgctgcagggtcgctcggtgttcgaggccacacgcgtcaccttaatatgcgaaACTAGTtatc |
| | | cactccgtgtaagggagagtgagcctcttacgaatgCGCGACATCGGCTACGCCgttccg |
| | | aggcgactgatacgCGCGACATCGGCTACGCCgtacgaaggcagtccgattgCGCG |
| | | ACATCGGCTACGCCgacctttactgagacgggagCGCGACATCGGCTACGC |
| | | CgaaggcgttgcgaatcctcatgcgattgttacgaaacccgTTAATTAAAGAGCGAGAT |
| | | TCCGTCTCAAAGAAAAAAAAAGTAATGAAATGAATAAAATGAG |
| | | TCCTAGAGCCAGTAAATGTCGTAAATGTCTCAGCTAGTCAGGTA |
| | | GTAAAAGGTCTCAACTAGGCAGTGGCAGAGCAGGATTCAAATTC |
| | | AGGGCTGTTGTGATGCCTCCGCAGACTCTGAGCGCCACCTGGTG |
| | | GTAATTTGTCTGTGCCTCTTCTGACGTGGAAGAACAGCAACTAA |
| | | CACACTAACACGGCATTTACTATGGGCCAGCCATTGT (SEQ ID |
| | | NO: 144) |
| SB02665 | CAR | See SB02661 sequences |
| SB02665 | IL-12 | See SEQ ID NO: 93 |
| SB02665 | IL-15 | See SEQ ID NO: 141 |
| SB02665 | 2A peptide | See SEQ ID NO: 142 |

### Results

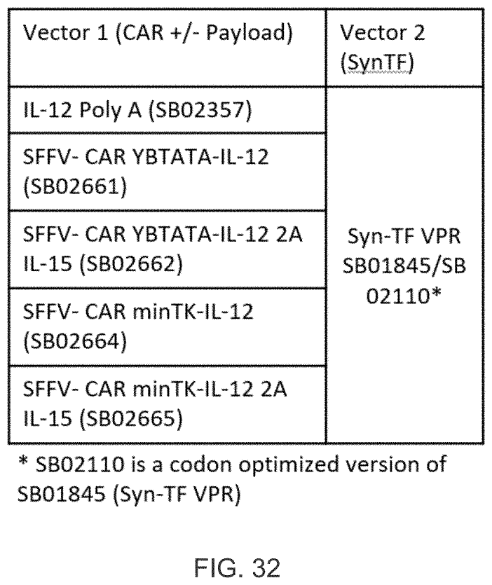

IL-12 and/or IL-15 payload expression was assessed in T cells for various regulatable TF expression system strategies and constructs. An drug-inducible format ACP (also referred to as “synTF”) using an NS3/NS4 protease cleavage site and a VPR transcriptional effector domain (SB01845) or a modified version of the ACP with linkers shortened and the entire construct codon optimized (SB02110) were assessed. All payload constructs (SB02661, SB02662, SB02664, SB02665) encoded a GPC3-CAR driven by an SFFV promoter and all cytokine payloads included an A2 insulator and promoter driving expression in the opposite direction as the CAR cassette (e.g., see orientation of reporter and CAR cassettes in construct “2235” in FIG. 20). The inducible synTF promoter was either derived from a YB-TATA promoter (SB02661, SB02662) or minTK (SB02664, SB02665). A summary of the systems assessed in shown in FIG. 32. SB00171 and SB01335 encoding constitutive hIL-12 and IL-15, respectively, each driven by SFFV were used as controls.

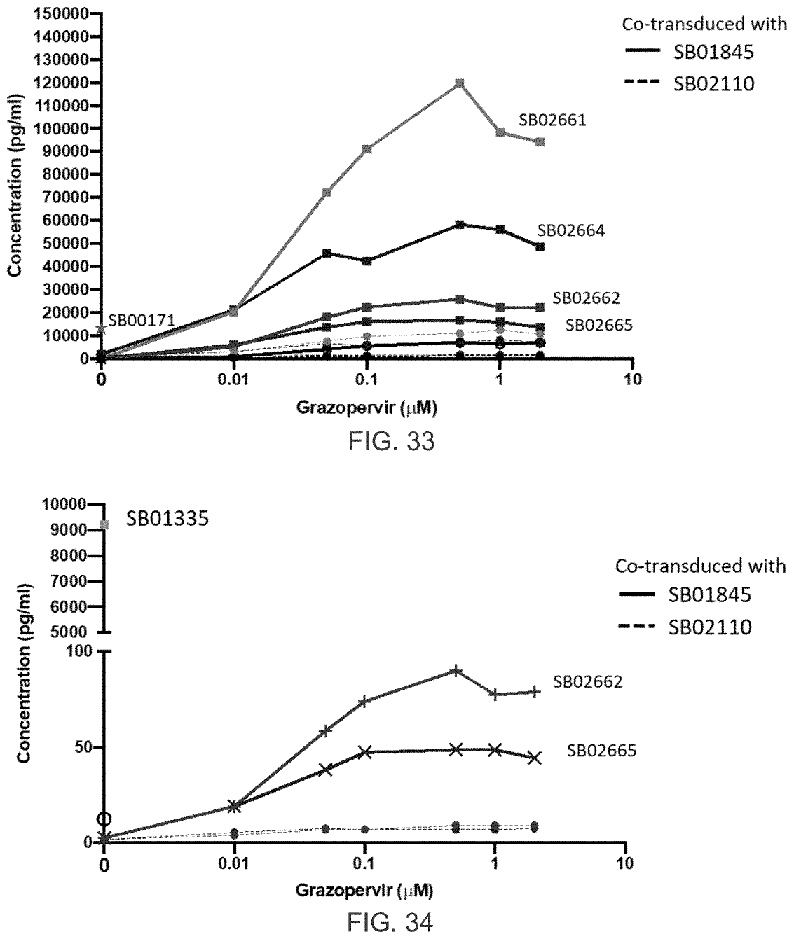

As shown in FIG. 33 and quantified in Table 18, IL-12 production was observed in a concentration dependent manner following addition of the NS3 protease inhibitor grazoprevir. As shown in FIG. 34 and quantified in Table 19, IL-15 production was observed for constructs encoding IL-15 using a ribosome skipping tag 2A multicistronic system in a concentration dependent manner for the NS3 protease inhibitor grazoprevir, with IL-15 expressed at lower levels relatively to IL-12. Production was notably greater when using ACP construct SB01845 compared to SB02110 for all combinations for both IL-12 and IL-15 (solid vs. dashed FIG. 33 and Table 18A vs Table 18B, respectively). In addition, the YB-TATA based inducible promoter demonstrated greater expression than the minTK promoter (SB02661 vs SB02664 and SB02662 vs SB02665) and less IL-12 was observed when expressed as part of a 2A/IL-15 peptide. Accordingly, regulated cytokine expression was observed in a drug-dependent in T cells for the various systems with ACP construct SB01845 together with SB02661 demonstrating the highest production of IL-12 alone and together with SB02662 IL-12 and IL-15 in a multicistronic system.

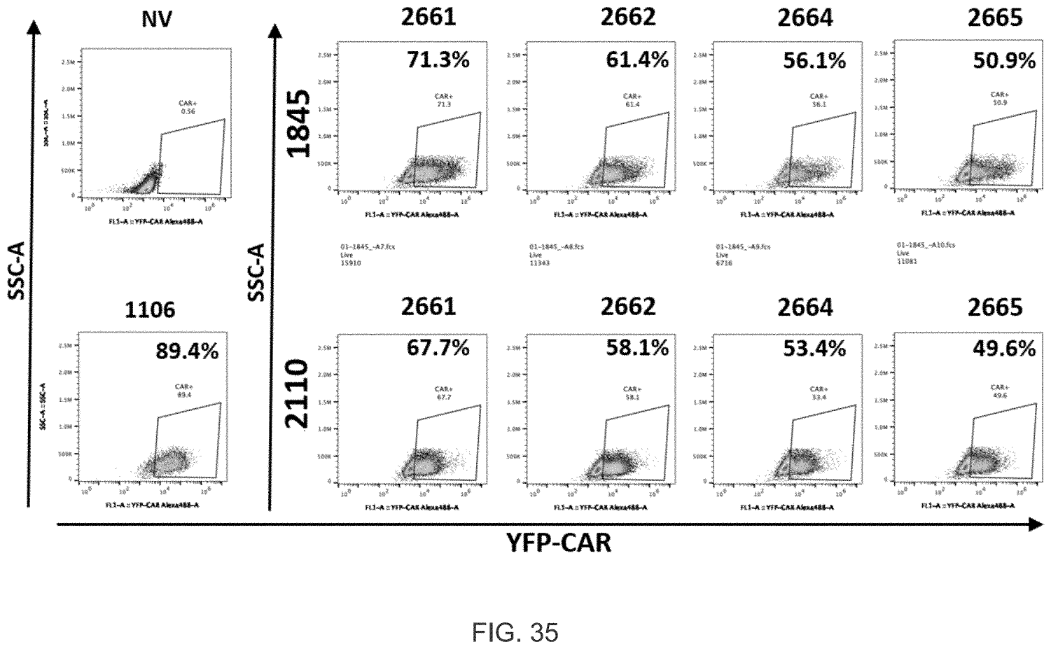

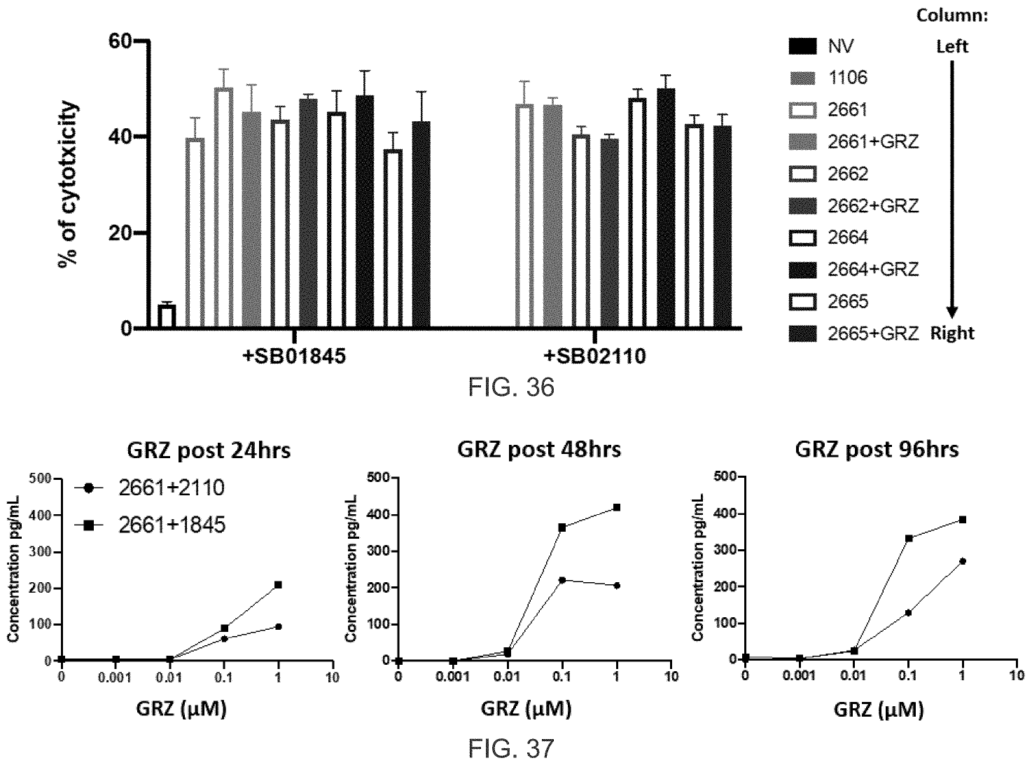

CAR profiles for the various constructs was also assessed. Expression was monitored by flow cytometry (YFP) for the various constructs, with a GPC3-CAR only construct (“1106”) as a control. As shown in FIG. 35, all cytokine payload constructs expressed the CAR ranging from ˜50-70%. As expected, the ACP construct choice did not noticeably affect expression (1845 vs 2110). CAR activity was then assessed through T cell killing. As shown in FIG. 36 and quantified in Table 20, killing (LDH release) was observed and comparable for all constructs tested that expressed the CAR, including the CAR only control, regardless of ACP used (1845 vs 2110). Killing was also assessed in the presence or absence of grazoprevir. No noticeable differences in killing were observed in the presence or absence of grazoprevir, although the assay was not designed to directly test cytokine armoring influence as the assay was performed in the absence of additional immune system components. Accordingly, CAR expression and killing activity was demonstrated for each regulatable TF expression system examined using drug-inducible ACP formats.

TABLE 18A
IL-12 production with ACP 1845 in T Cells

| [GRZ] uM | 2357 + 1845 | 2661 + 1845 | 2662 + 1845 | 2664 + 1845 | 2665 + 1845 | NV | SB00171 |
|---|---|---|---|---|---|---|---|
| 2 | 7001.8 | 94155.5 | 22296.0 | 48693.2 | 13746.7 | | |
| 1 | 6335.4 | 98314.1 | 22220.4 | 56098.2 | 15942.5 | | |
| 0.5 | 7085.0 | 119580.0 | 25885.7 | 58288.7 | 16808.8 | | |
| 0.1 | 5651.3 | 91022.2 | 22464.0 | 42428.9 | 16092.4 | | |
| 0.05 | 4194.9 | 72358.4 | 18026.4 | 45813.9 | 13788.3 | | |
| 0.01 | 892.5 | 20383.8 | 5233.6 | 21137.9 | 6018.5 | | |
| No drug | 32.0 | 51.2 | 32.0 | 2013.4 | 236.1 | 12.4 | 13165.0 |

TABLE 18B
IL-12 production with ACP 2110 in T Cells

| [GRZ] uM | 2357 + 2110 | 2661 + 2110 | 2662 + 2110 | 2664 + 2110 | 2665 + 2110 | NV | SB00171 |
|---|---|---|---|---|---|---|---|
| 2 | 1240.1 | 10790.5 | 1773.9 | 7118.3 | 1567.9 | | |
| 1 | 1101.5 | 12533.9 | 1893.7 | 8182.8 | 1516.3 | | |
| 0.5 | 1101.5 | 11006.3 | 1910.9 | 7134.9 | 1567.9 | | |
| 0.1 | 979.7 | 9785.9 | 1378.4 | 5668.0 | 1636.6 | | |
| 0.05 | 892.5 | 7634.1 | 1292.0 | 6585.4 | 1705.3 | | |
| 0.01 | 380.4 | 3134.1 | 344.5 | 3184.8 | 944.8 | | |
| No drug | 32.0 | 32.0 | 12.4 | 1378.4 | 163.0 | 12.4 | 13165.0 |

TABLE 19
IL-15 Production in T Cells

| [GRZ] uM | 2662 + 2110 | 2665 + 2110 | 2662 + 1845 | 2665 + 1845 | NV | SB01335 |
|---|---|---|---|---|---|---|
| 2 | 9.03 | 7.59 | 78.72 | 44.40 | | |
| 1 | 9.03 | 6.87 | 77.32 | 48.61 | | |
| 0.5 | 9.03 | 6.87 | 89.91 | 48.61 | | |
| 0.1 | 6.87 | 6.87 | 73.83 | 47.21 | | |
| 0.05 | 6.87 | 7.59 | 58.43 | 38.08 | | |
| 0.01 | 3.97 | 5.42 | 19.01 | 19.01 | | |
| No drug | 1.77 | 1.77 | 2.51 | 2.51 | 12.4 | 9213 |

TABLE 20
T cell Killing (LDH)

| | 1845 | 1845 | 1845 | 2110 | 2110 | 2110 |
|---|---|---|---|---|---|---|
| [GRZ] uM | Rep 1 | Rep 2 | Rep 3 | Rep 1 | Rep 2 | Rep 3 |
| NV | 5.57 | 4.43 | 5.10 | | | |
| 1106 | 44.55 | 37.87 | 36.82 | | | |
| 2661 | 53.82 | 50.67 | 46.18 | 51.03 | 41.96 | 47.76 |
| 2661 + Grz | 47.90 | 49.04 | 38.92 | 48.13 | 45.50 | 46.59 |
| 2662 | 41.59 | 46.66 | 42.36 | 41.60 | 38.70 | 41.33 |
| 2662 + Grz | 47.99 | 48.85 | 46.85 | 40.06 | 38.34 | 40.15 |
| 2664 | 40.16 | 47.23 | 48.09 | 48.04 | 46.68 | 49.94 |
| 2664 + Grz | 45.41 | 45.99 | 54.59 | 47.04 | 51.84 | 51.66 |
| 2665 | 36.43 | 34.62 | 41.31 | 41.06 | 42.96 | 44.32 |
| 2665 + Grz | 36.72 | 43.60 | 49.24 | 39.88 | 42.60 | 44.50 |

### Example 11: IL-12 Drug-Inducible Expression Systems in NK Cells

IL-12 payload expression was assessed in NK cells for various regulatable TF expression system strategies. The strategies included co-expression of CAR constructs and CAR expression was assessed.

### Materials and Methods

For in vitro assessment, starting on Day 0, NK cells (iCD3-CD56 cells isolated from health donor PBMCs) were expanded for 10 days with mitoC WT K562 cell, 500 U/ml rhIL-2, and 10 ng/ml rhIL-15. Day 10, cells were spun down in a large volume of PBS then resuspended in NK MACS medium (Miltenyi) without serum or supplements at 10e6/mL concentration. Cells were then seeded at 1e6 cells (100u1) cells+1 ul of 1:10 BX795 (Tocris Bioscience Cat #4318) for 30 mins in a 48-well retronectin coated non TC treated plate then virus added at an MOI=25 (IU) (MOI=12.5 for each virus for each of the constructs indicated; relevant sequences of the constructs are provided in Table 17) followed by 200u1 of NK MACS media (no serum/supplements). The cells and virus were then spinoculated (800g 2 hrs at 32C, rest the plate for 2 hrs at 37C incubator and transfer cells to a 24-well Grex plate in complete NK MACS media+500U/mL of rhIL-2). On Day 14, media was partially exchanged removing 5 ml media and supplemented with fresh media+rhIL-2 (500U/mL). On Day 17, cells were counted and the required number of cells were spun down in fresh complete media+500U/mL rhIL2. Cells were seeded at 0.2e6 cells/200u1/well in a U-bottom 96 well-plate in increasing concentration of Grazoprevir (1, 0.1, 0.01, 0.01 μM GRZ and no drug). On Days 12, 19, and 21 (24, 48, 96 hours, respectively), cells were transferred to a V-bottom plate, spun down, and supernatant saved for analysis using Luminex (IL-12 Milliplex kit). Cells were also assessed for CAR expression by flow cytometry.

### Results

IL-12 payload expression was assessed in NK cells for various regulatable TF expression system strategies and constructs. A drug-inducible format ACP (also referred to as “synTF”) using an NS3/NS4 protease cleavage site and a VPR transcriptional effector domain (SB01845) or a modified version of the ACP with linkers shortened and the entire construct codon optimized (SB02110) were assessed. The payload construct SB02661 encoded a GPC3-CAR driven by an SFFV promoter, with the IL-12 cytokine payload encoded in a cassette including an A2 insulator and YB-TATA promoter driving expression in the opposite direction as the CAR cassette (e.g., see orientation of reporter and CAR cassettes in construct “2235” in FIG. 20).

As shown in FIG. 37 and quantified in Table 21, IL-12 production was observed in a concentration dependent manner following addition of the NS3 protease inhibitor grazoprevir starting at 24 hours after treatment. IL-12 levels were similar between hours 48 and 96, suggesting production peaked at least 48 hours. Production was also notably greater when using ACP construct SB01845 compared to SB02110 for all combinations for both IL-12 and IL-15 (square vs. circle FIG. 37, respectively, and Table 21). CAR expression was also monitored by flow cytometry (YFP) with low overall YFP expression detected (data not shown). Accordingly, regulated cytokine expression was observed in a drug-dependent manner in NK cells for the various systems with ACP construct SB01845 together, with construct SB02661 demonstrating the highest production of IL-12.

TABLE 21
IL-12 Production in NK Cells (24 hr)

| | 2661 + 2110 | 2661 + 1845 | 2661 + 2110 | 2661 + 1845 | 2661 + 2110 | 2661 + 1845 |
|---|---|---|---|---|---|---|
| [GRZ] uM | (24 hr) | (24 hr) | (48 hr) | (48 hr) | (96 hr) | (96 hr) |
| 1 | 94.72 | 209.46 | 206.33 | 419.42 | 269.83 | 384.29 |
| 0.1 | 61.30 | 89.49 | 220.91 | 365.22 | 129.36 | 332.50 |
| 0.01 | 4.88 | 4.88 | 18.42 | 26.82 | 26.57 | 24.90 |
| 0.001 | 4.88 | 4.88 | 0.00 | 0.00 | 4.18 | 4.53 |
| No drug | 4.88 | 4.88 | 0.00 | 0.00 | 0.79 | 8.36 |

### Example 12: Additional IL-12 and IL-15 Drug-Inducible Systems in T Cells

IL-12 and/or IL-15 payload expression was assessed in T cells for additional various regulatable TF expression system strategies, including assessment of different transcription activators and production of cytokines encoded separately or in multicistronic systems.

### Materials and Methods

For in vitro assessment, on Day 0, CD4/CD8 T cells (donor derived) were thawed, seeded at 1e6 cells/mL/well in a 24 well-plate, and activated with anti-CD3/CD28 Dynabeads (3:1 bead to cell) in complete T cell media (Optimizer+Supplements—Gibco+5% human serum)+rhIL-2 (100 Units/ml; Peprotech). On Day 1, 0.5 ml media was removed and cells were transduced with 3e5 pg of virus for each the constructs indicated (relevant sequences of the constructs are provided in Table 22). On Day 2, 1.5 mL of Optimizer media+100U/mL rhIL-2 was added. Day 4 cells were counted and 1e6 cells/well were transferred to a 24 well Grex plate at a final volume of 8 mL. On Day 8, 6 mL of media was removed from the wells and cells counted then seeded at a concentration of 1e6 cells/mL in fresh media with grazoprevir (2, 1, 0.5, 0.1, 0.05, 0.01 μM and no drug). Transduced T cells were incubated for a further two days and the supernatant collected via centrifugation for IL-12 and IL-15 quantification. For IL-12 and IL-15, supernatants analyzed by Luminex (R&D IL12/IL15 kit).

TABLE 22
Additional IL-12 and IL-15 Regulatable TF Expression Constructs

| SB # | Domain | Sequence |
|---|---|---|
| SB02667 | p65-based | MSRPGERPFQCRICMRNFSRRHGLDRHTRTHTGEKPFQCRICMRNFS |
| | synTF | DHSSLKRHLRTHTGSQKPFQCRICMRNFSVRHNLTRHLRTHTGEKPF |
| | | QCRICMRNFSDHSNLSRHLKTHTGSQKPFQCRICMRNFSQRSSLVRH |
| | | LRTHTGEKPFQCRICMRNFSESGHLKRHLRTHLRGSTCRDYKDHDG |
| | | DYKDHDIDYKDDDDKMAPKKKRKVGIHGVPGGLEGGGGSGGTED |
| | | VVCCHSIYGKKKGDIDTYRYIGSSGTGCVVIVGRIVLSGSGTSAPITA |
| | | YAQQTRGLLGCIITSLTGRDKNQVEGEVQIVSTATQTFLATCINGVC |
| | | WAVYHGAGTRTIASPKGPVIQMYTNVDQDLVGWPAPQGSRSLTPC |
| | | TCGSSDLYLVTRHADVIPVRRRGDSRGSLLSPRPISYLKGSSGGPLLC |
| | | PAGHAVGLFRAAVCTRGVAKAVDFIPVENLETTMRSPVFTDNSSPP |
| | | AVTLTHPITKIDREVLYQEFDEMEECSQHYPYDVPDYAGGGGSGGT |
| | | DEFPTMVFPSGQISQASALAPAPPQVLPQAPAPAPAPAMVSALAQAP |
| | | APVPVLAPGPPQAVAPPAPKPTQAGEGTLSEALLQLQFDDEDLGALL |
| | | GNSTDPAVFTDLASVDNSEFQQLLNQGIPVAPHTTEPMLMEYPEAIT |
| | | RLVTGAQRPPDPAPAPLGAPGLPNGLLSGDEDFSSIADMDFSALLSQI |
| | | SSQL (SEQ ID NO: 145) |
| SB02667 | IL-12 | See SEQ ID NO: 93 |
| SB02667 | insulator + | tccccagcatgcctgctattctcttcccaatcctcccccttg |
| | inducible | ctgtcctgccccaccccaccccccagaatagaatgacacctac |
| | YB-TATA | tcagacaatgcgatgcaatttcctcattttattaggaaag |
| | promoter | gacagtgggagtggcaccttccagggtcaaggaaggcacgggg |
| | IL12 | gaggggcaaacaacagatggctggcaactagaaggcacagttaACT |
| | inverted | GGCGTTCAGGTAGGACATCACGCGGTCAATGGTCACGGCTCGAA |
| | strand, | TGCGGAAAGCGTGCAACAGGATGCACAGCTTGATCTTGGTCTTGT |
| | forward | AGAAGTCCGGTTCTTCCAGGCTGGACTTTTGAGGCACAGTCTCGG |
| | strand | AGTTGAAATTCAGGGCCTGCATCAGTTCATCAATCACGGCGAGC |
| | SFFV | ATATTCTGGTCCAGGAAGATCTGCCGCTTCGGGTCCATGAGCAGC |
| | synTF-p65 | TTGGCGTTCATGGTCTTGAACTCGACCTGATACATCTTCAGATCTT |
| | | CGTAGATCGAGGAAAGACAGAGCGCCATCATGAATGAGGTCTTT |
| | | CTCGACGCCAGGCAGCTGCCGTTAGTGATAAAGCTTGTCTCGCGG |
| | | GAGTTCAGACACGATTCGTTCTTGGTCAGTTCCAGCGGCAGGCAG |
| | | GCTTCCACGGTCGAGGTCTTGTCCTTGGTGATGTCCTCGTGATCA |
| | | ATTTCTTCCGAGGTGCAGGGGTAGAACTCAAGGGTCTGGCGGGC |
| | | CTTCTGCAACATGTTCGACACAGCCCTCAGGAGGTTTTGGGAGTG |
| | | GTGTAGGCACGGGAACATTCCAGGGTCGGGGGTTGCCACAGGGA |
| | | GGTTCCGGGAACCTCCTCCGGAGCCTCCTCCTGAACCTCCGCCTG |
| | | ATCCGCCACCGGAACAAGGCACGCTGGCCCATTCGCTCCAGGAG |
| | | GACGAGTAGTATCTATCCTGCGCCCGGACGCTGATTGACGCGTTC |
| | | TTCCGACAAATCACAGTGGCGGAGGTTTTGTCGGTGAACACCCG |
| | | GTCTTTCTTCTCCCGTTTGGACTTTCCCTGCACTTGCACACAGAAA |
| | | GTGAGCGAGAAGTATGAGTGCGGGGTGCTCCAAGTGTCTGGATA |
| | | TTCCCAAGACACTTCCACTTGGCGGGAGTTCTTGAGTGGCTTCAG |
| | | CTGCAAGTTCTTGGGGGGGTCAGGCTTGATGATGTCGCGGATAA |
| | | AGAAGGAGGAAGTGTAGTTCTCGTATTTCAGCTTATGCACGGCAT |
| | | CGACCATGACCTCGATAGGCAGGGACTCTTCCGCGGCAGGGCAG |
| | | GCGCTGTCCTCCTGGCATTCCACGGAGTACTCATATTCCTTGTTGT |
| | | CTCCCCTGACTCTCTCGGCGGACAGAGTGGCGGCTCCACAGGTCA |
| | | CGCCCTGAGGATCGCTTGATCCCCGTGACGACTTCACGGAGAAA |
| | | GTCAGGTCGGTGGAGATTGTCGTCAGCCACCAACAGGTGAACCG |
| | | ACCGCTGTAGTTCTTGGCTTCGCAGCGGAGGAAGGTCTTGTTCTT |
| | | CGGTTCTTTTTGGTCCTTGAGGATGTCAGTGGACCAGATTCCATC |
| | | CTCTTTCTTGTGCAGCAGCAGCAGGGAGTGGGACAGCACTTCGCC |
| | | ACCCTTGTGGCAAGTGTACTGGCCCGCGTCGCCGAACTCCTTGAC |
| | | TTGAATGGTCAGGGTCTTTCCGCTTCCGAGCACCTCGGAGCTCTG |
| | | ATCCAGGGTCCAGGTTATGCCGTCCTCTTCTGGCGTATCGCAAGT |
| | | CAGCACGACCATTTCTCCAGGGGCGTCCGGGTACCAATCCAGCTC |
| | | GACCACGTAGACGTCCTTCTTCAGTTCCCAAATGGCGACCAGAGG |
| | | GGAAGCGAGGAACACAAGGGAGAACCAGGAGATGACGAGTTGC |
| | | TGATGGCACATCATGGTGGCGACACCGGTACGCGTTGGCCCCCAT |
| | | TATATACCCTCTAGAACTAGTtatccactccgtgtaagggagag |
| | | tgagcctcttacgaatgCGCGACATCGGCTACGCCgttccgagg |
| | | cgactgatacgCGCGACATCGGCTACGCCgtacgaaggcagtc |
| | | cgattgCGCGACATCGGCTACGCCgacctttactgagacggga |
| | | gCGCGACATCGGCTACGCCgaaggcgttgcgaatcctcatgcga |
| | | ttgttacgaaacccgTTAATTAAAGAGCGAGATTCCGTCTCAA |
| | | AGAAAAAAAAAGTAATGAAATGAATAAAATGAGTCCTAGAGCC |
| | | AGTAAATGTCGTAAATGTCTCAGCTAGTCAGGTAGTAAAAGGTC |
| | | TCAACTAGGCAGTGGCAGAGCAGGATTCAAATTCAGGGCTGTT |
| | | GTGATGCCTCCGCAGACTCTGAGCGCCACCTGGTGGTAATTTGT |
| | | CTGTGCCTCTTCTGACGTGGAAGAACAGCAACTAACACACTA |
| | | ACACGGCATTTACTATGGGCCAGCCATTGTCCATCTAGATGGc |
| | | cgataaaataaaagattttatttagtctccagaaaaaggggg |
| | | gaatgaaagaccccacctgtaggtttggcaagctagctgcagt |
| | | aacgccattttgcaaggcatggaaaaataccaaaccaagaata |
| | | gagaagttcagatcaagggcgggtacatgaaaatagctaacgtt |
| | | gggccaaacaggatatctgcggtgagcagtttcggccccggcc |
| | | cggggccaagaacagatggtcaccgcagtttcggccccggcccg |
| | | aggccaagaacagatggtccccagatatggcccaaccctcag |
| | | cagtttcttaagacccatcagatgtttccaggctcccccaa |
| | | ggacctgaaatgaccctgcgccttatttgaattaaccaatcag |
| | | cctgcttctcgcttctgttcgcgcgcttctgcttcccgagctcta |
| | | taaaagagctcacaacccctcactcggcgcgccagtcctccgacag |
| | | actgagtcgcccgggGGATCCGCCACCATGAGTAGACCTGGCGAAA |
| | | GACCATTTCAGTGCAGAATTTGTATGCGGAACTTCAGCAGAAGG |
| | | CACGGCCTGGACAGACACACCAGAACACACACAGGCGAGAAGC |
| | | CTTTCCAGTGTAGAATCTGTATGCGCAATTTCAGCGACCACAGCA |
| | | GCCTGAAGCGGCACCTGAGAACCCATACCGGCAGCCAGAAACCA |
| | | TTTCAATGCCGCATCTGTATGAGAAACTTCTCCGTGCGGCACAAC |
| | | CTGACCAGACACCTGAGGACACACACCGGGGAGAAACCCTTCCA |
| | | GTGCCGGATATGCATGAGGAATTTCTCCGACCACTCCAACCTGAG |
| | | CCGCCACCTGAAAACTCACACCGGCTCTCAAAAGCCATTTCAGTG |
| | | TCGTATATGTATGCGGAATTTTTCCCAGCGGAGCAGCCTCGTGCG |
| | | CCATCTGAGGACTCATACTGGCGAAAAGCCCTTCCAATGTCGCAT |
| | | ATGCATGCGCAACTTTAGCGAGTCCGGCCACCTGAAGAGACATC |
| | | TGCGGACACACCTGAGAGGCAGCACCTGTAGAGACTACAAGGAC |
| | | CACGACGGCGATTATAAGGATCACGACATCGACTACAAAGACGA |
| | | CGATGACAAGATGGCCCCTAAGAAGAAGCGGAAAGTCGGCATCC |
| | | ATGGCGTGCCAGGTGGACTTGAAGGTGGCGGAGGATCTGGCGGC |
| | | ACAGAGGATGTTGTGTGCTGCCACAGCATCTACGGCAAGAAGAA |
| | | GGGCGACATCGATACCTATCGGTACATCGGCAGCAGCGGCACAG |
| | | GCTGCGTTGTGATCGTGGGAAGAATCGTGCTGAGCGGCTCTGGC |
| | | ACAAGCGCCCCTATTACAGCCTACGCTCAGCAGACAAGAGGCCT |
| | | GCTGGGCTGCATCATCACAAGCCTGACCGGCAGAGACAAGAACC |
| | | AGGTGGAAGGCGAGGTGCAGATCGTGTCTACAGCTACCCAGACC |
| | | TTCCTGGCCACCTGTATCAATGGCGTGTGCTGGGCCGTGTATCAC |
| | | GGCGCTGGAACCAGAACAATCGCCTCTCCTAAGGGACCCGTGAT |
| | | CCAGATGTACACCAACGTGGACCAGGACCTCGTTGGCTGGCCTG |
| | | CTCCTCAGGGAAGTAGAAGCCTGACACCTTGCACCTGTGGCTCCA |
| | | GCGATCTGTACCTGGTCACCAGACACGCCGACGTGATCCCTGTCA |
| | | GAAGAAGAGGAGATTCCAGAGGCAGCCTGCTGAGCCCTAGACCT |
| | | ATCAGCTACCTGAAGGGCAGCTCTGGCGGACCTCTGCTTTGTCCT |
| | | GCTGGACATGCCGTGGGCCTGTTTAGAGCCGCCGTGTGTACAAG |
| | | AGGCGTGGCAAAGGCCGTGGACTTCATCCCCGTGGAAAACCTGG |
| | | AAACCACCATGCGGAGCCCCGTGTTCACCGACAATTCTAGCCCTC |
| | | CAGCCGTGACACTGACACACCCCATCACCAAGATCGACAGAGAG |
| | | GTGCTGTACCAAGAGTTCGACGAGATGGAAGAGTGCAGCCAGCA |
| | | CTACCCCTACGACGTGCCAGATTATGCTGGCGGAGGTGGCAGCG |
| | | GAGGCACCGATGAATTTCCAACCATGGTGTTCCCCAGCGGCCAG |
| | | ATCTCTCAGGCATCTGCTCTTGCTCCAGCTCCACCTCAGGTTCTGC |
| | | CTCAAGCTCCTGCTCCGGCTCCTGCACCAGCTATGGTGTCTGCAC |
| | | TTGCTCAGGCACCAGCTCCAGTGCCTGTTCTTGCTCCTGGACCTC |
| | | CTCAGGCTGTTGCTCCACCAGCACCTAAACCTACACAGGCCGGCG |
| | | AGGGAACACTGTCTGAAGCCCTGCTGCAACTCCAGTTCGATGAC |
| | | GAAGATCTGGGCGCCCTGCTGGGAAACTCTACAGATCCTGCCGT |
| | | GTTTACCGATCTGGCCAGCGTGGACAACAGCGAGTTTCAGCAGCT |
| | | CCTGAACCAGGGCATCCCAGTGGCTCCTCACACCACAGAGCCTAT |
| | | GCTGATGGAATACCCCGAGGCCATCACCAGACTGGTCACCGGCG |
| | | CACAAAGACCACCTGATCCTGCTCCAGCACCGCTTGGAGCACCTG |
| | | GACTGCCTAATGGACTGCTGTCTGGCGACGAGGACTTCAGTTCTA |
| | | TCGCTGATATGGATTTCTCTGCCCTTCTGTCTCAGATTAGTAGCCA |
| | | GCTGTAA (SEQ ID NO: 146) |
| SB02668 | p65-based | See SEQ ID NO: 145 |
| | synTF | |
| SB02668 | IL-12 | See SEQ ID NO: 93 |
| SB02668 | IL-15 | See SEQ ID NO: 141 |
| SB02668 | 2A peptide | See SEQ ID NO: 142 |
| SB02668 | insulator + | tccccagcatgcctgctattctcttcccaatcctcccccttgct |
| | inducible | gtcctgccccaccccaccccccagaatagaatgacacctactcag |
| | YB-TATA | acaatgcgatgcaatttcctcattttattaggaaaggacagt |
| | promoter | gggagtggcaccttccagggtcaaggaaggcacgggggaggggc |
| | IL12_2A_ | aaacaacagatggctggcaactagaaggcacagttaGCT |
| | IL-15 | GGTGTTGATGAACATCTGCACGATGTGCACGAAGCTCTGCAGGA |
| | inverted | ACTCTTTGATATTCTTTTCTTCCAGTTCCTCGCATTCTTTGCAGCC |
| | strand, | GGACTCGGTCACATTGCCGTTGGAGGACAGGCTGTTGTTGGCCAG |
| | forward | GATGATCAGGTTTTCCACGGTATCGTGGATGGAGGCGTCGCCGCT |
| | strand | TTCCAGGCTGATCACTTGCAGCTCGAGCAGAAAGCACTTCATGGC |
| | SFFV | GGTCACTTTACAGCTAGGGTGCACGTCGCTCTCGGTGTACAGTGT |
| | synTF-p65 | GGCGTCGATGTGCATGCTCTGGATCAGATCCTCGATCTTCTTCAG |
| | | ATCGCTGATCACGTTGACCCAGTTGCCTGTAGAGCCGGGCACCCA |
| | | AAGAAGCAGCACCCACAGCAGCAGTGTGTCGGTTTCCATAGGTC |
| | | CAGGGTTTTCCTCCACGTCTCCACACGTCAGTAGACTCCCGCGTC |
| | | CCTCGCCAGATCCTCTCTTCCGTCTACTGGCGTTCAGGTAGGACA |
| | | TCACGCGGTCAATGGTCACGGCTCGAATGCGGAAAGCGTGCAAC |
| | | AGGATGCACAGCTTGATCTTGGTCTTGTAGAAGTCCGGTTCTTCC |
| | | AGGCTGGACTTTTGAGGCACAGTCTCGGAGTTGAAATTCAGGGC |
| | | CTGCATCAGTTCATCAATCACGGCGAGCATATTCTGGTCCAGGAA |
| | | GATCTGCCGCTTCGGGTCCATGAGCAGCTTGGCGTTCATGGTCTT |
| | | GAACTCGACCTGATACATCTTCAGATCTTCGTAGATCGAGGAAAG |
| | | ACAGAGCGCCATCATGAATGAGGTCTTTCTCGACGCCAGGCAGC |
| | | TGCCGTTAGTGATAAAGCTTGTCTCGCGGGAGTTCAGACACGATT |
| | | CGTTCTTGGTCAGTTCCAGCGGCAGGCAGGCTTCCACGGTCGAGG |
| | | TCTTGTCCTTGGTGATGTCCTCGTGATCAATTTCTTCCGAGGTGCA |
| | | GGGGTAGAACTCAAGGGTCTGGCGGGCCTTCTGCAACATGTTCG |
| | | ACACAGCCCTCAGGAGGTTTTGGGAGTGGTGTAGGCACGGGAAC |
| | | ATTCCAGGGTCGGGGGTTGCCACAGGGAGGTTCCGGGAACCTCC |
| | | TCCGGAGCCTCCTCCTGAACCTCCGCCTGATCCGCCACCGGAACA |
| | | AGGCACGCTGGCCCATTCGCTCCAGGAGGACGAGTAGTATCTAT |
| | | CCTGCGCCCGGACGCTGATTGACGCGTTCTTCCGACAAATCACAG |
| | | TGGCGGAGGTTTTGTCGGTGAACACCCGGTCTTTCTTCTCCCGTTT |
| | | GGACTTTCCCTGCACTTGCACACAGAAAGTGAGCGAGAAGTATG |
| | | AGTGCGGGGTGCTCCAAGTGTCTGGATATTCCCAAGACACTTCCA |
| | | CTTGGCGGGAGTTCTTGAGTGGCTTCAGCTGCAAGTTCTTGGGGG |
| | | GGTCAGGCTTGATGATGTCGCGGATAAAGAAGGAGGAAGTGTAG |
| | | TTCTCGTATTTCAGCTTATGCACGGCATCGACCATGACCTCGATA |
| | | GGCAGGGACTCTTCCGCGGCAGGGCAGGCGCTGTCCTCCTGGCA |
| | | TTCCACGGAGTACTCATATTCCTTGTTGTCTCCCCTGACTCTCTCG |
| | | GCGGACAGAGTGGCGGCTCCACAGGTCACGCCCTGAGGATCGCT |
| | | TGATCCCCGTGACGACTTCACGGAGAAAGTCAGGTCGGTGGAGA |
| | | TTGTCGTCAGCCACCAACAGGTGAACCGACCGCTGTAGTTCTTGG |
| | | CTTCGCAGCGGAGGAAGGTCTTGTTCTTCGGTTCTTTTTGGTCCTT |
| | | GAGGATGTCAGTGGACCAGATTCCATCCTCTTTCTTGTGCAGCAG |
| | | CAGCAGGGAGTGGGACAGCACTTCGCCACCCTTGTGGCAAGTGT |
| | | ACTGGCCCGCGTCGCCGAACTCCTTGACTTGAATGGTCAGGGTCT |
| | | TTCCGCTTCCGAGCACCTCGGAGCTCTGATCCAGGGTCCAGGTTA |
| | | TGCCGTCCTCTTCTGGCGTATCGCAAGTCAGCACGACCATTTCTC |
| | | CAGGGGCGTCCGGGTACCAATCCAGCTCGACCACGTAGACGTCC |
| | | TTCTTCAGTTCCCAAATGGCGACCAGAGGGGAAGCGAGGAACAC |
| | | AAGGGAGAACCAGGAGATGACGAGTTGCTGATGGCACATCATGG |
| | | TGGCGACACCGGTACGCGTTGGCCCCCATTATATACCCTCTAGAA |
| | | CTAGTtatccactccgtgtaagggagagtgagcctcttacgaa |
| | | tgCGCGACATCGGCTACGCCgttccgaggcgactgatacgCGCG |
| | | ACATCGGCTACGCCgtacgaaggcagtccgattgCGCGACAT |
| | | CGGCTACGCCgacctttactgagacgggagCGCGACAT |
| | | CGGCTACGCCgaaggcgttgcgaatcctcatgcgattgtta |
| | | cgaaacccgTTAATTAAAGAGCGAGATTCCGTCTCAAAGAAA |
| | | AAAAAAGTAATGAAATGAATAAAATGAGTCCTAGAGCCAGTA |
| | | AATGTCGTAAATGTCTCAGCTAGTCAGGTAGTAAAAGGTCTC |
| | | AACTAGGCAGTGGCAGAGCAGGATTCAAATTCAGGGCTG |
| | | TTGTGATGCCTCCGCAGACTCTGAGCGCCACCTGGTGGTAAT |
| | | TTGTCTGTGCCTCTTCTGACGTGGAAGAACAGCAACTAACAC |
| | | ACTAACACGGCATTTACTATGGGCCAGCCATTGTCCATCTAGA |
| | | TGGccgataaaataaaagattttatttagtctccagaaaaa |
| | | ggggggaatgaaagaccccacctgtaggtttggcaagctagc |
| | | tgcagtaacgccattttgcaaggcatggaaaaataccaaacc |
| | | aagaatagagaagttcagatcaagggcgggtacatgaaaatag |
| | | ctaacgttgggccaaacaggatatctgcggtgagcagtttcg |
| | | gccccggcccggggccaagaacagatggtcaccgcagtttc |
| | | ggccccggcccgaggccaagaacagatggtccccagatatgg |
| | | cccaaccctcagcagtttcttaagacccatcagatgtttcc |
| | | aggctcccccaaggacctgaaatgaccctgcgccttatttga |
| | | attaaccaatcagcctgcttctcgcttctgttcgcgcgct |
| | | tctgcttcccgagctctataaaagagctcacaacccctcac |
| | | tcggcgcgccagtcctccgacagactgagtcgcccgggG |
| | | GATCCGCCACCATGAGTAGACCTGGCGAAAGACCATTTCAGTGC |
| | | AGAATTTGTATGCGGAACTTCAGCAGAAGGCACGGCCTGGACAG |
| | | ACACACCAGAACACACACAGGCGAGAAGCCTTTCCAGTGTAGAA |
| | | TCTGTATGCGCAATTTCAGCGACCACAGCAGCCTGAAGCGGCAC |
| | | CTGAGAACCCATACCGGCAGCCAGAAACCATTTCAATGCCGCAT |
| | | CTGTATGAGAAACTTCTCCGTGCGGCACAACCTGACCAGACACCT |
| | | GAGGACACACACCGGGGAGAAACCCTTCCAGTGCCGGATATGCA |
| | | TGAGGAATTTCTCCGACCACTCCAACCTGAGCCGCCACCTGAAAA |
| | | CTCACACCGGCTCTCAAAAGCCATTTCAGTGTCGTATATGTATGC |
| | | GGAATTTTTCCCAGCGGAGCAGCCTCGTGCGCCATCTGAGGACTC |
| | | ATACTGGCGAAAAGCCCTTCCAATGTCGCATATGCATGCGCAACT |
| | | TTAGCGAGTCCGGCCACCTGAAGAGACATCTGCGGACACACCTG |
| | | AGAGGCAGCACCTGTAGAGACTACAAGGACCACGACGGCGATTA |
| | | TAAGGATCACGACATCGACTACAAAGACGACGATGACAAGATGG |
| | | CCCCTAAGAAGAAGCGGAAAGTCGGCATCCATGGCGTGCCAGGT |
| | | GGACTTGAAGGTGGCGGAGGATCTGGCGGCACAGAGGATGTTGT |
| | | GTGCTGCCACAGCATCTACGGCAAGAAGAAGGGCGACATCGATA |
| | | CCTATCGGTACATCGGCAGCAGCGGCACAGGCTGCGTTGTGATC |
| | | GTGGGAAGAATCGTGCTGAGCGGCTCTGGCACAAGCGCCCCTAT |
| | | TACAGCCTACGCTCAGCAGACAAGAGGCCTGCTGGGCTGCATCA |
| | | TCACAAGCCTGACCGGCAGAGACAAGAACCAGGTGGAAGGCGA |
| | | GGTGCAGATCGTGTCTACAGCTACCCAGACCTTCCTGGCCACCTG |
| | | TATCAATGGCGTGTGCTGGGCCGTGTATCACGGCGCTGGAACCA |
| | | GAACAATCGCCTCTCCTAAGGGACCCGTGATCCAGATGTACACC |
| | | AACGTGGACCAGGACCTCGTTGGCTGGCCTGCTCCTCAGGGAAG |
| | | TAGAAGCCTGACACCTTGCACCTGTGGCTCCAGCGATCTGTACCT |
| | | GGTCACCAGACACGCCGACGTGATCCCTGTCAGAAGAAGAGGAG |
| | | ATTCCAGAGGCAGCCTGCTGAGCCCTAGACCTATCAGCTACCTGA |
| | | AGGGCAGCTCTGGCGGACCTCTGCTTTGTCCTGCTGGACATGCCG |
| | | TGGGCCTGTTTAGAGCCGCCGTGTGTACAAGAGGCGTGGCAAAG |
| | | GCCGTGGACTTCATCCCCGTGGAAAACCTGGAAACCACCATGCG |
| | | GAGCCCCGTGTTCACCGACAATTCTAGCCCTCCAGCCGTGACACT |
| | | GACACACCCCATCACCAAGATCGACAGAGAGGTGCTGTACCAAG |
| | | AGTTCGACGAGATGGAAGAGTGCAGCCAGCACTACCCCTACGAC |
| | | GTGCCAGATTATGCTGGCGGAGGTGGCAGCGGAGGCACCGATGA |
| | | ATTTCCAACCATGGTGTTCCCCAGCGGCCAGATCTCTCAGGCATC |
| | | TGCTCTTGCTCCAGCTCCACCTCAGGTTCTGCCTCAAGCTCCTGCT |
| | | CCGGCTCCTGCACCAGCTATGGTGTCTGCACTTGCTCAGGCACCA |
| | | GCTCCAGTGCCTGTTCTTGCTCCTGGACCTCCTCAGGCTGTTGCTC |
| | | CACCAGCACCTAAACCTACACAGGCCGGCGAGGGAACACTGTCT |
| | | GAAGCCCTGCTGCAACTCCAGTTCGATGACGAAGATCTGGGCGC |
| | | CCTGCTGGGAAACTCTACAGATCCTGCCGTGTTTACCGATCTGGC |
| | | CAGCGTGGACAACAGCGAGTTTCAGCAGCTCCTGAACCAGGGCA |
| | | TCCCAGTGGCTCCTCACACCACAGAGCCTATGCTGATGGAATACC |
| | | CCGAGGCCATCACCAGACTGGTCACCGGCGCACAAAGACCACCT |
| | | GATCCTGCTCCAGCACCGCTTGGAGCACCTGGACTGCCTAATGGA |
| | | CTGCTGTCTGGCGACGAGGACTTCAGTTCTATCGCTGATATGGAT |
| | | TTCTCTGCCCTTCTGTCTCAGATTAGTAGCCAGCTGTAA |
| SB02670 | VPR-based | MSRPGERPFQCRICMRNFSRRHGLDRHTRTHTGEKPFQCRICMRNFS |
| | synTF | DHSSLKRHLRTHTGSQKPFQCRICMRNFSVRHNLTRHLRTHTGEKPF |
| | | QCRICMRNFSDHSNLSRHLKTHTGSQKPFQCRICMRNFSQRSSLVRH |
| | | LRTHTGEKPFQCRICMRNFSESGHLKRHLRTHLRGSTCRDYKDHDG |
| | | DYKDHDIDYKDDDDKMAPKKKRKVGIHGVPGGLEGGGGSGGTED |
| | | WCCHSIYGKKKGDIDTYRYIGSSGTGCVVIVGRIVLSGSGTSAPITA |
| | | YAQQTRGLLGCIITSLTGRDKNQVEGEVQIVSTATQTFLATCINGVC |
| | | WAVYHGAGTRTIASPKGPVIQMYTNVDQDLVGWPAPQGSRSLTPC |
| | | TCGSSDLYLVTRHADVIPVRRRGDSRGSLLSPRPISYLKGSSGGPLLC |
| | | PAGHAVGLFRAAVCTRGVAKAVDFIPVENLETTMRSPVFTDNSSPP |
| | | AVTLTHPITKIDREVLYQEFDEMEECSQHYPYDVPDYAGGGGSGGT |
| | | EASGSGRADALDDFDLDMLGSDALDDFDLDMLGSDALDDFDLDML |
| | | GSDALDDFDLDMLINSRSSGSPKKKRKVGSQYLPDTDDRHRIEEKR |
| | | KRTYETFKSIMKKSPFSGPTDPRPPPRRIAVPSRSSASVPKPAPQPYPF |
| | | TSSLSTINYDEFPTMVFPSGQISQASALAPAPPQVLPQAPAPAPAPAM |
| | | VSALAQAPAPVPVLAPGPPQAVAPPAPKPTQAGEGTLSEALLQLQFD |
| | | DEDLGALLGNSTDPAVFTDLASVDNSEFQQLLNQGIPVAPHTTEPML |
| | | MEYPEAITRLVTGAQRPPDPAPAPLGAPGLPNGLLSGDEDFSSIADM |
| | | DFSALLGSGSGSRDSREGMFLPKPEAGSAISDVFEGREVCQPKRIRPF |
| | | HPPGSPWANRPLPASLAPTPTGPVHEPVGSLTPAPVPQPLDPAPAVTP |
| | | EASHLLEDPDEETSQAVKALREMADTVIPQKEEAAICGQMDLSHPPP |
| | | RGHLDELTTTLESMTEDLNLDSPLTPELNEILDTFLNDECLLHAMHIS |
| | | TGLSIFDTSLF (SEQ ID NO: 147) |
| SB02670 | IL-12 | See SEQ ID NO: 93 |
| SB02670 | insulator + | tccccagcatgcctgctattctcttcccaatcctcccccttg |
| | inducible | ctgtcctgccccaccccaccccccagaatagaatgacacctact |
| | YB-TATA | cagacaatgcgatgcaatttcctcattttattaggaaaggac |
| | promoter | agtgggagtggcaccttccagggtcaaggaaggcacgggggag |
| | IL12 | gggcaaacaacagatggctggcaactagaaggcacagttaACT |
| | inverted | GGCGTTCAGGTAGGACATCACGCGGTCAATGGTCACGGCTCGAA |
| | strand, | TGCGGAAAGCGTGCAACAGGATGCACAGCTTGATCTTGGTCTTGT |
| | forward | AGAAGTCCGGTTCTTCCAGGCTGGACTTTTGAGGCACAGTCTCGG |
| | strand | AGTTGAAATTCAGGGCCTGCATCAGTTCATCAATCACGGCGAGC |
| | SFFV | ATATTCTGGTCCAGGAAGATCTGCCGCTTCGGGTCCATGAGCAGC |
| | synTF- | TTGGCGTTCATGGTCTTGAACTCGACCTGATACATCTTCAGATCTT |
| | VPR | CGTAGATCGAGGAAAGACAGAGCGCCATCATGAATGAGGTCTTT |
| | | CTCGACGCCAGGCAGCTGCCGTTAGTGATAAAGCTTGTCTCGCGG |
| | | GAGTTCAGACACGATTCGTTCTTGGTCAGTTCCAGCGGCAGGCAG |
| | | GCTTCCACGGTCGAGGTCTTGTCCTTGGTGATGTCCTCGTGATCA |
| | | ATTTCTTCCGAGGTGCAGGGGTAGAACTCAAGGGTCTGGCGGGC |
| | | CTTCTGCAACATGTTCGACACAGCCCTCAGGAGGTTTTGGGAGTG |
| | | GTGTAGGCACGGGAACATTCCAGGGTCGGGGGTTGCCACAGGGA |
| | | GGTTCCGGGAACCTCCTCCGGAGCCTCCTCCTGAACCTCCGCCTG |
| | | ATCCGCCACCGGAACAAGGCACGCTGGCCCATTCGCTCCAGGAG |
| | | GACGAGTAGTATCTATCCTGCGCCCGGACGCTGATTGACGCGTTC |
| | | TTCCGACAAATCACAGTGGCGGAGGTTTTGTCGGTGAACACCCG |
| | | GTCTTTCTTCTCCCGTTTGGACTTTCCCTGCACTTGCACACAGAAA |
| | | GTGAGCGAGAAGTATGAGTGCGGGGTGCTCCAAGTGTCTGGATA |
| | | TTCCCAAGACACTTCCACTTGGCGGGAGTTCTTGAGTGGCTTCAG |
| | | CTGCAAGTTCTTGGGGGGGTCAGGCTTGATGATGTCGCGGATAA |
| | | AGAAGGAGGAAGTGTAGTTCTCGTATTTCAGCTTATGCACGGCAT |
| | | CGACCATGACCTCGATAGGCAGGGACTCTTCCGCGGCAGGGCAG |
| | | GCGCTGTCCTCCTGGCATTCCACGGAGTACTCATATTCCTTGTTGT |
| | | CTCCCCTGACTCTCTCGGCGGACAGAGTGGCGGCTCCACAGGTCA |
| | | CGCCCTGAGGATCGCTTGATCCCCGTGACGACTTCACGGAGAAA |
| | | GTCAGGTCGGTGGAGATTGTCGTCAGCCACCAACAGGTGAACCG |
| | | ACCGCTGTAGTTCTTGGCTTCGCAGCGGAGGAAGGTCTTGTTCTT |
| | | CGGTTCTTTTTGGTCCTTGAGGATGTCAGTGGACCAGATTCCATC |
| | | CTCTTTCTTGTGCAGCAGCAGCAGGGAGTGGGACAGCACTTCGCC |
| | | ACCCTTGTGGCAAGTGTACTGGCCCGCGTCGCCGAACTCCTTGAC |
| | | TTGAATGGTCAGGGTCTTTCCGCTTCCGAGCACCTCGGAGCTCTG |
| | | ATCCAGGGTCCAGGTTATGCCGTCCTCTTCTGGCGTATCGCAAGT |
| | | CAGCACGACCATTTCTCCAGGGGCGTCCGGGTACCAATCCAGCTC |
| | | GACCACGTAGACGTCCTTCTTCAGTTCCCAAATGGCGACCAGAGG |
| | | GGAAGCGAGGAACACAAGGGAGAACCAGGAGATGACGAGTTGC |
| | | TGATGGCACATCATGGTGGCGACACCGGTACGCGTTGGCCCCCAT |
| | | TATATACCCTCTAGAACTAGTtatccactccgtgtaagggaga |
| | | gtgagcctcttacgaatgCGCGACATCGGCTACGCCgttccg |
| | | aggcgactgatacgCGCGACATCGGCTACGCCgtacgaaggca |
| | | gtccgattgCGCGACATCGGCTACGCCgacctttactgagacgg |
| | | gagCGCGACATCGGCTACGCCgaaggcgttgcgaatcctcatg |
| | | cgattgttacgaaacccgTTAATTAAAGAGCGAGATTCCGTCT |
| | | CAAAGAAAAAAAAAGTAATGAAATGAATAAAATGAGTCCTAGA |
| | | GCCAGTAAATGTCGTAAATGTCTCAGCTAGTCAGGTAGTAAAAG |
| | | GTCTCAACTAGGCAGTGGCAGAGCAGGATTCAAATTCAGGGCT |
| | | GTTGTGATGCCTCCGCAGACTCTGAGCGCCACCTGGTGGTAATT |
| | | TGTCTGTGCCTCTTCTGACGTGGAAGAACAGCAACTAACACA |
| | | CTAACACGGCATTTACTATGGGCCAGCCATTGTCCATCTAGAT |
| | | GGccgataaaataaaagattttatttagtctccagaaaaagg |
| | | ggggaatgaaagaccccacctgtaggtttggcaagctagctg |
| | | cagtaacgccattttgcaaggcatggaaaaataccaaaccaag |
| | | aatagagaagttcagatcaagggcgggtacatgaaaatagctaa |
| | | cgttgggccaaacaggatatctgcggtgagcagtttcggcccc |
| | | ggcccggggccaagaacagatggtcaccgcagtttcggccccgg |
| | | cccgaggccaagaacagatggtccccagatatggcccaaccct |
| | | cagcagtttcttaagacccatcagatgtttccaggctccccc |
| | | aaggacctgaaatgaccctgcgccttatttgaattaaccaatcagc |
| | | ctgcttctcgcttctgttcgcgcgcttctgcttcccgagctctat |
| | | aaaagagctcacaacccctcactcggcgcgccagtcctccgaca |
| | | gactgagtcgcccgggGGATCCGCCACCATGAGTAGACCTGGCGAAA |
| | | GACCATTTCAGTGCAGAATTTGTATGCGGAACTTCAGCAGAAGG |
| | | CACGGCCTGGACAGACACACCAGAACACACACAGGCGAGAAGC |
| | | CTTTCCAGTGTAGAATCTGTATGCGCAATTTCAGCGACCACAGCA |
| | | GCCTGAAGCGGCACCTGAGAACCCATACCGGCAGCCAGAAACCA |
| | | TTTCAATGCCGCATCTGTATGAGAAACTTCTCCGTGCGGCACAAC |
| | | CTGACCAGACACCTGAGGACACACACCGGGGAGAAACCCTTCCA |
| | | GTGCCGGATATGCATGAGGAATTTCTCCGACCACTCCAACCTGAG |
| | | CCGCCACCTGAAAACTCACACCGGCTCTCAAAAGCCATTTCAGTG |
| | | TCGTATATGTATGCGGAATTTTTCCCAGCGGAGCAGCCTCGTGCG |
| | | CCATCTGAGGACTCATACTGGCGAAAAGCCCTTCCAATGTCGCAT |
| | | ATGCATGCGCAACTTTAGCGAGTCCGGCCACCTGAAGAGACATC |
| | | TGCGGACACACCTGAGAGGCAGCACCTGTAGAGACTACAAGGAC |
| | | CACGACGGCGATTATAAGGATCACGACATCGACTACAAAGACGA |
| | | CGATGACAAGATGGCCCCTAAGAAGAAGCGGAAAGTCGGCATCC |
| | | ATGGCGTGCCAGGTGGACTTGAAGGTGGCGGAGGATCTGGCGGC |
| | | ACAGAGGATGTTGTGTGCTGCCACAGCATCTACGGCAAGAAGAA |
| | | GGGCGACATCGATACCTATCGGTACATCGGCAGCAGCGGCACAG |
| | | GCTGCGTTGTGATCGTGGGAAGAATCGTGCTGAGCGGCTCTGGC |
| | | ACAAGCGCCCCTATTACAGCCTACGCTCAGCAGACAAGAGGCCT |
| | | GCTGGGCTGCATCATCACAAGCCTGACCGGCAGAGACAAGAACC |
| | | AGGTGGAAGGCGAGGTGCAGATCGTGTCTACAGCTACCCAGACC |
| | | TTCCTGGCCACCTGTATCAATGGCGTGTGCTGGGCCGTGTATCAC |
| | | GGCGCTGGAACCAGAACAATCGCCTCTCCTAAGGGACCCGTGAT |
| | | CCAGATGTACACCAACGTGGACCAGGACCTCGTTGGCTGGCCTG |
| | | CTCCTCAGGGAAGTAGAAGCCTGACACCTTGCACCTGTGGCTCCA |
| | | GCGATCTGTACCTGGTCACCAGACACGCCGACGTGATCCCTGTCA |
| | | GAAGAAGAGGAGATTCCAGAGGCAGCCTGCTGAGCCCTAGACCT |
| | | ATCAGCTACCTGAAGGGCAGCTCTGGCGGACCTCTGCTTTGTCCT |
| | | GCTGGACATGCCGTGGGCCTGTTTAGAGCCGCCGTGTGTACAAG |
| | | AGGCGTGGCAAAGGCCGTGGACTTCATCCCCGTGGAAAACCTGG |
| | | AAACCACCATGCGGAGCCCCGTGTTCACCGACAATTCTAGCCCTC |
| | | CAGCCGTGACACTGACACACCCCATCACCAAGATCGACAGAGAG |
| | | GTGCTGTACCAAGAGTTCGACGAGATGGAAGAGTGCAGCCAGCA |
| | | CTACCCCTACGACGTGCCAGATTATGCTGGCGGAGGTGGCAGCG |
| | | GAGGCACCGAAGCCTCTGGAAGCGGCAGAGCTGACGCCCTGGAT |
| | | GACTTCGACCTGGATATGCTGGGCAGCGACGCTCTGGACGATTTT |
| | | GACCTCGACATGCTGGGATCTGATGCACTCGACGATTTCGATTTG |
| | | GACATGCTCGGCAGTGATGCCTTGGACGACTTTGATCTTGATATG |
| | | CTCATCAACAGCCGGTCCAGCGGCAGCCCCAAGAAAAAAAGAAA |
| | | AGTGGGCTCCCAGTACCTGCCTGACACCGACGACAGACACCGGA |
| | | TCGAGGAAAAGCGGAAGCGGACCTACGAGACATTCAAGAGCATC |
| | | ATGAAGAAGTCCCCATTCAGCGGCCCCACCGATCCTAGACCTCCA |
| | | CCTAGAAGAATCGCCGTGCCTAGCAGATCTAGCGCCTCCGTGCCT |
| | | AAACCTGCTCCTCAGCCTTATCCTTTCACCAGCAGCCTGAGCACC |
| | | ATCAACTACGACGAGTTCCCTACCATGGTGTTCCCCAGCGGCCAG |
| | | ATCTCTCAGGCTTCTGCTCTTGCTCCAGCTCCTCCTCAGGTTCTGC |
| | | CTCAAGCTCCTGCACCAGCACCGGCTCCAGCTATGGTTTCTGCTT |
| | | TGGCTCAGGCCCCTGCTCCTGTGCCTGTTCTTGCTCCTGGACCACC |
| | | TCAGGCTGTTGCTCCTCCTGCTCCAAAACCTACACAGGCCGGCGA |
| | | AGGCACACTGTCTGAAGCTCTGCTGCAGCTCCAGTTCGATGACGA |
| | | AGATCTGGGCGCCCTGCTGGGCAATTCTACAGATCCTGCCGTGTT |
| | | TACCGATCTGGCCAGCGTGGACAACAGCGAGTTTCAGCAGCTCCT |
| | | GAATCAGGGCATCCCTGTGGCTCCTCACACCACCGAACCTATGCT |
| | | GATGGAATACCCCGAGGCCATCACCAGACTGGTCACCGGTGCTC |
| | | AAAGACCACCTGATCCAGCTCCAGCACCACTGGGAGCACCTGGA |
| | | CTGCCTAATGGACTGCTGTCTGGCGACGAGGACTTCAGCTCTATC |
| | | GCCGACATGGATTTCTCTGCCCTGCTCGGCTCTGGCAGCGGCTCT |
| | | AGAGATAGCAGAGAAGGCATGTTCCTGCCTAAGCCTGAGGCCGG |
| | | CTCTGCCATCTCCGATGTGTTCGAGGGAAGAGAAGTGTGCCAGCC |
| | | TAAGCGGATCCGGCCTTTTCACCCTCCTGGAAGCCCTTGGGCCAA |
| | | CAGACCTCTGCCTGCTTCTCTGGCCCCTACACCAACAGGACCTGT |
| | | GCACGAACCTGTGGGCAGTCTGACCCCAGCTCCTGTTCCTCAACC |
| | | TCTGGATCCCGCTCCTGCTGTGACACCTGAAGCCTCTCATCTGCT |
| | | GGAAGATCCCGACGAAGAGACAAGCCAGGCCGTGAAGGCCCTG |
| | | AGAGAAATGGCCGACACAGTGATCCCTCAGAAAGAGGAAGCCGC |
| | | CATCTGCGGACAGATGGACCTGTCTCATCCTCCACCAAGAGGCCA |
| | | CCTGGACGAGCTGACAACCACACTGGAATCCATGACCGAGGACC |
| | | TGAACCTGGACAGCCCTCTGACACCCGAGCTGAACGAGATCCTG |
| | | GACACCTTCCTGAACGACGAGTGTCTGCTGCACGCCATGCACATC |
| | | TCTACCGGCCTGAGCATCTTCGACACCAGCCTGTTTTGA (SEQ ID |
| | | NO: 148) |
| SB02671 | IL-12 | See SEQ ID NO: 93 |
| SB02671 | IL-15 | See SEQ ID NO: 141 |
| SB02671 | 2A peptide | See SEQ ID NO: 142 |
| SB02671 | VPR-based | See SEQ ID NO: 147 |
| | synTF | |
| SB02671 | insulator + | tccccagcatgcctgctattctcttcccaatcctcccccttgct |
| | inducible | gtcctgccccaccccaccccccagaatagaatgacacctactcag |
| | YB-TATA | acaatgcgatgcaatttcctcattttattaggaaaggacagtg |
| | promoter | ggagtggcaccttccagggtcaaggaaggcacgggggaggg |
| | IL12_2A_ | gcaaacaacagatggctggcaactagaaggcacagttaGCT |
| | IL-15 | GGTGTTGATGAACATCTGCACGATGTGCACGAAGCTCTGCAGGA |
| | inverted | ACTCTTTGATATTCTTTTCTTCCAGTTCCTCGCATTCTTTGCAGCC |
| | strand, | GGACTCGGTCACATTGCCGTTGGAGGACAGGCTGTTGTTGGCCAG |
| | forward | GATGATCAGGTTTTCCACGGTATCGTGGATGGAGGCGTCGCCGCT |
| | strand | TTCCAGGCTGATCACTTGCAGCTCGAGCAGAAAGCACTTCATGGC |
| | SFFV | GGTCACTTTACAGCTAGGGTGCACGTCGCTCTCGGTGTACAGTGT |
| | synTF- | GGCGTCGATGTGCATGCTCTGGATCAGATCCTCGATCTTCTTCAG |
| | VPR | ATCGCTGATCACGTTGACCCAGTTGCCTGTAGAGCCGGGCACCCA |
| | | AAGAAGCAGCACCCACAGCAGCAGTGTGTCGGTTTCCATAGGTC |
| | | CAGGGTTTTCCTCCACGTCTCCACACGTCAGTAGACTCCCGCGTC |
| | | CCTCGCCAGATCCTCTCTTCCGTCTACTGGCGTTCAGGTAGGACA |
| | | TCACGCGGTCAATGGTCACGGCTCGAATGCGGAAAGCGTGCAAC |
| | | AGGATGCACAGCTTGATCTTGGTCTTGTAGAAGTCCGGTTCTTCC |
| | | AGGCTGGACTTTTGAGGCACAGTCTCGGAGTTGAAATTCAGGGC |
| | | CTGCATCAGTTCATCAATCACGGCGAGCATATTCTGGTCCAGGAA |
| | | GATCTGCCGCTTCGGGTCCATGAGCAGCTTGGCGTTCATGGTCTT |
| | | GAACTCGACCTGATACATCTTCAGATCTTCGTAGATCGAGGAAAG |
| | | ACAGAGCGCCATCATGAATGAGGTCTTTCTCGACGCCAGGCAGC |
| | | TGCCGTTAGTGATAAAGCTTGTCTCGCGGGAGTTCAGACACGATT |
| | | CGTTCTTGGTCAGTTCCAGCGGCAGGCAGGCTTCCACGGTCGAGG |
| | | TCTTGTCCTTGGTGATGTCCTCGTGATCAATTTCTTCCGAGGTGCA |
| | | GGGGTAGAACTCAAGGGTCTGGCGGGCCTTCTGCAACATGTTCG |
| | | ACACAGCCCTCAGGAGGTTTTGGGAGTGGTGTAGGCACGGGAAC |
| | | ATTCCAGGGTCGGGGGTTGCCACAGGGAGGTTCCGGGAACCTCC |
| | | TCCGGAGCCTCCTCCTGAACCTCCGCCTGATCCGCCACCGGAACA |
| | | AGGCACGCTGGCCCATTCGCTCCAGGAGGACGAGTAGTATCTAT |
| | | CCTGCGCCCGGACGCTGATTGACGCGTTCTTCCGACAAATCACAG |
| | | TGGCGGAGGTTTTGTCGGTGAACACCCGGTCTTTCTTCTCCCGTTT |
| | | GGACTTTCCCTGCACTTGCACACAGAAAGTGAGCGAGAAGTATG |
| | | AGTGCGGGGTGCTCCAAGTGTCTGGATATTCCCAAGACACTTCCA |
| | | CTTGGCGGGAGTTCTTGAGTGGCTTCAGCTGCAAGTTCTTGGGGG |
| | | GGTCAGGCTTGATGATGTCGCGGATAAAGAAGGAGGAAGTGTAG |
| | | TTCTCGTATTTCAGCTTATGCACGGCATCGACCATGACCTCGATA |
| | | GGCAGGGACTCTTCCGCGGCAGGGCAGGCGCTGTCCTCCTGGCA |
| | | TTCCACGGAGTACTCATATTCCTTGTTGTCTCCCCTGACTCTCTCG |
| | | GCGGACAGAGTGGCGGCTCCACAGGTCACGCCCTGAGGATCGCT |
| | | TGATCCCCGTGACGACTTCACGGAGAAAGTCAGGTCGGTGGAGA |
| | | TTGTCGTCAGCCACCAACAGGTGAACCGACCGCTGTAGTTCTTGG |
| | | CTTCGCAGCGGAGGAAGGTCTTGTTCTTCGGTTCTTTTTGGTCCTT |
| | | GAGGATGTCAGTGGACCAGATTCCATCCTCTTTCTTGTGCAGCAG |
| | | CAGCAGGGAGTGGGACAGCACTTCGCCACCCTTGTGGCAAGTGT |
| | | ACTGGCCCGCGTCGCCGAACTCCTTGACTTGAATGGTCAGGGTCT |
| | | TTCCGCTTCCGAGCACCTCGGAGCTCTGATCCAGGGTCCAGGTTA |
| | | TGCCGTCCTCTTCTGGCGTATCGCAAGTCAGCACGACCATTTCTC |
| | | CAGGGGCGTCCGGGTACCAATCCAGCTCGACCACGTAGACGTCC |
| | | TTCTTCAGTTCCCAAATGGCGACCAGAGGGGAAGCGAGGAACAC |
| | | AAGGGAGAACCAGGAGATGACGAGTTGCTGATGGCACATCATGG |
| | | TGGCGACACCGGTACGCGTTGGCCCCCATTATATACCCTCTAGAA |
| | | CTAGTtatccactccgtgtaagggagagtgagcctcttacgaatg |
| | | CGCGACATCGGCTACGCCgttccgaggcgactgatacgCGCGA |
| | | CATCGGCTACGCCgtacgaaggcagtccgattgCGCGACATCGG |
| | | CTACGCCgacctttactgagacgggagCGCGACATCGGCTACGCCg |
| | | aaggcgttgcgaatcctcatgcgattgttacgaaacccgTTAAT |
| | | TAAAGAGCGAGATTCCGTCTCAAAGAAAAAAAAAGTAATGAAATGA |
| | | ATAAAATGAGTCCTAGAGCCAGTAAATGTCGTAAATGTCTCAGCTAG |
| | | TCAGGTAGTAAAAGGTCTCAACTAGGCAGTGGCAGAGCAGGATTCAAA |
| | | TTCAGGGCTGTTGTGATGCCTCCGCAGACTCTGAGCGCCACCTGG |
| | | TGGTAATTTGTCTGTGCCTCTTCTGACGTGGAAGAACAGCAACTA |
| | | ACACACTAACACGGCATTTACTATGGGCCAGCCATTGTCCATCTA |
| | | GATGGccgataaaataaaagattttatttagtctccagaaaaa |
| | | ggggggaatgaaagaccccacctgtaggtttggcaagctagctg |
| | | cagtaacgccattttgcaaggcatggaaaaataccaaaccaagaa |
| | | tagagaagttcagatcaagggcgggtacatgaaaatagctaacgt |
| | | tgggccaaacaggatatctgcggtgagcagtttcggccccggcc |
| | | cggggccaagaacagatggtcaccgcagtttcggccccggcc |
| | | cgaggccaagaacagatggtccccagatatggcccaaccctcagca |
| | | gtttcttaagacccatcagatgtttccaggctcccccaaggac |
| | | ctgaaatgaccctgcgccttatttgaattaaccaatcagcctgc |
| | | ttctcgcttctgttcgcgcgcttctgcttcccgagctctataaaa |
| | | gagctcacaacccctcactcggcgcgccagtcctccgacagactga |
| | | gtcgcccgggGGATCCGCCACCATGAGTAGACCTGGCGAAAGAC |
| | | CATTTCAGTGCAGAATTTGTATGCGGAACTTCAGCAGAAGGCACGG |
| | | CCTGGACAGACACACCAGAACACACACAGGCGAGAAGCCTTTCCAGT |
| | | GTAGAATCTGTATGCGCAATTTCAGCGACCACAGCAGCCTGAAGCGG |
| | | CACCTGAGAACCCATACCGGCAGCCAGAAACCATTTCAATGCCGCAT |
| | | CTGTATGAGAAACTTCTCCGTGCGGCACAACCTGACCAGACACCT |
| | | GAGGACACACACCGGGGAGAAACCCTTCCAGTGCCGGATATGCA |
| | | TGAGGAATTTCTCCGACCACTCCAACCTGAGCCGCCACCTGAAAA |
| | | CTCACACCGGCTCTCAAAAGCCATTTCAGTGTCGTATATGTATGC |
| | | GGAATTTTTCCCAGCGGAGCAGCCTCGTGCGCCATCTGAGGACTC |
| | | ATACTGGCGAAAAGCCCTTCCAATGTCGCATATGCATGCGCAACT |
| | | TTAGCGAGTCCGGCCACCTGAAGAGACATCTGCGGACACACCTG |
| | | AGAGGCAGCACCTGTAGAGACTACAAGGACCACGACGGCGATTA |
| | | TAAGGATCACGACATCGACTACAAAGACGACGATGACAAGATGG |
| | | CCCCTAAGAAGAAGCGGAAAGTCGGCATCCATGGCGTGCCAGGT |
| | | GGACTTGAAGGTGGCGGAGGATCTGGCGGCACAGAGGATGTTGT |
| | | GTGCTGCCACAGCATCTACGGCAAGAAGAAGGGCGACATCGATA |
| | | CCTATCGGTACATCGGCAGCAGCGGCACAGGCTGCGTTGTGATC |
| | | GTGGGAAGAATCGTGCTGAGCGGCTCTGGCACAAGCGCCCCTAT |
| | | TACAGCCTACGCTCAGCAGACAAGAGGCCTGCTGGGCTGCATCA |
| | | TCACAAGCCTGACCGGCAGAGACAAGAACCAGGTGGAAGGCGA |
| | | GGTGCAGATCGTGTCTACAGCTACCCAGACCTTCCTGGCCACCTG |
| | | TATCAATGGCGTGTGCTGGGCCGTGTATCACGGCGCTGGAACCA |
| | | GAACAATCGCCTCTCCTAAGGGACCCGTGATCCAGATGTACACC |
| | | AACGTGGACCAGGACCTCGTTGGCTGGCCTGCTCCTCAGGGAAG |
| | | TAGAAGCCTGACACCTTGCACCTGTGGCTCCAGCGATCTGTACCT |
| | | GGTCACCAGACACGCCGACGTGATCCCTGTCAGAAGAAGAGGAG |
| | | ATTCCAGAGGCAGCCTGCTGAGCCCTAGACCTATCAGCTACCTGA |
| | | AGGGCAGCTCTGGCGGACCTCTGCTTTGTCCTGCTGGACATGCCG |
| | | TGGGCCTGTTTAGAGCCGCCGTGTGTACAAGAGGCGTGGCAAAG |
| | | GCCGTGGACTTCATCCCCGTGGAAAACCTGGAAACCACCATGCG |
| | | GAGCCCCGTGTTCACCGACAATTCTAGCCCTCCAGCCGTGACACT |
| | | GACACACCCCATCACCAAGATCGACAGAGAGGTGCTGTACCAAG |
| | | AGTTCGACGAGATGGAAGAGTGCAGCCAGCACTACCCCTACGAC |
| | | GTGCCAGATTATGCTGGCGGAGGTGGCAGCGGAGGCACCGAAGC |
| | | CTCTGGAAGCGGCAGAGCTGACGCCCTGGATGACTTCGACCTGG |
| | | ATATGCTGGGCAGCGACGCTCTGGACGATTTTGACCTCGACATGC |
| | | TGGGATCTGATGCACTCGACGATTTCGATTTGGACATGCTCGGCA |
| | | GTGATGCCTTGGACGACTTTGATCTTGATATGCTCATCAACAGCC |
| | | GGTCCAGCGGCAGCCCCAAGAAAAAAAGAAAAGTGGGCTCCCA |
| | | GTACCTGCCTGACACCGACGACAGACACCGGATCGAGGAAAAGC |
| | | GGAAGCGGACCTACGAGACATTCAAGAGCATCATGAAGAAGTCC |
| | | CCATTCAGCGGCCCCACCGATCCTAGACCTCCACCTAGAAGAATC |
| | | GCCGTGCCTAGCAGATCTAGCGCCTCCGTGCCTAAACCTGCTCCT |
| | | CAGCCTTATCCTTTCACCAGCAGCCTGAGCACCATCAACTACGAC |
| | | GAGTTCCCTACCATGGTGTTCCCCAGCGGCCAGATCTCTCAGGCT |
| | | TCTGCTCTTGCTCCAGCTCCTCCTCAGGTTCTGCCTCAAGCTCCTG |
| | | CACCAGCACCGGCTCCAGCTATGGTTTCTGCTTTGGCTCAGGCCC |
| | | CTGCTCCTGTGCCTGTTCTTGCTCCTGGACCACCTCAGGCTGTTGC |
| | | TCCTCCTGCTCCAAAACCTACACAGGCCGGCGAAGGCACACTGTC |
| | | TGAAGCTCTGCTGCAGCTCCAGTTCGATGACGAAGATCTGGGCGC |
| | | CCTGCTGGGCAATTCTACAGATCCTGCCGTGTTTACCGATCTGGC |
| | | CAGCGTGGACAACAGCGAGTTTCAGCAGCTCCTGAATCAGGGCA |
| | | TCCCTGTGGCTCCTCACACCACCGAACCTATGCTGATGGAATACC |
| | | CCGAGGCCATCACCAGACTGGTCACCGGTGCTCAAAGACCACCT |
| | | GATCCAGCTCCAGCACCACTGGGAGCACCTGGACTGCCTAATGG |
| | | ACTGCTGTCTGGCGACGAGGACTTCAGCTCTATCGCCGACATGGA |
| | | TTTCTCTGCCCTGCTCGGCTCTGGCAGCGGCTCTAGAGATAGCAG |
| | | AGAAGGCATGTTCCTGCCTAAGCCTGAGGCCGGCTCTGCCATCTC |
| | | CGATGTGTTCGAGGGAAGAGAAGTGTGCCAGCCTAAGCGGATCC |
| | | GGCCTTTTCACCCTCCTGGAAGCCCTTGGGCCAACAGACCTCTGC |
| | | CTGCTTCTCTGGCCCCTACACCAACAGGACCTGTGCACGAACCTG |
| | | TGGGCAGTCTGACCCCAGCTCCTGTTCCTCAACCTCTGGATCCCG |
| | | CTCCTGCTGTGACACCTGAAGCCTCTCATCTGCTGGAAGATCCCG |
| | | ACGAAGAGACAAGCCAGGCCGTGAAGGCCCTGAGAGAAATGGC |
| | | CGACACAGTGATCCCTCAGAAAGAGGAAGCCGCCATCTGCGGAC |
| | | AGATGGACCTGTCTCATCCTCCACCAAGAGGCCACCTGGACGAG |
| | | CTGACAACCACACTGGAATCCATGACCGAGGACCTGAACCTGGA |
| | | CAGCCCTCTGACACCCGAGCTGAACGAGATCCTGGACACCTTCCT |
| | | GAACGACGAGTGTCTGCTGCACGCCATGCACATCTCTACCGGCCT |
| | | GAGCATCTTCGACACCAGCCTGTTTTGA (SEQ ID NO: 149) |
| SB02675 | IL-15 | See SEQ ID NO: 141 |
| SB02675 | p65-based | See SEQ ID NO: 145 |
| | synTF | |
| SB02675 | insulator + | tccccagcatgcctgctattctcttcccaatcctcccccttgctgtc |
| | inducible | ctgccccaccccaccccccagaatagaatgacacctactcagacaa |
| | YB-TATA | tgcgatgcaatttcctcattttattaggaaaggacagtgggagtggc |
| | promoter | accttccagggtcaaggaaggcacgggggaggggcaaacaac |
| | IL-15 | agatggctggcaactagaaggcacagttaGCTGGTGTTGAT |
| | inverted | GAACATCTGCACGATGTGCACGAAGCTCTGCAGGAACTCTTT |
| | strand, | GATATTCTTTTCTTCCAGTTCCTCGCATTCTTTGCAGCC |
| | forward | GGACTCGGTCACATTGCCGTTGGAGGACAGGCTGTTGTTGGCCA |
| | strand | GGATGATCAGGTTTTCCACGGTATCGTGGATGGAGGCGTCGCCGCT |
| | SFFV | TTCCAGGCTGATCACTTGCAGCTCGAGCAGAAAGCACTTCATGGC |
| | synTF-p65 | GGTCACTTTACAGCTAGGGTGCACGTCGCTCTCGGTGTACAGTGT |
| | | GGCGTCGATGTGCATGCTCTGGATCAGATCCTCGATCTTCTTCAG |
| | | ATCGCTGATCACGTTGACCCAGTTGCCTGTAGAGCCGGGCACCCA |
| | | AAGAAGCAGCACCCACAGCAGCAGTGTGTCGGTTTCCATCATGG |
| | | TGGCGACACCGGTACGCGTTGGCCCCCATTATATACCCTCTAGAA |
| | | CTAGTtatccactccgtgtaagggagagtgagcctcttacgaatgC |
| | | GCGACATCGGCTACGCCgttccgaggcgactgatacgCGCGACAT |
| | | CGGCTACGCCgtacgaaggcagtccgattgCGCGACATCGGCTACG |
| | | CCgacctttactgagacgggagCGCGACATCGGCTACGCCgaaggcg |
| | | ttgcgaatcctcatgcgattgttacgaaacccgTTAATTAAAGAGCG |
| | | AGATTCCGTCTCAAAGAAAAAAAAAGTAATGAAATGAATAAAAT |
| | | GAGTCCTAGAGCCAGTAAATGTCGTAAATGTCTCAGCTAGTCAG |
| | | GTAGTAAAAGGTCTCAACTAGGCAGTGGCAGAGCAGGATTCAAA |
| | | TTCAGGGCTGTTGTGATGCCTCCGCAGACTCTGAGCGCCACCTGG |
| | | TGGTAATTTGTCTGTGCCTCTTCTGACGTGGAAGAACAGCAACTA |
| | | ACACACTAACACGGCATTTACTATGGGCCAGCCATTGTCCATCTA |
| | | GATGGccgataaaataaaagattttatttagtctccagaaaaag |
| | | gggggaatgaaagaccccacctgtaggtttggcaagctagctgca |
| | | gtaacgccattttgcaaggcatggaaaaataccaaaccaagaat |
| | | agagaagttcagatcaagggcgggtacatgaaaatagctaacgttg |
| | | ggccaaacaggatatctgcggtgagcagtttcggccccggcccg |
| | | gggccaagaacagatggtcaccgcagtttcggccccggcccgagg |
| | | ccaagaacagatggtccccagatatggcccaaccctcagcagtttct |
| | | taagacccatcagatgtttccaggctcccccaaggacctgaaatg |
| | | accctgcgccttatttgaattaaccaatcagcctgcttctcgct |
| | | tctgttcgcgcgcttctgcttcccgagctctataaaagagctcacaa |
| | | cccctcactcggcgcgccagtcctccgacagactgagtcgcccgggG |
| | | GATCCGCCACCATGAGTAGACCTGGCGAAAGACCATTTCAGTGC |
| | | AGAATTTGTATGCGGAACTTCAGCAGAAGGCACGGCCTGGACAG |
| | | ACACACCAGAACACACACAGGCGAGAAGCCTTTCCAGTGTAGAA |
| | | TCTGTATGCGCAATTTCAGCGACCACAGCAGCCTGAAGCGGCAC |
| | | CTGAGAACCCATACCGGCAGCCAGAAACCATTTCAATGCCGCAT |
| | | CTGTATGAGAAACTTCTCCGTGCGGCACAACCTGACCAGACACCT |
| | | GAGGACACACACCGGGGAGAAACCCTTCCAGTGCCGGATATGCA |
| | | TGAGGAATTTCTCCGACCACTCCAACCTGAGCCGCCACCTGAAAA |
| | | CTCACACCGGCTCTCAAAAGCCATTTCAGTGTCGTATATGTATGC |
| | | GGAATTTTTCCCAGCGGAGCAGCCTCGTGCGCCATCTGAGGACTC |
| | | ATACTGGCGAAAAGCCCTTCCAATGTCGCATATGCATGCGCAACT |
| | | TTAGCGAGTCCGGCCACCTGAAGAGACATCTGCGGACACACCTG |
| | | AGAGGCAGCACCTGTAGAGACTACAAGGACCACGACGGCGATTA |
| | | TAAGGATCACGACATCGACTACAAAGACGACGATGACAAGATGG |
| | | CCCCTAAGAAGAAGCGGAAAGTCGGCATCCATGGCGTGCCAGGT |
| | | GGACTTGAAGGTGGCGGAGGATCTGGCGGCACAGAGGATGTTGT |
| | | GTGCTGCCACAGCATCTACGGCAAGAAGAAGGGCGACATCGATA |
| | | CCTATCGGTACATCGGCAGCAGCGGCACAGGCTGCGTTGTGATC |
| | | GTGGGAAGAATCGTGCTGAGCGGCTCTGGCACAAGCGCCCCTAT |
| | | TACAGCCTACGCTCAGCAGACAAGAGGCCTGCTGGGCTGCATCA |
| | | TCACAAGCCTGACCGGCAGAGACAAGAACCAGGTGGAAGGCGA |
| | | GGTGCAGATCGTGTCTACAGCTACCCAGACCTTCCTGGCCACCTG |
| | | TATCAATGGCGTGTGCTGGGCCGTGTATCACGGCGCTGGAACCA |
| | | GAACAATCGCCTCTCCTAAGGGACCCGTGATCCAGATGTACACC |
| | | AACGTGGACCAGGACCTCGTTGGCTGGCCTGCTCCTCAGGGAAG |
| | | TAGAAGCCTGACACCTTGCACCTGTGGCTCCAGCGATCTGTACCT |
| | | GGTCACCAGACACGCCGACGTGATCCCTGTCAGAAGAAGAGGAG |
| | | ATTCCAGAGGCAGCCTGCTGAGCCCTAGACCTATCAGCTACCTGA |
| | | AGGGCAGCTCTGGCGGACCTCTGCTTTGTCCTGCTGGACATGCCG |
| | | TGGGCCTGTTTAGAGCCGCCGTGTGTACAAGAGGCGTGGCAAAG |
| | | GCCGTGGACTTCATCCCCGTGGAAAACCTGGAAACCACCATGCG |
| | | GAGCCCCGTGTTCACCGACAATTCTAGCCCTCCAGCCGTGACACT |
| | | GACACACCCCATCACCAAGATCGACAGAGAGGTGCTGTACCAAG |
| | | AGTTCGACGAGATGGAAGAGTGCAGCCAGCACTACCCCTACGAC |
| | | GTGCCAGATTATGCTGGCGGAGGTGGCAGCGGAGGCACCGATGA |
| | | ATTTCCAACCATGGTGTTCCCCAGCGGCCAGATCTCTCAGGCATC |
| | | TGCTCTTGCTCCAGCTCCACCTCAGGTTCTGCCTCAAGCTCCTGCT |
| | | CCGGCTCCTGCACCAGCTATGGTGTCTGCACTTGCTCAGGCACCA |
| | | GCTCCAGTGCCTGTTCTTGCTCCTGGACCTCCTCAGGCTGTTGCTC |
| | | CACCAGCACCTAAACCTACACAGGCCGGCGAGGGAACACTGTCT |
| | | GAAGCCCTGCTGCAACTCCAGTTCGATGACGAAGATCTGGGCGC |
| | | CCTGCTGGGAAACTCTACAGATCCTGCCGTGTTTACCGATCTGGC |
| | | CAGCGTGGACAACAGCGAGTTTCAGCAGCTCCTGAACCAGGGCA |
| | | TCCCAGTGGCTCCTCACACCACAGAGCCTATGCTGATGGAATACC |
| | | CCGAGGCCATCACCAGACTGGTCACCGGCGCACAAAGACCACCT |
| | | GATCCTGCTCCAGCACCGCTTGGAGCACCTGGACTGCCTAATGGA |
| | | CTGCTGTCTGGCGACGAGGACTTCAGTTCTATCGCTGATATGGAT |
| | | TTCTCTGCCCTTCTGTCTCAGATTAGTAGCCAGCTGTAA (SEQ ID |
| | | NO: 150) |
| SB02676 | VPR-based | See SEQ ID NO: 147 |
| | synTF | |
| SB02676 | IL-15 | See SEQ ID NO: 141 |
| SB02676 | insulator + | tccccagcatgcctgctattctcttcccaatcctcccccttgctgt |
| | inducible | cctgccccaccccaccccccagaatagaatgacacctactcagacaa |
| | YB-TATA | tgcgatgcaatttcctcattttattaggaaaggacagtggga |
| | promoter | gtggcaccttccagggtcaaggaaggcacgggggaggggcaaa |
| | IL-15 | caacagatggctggcaactagaaggcacagttaGCTGGTG |
| | inverted | TTGATGAACATCTGCACGATGTGCACGAAGCTCTGCAGGA |
| | strand, | ACTCTTTGATATTCTTTTCTTCCAGTTCCTCGCATTCTTTGCAGCC |
| | forward | GGACTCGGTCACATTGCCGTTGGAGGACAGGCTGTTGTTGGCCAG |
| | strand | GATGATCAGGTTTTCCACGGTATCGTGGATGGAGGCGTCGCCGCT |
| | SFFV | TTCCAGGCTGATCACTTGCAGCTCGAGCAGAAAGCACTTCATGGC |
| | synTF- | GGTCACTTTACAGCTAGGGTGCACGTCGCTCTCGGTGTACAGTGT |
| | VPR | GGCGTCGATGTGCATGCTCTGGATCAGATCCTCGATCTTCTTCAG |
| | | ATCGCTGATCACGTTGACCCAGTTGCCTGTAGAGCCGGGCACCCA |
| | | AAGAAGCAGCACCCACAGCAGCAGTGTGTCGGTTTCCATCATGG |
| | | TGGCGACACCGGTACGCGTTGGCCCCCATTATATACCCTCTAGAA |
| | | CTAGTtatccactccgtgtaagggagagtgagcctcttacgaatgC |
| | | GCGACATCGGCTACGCCgttccgaggcgactgatacgCGCGACAT |
| | | CGGCTACGCCgtacgaaggcagtccgattgCGCGACATCGGCTACG |
| | | CCgacctttactgagacgggagCGCGACATCGGCTACGCCgaaggcg |
| | | ttgcgaatcctcatgcgattgttacgaaacccgTTAATTAAAGAGCG |
| | | AGATTCCGTCTCAAAGAAAAAAAAAGTAATGAAATGAATAAAAT |
| | | GAGTCCTAGAGCCAGTAAATGTCGTAAATGTCTCAGCTAGTCAG |
| | | GTAGTAAAAGGTCTCAACTAGGCAGTGGCAGAGCAGGATTCAAA |
| | | TTCAGGGCTGTTGTGATGCCTCCGCAGACTCTGAGCGCCACCTGG |
| | | TGGTAATTTGTCTGTGCCTCTTCTGACGTGGAAGAACAGCAACTA |
| | | ACACACTAACACGGCATTTACTATGGGCCAGCCATTGTCCATCTA |
| | | GATGGccgataaaataaaagattttatttagtctccagaaaaag |
| | | gggggaatgaaagaccccacctgtaggtttggcaagctagctgcag |
| | | taacgccattttgcaaggcatggaaaaataccaaaccaagaatag |
| | | agaagttcagatcaagggcgggtacatgaaaatagctaacgttggg |
| | | ccaaacaggatatctgcggtgagcagtttcggccccggcccggggcc |
| | | aagaacagatggtcaccgcagtttcggccccggcccgaggccaag |
| | | aacagatggtccccagatatggcccaaccctcagcagtttcttaag |
| | | acccatcagatgtttccaggctcccccaaggacctgaaatgacc |
| | | ctgcgccttatttgaattaaccaatcagcctgcttctcgcttctgt |
| | | tcgcgcgcttctgcttcccgagctctataaaagagctcacaacc |
| | | cctcactcggcgcgccagtcctccgacagactgagtcgcccgggG |
| | | GATCCGCCACCATGAGTAGACCTGGCGAAAGACCATTTCAGTGC |
| | | AGAATTTGTATGCGGAACTTCAGCAGAAGGCACGGCCTGGACAG |
| | | ACACACCAGAACACACACAGGCGAGAAGCCTTTCCAGTGTAGAA |
| | | TCTGTATGCGCAATTTCAGCGACCACAGCAGCCTGAAGCGGCAC |
| | | CTGAGAACCCATACCGGCAGCCAGAAACCATTTCAATGCCGCAT |
| | | CTGTATGAGAAACTTCTCCGTGCGGCACAACCTGACCAGACACCT |
| | | GAGGACACACACCGGGGAGAAACCCTTCCAGTGCCGGATATGCA |
| | | TGAGGAATTTCTCCGACCACTCCAACCTGAGCCGCCACCTGAAAA |
| | | CTCACACCGGCTCTCAAAAGCCATTTCAGTGTCGTATATGTATGC |
| | | GGAATTTTTCCCAGCGGAGCAGCCTCGTGCGCCATCTGAGGACTC |
| | | ATACTGGCGAAAAGCCCTTCCAATGTCGCATATGCATGCGCAACT |
| | | TTAGCGAGTCCGGCCACCTGAAGAGACATCTGCGGACACACCTG |
| | | AGAGGCAGCACCTGTAGAGACTACAAGGACCACGACGGCGATTA |
| | | TAAGGATCACGACATCGACTACAAAGACGACGATGACAAGATGG |
| | | CCCCTAAGAAGAAGCGGAAAGTCGGCATCCATGGCGTGCCAGGT |
| | | GGACTTGAAGGTGGCGGAGGATCTGGCGGCACAGAGGATGTTGT |
| | | GTGCTGCCACAGCATCTACGGCAAGAAGAAGGGCGACATCGATA |
| | | CCTATCGGTACATCGGCAGCAGCGGCACAGGCTGCGTTGTGATC |
| | | GTGGGAAGAATCGTGCTGAGCGGCTCTGGCACAAGCGCCCCTAT |
| | | TACAGCCTACGCTCAGCAGACAAGAGGCCTGCTGGGCTGCATCA |
| | | TCACAAGCCTGACCGGCAGAGACAAGAACCAGGTGGAAGGCGA |
| | | GGTGCAGATCGTGTCTACAGCTACCCAGACCTTCCTGGCCACCTG |
| | | TATCAATGGCGTGTGCTGGGCCGTGTATCACGGCGCTGGAACCA |
| | | GAACAATCGCCTCTCCTAAGGGACCCGTGATCCAGATGTACACC |
| | | AACGTGGACCAGGACCTCGTTGGCTGGCCTGCTCCTCAGGGAAG |
| | | TAGAAGCCTGACACCTTGCACCTGTGGCTCCAGCGATCTGTACCT |
| | | GGTCACCAGACACGCCGACGTGATCCCTGTCAGAAGAAGAGGAG |
| | | ATTCCAGAGGCAGCCTGCTGAGCCCTAGACCTATCAGCTACCTGA |
| | | AGGGCAGCTCTGGCGGACCTCTGCTTTGTCCTGCTGGACATGCCG |
| | | TGGGCCTGTTTAGAGCCGCCGTGTGTACAAGAGGCGTGGCAAAG |
| | | GCCGTGGACTTCATCCCCGTGGAAAACCTGGAAACCACCATGCG |
| | | GAGCCCCGTGTTCACCGACAATTCTAGCCCTCCAGCCGTGACACT |
| | | GACACACCCCATCACCAAGATCGACAGAGAGGTGCTGTACCAAG |
| | | AGTTCGACGAGATGGAAGAGTGCAGCCAGCACTACCCCTACGAC |
| | | GTGCCAGATTATGCTGGCGGAGGTGGCAGCGGAGGCACCGAAGC |
| | | CTCTGGAAGCGGCAGAGCTGACGCCCTGGATGACTTCGACCTGG |
| | | ATATGCTGGGCAGCGACGCTCTGGACGATTTTGACCTCGACATGC |
| | | TGGGATCTGATGCACTCGACGATTTCGATTTGGACATGCTCGGCA |
| | | GTGATGCCTTGGACGACTTTGATCTTGATATGCTCATCAACAGCC |
| | | GGTCCAGCGGCAGCCCCAAGAAAAAAAGAAAAGTGGGCTCCCA |
| | | GTACCTGCCTGACACCGACGACAGACACCGGATCGAGGAAAAGC |
| | | GGAAGCGGACCTACGAGACATTCAAGAGCATCATGAAGAAGTCC |
| | | CCATTCAGCGGCCCCACCGATCCTAGACCTCCACCTAGAAGAATC |
| | | GCCGTGCCTAGCAGATCTAGCGCCTCCGTGCCTAAACCTGCTCCT |
| | | CAGCCTTATCCTTTCACCAGCAGCCTGAGCACCATCAACTACGAC |
| | | GAGTTCCCTACCATGGTGTTCCCCAGCGGCCAGATCTCTCAGGCT |
| | | TCTGCTCTTGCTCCAGCTCCTCCTCAGGTTCTGCCTCAAGCTCCTG |
| | | CACCAGCACCGGCTCCAGCTATGGTTTCTGCTTTGGCTCAGGCCC |
| | | CTGCTCCTGTGCCTGTTCTTGCTCCTGGACCACCTCAGGCTGTTGC |
| | | TCCTCCTGCTCCAAAACCTACACAGGCCGGCGAAGGCACACTGTC |
| | | TGAAGCTCTGCTGCAGCTCCAGTTCGATGACGAAGATCTGGGCGC |
| | | CCTGCTGGGCAATTCTACAGATCCTGCCGTGTTTACCGATCTGGC |
| | | CAGCGTGGACAACAGCGAGTTTCAGCAGCTCCTGAATCAGGGCA |
| | | TCCCTGTGGCTCCTCACACCACCGAACCTATGCTGATGGAATACC |
| | | CCGAGGCCATCACCAGACTGGTCACCGGTGCTCAAAGACCACCT |
| | | GATCCAGCTCCAGCACCACTGGGAGCACCTGGACTGCCTAATGG |
| | | ACTGCTGTCTGGCGACGAGGACTTCAGCTCTATCGCCGACATGGA |
| | | TTTCTCTGCCCTGCTCGGCTCTGGCAGCGGCTCTAGAGATAGCAG |
| | | AGAAGGCATGTTCCTGCCTAAGCCTGAGGCCGGCTCTGCCATCTC |
| | | CGATGTGTTCGAGGGAAGAGAAGTGTGCCAGCCTAAGCGGATCC |
| | | GGCCTTTTCACCCTCCTGGAAGCCCTTGGGCCAACAGACCTCTGC |
| | | CTGCTTCTCTGGCCCCTACACCAACAGGACCTGTGCACGAACCTG |
| | | TGGGCAGTCTGACCCCAGCTCCTGTTCCTCAACCTCTGGATCCCG |
| | | CTCCTGCTGTGACACCTGAAGCCTCTCATCTGCTGGAAGATCCCG |
| | | ACGAAGAGACAAGCCAGGCCGTGAAGGCCCTGAGAGAAATGGC |
| | | CGACACAGTGATCCCTCAGAAAGAGGAAGCCGCCATCTGCGGAC |
| | | AGATGGACCTGTCTCATCCTCCACCAAGAGGCCACCTGGACGAG |
| | | CTGACAACCACACTGGAATCCATGACCGAGGACCTGAACCTGGA |
| | | CAGCCCTCTGACACCCGAGCTGAACGAGATCCTGGACACCTTCCT |
| | | GAACGACGAGTGTCTGCTGCACGCCATGCACATCTCTACCGGCCT |
| | | GAGCATCTTCGACACCAGCCTGTTTTGA (SEQ ID NO: 151) |

### Results

IL-12 and/or IL-15 payload expression was assessed in T cells for various regulatable TF expression system strategies and constructs. A drug-inducible format ACP (also referred to as “synTF”) using an NS3/NS4 protease cleavage site and either a VPR transcriptional effector domain (constructs SB02667 and SB02668) or a p65 transcriptional effector domain (constructs SB02670 and SB02671) were assessed. All constructs encoded cytokine payloads included an A2 insulator and YB-TATA promoter driving expression in the opposite direction as the ACP cassette (e.g., see orientation of reporter and ACP/synTF cassettes in construct “1560” in FIG. 20). A summary of the systems assessed in shown in FIG. 38.

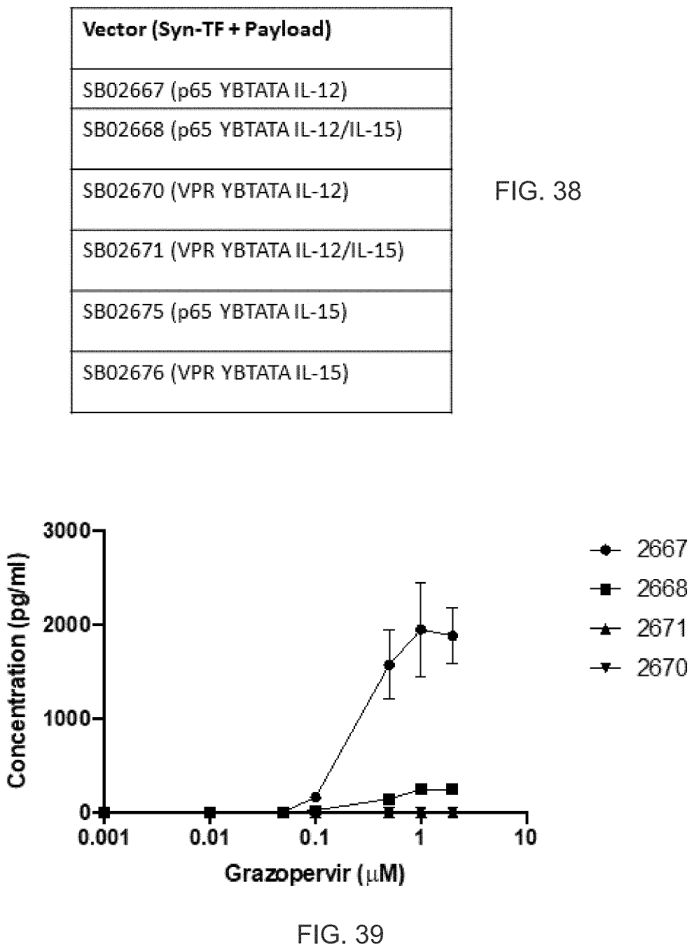

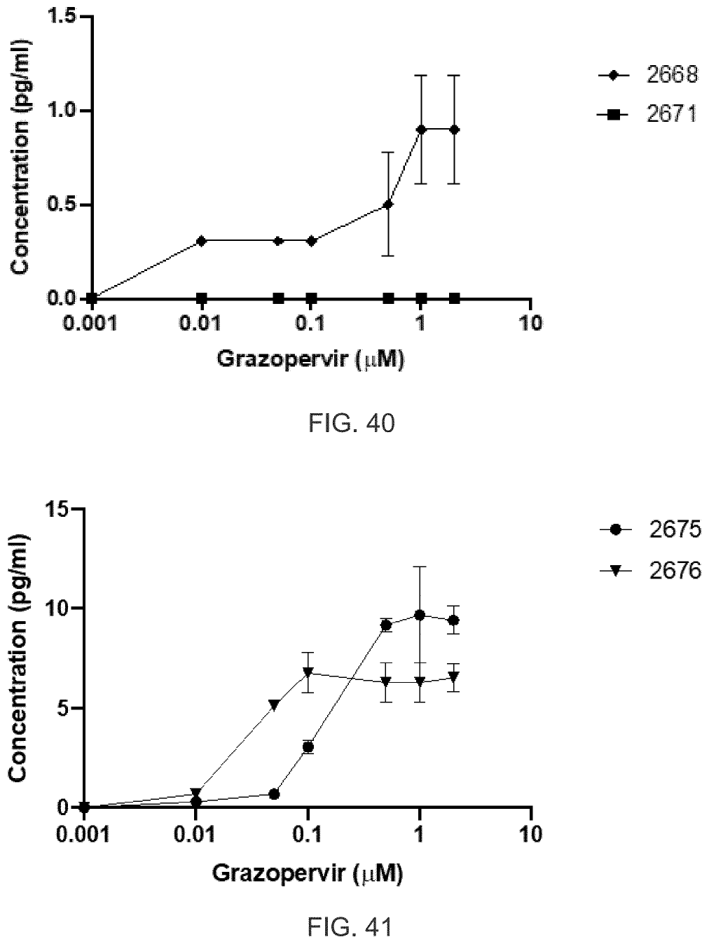

As shown in FIG. 39 and quantified in Table 23, IL-12 production was observed in a concentration dependent manner following addition of the NS3 protease inhibitor grazoprevir when using a p65 transcriptional effector domain, with greater production (˜8-fold) observed when expressed alone (SB02667) as compared to in a ribosome skipping tag 2A multicistronic system (SB02668), see circle vs. square in FIG. 39, respectively, and Table 23. As shown in FIG. 40 and FIG. 41 and quantified in Table 24, IL-15 production was also observed for constructs encoding IL-15 in a concentration dependent manner for the NS3 protease inhibitor grazoprevir when using a p65 transcriptional effector domain in both a ribosome skipping tag 2A multicistronic system (SB02668; FIG. 40 diamond) and expressed as a single payload (SB02675; FIG. 41 circle). IL-15 expressed at greater levels when encoded as a single payload, though at notably low levels in the 2A multicistronic system. Notably, there was no observable cytokine expression for constructs using a VPR transcriptional effector domain other than for IL-15 encoded as a single payload (SB02676; FIG. 41 triangle). Accordingly, regulated cytokine expression was observed in a drug-dependent in T cells for the various systems.

TABLE 23
IL-12 (pg/mL) Production in T cells for Additional Constructs

| [GRZ] uM | 2667 #1 | 2667 #2 | 2668 #1 | 2668 #2 | 2671 #1 | 2671 #2 | 2670 #1 | 2670 #2 |
|---|---|---|---|---|---|---|---|---|
| 2 | 2088.21 | 1672.93 | 251.45 | 239.53 | 2.96 | 2.96 | 2.96 | 2.96 |
| 1 | 2304.49 | 1593.19 | 251.45 | 239.53 | 2.96 | 2.96 | 2.96 | 2.96 |
| 0.5 | 1833.06 | 1315.96 | 145.52 | 145.52 | 2.96 | 2.96 | 2.96 | 2.96 |
| 0.1 | 192.19 | 133.97 | 22.96 | 22.96 | 2.96 | 2.96 | 2.96 | 2.96 |
| 0.05 | 12.70 | 2.96 | 0 | 0 | 2.96 | 2.96 | 2.96 | 2.96 |
| 0.01 | 0 | 0 | 0 | 0 | 0 | 0 | 0 | 0 |
| No drug | 0 | 0 | 0 | 0 | 0 | 0 | 0 | 0 |

TABLE 24
IL-15 (pg/mL) Production in T cells for Additional Constructs

| [GRZ] uM | 2668 #1 | 2668 #2 | 2671 #1 | 2671 #2 | 2675 #1 | 2675 #2 | 2676 #1 | 2676 #2 |
|---|---|---|---|---|---|---|---|---|
| 2 | 1.102 | 0.694 | 0 | 0 | 9.91 | 8.93 | 7.00 | 6.05 |
| 1 | 1.102 | 0.694 | 0 | 0 | 11.39 | 7.97 | 7.00 | 5.58 |
| 0.5 | 0.305 | 0.694 | 0 | 0 | 9.42 | 8.93 | 7.00 | 5.58 |
| 0.1 | 0.305 | 0.305 | 0 | 0 | 3.28 | 2.83 | 7.48 | 6.05 |
| 0.05 | 0.305 | 0.305 | 0 | 0 | 0.69 | 0.69 | 5.11 | 5.11 |
| 0.01 | 0.305 | 0.305 | 0 | 0 | 0.31 | 0.31 | 0.69 | 0.69 |
| No drug | 0 | 0 | 0 | 0 | 0 | 0 | 0 | 0 |

### Example 13: In Vivo Assement of Various Grazoprevir Dosing Regimens

Regulatable TF expression systems, including various Grz dosing and T cell injection regimens, were assessed both in vitro and in vivo.

### Materials and Methods

For in vivo assessment, T cells were transduced with SB01845 & SB02357 (“inducible IL-12”), each at an estimated MOI of 5 based on viral titering in HEK cells. Control T cells (“constitutive IL-12”) were transduced with 5 MOI of SB00171, encoding constitutive hIL-12 driven by SFFV. Negative controls were untransduced T cells. The number of lentiviral genomes integrated into the T cells was analyzed in bulk by PCR (copy #assay). NSG mice were randomized on day −2 and vehicle or Grazoprevir (Grz) dosing began in the afternoon on day 1 (Vehicle: 2.5% DMSO, 30% PEG400, 67.5% PBS). Grazoprevir potassium salt was dissolved sequentially in DMSO, PEG400, and PBS to reach 10 mg/mL. All Grz dosing was administered IP. 20e6 T cells per mouse were injected by tail vein injection, as indicated. Specific Grz dosing and T cell injection regimens are indicated in the tables below. Luminex assay was run to assess levels of hIL-12 in mouse plasma. Presence of human T cells in mouse blood was analyzed by flow cytometry.

### Results

A first series of Grazoprevir (Grz) dosing regimens were assessed, as described in Table 25.

TABLE 25
Grazoprevir (Grz) dosing regimens

| Dosing | | | | | | |
|---|---|---|---|---|---|---|
| schedule | Day-2 | Day 1 | Day 2 | Day 3 | Day 4 | Day 8/12 |
| Previous- | Randomize | 25 mg/kg | 25 mg/kg Grz | 25 mg/kg | 25 mg/kg GRZ | Harvest |
| 25 BID | mice | Grz, PM | BID | GRZ BID | BID | plasma |
| | | | | | Harvest plasma | |
| 1× day- | Randomize | 25 mg/kg | 50 mg/kg Grz | 50 mg/kg | 50 mg/kg Grz | Harvest |
| 50 mg/kg | mice | Grz, PM | AM, | Grz AM | AM | plasma |
| | | | Inject T cells | | Harvest plasma | |
| 2× day- | Randomize | 25 mg/kg | 25 mg/kg Grz | 50 mg/kg | 25 mg/kg GRZ | Harvest |
| 25/50 | mice | Grz, PM | AM, | Grz AM, | AM & PM | plasma |
| | | | Inject T cells, | 25 mg/kg | Harvest plasma | |
| | | | 50 mg/kg Grz | Grz PM | | |
| | | | PM | | | |
| 1× day- | Randomize | 25 mg/kg | 75 mg/kg Grz | 75 mg/kg | 50 mg/kg Grz | Harvest |
| 75 mg/kg | mice | Grz, PM | AM, Inject T | Grz AM | AM | plasma |
| | | | cells | | Harvest plasma | |

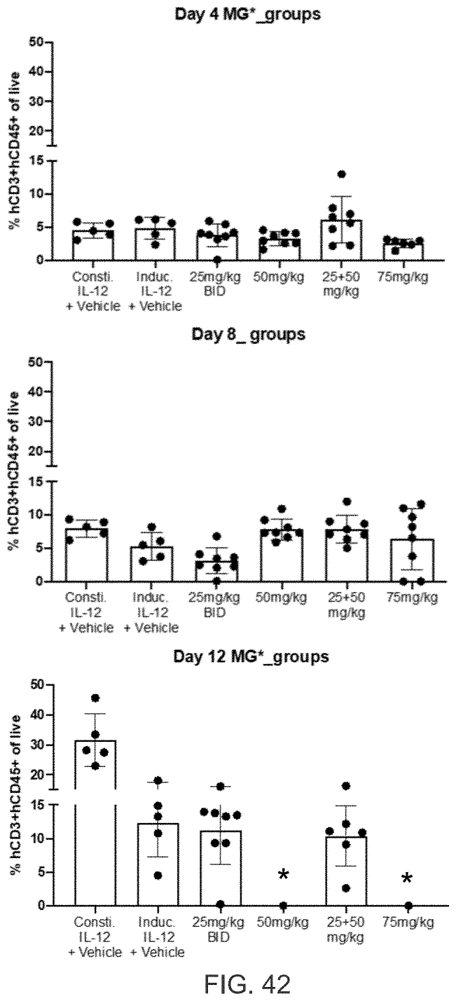

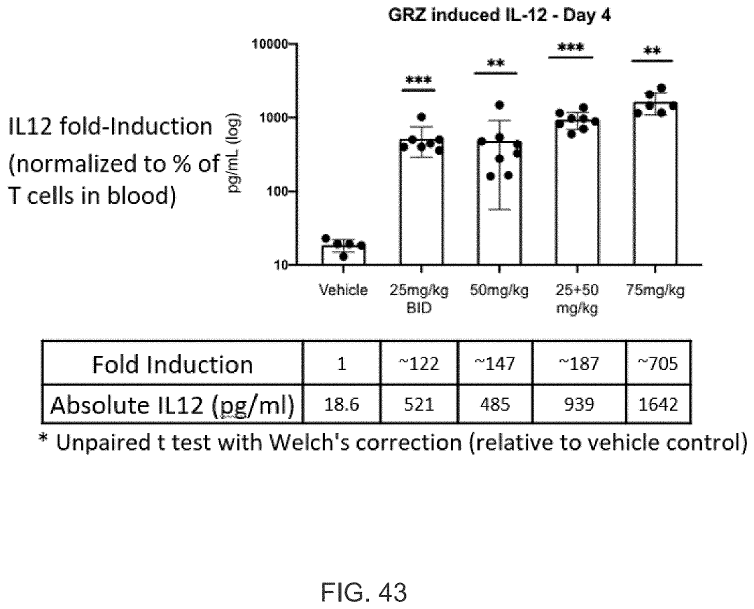

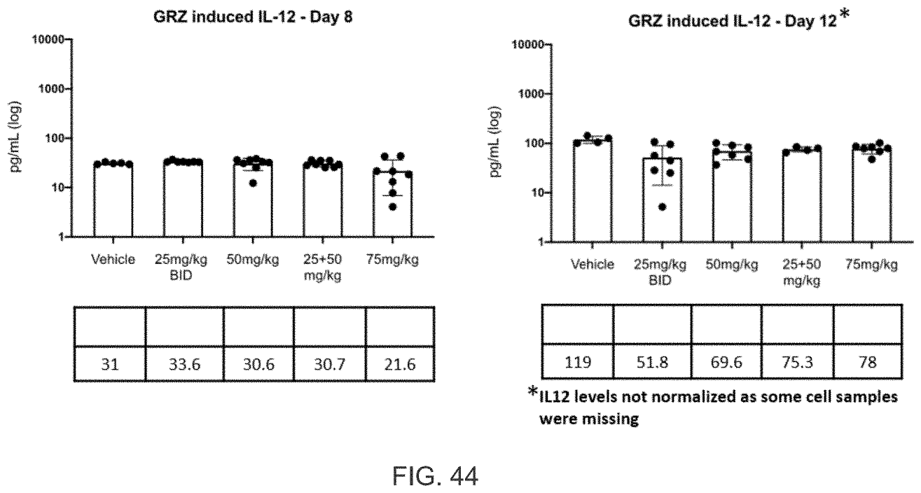

Persistence of the injected T cells was assessed. As shown in FIG. 42, and quantitated in Table 26A (Day 4), Table 26B (Day 8), and Table 26C (Day 12), populations of injected human T cells were observed in the blood through Day 12 (note 50 mg/kg and 75 mg/kg groups of cells from Day 12 were not included in the analysis due to loss of the samples). IL-12 levels were assessed in plasma. As shown in FIG. 43, significant Grazoprevir dose dependent induction of IL-12 was observed on Day 4 (2 days post Grz dosing), ranging from ˜100-fold to ˜700-fold above vehicle. As shown in FIG. 44, serum IL-12 levels returned to baseline by Day 8 (left panel) and remained stable at Day 12 (right panel). Notably, mice receiving 75 mg/kg of GRZ experienced toxicity with moderate to severe weight loss following the 75 mg/kg daily dose on days 2-3 (mice recovered mildly 24 hours after each dose). In addition, 2 of 8 mice in the 75 mg/kg group were found dead on the final day (Day 12) with no death seen in the other groups, with necropsy revealing abnormalities in the liver and ovarian ducts, including one mouse having fused organs. All mice in the 75 mg/kg group presented with enlarged spleens, many with inflamed oviducts, at Day 12. Accordingly, the Grazoprevir (Grz) dosing regimens assessed demonstrated drug-dependent IL-12 production, with toxicity observed in certain dosing schemes.

TABLE 26A
Human T cells in the blood (hCD3+: hCD45 + shown as % of live cells); Day 4

| | Mouse | Mouse | Mouse | Mouse | Mouse | Mouse | Mouse | Mouse |
|---|---|---|---|---|---|---|---|---|
| Treatment | #1 | #2 | #3 | #4 | #5 | #6 | #7 | #8 |
| Constitutive | 3.0 | 4.5 | 5.77 | 3.86 | 5.56 | | | |
| IL-12 + | | | | | | | | |
| Vehicle | | | | | | | | |
| Inducible | 3.93 | 2.38 | 6.12 | 5.63 | 6.18 | | | |
| IL-12 + | | | | | | | | |
| Vehicle | | | | | | | | |
| 25 mg/kg | 5.42 | 0.08 | 4.07 | 3.61 | 3.88 | 5.91 | 4.2 | 3.14 |
| BID | | | | | | | | |
| 25 + 50 | 2.18 | 6.98 | 6.53 | 13 | 2.24 | 4.72 | 5.64 | 7.89 |
| mg/kg | | | | | | | | |
| 50 mg/kg | 4.09 | 2.96 | 3.7 | 1.62 | 4.16 | 2.57 | 4.55 | 2.58 |
| 75 mg/kg | 2.95 | 3.14 | 2.6 | 0* | 2.41 | 0* | 1.43 | 2.87 |

TABLE 26B
Human T cells in the blood (hCD3+: hCD45+ shown as % of live cells); Day 8

| | Mouse | Mouse | Mouse | Mouse | Mouse | Mouse | Mouse | Mouse |
|---|---|---|---|---|---|---|---|---|
| Treatment | #1 | #2 | #3 | #4 | #5 | #6 | #7 | #8 |
| Constitutive | 7.2 | 9.34 | 6.2 | 8.89 | 8.19 | | | |
| IL-12 + | | | | | | | | |
| Vehicle | | | | | | | | |
| Inducible | 3.05 | 8.17 | 6.04 | 5.45 | 3.71 | | | |
| IL-12 + | | | | | | | | |
| Vehicle | | | | | | | | |
| 25 mg/kg | 6.78 | 0.077 | 3.63 | 2.46 | 2.07 | 3.45 | 2.27 | 4.09 |
| BID | | | | | | | | |
| 25 + 50 | 7.09 | 7.85 | 9 | 12 | 6.34 | 7.02 | 8.65 | 5.04 |
| mg/kg | | | | | | | | |
| 50 mg/kg | 7.36 | 10.9 | 7.31 | 8.1 | 9.23 | 5.88 | 6.63 | 7.34 |
| 75 mg/kg | 8.19 | 9.67 | 6.55 | 0.028 | 11.6 | 4.7e−003 | 11 | 3.79 |

TABLE 26C
Human T cells in the blood (hCD3+: hCD45+ shown as % of live cells); Day 12

| | Mouse | Mouse | Mouse | Mouse | Mouse | Mouse | Mouse | Mouse |
|---|---|---|---|---|---|---|---|---|
| Treatment | #1 | #2 | #3 | #4 | #5 | #6 | #7 | #8 |
| Constitutive | 23 | 33.5 | 27.5 | 28.2 | 45.7 | | | |
| IL-12 + | | | | | | | | |
| Vehicle | | | | | | | | |
| Inducible | 4.52 | 10.8 | 18.1 | 14.9 | 13.3 | | | |
| IL-12 + | | | | | | | | |
| Vehicle | | | | | | | | |
| 25 mg/kg | 13.5 | 0.21 | 13.3 | 9.36 | 9.33 | 14 | 16.1 | 13.8 |
| BID | | | | | | | | |
| 25 + 50 | 10.9 | 12.2 | 11.1 | 16.3 | 2.61 | 9.13 | | |
| mg/kg | | | | | | | | |
| 50 mg/kg | ** | | | | | | | |
| 75 mg/kg | ** | | | | | | | |

** Lost to technical error

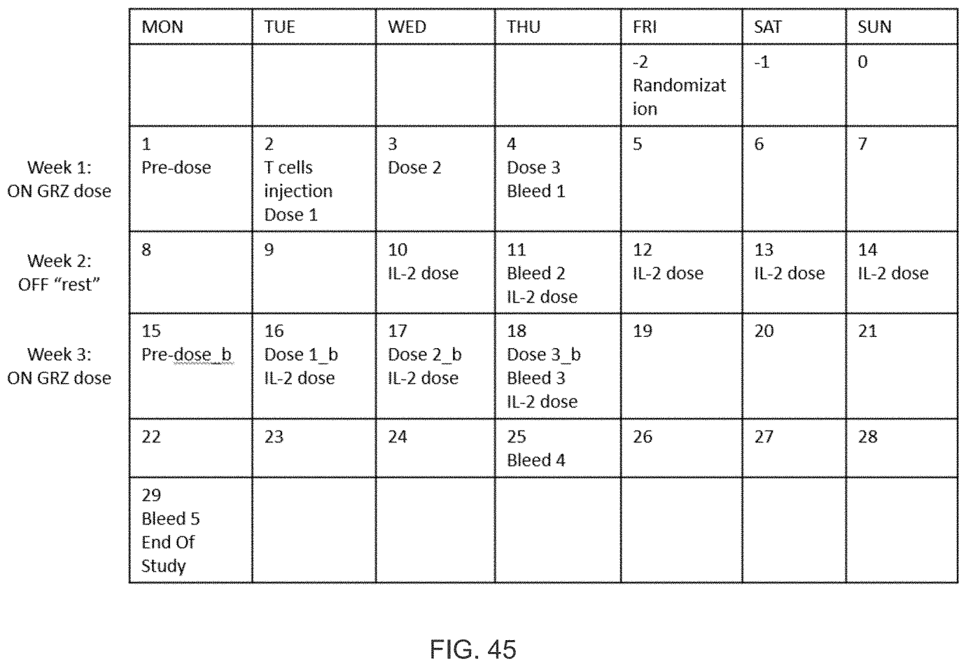

An “on/off/on” Grazoprevir (Grz) dosing regimen was next assessed, with the calendar illustrating the particular dosing regimen in FIG. 45. Following the results of the experiments above, animals were dosed (IP) 60 mg/kg for 3 days once a day. Recombinant (hIL-2 1×10\hat{ }4 IU) was also administered as indicated.

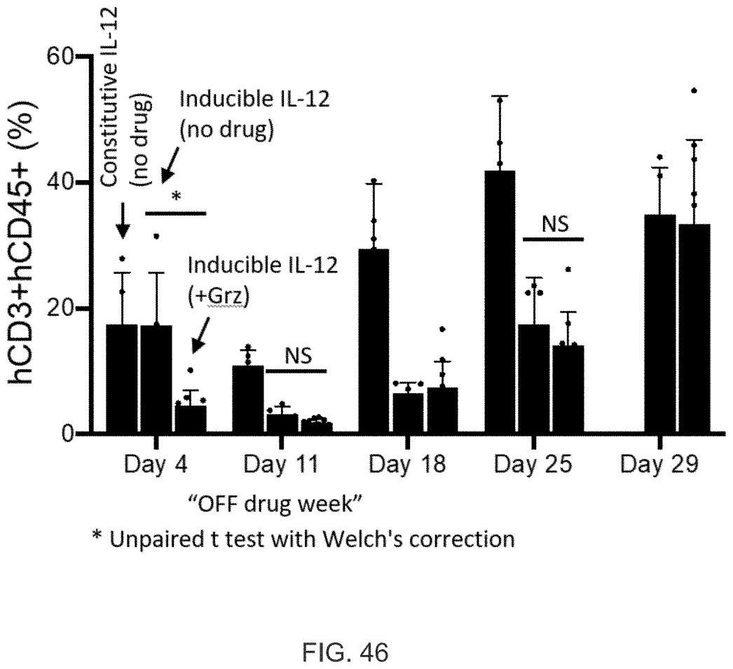

Persistence of the injected T cells was assessed. As shown in FIG. 46, and quantitated in Table 27A (without drug) and Table 27B (with drug), populations of injected human T cells were significantly less at Day 4 when Grz was administered, with no difference seen in the inducible system groups at Days 11, 18, and 25. As previously observed, the constitutive IL-12 group demonstrated increased human T cell numbers in blood, though notably mice in this group demonstrated toxicity and required sacrificing by Day 29.

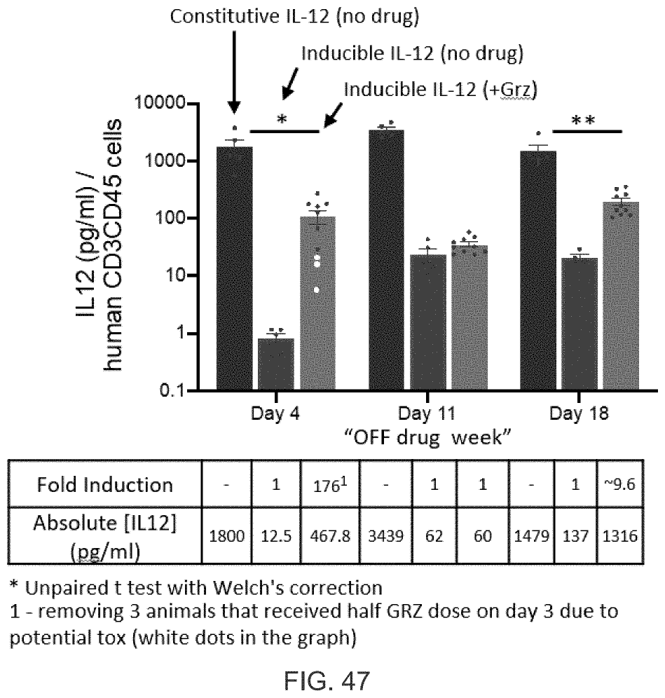

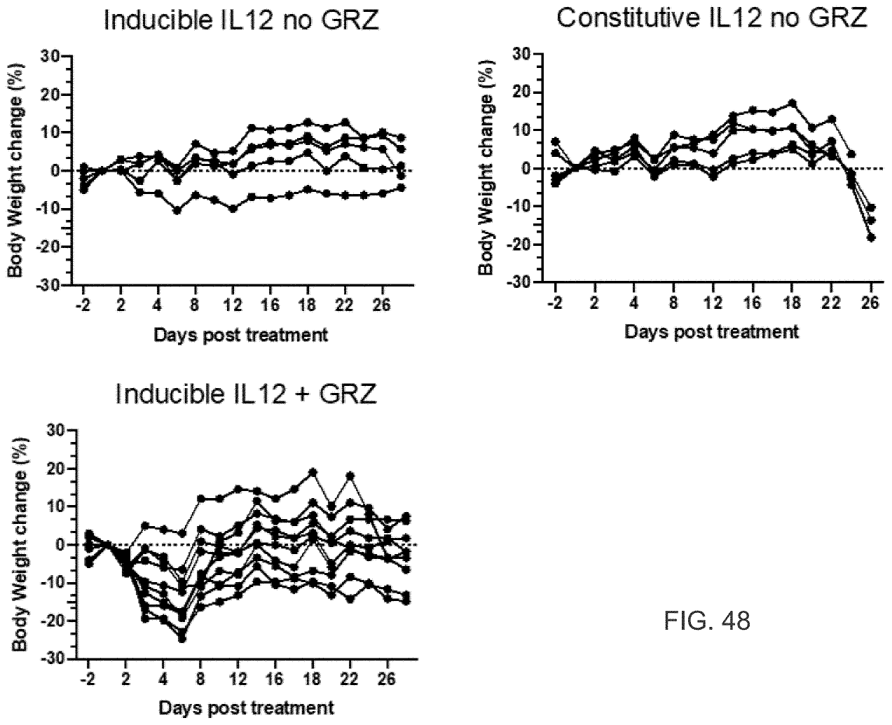

IL-12 cytokine levels were assessed in plasma for the various groups. As shown in FIG. 47, significant Grazoprevir dose dependent induction of IL-12 was observed on Day 4 in the inducible system, with levels between the groups equivalent during the “off” week (Day 11), and further significant Grazoprevir dose dependent induction of IL-12 during the second “on” week (Day 18). During the first week of dosing, 3 of 10 mice in Grz treatment group lost ˜15% weight and received only 30 mg/kg on day 3. IL-12 serum levels in the no-drug did also demonstrate increased serum IL-12 levels over time, suggesting leaky expression of the inducible cassette. Toxicity was also monitored in the different groups. As shown in FIG. 48, body weights remained generally stable for the inducible IL-12 group not administered drug, with a single enlarged spleen and no other abnormalities observed in this group at end time point. Body weights for the constitutive IL-12 group were also generally stable (or slightly increasing) until Day 22 with a decline in body weights afterwards and eventual euthanasia of the group by Day 29, 2 with mice found dead at Day 24, 3 mice euthanized at Day 26 due to weight loss, and spleens for all the mice significantly enlarged compared to the normal NSG mouse spleen (data not shown). In contrast, body weights for the inducible IL-12 group administered Grazoprevir demonstrated transient weight loss for most of the group followed by general weight stabilization. The Grazoprevir also showed signs of toxicity at the end time point, including pale kidneys, thick diaphragm, enlarged spleen, thick liver lobe, white/yellow lump growing inside the liver, and organs (liver, stomach, spleen) sticking with peritoneal wall and each other. The results indicate the Grazoprevir regimen demonstrated drug-dependent IL-12 production with acute toxicity in contrast to the long term lethality of constitutive IL-12 expression in the systems examined.

TABLE 27A
Human T cells in the blood (hCD3+: hCD45+ shown as % of live cells); no drug

| | Constitutive IL-12 | | | Inducible IL-12 (no drug) | | | | |
|---|---|---|---|---|---|---|---|---|
| Mouse | #1 | #2 | #3 | #1 | #2 | #3 | #4 | #5 |
| Day 4 | 6.68 | 27.9 | 16.6 | 17.5 | 31.5 | 13.9 | 9.17 | 14.2 |
| Day 11 | 8.84 | 12.4 | 13.9 | 1.54 | 3.83 | 4.84 | 2.89 | 2.84 |
| Day 18 | 12.2 | 31 | 33.9 | 4.98 | 8.01 | 8.08 | 4.35 | 7.17 |
| Day 25 | 25 | 46.3 | 43 | 22.5 | 22.5 | 23.6 | 10 | 9 |
| Day 29 | nd | nd | nd | 25.4 | 41.1 | 44.1 | 32.1 | 31.6 |

TABLE 27B
Human T cells in the blood (hCD3+: hCD45+ shown as % of live cells); w/Grz

| | Inducible IL-12 (Grz administered) | | | | | | | | | |
|---|---|---|---|---|---|---|---|---|---|---|
| Mouse | #1 | #2 | #3 | #4 | #5 | #6 | #7 | #8 | #9 | #10 |
| Day 4 | 10.2 | 0.82 | 5.8 | 2.11 | 5.39 | 4.92 | 4.03 | 4.04 | 3.76 | 3.6 |
| Day 11 | 2 | 0.95 | 2.55 | 1.53 | 1.59 | 1.07 | 1.51 | 1.98 | 2.27 | 2.71 |
| Day 18 | 6.36 | 3.74 | 4.73 | 11.8 | 16.7 | 9.5 | 5.18 | 4.87 | 7.61 | 4.26 |
| Day 25 | 17.6 | 8 | 14.2 | nd | 11.7 | 14.4 | 11.7 | 12.9 | 9.8 | 26.2 |
| Day 29 | 43.7 | 24.2 | 21 | nd | 45.9 | 18.2 | 36.4 | 17.7 | 38.2 | 54.6 |

### Example 14: Activation Inducible System

Promoters that turn on transcription when CAR cells are activated by target cells are assessed.

### Materials and Methods

Candidate Selection: Using single-cell RNA-seq data for CAR-T cells cultured with or without cognate target cells (Xhangolli et al. Genomics Proteomics Bioinformatics. 2019 April; 17(2):129-139. doi: 10.1016/j.gpb.2019.03.002; herein incorporated by reference for all purposes), top genes upregulated in activated CAR-T cells relative to resting CAR-T cells were identified and regions from 2 kb upstream to −100 bp for those genes identified were chosen as candidate promoters.

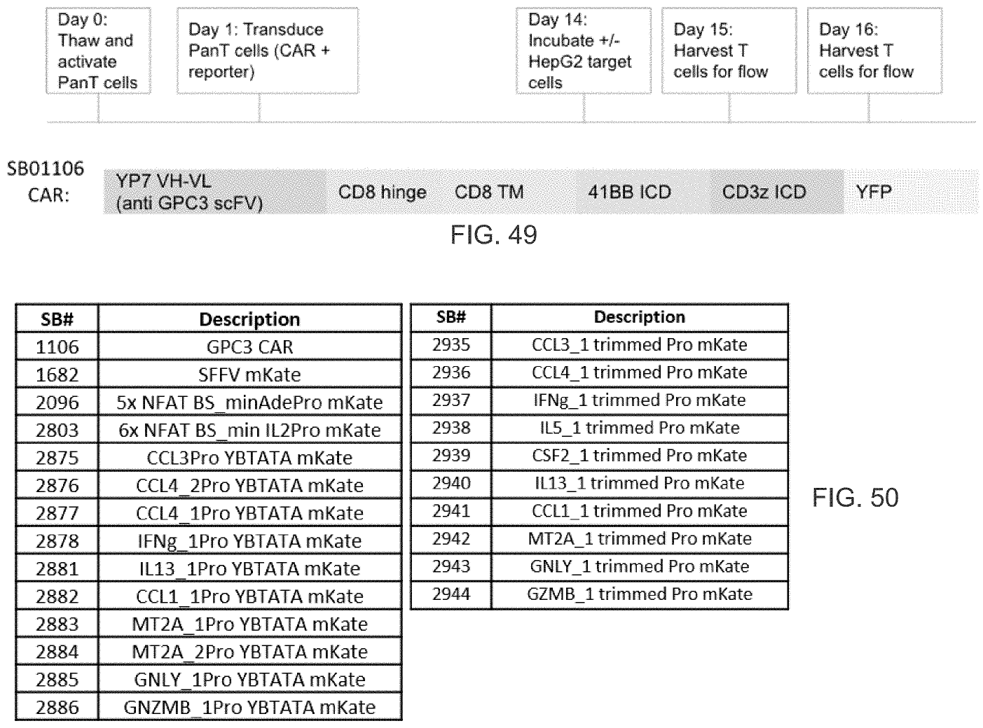

Screening: Pan T cells were virally transduced as described with a GPC3-CAR only construct (“1106”) and viral vector encoding The candidate promoters were either (1) paired with YBTATA (SEQ ID NO: 155; “YBTATA” constructs; or (2) trimmed to the translational start site of the respective gene (“trimmed”) constructs to direct transcription initiation upstream of an mKate reporter. Candidate constructs were screened by flow sorting for mKate expression (gated by YFP CAR+ cells) following either 24 or 48 hours of culturing alone or activated through co-culturing with HepG2 target cells. In addition, candidates based on NFAT transcription factor binding sites was also assessed. The screen workflow is shown in FIG. 49. Constructs assessed are shown in FIG. 50.

### Results

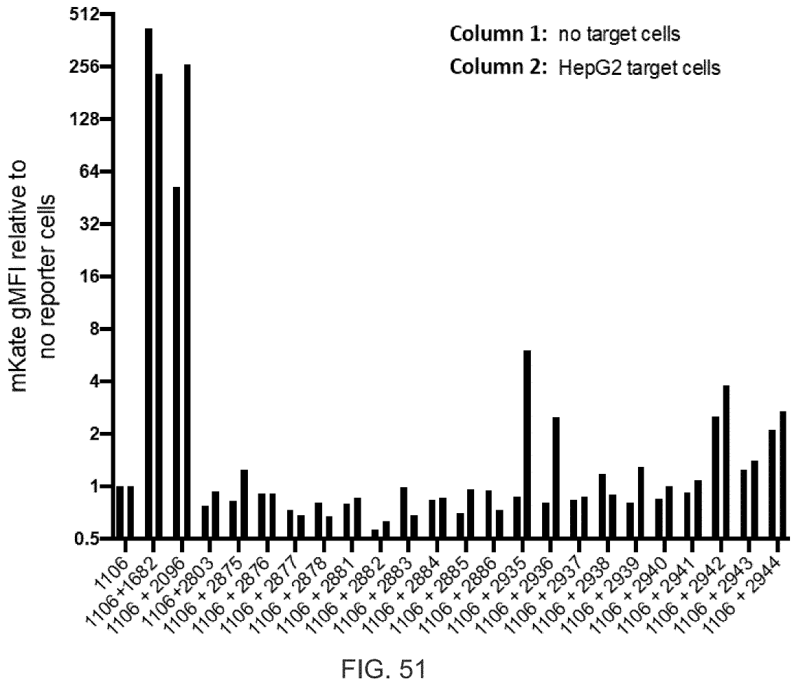

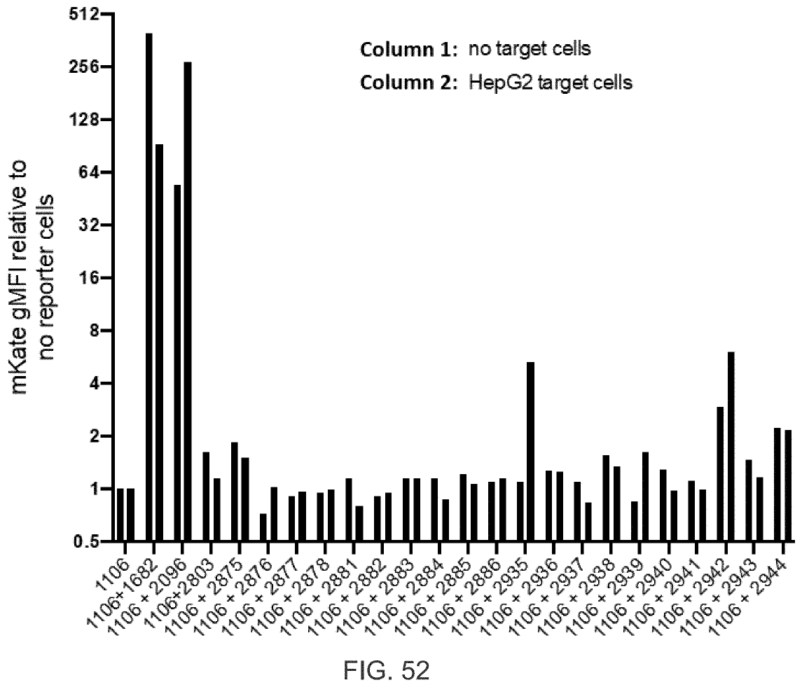

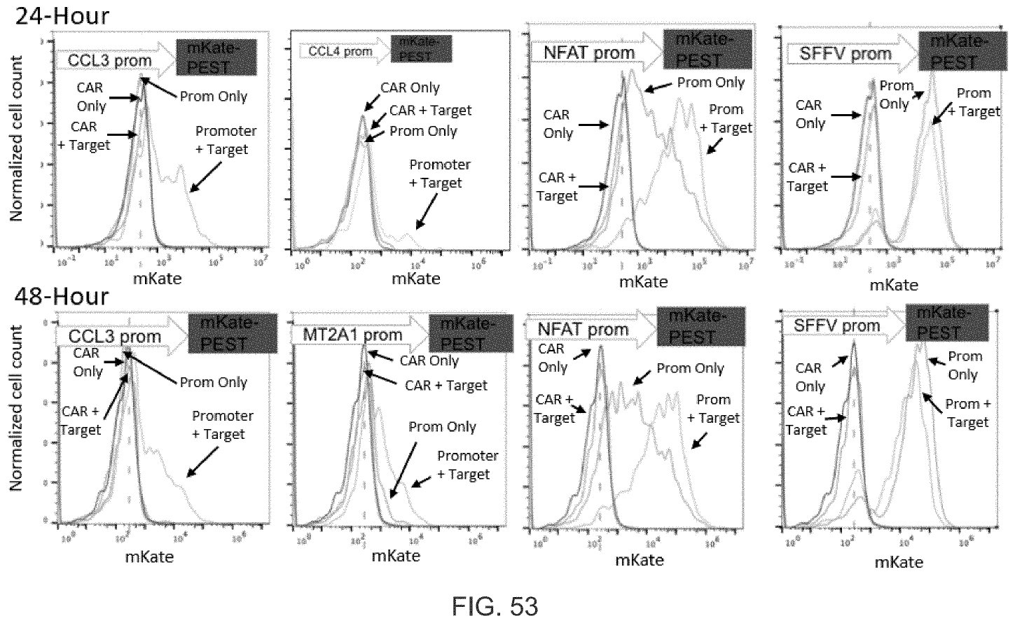

Candidates were screened for enhancing transcription when CAR-T were co-cultured with target cells, with flow-cytometry results for culturing with target cells for 24 hours shown in FIG. 51 and 48 hours shown in FIG. 52. The 5× NFAT BS min AdePro construct (“2096”; SEQ ID NO: 154) demonstrated increased reporter expression when cultured with target cells at both 24 and 48 hours, though notably the construct had a high basal level of reporter expression in the absence of target cells. The 24 hour and 48 hours timepoints identified trimmed CCL3 (“2935”) as demonstrating increased reporter expression when cultured with target cells, notably with reporter expression generally equivalent to background in the absence of target cells. The 24 hour timepoint also identified trimmed CCL4 (“2936”) as demonstrating increased reporter expression when cultured with target cells, notably with reporter expression generally equivalent to background in the absence of target cells. The 48 hour timepoint also identified trimmed MT2a (“2942”) as demonstrating increased reporter expression when cultured with target cells, with reporter expression ˜3-fold above background in the absence of target cells. Histograms of the identified promoters demonstrating reporter expression and their controls (CAR only cultured without HepG2 targets, CAR only cultured with HepG2 targets, CAR+promoter cultured without HepG2 targets) are shown in FIG. 53 and fold-changes as assessed by MFI in Table 28. Sequences for the identified promoters are provided in Table 29. The data demonstrated promoters that turn on transcription when CAR cells are activated by target cells were identified.

TABLE 28
Fold change in mKate gMFI target cells/no treatment

| | 24 h Fold | 48 h Fold |
|---|---|---|
| Constructs | change | change |
| GPC3 CAR only | 1.77 | 1.89 |
| GPC3 CAR + SFFV mKate | 0.97 | 0.43 |
| GPC3 CAR + 5x NF AT BS min | 8.81 | 9.51 |
| AdePro mKate | | |
| GPC3 CAR + CCL3 Pro mKate | 12.12 | 9.03 |
| GPC3 CAR + CCL4 Pro mKate | 5.52 | 1.85 |
| GPC3 CAR + MT2A1 Pro mKate | 2.63 | 3.91 |

TABLE 29
Activation Inducible Promoter Sequences

| SB # | Gene | Sequence |
|---|---|---|
| SB02935 | CCL3 | GGACAGAATTCCAAAGGCATGGTCGCACTTGGCTTCTGTCCTCTGTTATTCTCC |
| | | AGCATCAAATGTATCAACTCTAACCCCTTTGGGGGGAATACAAGGCCTGTCCT |
| | | GGTTTGGTCCCAATTTAGCTTTATCATCCATATTCACCCCCACTGCTCTGCAGCT |
| | | CCACTGAAGCACCCCCTCTTTCCTCTGAACCCACAATGTCACACTCAGGACTCT |
| | | GCCTCAGCTGGGCACTCATCTATAGATGCCTAAATCCCGGGCAGTTATCCAGA |
| | | CACAACTAAAGTTCCATCCCTTCCATGAAGCCTTCCCCAACCCTCTGGTGGAAG |
| | | GTCACTTCTTCCCCTCGTGGGATTCTGAGCTTTCATTTCTTTTTCTACTAGGAGT |
| | | CCTAGCACTTTCGGCTAAATGCTACAATTACCTGTTCATACACTCTACCTGCCC |
| | | CCACGAGATCAGGGGCATCTCAGAAACAAAGATCATTAAAACCAACTAAATCT |
| | | ATTTCTCATTATAAAATGAGGTATGCTGATTGATTGTGAAAGAATAAAATAAC |
| | | AAAGTATGGAAAAGAAAAAAAAGCATATAATCTGGCTGAGAAGGTAGAGACC |
| | | CTTCCACACCACTGAAATTATGTATTGAAAAGAATAAGTAAAAAACTGCTTCA |
| | | ATTTGGCATGATTTATGTAAGTATAGTATAGGATCCTTAAAATGGTTCAAAGA |
| | | AATGGGAAATCAAGACTTCATTTTGGCCAAAACCATTGAACAGAAACTTCAGC |
| | | ATATTTATCAATAATTTCTTTCAGATTAAACAACTGACAACAACCTATTTTTCA |
| | | ACCAGTGATGTTGGAAATGTTTTTTTAAAAATTAGTTTATAAATTTGTGGGCTG |
| | | ACCAAGAAGGTAATAAAGTCTAACTAAGTAAAATGAGAAAAATTCAGAAAAA |
| | | GAAAAAAATAAGAAAATAAATCACCCAGGGACCTATCACACAAATATAAGAA |
| | | CTATTCATTCTTTAAGGCATGTATTTCCAAGCCTTTGTATTTTTTTCCATGCTTA |
| | | GGGTTGGCAAGGAATATATATATATTTGTACAAATATATATGTGTATATGTACA |
| | | AATACATGTATATATAGTACAAATATATATATATATTTGTACAATTCTTCAGAC |
| | | TTTGTAGAATTTGTATAATGTCGTATCTTGCTTTTTTTAACCACTGATGTTATAA |
| | | GCATATTTATGCCACTTCATTCATTTTAGAGACTTAATAATAAATGATCTAGTG |
| | | GATAATTTATCATTCCCTGATGGAGAAAAATTTAGCTTTGTTTATTTTAGAGTT |
| | | ATAAACGATGCTGGGTCAGGTATCTTTATGTTTGAAGATGGCTCCATATTTGGG |
| | | TTGTTTCCACAGAACTCTTTCCTAGAAATGCTTTTTCTAGGTTAATGGCTACAG |
| | | ATATTTCTAGGCACCTGACATATTGACACCCACCTCTAAAGTATTTTTATGATC |
| | | CACAACTAGCGTTTAACACAGCGCCCTAGTCACTACATGACTAATAAATAGAC |
| | | AAATGACTGAAACATGACCTCATGCTTTCTATTCCTCCAGCTTTCATTCAGTTC |
| | | TTTGCCTCTGGGAGGAGGAAGGGTTGTGCAGCCCTCCACAGCATCAGCCCATC |
| | | AACCCTATCCCTGTGGTTATAGCAGCTGAGGAAGCAGAATTGCAGCTCTGTGG |
| | | GAAGGAATGGGGCTGGAGAGTTCATGCACAGACCAGTTCTTATGAGAAGGGA |
| | | CTGACTAAGAATAGCCTTGGGTTGACATATACCCCTCTTCACACTCACAGGAG |
| | | AAACCATTTCCCTATGAAACTATAACAAGTCATGAGTTGAGAGCTGAGAGTTA |
| | | GAGAATAGCTCAAAGATGCTATTCTTGGATATCCTGAGCCCCTGTGGTCACCA |
| | | GGGACCCTGAGTTGTGCAACTTAGCATGACAGCATCACTACGCTTAAAAATTT |
| | | CCCTCCTCACCCCCAGATTCCATTTCCCCATCCGCCAGGGCTGCCTATAAAGAG |
| | | GAGAGCTGGTTTCAGACTTCAGAAGGACACGGGCAGCAGACAGTGGTCAGTCC |
| | | TTTCTTGGCTCTGCTGACACTCGAGCCCACATTCCGTCACCTGCTCAGAATC |
| | | (SEQ ID NO: 156) |
| SB02936 | CCL4 | TTAAGTCTTCAACCCATTGTGAGTTGATTTTTACAGATGGTGTAAGGAAGGGGT |
| | | CCAGTTTCAATCTTTTGCATACAGCTAGCCAGTTATCCCAGTACCATTTATTGA |
| | | CTGGGAAGTCCTTTCCCTGTTGCTTGTTTTTGTTAACTTTGTCAAAGTTCGGATG |
| | | GTTGCAGGTATGCAGCATTATTTCTGAGCTCTCTATTTTGTTCTATTGGTCTATG |
| | | AGTCTGTTTTTGTACCAAAGCCATGCTGTTTTGGTTACTGCAGCCCTGTGGTAT |
| | | AGTTTGAAGTCATGGCTCCCTTTTTTTACTCTTCTTTTTTCTTTCTCAGAGAAGA |
| | | GCAGGTAACAATGTGGGGTTAAAGGTGAGCAGGTGGGTTAGAGTAGTGGAGC |
| | | TAAAGGCAGATAGAGGTCTTGATCCAATTATCCTCATTCCCTTCATTTGTCCAA |
| | | CCTTGCACATTCCTGTATGCAGGGCCTTTGTAAGCTTCACCTTCTGTACAGGTG |
| | | CCCAGCAACTGTCAAACCAGCCAGGGCTTAACATGAGCATGGGCATTCCCTAT |
| | | GGACTGGCTGGGACACACCCACATCCTTAGATTACACATTTTGCCTGTAACATA |
| | | GATAAACTAATTACTAGGTATATAATTCTGTTTCGTTTTGTTTTGCTTTGTTTTG |
| | | TTTTTGGGACAGGGTCTCATTCTGTCAAACAAGCGGGAGTGCAGTGGCACAAT |
| | | CACCACTCACTGCAGCCTGGACTTTTCTGGGCTCAGGTGATCTTCCCACCTCAG |
| | | CCTACTGAGCAGCTGGGACTACAGGTGTGTATCACCACTCCCAGCCAAAATAT |
| | | TTTTTTATAGAGACAGTGTTTCACCATGTTGCCCAGTCTGGTATCGAACTTCTG |
| | | GGCTCAAGCTATCTGCCTGCCTTGACCTCCCAAAGTGCTGGGATTACAGGTGTG |
| | | AGGCCCTGTACCCAGCTCAATCCTGTTTTTATACTAGAAGAACATTCTCTATCT |
| | | GGGTCTTCCAAACTTGATTCTTACTGGTTAATTTGTCCTGTTCTTTTGCTGGTCC |
| | | AAGAAAATATCCTGAAATCTTTATTTCTTCTCCGTTTCCTCCTTGTCCTAGGACA |
| | | GACTAGCCCTATCCCCTTCCTGAATTAAGTCCGAATATAGTCAGTCTTTGAGTG |
| | | TGGAATAGCTCCTAGCAGTCTATCAGTCAACGGGTTCTCTTTGTGGTCACATTC |
| | | TATGTTTATTCAGGAGACTACAGCATGGAAAGAAAATGGACTTTGGAGTCAGG |
| | | TGAATCTTGATTCAAATCTTGGCAGTGCCATTCATCAGCTCTGTGGCATTGAGC |
| | | GCATCAGTGGACCACTCTCTGATCCCCAATTGCCTTGTCTGTAAAATGAGATTA |
| | | GACACCCCTGCTCAAGATTATGGTAGGATTATATATAATGTGTGTAACACAGC |
| | | TTTTCCAGTGCCTGGTACAAGGTAAGTGTCCAATAAGAAGTTACAAATGTTCCT |
| | | GAGCCACCCTACTGGGTCCCTCCTCACATCCAATGCTGGACTCTTCATGCCCAA |
| | | GGAAACATATAAGGCACAAAAGTCAGTGGCACTCAGCTCAGCAGAAGAAGGC |
| | | ACAGGAAGGGGATGGGGGAGTCCTGGCTCCCACTTTGTTCTTGGGGTCAGGCT |
| | | TGTGGCCAGCCTGGAAAGACTGACATGAACCCTCCAGCATCTGAGGGTGCCAA |
| | | CCACCAAGAGCAGCACAGTTCTTGTCTACAAGCCACTTGTAGCAGGTGTGAAC |
| | | ATTTCATCTTTTCCTCTGGGGTTTGCAACTCTCTCCCCAGTCTCGGTCACTGCCT |
| | | TTGGCTGTACCACTTCCCTTTTCTTCTCGCCTGCACCTCCCTCATCTTTCCTCTAT |
| | | GATGACATCGCCCTGGGGAAGAGAAGCTGAGAGGAACTCCTCACTCAGCTAGC |
| | | TTCAGGAGCATGACGTCATCTCTACCATGGAAATTCCACTCACTCTCCTGTGCC |
| | | CCCACATTTGTCCTAGGCCTCAGAGTCCCTATAAAGAGAGATTCCCAAGTCAG |
| | | TATCAGCACAGGACACAGCTGGGTTCTGAAGCTTCTGAGTTCTGCAGCCTCAC |
| | | CTCTGAGAAAACCTCTTTTCCACCAATACC (SEQ ID NO: 157) |
| SB02942 | MT2A | GGGATTTGGTATATAATACTGTGCATACATAAATAACAACAACAAAATTGGTC |
| | | CACTGCCCAGAGAGCTCTCGATCCACAGAGCTGCCAGACATCATGAGATTTCA |
| | | AAAGATCTTGGCTCTAAAATCAGAGGATGTTTGGATATAAATTCTAGTACAAC |
| | | CCAGTCATTTGACAGATGAGTAAACCAGGGATCAGAAGAGTCCAGGCATGTAA |
| | | TTGCCCCAAGGTCACTGAGTAAGTGAGTGGCCGAGTGACCACTGAAATTTAGT |
| | | CCATCTGAGAGCTCGCCCCCCTGCCCTGTGCGGTGTTTTTACAAGAGTGAGCAC |
| | | GCAGGTTCCAGGGTGGTCAAGAGGTGTTTACTTTCATTTTGTAGTCGGCCTTCA |
| | | TACAAGGTGGGGGCTGGGAAGGGAAGAGCAGTCCAGGCCATTACCCCCATGA |
| | | ATTCATTTGACAAATATTTATTAAGCGTCTACTGCAAACTTGGCCCGGTGCTGG |
| | | AAGCTGAAGACACAATTCCAGAGGCAGACAATGACTGCCCTTGGCATTTCCAG |
| | | TGGTCAGGGGTTGGTGAGGAGGGGCAGAGACACCAATCCAAGAGTCCCCGCA |
| | | GTTTATACATACCACAGGCACGGAAAGCGCCAGGAGGGGAAAGAACAGGATG |
| | | TTTACCAGCACCTTCCACAGGCAGCCACGGTCATGGGGGTCAGGACAGGGTCC |
| | | CCAGAGGAAGTGACGATTCGGCTGAGCTAGAAAGACATTTCCACAGGAACTTA |
| | | CAGTAAGGGCTGCAAGGACAGCCTGTCCTCCCCCGCCCACCACCTCACTAAAC |
| | | TTTCACTGTGGCAATCGGCATTCCCTAAGCCTGCCAGGAAGCTTCCAGTAACCA |
| | | CTTCCTGACTCCTAGCATGACACACTTCGGGCCTTCCAAGGTTGACGATCTAAG |
| | | GCCCTTACACAGCGCCAGACACGCGCAGGCAGGGAGGCAGGATGTGCCTCTCC |
| | | AGACAGGAATGTGGACGCCACCTGGGCTCTTCCCCTCAGCCTCCACAGGGGAA |
| | | GAATATTCTTGTGGGGTTTTTCCCTTCCAAATGTCTCAGGGCGATTCCAGTGTT |
| | | CCCGCTAGTTCCTCCCTCCCAGGCTAGAACACAAATCCTTCCCACTCCCTGCCT |
| | | GGCAAACACCTTCTGACCCTCAGGCCCAAGGCAATGGCCCACCTCCTCCCAGG |
| | | CTGGATGGGGTCTCCTCCTCTCTGTTCCCCCAGCCCCTGAGCTTCCTGAGGACC |
| | | AAGCTTGTGGCTTCTTCTCCTTACTCTTCCTCCTTGGTGTCTCTATGTTAGAGGG |
| | | CCGTTAGCATCTGCTGGGGCCTGGTCGCATTCACCCTGCTCTGCCACTCACTGG |
| | | CTGTGTGACTCTGGACAAATTAACTTCTCTGGACCTGCAGTTTCTCCTCTCTAC |
| | | AATGAGAATACTGGAGAGTCCTTATCTTATGGGTTGCTACAGAATTAAGTGAC |
| | | ATCTCACACACAACACACTTCCTACAGTCCCTGTTACACGCTAAAAGTACTCAA |
| | | CATGCAACGGATACGTCATCAGTAACCACCCCACGGGTTTACTGTGATGCTGC |
| | | ACAATTATTAAGCCTTGGCTGCTACAGAGTTGTAACCTGTCTGCACTTCCAACC |
| | | GGTTTGGGAATGCAAGCAGCATTCCCAAGTCCCGCTTTCACCCGCGCGCTAAC |
| | | GGCTCAGGTTCGAGTACAGGACAGGAGGGAGGGGAGCTGTGCACACGGCGGA |
| | | GGCGCACGGCGTGGGCACCCAGCACCCGGTACACTGTGTCCTCCCGCTGCACC |
| | | CAGCCCCTTCCGCGCCGAGGCGTCCCCGAGGCGCAAGTGGGCCGCCTTCAGGG |
| | | AACTGACCGCCCGCGGCCCGTGTGCAGAGCCGGGTGCGCCCGGCCCAGTGCGC |
| | | GCGGCCGGGTGTTTCGCCTGGAGCCGCAAGTGACTCAGCGCGGGGCGTGTGCA |
| | | GGCAGCGCCCGGCCGGGGCGGGGCTTTTGCACTCGTCCCGGCTCTTTCTAGCTA |
| | | TAAACACTGCTTGCCGCGCTGCACTCCACCACGCCTCCTCCAAGTCCCAGCGA |
| | | ACCCGCGTGCAACCTGTCCCGACTCTAGCCGCCTCTTCAGCTCGCC (SEQ ID |
| | | NO: 158) |

### Example 15: IL-12 Drug-Inducible Systems in T Cells with Grazoprevir/Elbasvir

IL-12 payload expression was assessed in T cells for various regulatable TF expression system strategies using either grazoprevir or the combination grazoprevir/elbasvir.

### Materials and Methods

For in vitro assessment, on Day 0, CD4/CD8 T cells (donor derived) were thawed, seeded at 1e6 cells/mL/well in a 24 well-plate, and activated with anti-CD3/CD28 Dynabeads (3:1 bead to cell) in complete T cell media (Optimizer+Supplements—Gibco+5% human serum)+rhIL-2 (100 Units/ml; Peprotech). On Day 1, 0.5 ml media was removed and cells were transduced with 3e5 pg of virus for each the constructs indicated (relevant sequences of the constructs are provided in Table 17). On Day 2, 1.5 mL of Optimizer media+100U/mL rhIL-2 was added. Day 7 cells were treated with grazoprevir (2, 1, 0.5, 0.1, 0.05, 0.01, 0.00504 and no drug) with or without elbasvir at a ratio of 2:1, respectively, in line with the ratio of compounds in Zepatier®. Transduced T cells were incubated for a further two days and the supernatant collected via centrifugation for IL-12 quantification and assessed by Luminex (R&D IL12).

### Results

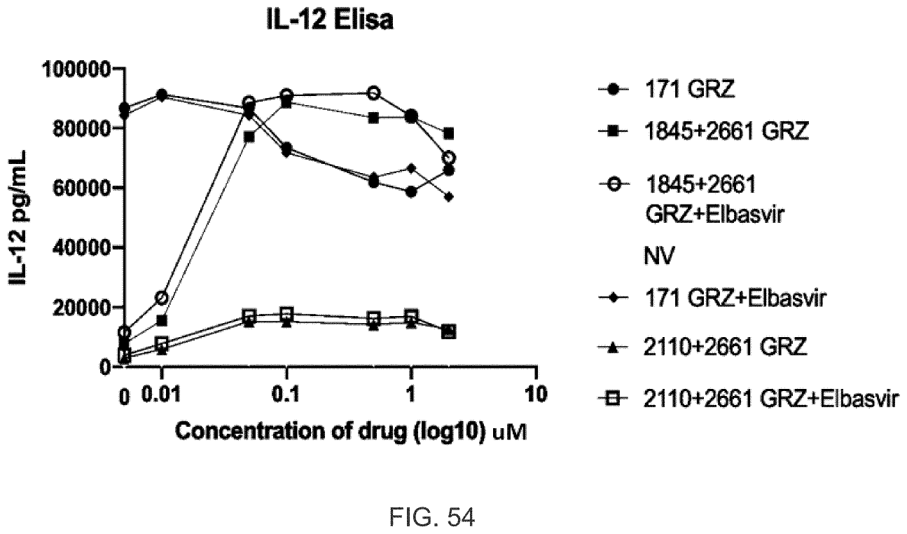

IL-12 payload expression was assessed in T cells for various regulatable TF expression system strategies and constructs. An drug-inducible format ACP (also referred to as “synTF”) using an NS3/NS4 protease cleavage site and a VPR transcriptional effector domain (SB01845) or a modified version of the ACP with linkers shortened and the entire construct codon optimized (SB02110) were assessed. The payload construct SB02661 encoded a GPC3-CAR driven by an SFFV promoter, with the IL-12 cytokine payload encoded in a cassette including an A2 insulator and YB-TATA promoter driving expression in the opposite direction as the CAR cassette (e.g., see orientation of reporter and CAR cassettes in construct “2235” in FIG. 20). Control T cells were transduced with constitutive IL-12 construct SB00171. As shown in FIG. 54 and quantified in Table 30, IL-12 production was observed in a concentration dependent manner following addition of the NS3 protease inhibitor grazoprevir, in line with those seen above. The kinetics of IL-12 production were generally equivalent regardless of elbasvir addition. Accordingly, elbasvir did not impact grazoprevir potency in the induction of IL-12 in a drug-inducible format ACP TF expression system.

TABLE 30
IL-12 Production with Grazoprevir (Grz) With or Without Elbasvir

| GRZ conc | 171 | 1845 + 2661 | 2110 + 2661 | | 171 GRZ + | 1845 + 2661 | 2110 + 2661 |
|---|---|---|---|---|---|---|---|
| (uM) | GRZ | GRZ | GRZ | NV | Elbasvir | GRZ + Elbasvir | GRZ + Elbasvir |
| 2 | 65992 | 78330 | 12647 | 1314 | 57054 | 70010 | 11823 |
| 1 | 58677 | 83739 | 14837 | 1133 | 66533 | 84048 | 16897 |
| 0.5 | 61896 | 83559 | 14193 | 1288 | 63545 | 91776 | 16228 |
| 0.1 | 73436 | 88685 | 15146 | 1159 | 71788 | 90926 | 17825 |
| 0.05 | 86701 | 77119 | 15249 | 1468 | 84332 | 88556 | 17026 |
| 0.01 | 91312 | 15558 | 5847 | 1133 | 90488 | 23156 | 7779 |
| 0.005 | 86701 | 7779 | 2924 | 567 | 84332 | 11578 | 3889 |
| 0 | 86701 | 1005 | 902 | 953 | 84332 | 1133 | 927 |

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

1. An engineered expression system comprising:
(a) a first expression cassette comprising a first promoter and a first exogenous polynucleotide sequence encoding an activation-conditional control polypeptide (ACP), wherein the first promoter is operably linked to the first exogenous polynucleotide; and
(b) a second expression cassette comprising an ACP-responsive promoter and a second exogenous polynucleotide sequence having the formula:
(L-E)ₓ
wherein
E comprises a polynucleotide sequence encoding an effector molecule,
L comprises a linker polynucleotide sequence,
X=1 to 20,
wherein the ACP-responsive promoter is operably linked to the second exogenous polynucleotide, wherein for the first iteration of the (L-E) unit, L is absent, and
wherein the ACP is capable of inducing expression of the second expression cassette by binding to the ACP-responsive promoter,
optionally wherein when the second expression cassette comprises two or more units of (L-E)ₓ, each linker polynucleotide sequence is operably associated with the translation of each effector molecule as a separate polypeptide,
optionally wherein the second expression cassette comprising one or more units of (L-E)ₓ further comprises a polynucleotide sequence encoding a secretion signal peptide for each X,
optionally wherein for each X the corresponding secretion signal peptide is operably associated with the effector molecule,
optionally wherein each secretion signal peptide comprises a native secretion signal peptide native to the corresponding effector molecule,
optionally wherein each secretion signal peptide comprises a non-native secretion signal peptide that is non-native to the corresponding effector molecule,
optionally wherein each secretion signal peptide comprises a non-native secretion signal peptide that is non-native to the corresponding effector molecule and optionally wherein the non-native secretion signal peptide is a secretion signal peptide of a molecule selected from the group consisting of: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12, and
optionally wherein the first expression cassette is comprised within a first nucleic acid and the second expression cassette is comprised within a second nucleic acid, or wherein the first expression cassette and the second expression cassette are comprised within a single nucleic acid.

2. The engineered expression system of claim 1, further comprising a linker polynucleotide sequence localized between the first expression cassette and the second expression cassette,
optionally wherein the linker polynucleotide sequence is operably associated with the translation of the ACP and each effector molecule as separate polypeptides,
optionally wherein the linker polynucleotide sequence encodes a 2A ribosome skipping tag and optionally wherein the 2A ribosome skipping tag is selected from the group consisting of: P2A, T2A, E2A, and F2A,
optionally wherein the linker polynucleotide sequence encodes an Internal Ribosome Entry Site (IRES), and
optionally wherein the linker polynucleotide sequence encodes a cleavable polypeptide and optionally wherein the cleavable polypeptide comprises a furin polypeptide sequence.

3. The engineered expression system of claim 1, wherein:
(a) the ACP-responsive promoter comprises an ACP-binding domain sequence and a promoter sequence,
optionally wherein the promoter sequence is derived from a promoter selected from the group consisting of: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof,
optionally wherein the ACP-responsive promoter comprises a synthetic promoter,
optionally wherein the ACP-responsive promoter comprises a minimal promoter, and
optionally wherein the ACP-binding domain comprises one or more zinc finger binding sites;
(b) the first promoter comprises a constitutive promoter, an inducible promoter, or a synthetic promoter,
optionally wherein the constitutive promoter is selected from the group consisting of:
CMV, EFS, SFFV, SV40, MND, PGK, UbC, hEF1aV1, hCAGG, hEF1aV2, hACTb,
heIF4A1, hGAPDH, hGRP78, hGRP94, hHSP70, hKINb, and hUBIb; and/or
(c) each effector molecule is independently selected from a therapeutic class, wherein the therapeutic class is selected from the group consisting of: a cytokine, a chemokine, a homing molecule, a growth factor, a co-activation molecule, a tumor microenvironment modifier a, a receptor, a ligand, an antibody, a polynucleotide, a peptide, and an enzyme, optionally wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha,
optionally wherein the chemokine is selected from the group consisting of: CCL21a, CXCL10, CXCL11, CXCL13, a CXCL10-CXCL11 fusion protein, CCL19, CXCL9, and XCL1,
optionally wherein the homing molecule is selected from the group consisting of: anti-integrin alpha4,beta7; anti-MAdCAM; CCR9; CXCR4; SDF1; MMP-2; CXCR1;
CXCR7; CCR2; CCR4; and GPR15,
optionally wherein the growth factor is selected from the group consisting of: FLT3L and GM-CSF,
optionally wherein the co-activation molecule is selected from the group consisting of: c-Jun, 4-1BBL and CD40L,
optionally wherein the tumor microenvironment modifier is selected from the group consisting of: adenosine deaminase, TGFbeta inhibitors, immune checkpoint inhibitors, VEGF inhibitors, and HPGE2, optionally wherein the TGFbeta inhibitors are selected from the group consisting of: an anti-TGFbeta peptide, an anti-TGFbeta antibody, a TGFb-TRAP, and combinations thereof,
optionally wherein the immune checkpoint inhibitors are selected from the group consisting of: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA, antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies,
optionally wherein the VEGF inhibitors comprise anti-VEGF antibodies, anti-VEGF peptides, or combinations thereof, and
optionally wherein each effector molecule is a human-derived effector molecule.

4. The engineered expression system of claim 1, wherein:
(a) the first expression cassette and/or the second expression cassette further comprises an additional exogenous polynucleotide sequence encoding an antigen recognizing receptor, or
(b) the engineered expression system further comprises an additional expression cassette comprising an additional promoter and an additional exogenous polynucleotide sequence encoding an antigen recognizing receptor,
wherein the additional promoter is operably linked to the additional exogenous polynucleotide, optionally wherein the additional exogenous polynucleotide sequence are encoded by the same polynucleotide as the first expression cassette or the second expression cassette, and
optionally wherein the antigen recognizing receptor recognizes GPC3,
optionally wherein the antigen recognizing receptor comprises an antigen-binding domain,
optionally wherein the antigen-binding domain that binds to GPC3 comprises a heavy chain variable (VH) region and a light chain variable (VL) region,
wherein the VH comprises:
a heavy chain complementarity determining region 1 (CDR-H1) having the amino acid sequence of KNAMN (SEQ ID NO: 119),
a heavy chain complementarity determining region 2 (CDR-H2) having the amino acid sequence of RIRNKTNNYATYYADSVKA (SEQ ID NO: 120), and
a heavy chain complementarity determining region 3 (CDR-H3) having the amino acid sequence of GNSFAY (SEQ ID NO: 121), and
wherein the VL comprises:
a light chain complementarity determining region 1 (CDR-L1) having the amino acid sequence of KSSQSLLYSSNQKNYLA (SEQ ID NO: 122),
a light chain complementarity determining region 2 (CDR-L2) having the amino acid sequence of WASSRES (SEQ ID NO: 123), and
a light chain complementarity determining region 3 (CDR-L3) having the amino acid sequence of QQYYNYPLT (SEQ ID NO: 124),
optionally wherein the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of
(SEQ ID NO: 125)
EVQLVETGGGMVQPEGSLKLSCAASGFTFNKNAMNWVRQAPGKGLEW
VARIRNKTNNYATYYADSVKARFTISRDDSQSMLYLQMNNLKIEDTA
MYYCVAGNSFAYWGQGTLVTVSA
or
(SEQ ID NO: 126)
EVQLVESGGGLVQPGGSLRLSCAASGFTFNKNAMNWVRQAPGKGLEW
VGRIRNKTNNYATYYADSVKARFTISRDDSKNSLYLQMNSLKTEDTA
VYYCVAGNSFAYWGQGTLVTVSA,
optionally wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of
(SEQ ID NO: 127)
DIVMSQSPSSLVVSIGEKVTMTCKSSQSLLYSSNQKNYLAWYQQKPG
QSPKLLIYWASSRESGVPDRFTGSGSGTDFTLTISSVKAEDLAVYYC
QQYYNYPLTFGAGTKLELK,
or
(SEQ ID NO: 128)
DIVMTQSPDSLAVSLGERATINCKSSQSLLYSSNQKNYLAWYQQKPG
QPPKLLIYWASSRESGVPDRFSGSGSGTDFTLTISSLQAEDVAVYYC
QQYYNYPLTFGQGTKLEIK,
optionally wherein the antigen-binding domain comprises an antibody, an antigen-binding fragment of an antibody, a F(ab) fragment, a F(ab′) fragment, a single chain variable fragment (scFv), or a single-domain antibody (sdAb),
optionally wherein the VH and VL are separated by a peptide linker,
optionally wherein when the antigen-binding domain comprises an scFv, the scFv comprises the structure VH-L-VL or VL-L-VH, wherein VH is the heavy chain variable domain, L is the peptide linker, and VL is the light chain variable domain,
optionally wherein the antigen recognizing receptor is a chimeric antigen receptor (CAR) or T cell receptor (TCR),
optionally wherein when the antigen recognizing receptor is a CAR, the CAR comprises one or more intracellular signaling domains, and each of the one or more intracellular signaling domains is selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain,
optionally wherein the CAR comprises a transmembrane domain, and the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain, and
optionally wherein the CAR comprises a spacer region between the antigen-binding domain and the transmembrane domain.

5. The engineered expression system of claim 1, wherein the ACP is a transcriptional modulator,
optionally wherein the ACP is a transcriptional repressor or the ACP is a transcriptional activator,
optionally wherein the ACP further comprises a repressible protease and one or more cognate cleavage sites of the repressible protease,
optionally wherein the ACP further comprises a hormone-binding domain of estrogen receptor (ERT2 domain), optionally wherein the ACP is capable of undergoing nuclear localization upon binding of the ERT2 domain to tamoxifen or a metabolite thereof, and
optionally wherein the tamoxifen metabolite is selected from the group consisting of: 4-hydroxytamoxifen, N-desmethyltamoxifen, tamoxifen-N-oxide, and endoxifen optionally wherein the ACP is a transcription factor, optionally, wherein the transcription factor is a zinc-finger-containing transcription factor
optionally wherein the ACP comprises a DNA-binding zinc finger protein domain (ZF protein domain) and a transcriptional effector domain, optionally wherein the ZF protein domain is modular in design and is composed of zinc finger arrays (ZFA), and optionally wherein the ZF protein domain comprises one to ten ZFA,
optionally wherein the effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a histone acetyltransferase (HAT) core domain of the human E1A-associated protein p300 (p300 HAT core activation domain); a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain,
optionally wherein the one or more cognate cleavage sites of the repressible protease are localized between the ZF protein domain and the effector domain,
optionally wherein the repressible protease is hepatitis C virus (HCV) nonstructural protein 3 (NS3),
optionally wherein the cognate cleavage site comprises an NS3 protease cleavage site, optionally wherein the NS3 protease cleavage site comprises a NS3/NS4A, a NS4A/NS4B, a NS4B/NS5A, or a NS5A/NS5B junction cleavage site,
optionally wherein the NS3 protease can be repressed by a protease inhibitor, optionally wherein the protease inhibitor is selected from the group consisting of:
simeprevir, danoprevir, asunaprevir, ciluprevir, boceprevir, sovaprevir, paritaprevir, telaprevir, grazoprevir, glecaprevir, and voxiloprevir, or the protease inhibitor is grazoprevir, or the protease inhibitor comprises grazoprevir and elbasvir,
optionally wherein the grazoprevir and the elbasvir is co-formulated in a pharmaceutical composition,
optionally wherein the pharmaceutical composition is a tablet,
optionally wherein the grazoprevir and the elbasvir are at a 2 to 1 weight ratio, optionally wherein the grazoprevir is 100 mg per unit dose and the elbasvir is 50 mg per unit dose,
optionally wherein the ACP further comprises a degron, and wherein the degron is operably linked to the ACP,
optionally wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box,
optionally wherein the degron comprises a cereblon (CRBN) polypeptide substrate domain capable of binding CRBN in response to an immunomodulatory drug (IMiD) thereby promoting ubiquitin pathway-mediated degradation of the ACP,
optionally wherein the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN,
optionally wherein the CRBN polypeptide substrate domain is a chimeric fusion product of native CRBN polypeptide sequences,
optionally wherein the IMiD is an FDA-approved drug,
optionally wherein the IMiD is selected from the group consisting of: thalidomide, lenalidomide, and pomalidomide, and
optionally wherein the degron is localized 5′ of the repressible protease, 3′ of the repressible protease, 5′ of the ZF protein domain, 3′ of the ZF protein domain, 5′ of the effector domain, or 3′ of the effector domain.

6. The engineered expression system of claim 1, wherein:
(a) the engineered expression system further comprises an insulator, optionally wherein the insulator is localized between the first expression cassette, the second expression cassette, and/or the additional expression cassette if present;
(b) the first expression cassette is localized in the same orientation relative to the second expression cassette or the first expression cassette is localized in the opposite orientation relative to the second expression cassette; and/or
(c) the engineered expression system is a nucleic acid selected from the group consisting of: a DNA, a cDNA, an RNA, an mRNA, and a naked plasmid.

7. One or more expression vectors comprising the first expression cassette, the second expression cassette, and/or the additional expression cassette if present of the engineered expression system of claim 1, and
optionally wherein (a) a first vector comprises the first expression cassette and the additional expression cassette if present, and a second vector comprises the second expression cassette, (b) a first vector comprises the first expression cassette, and a second vector comprises the second expression cassette and the additional expression cassette if present, (c) a first vector comprises the first expression cassette and the second expression cassette, and a second vector comprises the additional expression cassette if present, or (d) a vector comprises the first expression cassette, the second expression cassette, and the additional expression cassette if present.

8. An isolated cell comprising the engineered expression system of claim 1,
optionally wherein the engineered expression system is recombinantly expressed,
optionally wherein the engineered expression system is expressed from a one or more vectors or one or more selected loci from the genome of the cell,
optionally wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell, and
optionally wherein the cell is autologous or the cell is allogeneic.

9. A pharmaceutical composition comprising the engineered expression system of claim 1, and a pharmaceutically acceptable carrier, pharmaceutically acceptable excipient, or a combination thereof.

10. A method of treating a subject in need thereof, the method comprising administering a therapeutically effective dose of the isolated cell of claim 8.

11. A method of stimulating a cell-mediated immune response to a tumor cell in a subject, the method comprising administering to a subject having a tumor a therapeutically effective dose of the isolated cell of claim 8.

12. A method of providing an anti-tumor immunity in a subject, the method comprising administering to a subject in need thereof a therapeutically effective dose of the isolated cell of claim 8.

13. A method of reducing tumor volume in a subject, the method comprising administering to a subject having a tumor a composition comprising the isolated cell of claim 8.

14. The method of claim 10, wherein:
(a) the administering comprises systemic administration or intratumoral administration;
(b) the isolated cell is derived from the subject or the isolated cell is allogeneic with reference to the subject;
(c) the method further comprises administering a checkpoint inhibitor;
(d) the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor;
(e) the method further comprises administering a protease inhibitor,
optionally wherein the protease inhibitor is administered in a sufficient amount to repress a repressible protease,
optionally wherein the protease inhibitor is administered prior to, concurrently with, subsequent to administration of the engineered cells or the composition comprising the engineered cells,
optionally wherein the protease inhibitor is selected from the group consisting of:
simeprevir, danoprevir, asunaprevir, ciluprevir, boceprevir, sovaprevir, paritaprevir, telaprevir, grazoprevir, glecaprevir, and voxiloprevir, or the protease inhibitor is grazoprevir, or the protease inhibitor comprises grazoprevir and elbasvir, optionally wherein when the protease inhibitor comprises grazoprevir and elbasvir, the grazoprevir and the elbasvir is co-formulated in a pharmaceutical composition,
optionally wherein the pharmaceutical composition is a tablet
optionally wherein the grazoprevir and the elbasvir are at a 2 to 1 weight ratio, and
optionally wherein the grazoprevir is 100 mg per unit dose and the elbasvir is 50 mg per unit dose; and/or
(f) the method further comprises administering tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen.

15. A kit for treating and/or preventing cancer, comprising the isolated cell of claim 8, optionally wherein the kit further comprises written instructions for using the cell or composition for treating and/or preventing cancer in a subject.

16. The method of claim 11, wherein:
(a) the administering comprises systemic administration or intratumoral administration;
(b) the isolated cell is derived from the subject or the isolated cell is allogeneic with reference to the subject;
(c) the method further comprises administering a checkpoint inhibitor;
(d) the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor;
(e) the method further comprises administering a protease inhibitor,
optionally wherein the protease inhibitor is administered in a sufficient amount to repress a repressible protease,
optionally wherein the protease inhibitor is administered prior to, concurrently with, subsequent to administration of the engineered cells or the composition comprising the engineered cells,
optionally wherein the protease inhibitor is selected from the group consisting of:
simeprevir, danoprevir, asunaprevir, ciluprevir, boceprevir, sovaprevir, paritaprevir, telaprevir, grazoprevir, glecaprevir, and voxiloprevir, or the protease inhibitor is grazoprevir, or the protease inhibitor comprises grazoprevir and elbasvir, optionally wherein when the protease inhibitor comprises grazoprevir and elbasvir, the grazoprevir and the elbasvir is co-formulated in a pharmaceutical composition, optionally wherein the pharmaceutical composition is a tablet
optionally wherein the grazoprevir and the elbasvir are at a 2 to 1 weight ratio, and
optionally wherein the grazoprevir is 100 mg per unit dose and the elbasvir is 50 mg per unit dose; and/or
(f) the method further comprises administering tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen.

17. The method of claim 12, wherein:
(a) the administering comprises systemic administration or intratumoral administration;
(b) the isolated cell is derived from the subject or the isolated cell is allogeneic with reference to the subject;
(c) the method further comprises administering a checkpoint inhibitor;
(d) the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor;
(e) the method further comprises administering a protease inhibitor,
optionally wherein the protease inhibitor is administered in a sufficient amount to repress a repressible protease,
optionally wherein the protease inhibitor is administered prior to, concurrently with, subsequent to administration of the engineered cells or the composition comprising the engineered cells,
optionally wherein the protease inhibitor is selected from the group consisting of:
simeprevir, danoprevir, asunaprevir, ciluprevir, boceprevir, sovaprevir, paritaprevir, telaprevir, grazoprevir, glecaprevir, and voxiloprevir, or the protease inhibitor is grazoprevir, or the protease inhibitor comprises grazoprevir and elbasvir, optionally wherein when the protease inhibitor comprises grazoprevir and elbasvir, the grazoprevir and the elbasvir is co-formulated in a pharmaceutical composition;
optionally wherein the pharmaceutical composition is a tablet
optionally wherein the grazoprevir and the elbasvir are at a 2 to 1 weight ratio, and
optionally wherein the grazoprevir is 100 mg per unit dose and the elbasvir is 50 mg per unit dose; and/or
(f) the method further comprises administering tamoxifen or a metabolite thereof,
optionally wherein the tamoxifen metabolite is selected from the group consisting of: 4-hydroxytamoxifen, N-desmethyltamoxifen, tamoxifen-N-oxide, and endoxifen.

18. The method of claim 13, wherein:
(a) the administering comprises systemic administration or intratumoral administration;
(b) the isolated cell is derived from the subject or the isolated cell is allogeneic with reference to the subject;
(c) the method further comprises administering a checkpoint inhibitor;
(d) the tumor is selected from the group consisting of: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor;
(e) the method further comprises administering a protease inhibitor,
optionally wherein the protease inhibitor is administered in a sufficient amount to repress a repressible protease,
optionally wherein the protease inhibitor is administered prior to, concurrently with, subsequent to administration of the engineered cells or the composition comprising the engineered cells,
optionally wherein the protease inhibitor is selected from the group consisting of:
simeprevir, danoprevir, asunaprevir, ciluprevir, boceprevir, sovaprevir, paritaprevir, telaprevir, grazoprevir, glecaprevir, and voxiloprevir, or the protease inhibitor is grazoprevir, or the protease inhibitor comprises grazoprevir and elbasvir, optionally wherein when the protease inhibitor comprises grazoprevir and elbasvir, the grazoprevir and the elbasvir is co-formulated in a pharmaceutical composition,
optionally wherein the pharmaceutical composition is a tablet
optionally wherein the grazoprevir and the elbasvir are at a 2 to 1 weight ratio, and
optionally wherein the grazoprevir is 100 mg per unit dose and the elbasvir is 50 mg per unit dose; and/or
(f) the method further comprises administering tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen.

19. A pharmaceutical composition comprising the isolated cell of claim 8, and a pharmaceutically acceptable carrier, pharmaceutically acceptable excipient, or a combination thereof.

20. An isolated cell comprising the one or more expression vectors of claim 7, optionally wherein the engineered expression system is recombinantly expressed, optionally wherein the engineered expression system is expressed from a one or more vectors or one or more selected loci from the genome of the cell,
optionally wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell, and
optionally wherein the cell is autologous or the cell is allogeneic.
